(19) United States
(12) Patent Application Publication
(10) Pub. No.: US 2021/0379192 A1
(43) Pub. Date: Dec. 9, 2021

(54) SKELETAL MUSCLE TARGETING MOIETIES AND USES THEREOF

(71) Applicant: Mantra Bio, Inc., San Francisco, CA (US)

(72) Inventors: Linda Anne Szabo, San Francisco, CA (US); Catherine RoseMary Planey, San Francisco, CA (US); Terry Gaige, San Francisco, CA (US); Colin David Gottlieb, San Francisco, CA (US)

(21) Appl. No.: 17/300,507

(22) PCT Filed: Jan. 27, 2020
(86) PCT No.: PCT/US2020/015277

§ 371 Date: Jul. 26, 2021

Related U.S. Application Data
(60) Provisional application No. 62/797,153, filed on Jan. 25, 2019.

(51) Int. Cl. A61K 47/64; A61K 9/127; A61K 47/69

(52) U.S. Cl. CPC A61K 47/642; A61K 9/127; A61K 47/64; A61K 47/6425; A61K 47/6911

(57) ABSTRACT

Disclosed herein are extracellular vesicles such as exosomes that selectively target cells such as skeletal muscle cells. Such vesicles include skeletal muscle targeting moieties and can be used to selectively deliver a payload to skeletal muscle cells or tissue.

### CROSS REFERENCE TO RELATED APPLICATIONS

This application claims benefit to U.S. Provisional Application No. 62/797,153, filed on Jan. 25, 2019, which is incorporated herein by reference in its entirety for all purposes.

### BACKGROUND OF THE INVENTION

Many of the newer drug candidates such as proteins and nucleic acids are highly unstable inside an animal. This poses significant challenges to successful therapeutic outcomes. Recently, there has been a great interest in gene therapy and genetic editing as therapeutics. The use of vesicles has been suggested as a way to deliver therapeutics and other cargos to a cell.

There are many different types of vesicles. Extracellular vesicles (EVs) are membrane-based structures. In nature, EVs serve as vehicles that carry different types of cellular cargo—such as lipids, proteins, receptors and effector molecules—to the recipient cells. Exosomes are a type of EV that are released into the extracellular environment following fusion of multivesicular bodies with the plasma membrane. Exosome production has been described in many cells including B cells, T cells, and dendritic cells (DCs). The potential of vesicle-mediated delivery of cargo has been suggested. However, using naturally occurring unmodified vesicles may result in an increased off-target effect and require higher dosages to elicit desired effect.

There are many different types of cells in the body and there is a need for vesicles that can preferentially target skeletal muscle cell type over another cell type. Preferential binding of a vesicle to skeletal muscle cell type, tissue or organ of interest could be beneficial in reducing off-target effects of a payload and/or reducing the dose of the therapy used.

### SUMMARY OF THE INVENTION

Provided herein is a vesicle comprising one or more skeletal muscle targeting moieties. In one embodiment of the invention, the skeletal muscle targeting moiety is a binding partner of a skeletal muscle marker such as ART1, CACNA1C, CACNA1D, CACNA1F, CACNA1S, CACNA2D1, CHRNA1, CHRNB1, CHRND, CHRNE, CHRNG, or FGF6 or an homologue and fragment thereof. In another embodiment of the invention, the skeletal muscle targeting moiety is an isoform of ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS:99-100), or a homologue or functional fragment thereof. In yet another embodiment, the skeletal muscle targeting moiety is a binding partner of a skeletal muscle marker such as CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) or any homologue or fragment thereof. In a further embodiment, the skeletal muscle targeting moiety is a peptide such as any one or more of CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ ID NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and/or THVSPNQGGLPS (e.g., SEQ ID NO: 211).

The vesicle can be non-naturally occurring. The vesicle can be isolated or ex vivo. The vesicle can be an exosome, a liposome, a lipid nanoparticle, a microvesicle, an ectosome, a nanoparticle, a nanocarrier, a microparticle or an apoptotic body. The vesicle can be an exosome. The exosome can be derived from cells, cell lines, primary cells, tissues or bodily fluids.

Furthermore, provide herein is a vesicle wherein one or more skeletal muscle targeting moieties can be coupled to the vesicle via a targeting moiety (such as a vesicle targeting moiety or a chimeric vesicle targeting moiety) selected from the group comprising of any one or more of the following: lysosome-associated membrane protein (LAMP), Lamp2a, Lamp2b, Lamb2c, syndecan, synaptotagmin, ALIX (CHAMP 4) domain, ALIX-syntenin binding domain, ESCRT-proteins, PDGF, syntenin-PDZ, P6- and P9-domain, CD53, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, Tsg101, LIRB4, FPRP, ITA3, CD166, ITA5, ADAM10, CD81, CD9, CD63, FASN, sodium/potassium-transporting ATPase subunit alpha-1, sodium/potassium-transporting ATPase subunit beta-3, FN1, PTGFRN, ITGA3, IL3RA, SELPL, ITGB1, and CSTN1, or a homologue or fragment thereof.

One or more of the skeletal muscle targeting moieties can be an antibody, a fragment of an antibody such as a single light chain fragment or an antigen-binding antibody fragment, a peptide, an aptamer, protein or protein fragment. One or more of the skeletal muscle targeting moieties can be a homologue comprising a non-naturally occurring sequence having sequence identity of at least 30%, at least 40%, at least 50%, at least 60% or at least 80% to ENO2 (e.g., SEQ ID NOS:39, 41, 43-46), JSRP1 (e.g., SEQ ID NOS:63), VAPA (e.g., SEQ ID NO: 113-115, 192, TMOD1 (e.g., SEQ ID NOS: 99-100). or a functional fragment thereof. One or more skeletal muscle targeting moieties can comprise a functional fragment that is at least 10, 20, 50, 100, 200, 500, or 1000 amino acids in length of any of ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS: 99-100).

Furthermore, the vesicle can comprise a payload. The payload can be a therapeutic moiety or a reporter molecule. The payload can be a nucleic acid, a reporter, a drug, a biologic, or a transgene or genome editing system. The payload can comprise a CRISPR gene editing system. The payload can comprise a nucleic acid comprising a transgene or a miRNA (miR). The CRISPR gene editing system can be selected from the group consisting of a nucleotide sequence that targets the dystrophin gene (DMD), and a nucleotide sequence that targets the myostatin gene (MSTN). The miRNA can be selected from the group consisting of miR-133a, miR-1, miR-133, miR-133b, miR-181a-5p, miR-206, and miR-499. The payload can be selected from the group consisting of fenretinide, meprobamate, chlorphenesin, fludiazepam, xylazine, Baclofen, Chlorzoxanone, Carisoprodol, Methocarbamol, Tizanidine, Cyclobenzaprine, acetyl-salicylic acid, ibuprofen, metocurine, boltilinum TYpe A, botulinum Type B, succinylcholine, cisatracurium, Rocuronium, Hexafluronium, Doxacurium, Tubocurarine, Mivacurium, diazepam, succinylcholine, atracurium besylate, pancuronium, vecuronium, metocurine iodide, gallamine Triethiodie, Prenylamine, Fostamatinib, Baclofen, Octylphenoxy Polyethoxy ethanol, Felodipine, Calcium, magnesium sulfate, verpamil, Dronedarone, clevidipine, Isradepini, Amlodipine, Felodipine, Nifedipine, Nisoldipine, Levosimendan, Bepridil, Aluminium monostearate, blebbistatin, dantrolene, calsequestrin, topiramate, gallamine triethiodie, reidisopngiolide C, Ulapualide, agonists for REV-ERBα (e.g. SR9009, SR9011, SR10067, GSK4112), sirtuin 1 (e.g. SRT1720, SRT2104), adenosine monophosphate-activated protein kinase (AMPK, e.g. AICAR), peroxisome proliferator-activated receptor (PPAR)δ (e.g. GW1516, GW0742, L165041), and inhibitory/antagonistic agents targeting the methionine-folate cycle (MOTS-c), the general control non-derepressible 5 (GCN5) acetyl transferase (e.g. CPTH2,-3), myostatin (e.g. MYO-029), the myostatin receptor (bimagrurnab), and myostatin receptor ligands (e.g. sotatercept, ACE-031), Eteplirsen (Exondys 51), corticosteroids, Lumizyme, Myozyme, Rituximab, antimalarial medications, cholinesterase inhibitors, immunosuppressants. The skeletal muscle targeting moiety can be heterologously expressed on the surface of the exosome.

Also provided herein is a vesicle that selectively binds a skeletal muscle and comprises one or more skeletal muscle targeting moieties. In one embodiment, the skeletal muscle targeting moiety is a binding partner of a skeletal muscle marker coupled to the vesicle by a vesicle targeting moiety. In another embodiment, the skeletal muscle targeting moiety is a binding partner of a skeletal muscle marker such as any of ART1, CACNA1C, CACNA1D, CACNA1F, CACNA1S, CACNA2D1, CHRNA1, CHRNB1, CHRND, CHRNE, CHRNG, and FGF6 or any homologue and fragment thereof.

In yet another embodiment, the skeletal muscle targeting moiety is a peptide such as any one or more of CLVSGGMAC (SEQ ID NO: 118), CLVSGCNTC (SEQ ID NO: 119), CDLVSGYGC (SEQ ID NO: 120), CLVSTSATC (SEQ ID. NO: 121), CTALVSQTC (SEQ ID NO: 122), CWLVSGIGC (SEQ ID NO: 123), CLVSSVFPC (SEQ ID NO: 124), CPSLVSSVC (SEQ ID NO: 125), CGVSLVSTC (SEQ ID NO: 126), CQLVSGEPC (SEQ ID NO: 127), CNLVSRRLC (SEQ ID NO: 128), CLVSWRGSC (SEQ ID NO: 129), CDHFLVSPC (SEQ ID NO: 130), CGRGLVSLC (SEQ ID NO: 131), CFPVALVSC (SEQ ID NO: 132), CRWSSLVSC (SEQ ID NO: 133), CWSKSLVSC (SEQ ID NO: 134), CPGRSLVSC (SEQ ID NO: 135), THRPPMWSPVWP (e.g., SEQ ID NO: 211) and THVSPNQGGLPS (e.g., SEQ ID NO: 211).

In yet a further embodiment, the vesicle-targeting moiety is any of lysosome-associated membrane protein (LAMP), Lamp2a, Lamp2b, Lamb2c, syndecan, synaptotagmin, ALIX (CHAMP 4) domain, ALIX-syntenin binding domain, ESCRT-proteins, PDGF, syntenin-PDZ, P6- and P9-domain, CD53, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, Tsg101, LIRB4, FPRP, ITA3, CD166, ITA5, ADAM10, CD81, CD9, CD63, FASN, sodium/potassium-transporting ATPase subunit alpha-1, sodium/potassium-transporting ATPase subunit beta-3, FN1, PTGFRN, ITGA3, IL3RA, SELPL, ITGB1, and CSTN1, or a homologue or fragment thereof.

Furthermore, provided herein is a method for delivering a payload to a skeletal muscle cell, comprising contacting a skeletal muscle cell with any of the non-naturally occurring vesicles described above. Furthermore, the method can comprise delivering a payload to a skeletal muscle cell in a subject, comprising administering to the subject any of the vesicles described above. The skeletal muscle cell can be a myocyte, a myosatellite cell, or a myoblast. The subject has one or more of the following conditions: obesity, muscle fibrosis, Duchenne muscular dystrophy, Becker muscular dystrophy, Emery-Dreifuss muscular dystrpohy, Limb Girdle muscular dystrophy, Oculopharyngeal muscular dystrophy, congenital Facioscapulohumeral, distal Facioscapulohumeral, Central core disease, Centronuclear myopathies, Congenital fiber type disproportion myopathy, Nemaline myopathy, Multiminicore disease, Myotubular myopathy, autophagic vacuolar myopathy, cap disease, congenital myopathy with arrest of myogenesis, myosin storage (hyaline body) myopathy, zebra body, acid maltase deficiency (AMD, Pompe disease, glycogenosis type 2, lysosomal storage disease), carnitine deficiency, carnitine palmityl transferase deficiency (CPT deficiency), debrancher enzyme deficiency (Cori or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5), polymyositis, dermatomyositis, inclusion body myositis, necrotizing autoimmune myopathy, Myasthenia gravis, Botulism, Eaton-Lambert syndrome, Isaacs syndrome, Stiff-person syndrome, Spinal Muscular Atrophy, or infantile motor neuron disease, Becker muscular dystrophy (BMD), Congenitcal muscular distrophies (CMD) such as Bethlem CMD, Fukuyama CMD, Muscle-eye-brain diseases (MEBs), Rigid spind syndromes, Ullrich CMD, and Walker-Warburg syndromes (WWS), limb-girdle muscular dystrophies (LGMD), myotonic dystrophy (DM), oculopharyngeal muscular dystrophy (OPMD), spinarl-bulbar muscular atrophy (SBMA), Anderson-Tawil syndrome, hyperkalemic periodic paralysis, hypokalemic periodic analysis, becker myotonia, thomsen myotonia, paramyotonia congenita, potassium-aggravated myotonia, congenital myopathies such as cap myopathies, centronuclear myopathies, congenital myopathies with fiber type disproportion, central core disease myopathy, muliminicore myopathies, myosin storage myopathies, myotubular myopathy, or nemaline myopathies, distal myopathies such as GNE myopathy/Nonaka myopathy/hereditary inclusion-body myopathy (HIBM), Laing distal myopathy, Markesbery-Griggs late-onset distal myopathy, Miyoshi myopathy, Udd myopathy/tibial muscular dystrophy, VCP Myopathy/IBMPFD, Vocal cord and pharyngeal distal myopathy, Welander distal myopathy, Endocrine myopathies such as Hyperthyroid myopathy, Hypothyroid myopathy, Inflammatory myopathies, Dermatomyositis, Inclusion-body myositis, Polymyositis, Metabolic myopathies such as Acid maltase deficiency (AMD, Pompe disease), Carnitine deficiency, Carnitine palmitoyltransferase dificiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), and other myopathies such as Myofibrillar myopathies (MFM), Scapuloperoneal myopathy, skeletal muscle myochondrial diseases such as Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (HARP), Pearson syndrome, Progressive external opthalmoplegia (PEO), neuromuscular junction diseases such as Congenital myasthenic syndromes (CMS), Lambert-Eaton myasthenic syndrome (LEMS), Myasthenia gravis (MG), Peripheral nerve diseases such as Charcot-Marie-Tooth disease (CMT) or Giant axonal neuropathy (GAN).

The skeletal muscle cell can be a myocyte, a myosatellite cell, or a myoblast. The payload can be a drug, a biologic, a nucleic acid, a transgene or a genome editing system. The subject can be a mammal. The mammal can be a human.

Furthermore, provided herein is a fusion protein comprising: a vesicle targeting moiety coupled to any one or more skeletal muscle targeting moieties. In one embodiment, the skeletal muscle targeting moiety is a binding partner of skeletal muscle marker such as ART1, CACNA1C, CACNA1D, CACNA1F, CACNA1S, CACNA2D1, CHRNA1, CHRNB1, CHRND, CHRNE, CHRNG, or FGF6 or a homologue or fragment thereof. In another embodiment, the skeletal muscle targeting moiety is an isoform of ENO2, JSRP1, VAPA, TMOD1, or a homologue or functional fragment thereof. In another embodiment, the one or more skeletal muscle targeting moieties is a binding partner of skeletal muscle marker selected from the group consisting of: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or fragment thereof. In yet another embodiment, the one or more skeletal muscle targeting moieties is a peptide group consisting of: CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ ID NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and THVSPNQGGLPS (e.g., SEQ ID NO: 211).

The vesicle targeting moiety in the fusion protein can be an exosome targeting moiety. The exosome targeting moiety can be C1C2. The vesicle targeting moiety can be a lysosome targeting moiety. The fusion protein can further comprise a linker between the skeletal muscle targeting moiety and the vesicle targeting moiety. The lysosome targeting moiety can be a lysosome-associated membrane protein (LAMP), Lamp2a, Lamp2b, Lamb2c, CD63, syndecan, synaptotagmin, ALIX (CHAMP 4) domain, ALIX-syntenin binding domain, ESCRT-proteins, PDGF, syntenin-PDZ, P6- and P9-domain, CD81, CD9, CD53, CD81, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8 (lactadherin—C1C2 domain), SLIT2, STX3, TCRA, TCRB, TCRD, TCRG, VTI1A, or VTI1B, Tsg101, or glycosylphosphatidylinositol.

Furthermore, provided herein is a vector comprising: a nucleic acid sequence encoding a vesicle targeting moiety coupled to a nucleic acid sequence encoding one or more skeletal muscle targeting moieties. In one embodiment, the skeletal muscle targeting moiety is a binding partner of skeletal muscle marker such as any of ART1, CACNA1C, CACNA1D, CACNA1F, CACNA1S, CACNA2D1, CHRNA1, CHRNB1, CHRND, CHRNE, CHRNG, FGF6 and any homologue and fragment thereof. In another embodiment, the one or more skeletal muscle targeting moieties is an isoform of ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS: 99-100), or a homologue or functional fragment thereof. In another embodiment, the one or more skeletal muscle targeting moieties is a binding partner of skeletal muscle marker selected from the group consisting of: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or fragment thereof. In yet another embodiment, the one or more skeletal muscle targeting moieties is a peptide group consisting of: CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ ID NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and THVSPNQGGLPS (e.g., SEQ ID NO: 211).

The vector can further comprise a promoter sequence and optionally one or more other regulatory elements. The vector can comprise a nucleic acid sequence encoding a linker disposed between the nucleic acid sequence encoding the vesicle targeting moiety and the nucleic acid sequence encoding the one or more skeletal muscle targeting moieties.

Provided herein is a genetically modified cell comprising nucleic acid sequences encoding a vesicle targeting moiety and one or more skeletal muscle targeting moieties. In an embodiment of the invention, the skeletal muscle targeting moiety is a binding partner of a skeletal muscle marker such as ART1, CACNA1C, CACNA1D, CACNA1F, CACNA1S, CACNA2D1, CHRNA1, CHRNB1, CHRND, CHRNE, CHRNG, or FGF6 or any homologue and fragment thereof. In another embodiment, the one or more skeletal muscle targeting moieties is an isoform of ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS: 99-100), or a homologue or functional fragment thereof. In another embodiment, the one or more skeletal muscle targeting moieties is a binding partner of skeletal muscle marker selected from the group consisting of: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or fragment thereof. In yet another embodiment, the one or more skeletal muscle targeting moieties is a peptide group consisting of: CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ ID NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and THVSPNQGGLPS (e.g., SEQ ID NO: 211).

The genetically modified cell can comprise a heterologously expressed nucleic acid sequences encoding the one or more skeletal muscle targeting moieties. The genetically modified cell can be HEK293 (ATCC® CRL-1573), variants of HEK293 (e.g. HEK293T (293T/17 SF [HEK 293T/17 SF] (ATCC® ACS-4500) and 293T/17 [HEK 293T/17] (ATCC® CRL-11268)), HEK 293-F (293T/17 SF [HEK 293T/17 SF] (ATCC® ACS-4500) and HEK 293 STF (ATCC® CRL-3249)), HEK 293-H (Gibco® 293-H), OAT1 HEK 293T/17 (ATCC® CRL-11268G-1™), HEK293 Cas9 (293[HEK-293] Cas9 (ATCC® CRL-1573Cas9™)), and HEK-293.2sus (ATCC® CRL-1573.3)), HT-1080 (HT-1080 [HT1080] (ATCC® CCL-121), HP [HT1080 poly] (ATCC® CRL-12012™), and HX [HT1080 xeno] (ATCC® CRL-12011™)), PER.C6 (PER.C6® Cells, Crucell Holland B.V.), HeLa (HeLa (ATCC® CCL-2), HeLa (ATCC® CRM-CCL-2), and HeLa S3 (ATCC® CCL-2.2)), CHO-K1 (ATCC® CCL-61), variants of CHO (e.g. GS-CHO (Invitrogen/Thermo Fisher Scientific, Lonza Biologics), and CHO-DG44 (Thermo Fisher Scientific)) Sf9 (ATCC® CRL-1711), NS0 (TRAIL (NS0) 14G03 #39:14 7/2/01 (PTA-3570)), NS1 (P3/NSI/1-Ag4-1 [NS-1] (ATCC® TIB-18)), human skeletal myoblasts (HSkM), Hs 235.Sk (ATCC® CRL-7201™), Hs 792(C).M (ATCC® CRL-7522™), or Hs 1.Sk/Mu (ATCC® CRL-7001™), or variants of these cells.

Furthermore, provided herein is a method for interfering with an interaction between a skeletal-muscle targeting vesicle and a skeletal muscle cell, comprising: contacting the skeletal-muscle targeting vesicle with one or more skeletal muscle markers or homologues or functional fragments thereof prior to contact of the skeletal-muscle targeting vesicle and the skeletal muscle cell. The one or more skeletal muscle markers can be any isoform of CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80). The one or more skeletal muscle markers can be an antibody or antibody fragment, ligand or peptide that selectively binds ENO2, JSRP1, VAPA, TMOD1.

Furthermore, provided herein is a conjugate comprising a payload coupled to one or more skeletal muscle targeting moieties. In one embodiment, the one or more skeletal muscle targeting moieties is a binding partner of skeletal muscle marker which is selected from the group consisting of: ART1, CACNA1C, CACNA1D, CACNA1F, CACNA1S, CACNA2D1, CHRNA1, CHRNB1, CHRND, CHRNE, CHRNG and FGF6 or any homologue or fragment thereof. In another embodiment, the one or more skeletal muscle targeting moieties is an isoform of ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS: 99-100), or a homologue or functional fragment thereof. In another embodiment, the one or more skeletal muscle targeting moieties is a binding partner of skeletal muscle marker, wherein the skeletal muscle marker is selected from the group consisting of: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or fragment thereof. In yet another embodiment, the one or more skeletal muscle targeting moieties is a peptide group consisting of: CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ ID NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and THVSPNQGGLPS (e.g., SEQ ID NO: 211).

The skeletal muscle targeting moiety can be an antibody or antibody fragment that binds to: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or fragment thereof. The skeletal muscle targeting moiety can be coupled to an AAV capsid.

### BRIEF DESCRIPTION OF FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a map of EV-localizing fusion proteins produced from expression vectors 91, 112, 135, 140, 141, and 142. Numbers represent length in nucleotides for the marks on the line above. Arrangement of notable biological sequences are indicated by various arrows used to represent signal sequence, epitope sequence, affinity peptide, linker, glycosylation site, and a vesicle targeting moiety (vector #91 for LAMP2B; vector #112 for CSTN1 or a chimeric vesicle targeting moiety comprising LAMP2B surface-and-transmembrane domain and cytosolic domain of PTGFRN or Prostaglandin F2 Receptor Inhibitor (vector #135), ITGA3 or Integrin Subunit Alpha 3 (vector #140), IL3RA or Interleukin 3 Receptor Subunit Alpha (vector #141), SELPL or P-Selectin Glycoprotein Ligand 1 (vector #142). Note that the coding sequence for LAMP2B in vector #91 and that for CSTN1 in vector #121 are for the respective mature protein which lacks the signal sequence (first 28 amino acid) present in the native LAMP2B nascent protein and native CSTN1 nascent protein, respectively.

FIG. 2 is a map of EV-localizing fusion proteins produced from expression vectors 143, 144, and 145. Numbers represent length in nucleotides for the marks on the line above. Arrangement of notable biological sequences are indicated by various arrows used to represent signal sequence, epitope sequence, affinity peptide, linker, glycosylation site, and a LAMP2B vesicle targeting moiety lacking LAMP2B cytosolic domain but retaining LAMP2 surface-and-transmembrane domain (vector #145) or a chimeric vesicle targeting moiety comprising LAMP2B surface-and-transmembrane domain and cytosolic domain of ITGB1 or Integrin Beta-1 (vector #143) or CSTN1 or Calsyntenin-1 (vector #144).

FIG. 3 provides the amino acid sequence of EV-localizing fusion proteins encoded by expression vector 91 (LAMP2B) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide. The signal sequence used here is Secrecon signal peptide sequence, and the epitope tag used here is 3×FLAG epitope tag.

FIG. 4 provides the amino acid sequence of EV-localizing fusion proteins encoded by expression vector 112 (CSTN1) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide: THRPPMWSPVWP (SEQ ID NO.: 211). The signal sequence used here is Secrecon signal peptide sequence, and the epitope tag used here is 3×FLAG epitope tag.

FIG. 5 provides the amino acid sequence of EV-localizing fusion proteins encoded by expression vector 135 (a chimeric vesicle targeting moiety comprising LAMP2B surface-and-transmembrane domain and cytosolic domain of PTGFRN) and vector 140 (a chimeric vesicle targeting moiety comprising LAMP2B surface-and-transmembrane domain and cytosolic domain of ITGA3) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide: THVSPNQGGLPS (SEQ ID NO.: 212). The signal sequence used here is Secrecon signal peptide sequence, and the epitope tag used here is 3×FLAG epitope tag.

FIG. 6 provides the amino acid sequence of EV-localizing fusion proteins encoded by expression vector 141 (a chimeric vesicle targeting moiety comprising LAMP2B surface-and-transmembrane domain and cytosolic domain of IL3RA) and vector 142 (a chimeric vesicle targeting moiety comprising LAMP2B surface-and-transmembrane domain and cytosolic domain of SELPL) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide: THVSPNQGGLPS (SEQ ID NO.: 212). The signal sequence used here is Secrecon signal peptide sequence, and the epitope tag used here is 3×FLAG epitope tag.

FIG. 7 provides the amino acid sequence of EV-localizing fusion proteins encoded by expression vector 143 (a chimeric vesicle targeting moiety comprising LAMP2B surface-and-transmembrane domain and cytosolic domain of ITGB1) and vector 144 (a chimeric vesicle targeting moiety comprising LAMP2B surface-and-transmembrane domain and cytosolic domain of CSTN1) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide: THVSPNQGGLPS (SEQ ID NO.: 212). The signal sequence used here is Secrecon signal peptide sequence, and the epitope tag used here is 3×FLAG epitope tag.

FIG. 8 provides the amino acid sequence of EV-localizing fusion proteins encoded by expression vector 145 (truncated LAMP2 having surface-and-transmembrane domain but lacking a cytosolic domain) and produced when the expression vector is introduced into HEK293F cells along with the location of notable biological sequences. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The highlighted text signifies an affinity peptide: THVSPNQGGLPS (SEQ ID NO.: 212). The signal sequence used here is Secrecon signal peptide sequence, and the epitope tag used here is 3×FLAG epitope tag

FIGS. 9-12 provide amino acid sequence of the vesicle targeting moiety and chimeric vesicle targeting moiety encoded and produced by the indicated expression vector constructs of FIGS. 1 and 2. The italicized caps signifies a surface domain. The italicized, bold caps signifies a transmembrane domain. The italicized, underlined caps signifies a cytosolic domain (also considered to be lumenal domain when at an EV). Amino acid sequences in FIG. 9 provided for expression vector #91 and 112 correspond to mature LAMP2B protein and mature CSTN1 protein, respectively, with the three contiguous domains (surface, transmembrane and cytosolic domains) indicated. Amino acid sequence provided in FIG. 12 for expression vector #145 corresponds to a truncated LAMP2B protein lacking its native LAMP2B cytosolic domain but in place does have a short highly charged tetrapeptide as a C-terminal cytoplasmic/lumenal extension. Note that the amino acid sequences of the chimeric vesicle targeting moieties for the remaining expression vectors correspond to those of a chimeric vesicle targeting moiety with amino terminal surface-and-transmembrane domain of LAMP2B and the cytosolic domain of LAMP2B replaced by a cytosolic domain of PTGFRN or Prostaglandin F2 Receptor Inhibitor (vector #135), ITGA3 or Integrin Subunit Alpha 3 (vector #140), IL3RA or Interleukin 3 Receptor Subunit Alpha (vector #141), SELPL or P-Selectin Glycoprotein Ligand 1 (vector #140), ITGB1 or Integrin Beta-1 (vector #143) and CSTN1 or Calsyntenin-1 (vector #144).

FIG. 13 provides density (or concentration) of the fusion protein at an EV (A) and fraction (or percent) of total EVs positive for the fusion protein (B) produced by the expression vector constructs of FIGS. 1 and 2 following transfection into HEK293F cells.

FIG. 14A shows fold increase in fusion protein density (or concentration) on EV surface relative to fusion protein produced by vector #91 construct (fusion protein with a mature LAMP2B protein having a contiguous surface-transmembrane-and-cytosolic domain but no LAMP2B signal sequence). Compared to the fusion protein produced by vector #91, the fusion protein produced by vector #112 (fusion protein with a mature CSTN1 protein having its surface-transmembrane-and-cytosolic domain but no CSTN1 signal sequence) concentrates at a much lower level, about 25% the density of the mature LAMP2B-containing fusion protein (compare value of #91 and #112 in FIG. 14A). Surprisingly, when the cytosolic domain of the mature LAMP2B is replaced with the cytosolic domain of the mature CSTN1, the new chimeric vesicle targeting moiety increases by about 2-fold the density of the fusion protein over its parental LAMP2B (compare value of #91 and #144) or over 8-fold the density of the fusion protein over its parental CSTN1 (compare value of #112 and #144), indicative of synergistic interaction involving the surface-and-transmembrane domain of LAMP2B and the cytosolic domain of CSTN1 that leads to increased EV localization.

FIG. 15 is a map of EV-localizing fusion proteins targeting skeletal muscle cells comprising a single chain Fv (scFv) as a cell targeting moiety that targets the nicotinic acetylcholine receptor in skeletal muscle cells and a vesicle targeting moiety or a chimeric vesicle targeting moiety produced from expression vectors 117, 178, 179, 140, 180, and 181. Numbers represent length in nucleotides for the marks on the line above. Arrangement of notable biological sequences are indicated by various arrows used to represent signal sequence, epitope sequence, scFv as a cell targeting moiety for a skeletal muscle cell, linker, glycosylation site, and a vesicle targeting moiety corresponding to IGSF8 (Immunoglobulin Superfamily Member 8) protein (mature IGSF8 protein with contiguous surface-transmembrane-and-cytosolic domain of IGSF8; as in vector #117, 178, 179, 140 and 180) or a chimeric vesicle targeting moiety comprising LAMP2B surface-and-transmembrane domain and cytosolic domain of PTGFRN or Prostaglandin F2 Receptor Inhibitor (vector #181). scFv segments derived from the CDRs of Ig heavy chain and light chain directed to nicotinic acetylcholine receptors in skeletal muscle cells are indicated along with the arrangement within the scFv.

FIGS. 16-20 provide amino acid sequence of the EV-localizing fusion proteins targeting the nicotinic acetylcholine receptor in skeletal muscle cells produced from the expression vector constructs of FIG. 15. The bold text signifies a signal sequence (a portion of the translated sequence that helps the polypeptide be synthesized by the cell but is not present in the mature protein that gets incorporated into an EV). The lower case text signifies a glycosylation site. The underlined text signifies an epitope sequence. The boxed text signifies linker sequence. The italicized caps signifies a surface domain. The italicized, bold caps signifies a transmembrane domain. The italicized, underlined caps signifies a cytosolic domain (also considered to be lumenal domain when at an EV). The scFV is broken down into heavy chain and a light chain variable regions indicated by different shades of highlighting, with a linker region between the two regions.

FIG. 21 shows values obtained from a custom sandwich ELISA with a colorimetric readout using a matched pair of anti-VAPA antibodies for capture of the purified protein standard or intact vesicles. The capture antibody immunogen is a synthetic peptide corresponding to region between amino acids 125 to 175 of human VAPA. The primary antibody immunogen is a recombinant protein fragment produced in E. coli corresponding to amino acids 2 to 227 of human VAPA. The samples for columns 1-3: standard—purified recombinant protein with N-terminal His-tag and corresponding to the amino acids 1-227 of human VAPA, 2× serial dilution; columns 4-6: transfection control NG_C1C2—neon green fluorescent protein fused with C1C2 domain transfected into HEK293 (FreeStyle™ 293-F cells) [Construct 2]; columns 7-9: engineered material VAPA_NG_C1C2—VAPA construct (without the transmembrane domain) fused with neon green fluorescent protein and C1C2 domain, transfected into FreeStyle™ 293-F cells [Construct 1]; and columns 10-12: no transfection control—i.e. FreeStyle™ 293-F cells. All dilutions were done in blocking buffer.

FIG. 22 shows a standard curve using anti-VAPA antibody and a purified recombinant protein with N-terminal His-tag and corresponding to the amino acids 1-227 of human VAPA.

### DETAILED DESCRIPTION OF THE INVENTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described. References to any amino acid sequences and their respective SEQ ID NOs are provided in Table 1.

### Introduction

The present disclosure describes compositions and methods for selective targeting of a cell type, tissue, or organ by a vesicle having a cell-type, or tissue type, or organ-type specific targeting moiety or targeting moieties. Such targeting moiety can be naturally occurring on the vesicle's surface or engineered to be included on the vesicle's surface. In particular, the present disclosure describes selectively targeting skeletal muscle cells and tissue. The vesicles contemplated herein may include a payload. Such payload is preferably one that is not naturally present in the vesicle. Such payload can be a natural or synthetic bioactive molecule for eliciting a phenotypic modification in the target cell or tissue of interest. In some embodiments, a payload is useful for the treatment of a condition. In some embodiments, a payload is a reporter for screening, detecting, and/or diagnosing a condition in a cell or a subject. The targeting moieties herein allow selective targeting or focused delivery of appropriate payloads to the cells of interest (e.g., skeletal muscle cells). This can reduce off-target effect, toxicity of the treatment.

### Vesicles

A vesicle refers to a membrane that encloses an internal space. Vesicles may be cell-derived or synthetic bubbles made of the same material as cell membranes, such as phospholipids. Cell-derived extracellular vesicles are smaller than the cell from which they are derived and range in diameter from 20 nm to 5000 nm. Such vesicles can be created through the outward budding and fission from plasma membranes, assembled at and released from a plasma membrane, or derived from cells or vesiculated organelles having undergone apoptosis and may contain organelles. They may be derived from cells by direct and indirect manipulation that may involve the destruction of said cells. They may also be derived from a living or dead organism, explanted tissues or organs, and/or cultured cells. Examples of vesicles include but are not limited to exosomes, apoptotic bodies, and microvesicle, microparticles, liposome, lipid nanoparticles. Cell-derived vesicle may also include ectosomes, shedding vesicle, plasma membrane-derived vesicles and exovesicles.

Exosomes are secreted membrane-enclosed vesicles that originate from the endosome compartment in cells. The endosome compartment, or the multi-vesicular body, can be exocytosed from the cell, with ensuing release to the extracellular space of their vesicles as exosomes. Further, an exosome comprises a bilayer membrane, and can comprise various macromolecular cargo either within the internal space, displayed on the external surface of the extracellular vesicle, and/or spanning the membrane. Cargo can comprise nucleic acids, proteins, carbohydrates, lipids, small molecules, and/or combinations thereof. Exosomes can range in size from 20 nm to 150 nm.

In some embodiments, exosomes and other extracellular vesicles can be characterized and marked based on their protein compositions, such as integrins and tetraspanins. Other protein markers that used to characterize exosomes and other extracellular vesicles include TSG101, ALG-2 interacting protein X (ALIX), flotillin 1, and cell adhesion molecules which are derived from the parent cells in which the exosome and/or EV is formed. Similar to proteins, lipids are major components of exosomes and EVs and can be utilized to characterize them.

Further, naturally occurring exosomes originate from the endosome and may contain proteins such as heat shock proteins (Hsp70 and Hsp90), membrane transport and fusion proteins (GTPases, Annexins and flotillin), tetraspanins (CD9, CD63, CD81, and CD82) and proteins such as CD47. Among these proteins, heat shock proteins, annexins, and proteins of the Rab family are abundantly detected in exosomes and are involved in their intracellular assembly and trafficking. Tetraspanins, a family of transmembrane proteins, are also commonly detected in exosomes. In a cell, tetraspanins mediate fusion, cell migration, cell-cell adhesion, and signaling. Other abundant proteins found in exosomes are the integrins, which are adhesion molecules that facilitate cell binding to the extracellular matrix. Integrins are involved in adhering the vesicles to their target cells. Certain proteins found on the surface of exosomes, such as CD55 and CD59, protect exosomes from lysis by circulating immune cells, while CD47 on exosomes acts as an anti-phagocytic signal that blocks the uptake of exosomes by immune cells. Other proteins associated with exosomes include thrombospondin, lactadherin, ALIX (also known as PDCD6IP), TSG1012, and SDCB1. Classes of membrane proteins that naturally occur on the surface of exosomes and other extracellular vesicles include ICAMs, MHC Class I, Lamp2b, lactadherin (C1C2 domain), tetraspannins (CD63, CD81, CD82, CD53, and CD37), Tsg101, Rab proteins, integrins, Alix, and lipid raft-associated proteins such as glycosylphosphatidylinositol and flotillin.

Besides proteins, exosomes are also rich in lipids, with different types of exosomes containing different types of lipids. The lipid bilayer of exosomes is mainly constituted of cell plasma membrane types of lipids such as sphingomyelin, phosphatidylcholine, phosphatidylethanolamine, phosphatidylserine, monosialotetrahexosylganglioside (GM3), and phosphatidylinositol. Other types of lipids that can be found in exosomes are cholesterol, ceramide, and phosphoglycerides, along with saturated fatty-acid chains. Additional optional constituents of exosomes include nucleic acids such as micro RNA (miRNA), messenger RNA (mRNA), and non-coding RNAs. They may also contain a sugar (e.g. a simple sugar, polysaccharide, or glycan) or other molecules.

A vesicle preferably has a longest dimension, such as a cross-sectional diameter of at least 10, 20, 30, 40, 50, 60, 70, 80, 90, 100, 200, 300, 400, or 500 nm and/or at most 2000, 1000, 500, 400, 300, 200, 100, 90, 80, 70, 60, or 50 nm. In some embodiments, a longest dimension of a vesicle can range from 10 nm to 1000 nm, 20 nm to 1000 nm, 30 nm to 1000 nm, 10 nm to 100 nm, 20 nm to 100 nm, 30 nm to 100 nm, 40 nm to 100 nm, 10 nm to 200 nm, 20 nm to 200 nm, 30 nm to 200 nm, 40 nm to 200 nm, 10 nm to 120 nm, 20 nm to 120 nm, such as 30 nm to 120 nm, 40 nm to 120 nm, 10 nm to 300 nm, nm to 300 nm, 30 nm to 300 nm, 40 nm to 300 nm, 50 nm to 1000 mu, 500 nm to 2000 nm, 100 nm to 500 nm, 500 nm to 1000 nm, and such as 40 nm to 500 nm, each range inclusive. When referring to a plurality of vesicles, such ranges represent the average of all vesicles, including naturally occurring and modified vesicles in the mix.

Naturally-occurring vesicles that arise from skeletal muscle cells or tissue can have one or more of the following markers associated with it: adhesion proteins, integrins, tetraspanins, fusion proteins, receptors, transporters, and CD proteins. Furthermore, vesicles arising from skeletal muscle cells or tissue can have one or more of the following expressed on their surface: insulin-like growth factors (IGF1, IGF2), hepatocyte growth factor (I-IGF), fibroblast growth factor-2 (FGF2), and platelet-derived growth factors (PDGFRB, PDGFRA, PDGFB, PDGFA).

Without being bound by any theory, a “vesicle targeting moiety” may be a macromolecule that localizes at an extracellular vesicle. In an embodiment, the vesicle targeting moiety is a protein that localizes at an extracellular vesicle. In an embodiment, the vesicle targeting moiety is a membrane protein. In an embodiment, the vesicle targeting moiety is a transmembrane protein comprising a surface domain, a transmembrane domain and a cytosolic domain. Localization of the such a transmembrane protein at an extracellular vesicle results in the surface domain at the outer surface of the vesicle, the transmembrane domain with the lipid bilayer of the vesicle and the cytosolic domain in the lumen of the vesicle.

A “chimeric vesicle targeting moiety” is a vesicle targeting moiety produced by combining one vesicle targeting domain with another vesicle targeting domain. The combination may comprise two or more vesicle targeting domains. In an embodiment, one combination may be through the fusion of two or more proteins which are vesicle targeting moieties. The combination may be a combination between a portion or fragment of a vesicle targeting moiety with a portion or fragment of a second vesicle targeting moiety. In an embodiment; one protein domain may be substituted with another protein domain. Such protein domains are known in the art. For transmembrane proteins, these proteins often have a surface domain, a transmembrane domain and a cytosolic domain.

“Surface domain” is a subset of the protein or polypeptide primary sequence that is exposed to the extra-EV environment. The surface domain can be a loop between two transmembrane domains or it can contain one of the termini (amino or carboxy) of the protein. Protein domain topology relative to the membrane bi-layer can be determined empirically by assessing what portions of the protein are digested by an external protease. More recently, characteristic amino acid patterns, such as basic or acidic residues in the juxta-membrane regions of the protein have been used to algorithmically assign probable topologies (extracellular vs cytosolic) to integral membrane proteins. Since EVs have the same membrane topology orientation as the plasma membrane of the whole cell (the outer leaflet of the membrane is the same between cells and EVs), these algorithms can be applied to EV resident proteins as well. In an embodiment, the “surface domain” may be a short peptide of approximately 10-15 amino acids. In an embodiment, the “surface domain” may be a unstructured polypeptide. In an embodiment, the “surface domain” is the entire surface domain of an integral membrane protein. In an embodiment, the “surface domain” is part of the surface domain of an integral membrane protein.

“Transmembrane domain” may be a span of about 18-40 aliphatic, apolar and hydrophobic amino acids that assembles into an alpha-helical secondary structure and spans from one face of a membrane bilayer to the other face, meaning that the N-terminus of the helix extends at least to and in many cases beyond the phospholipid headgroups of one membrane leaflet while the C-terminus extends to the phospholipid headgroups of the other leaflet.

As used herein, the term “about” when used before a numerical designation, e.g., temperature, time, amount, concentration, and such other, including a range, indicates approximations which may vary by (+) or (−) 10%, 5% or 1%.

“Cytosolic domain” is a subset of the protein or polypeptide primary sequence that is exposed to the intra-EV or intracellular environment. The cytosolic domain can be a loop between two transmembrane domains or it can contain one of the termini (amino or carboxy) of the protein. Its topology is distinct from that of the transmembrane and the surface domains. In an embodiment, the cytosolic domain is in the cytoplasmic side of a cell. In another embodiment, the cytosolic domain is in the lumen of a vesicle.

In a preferred embodiment, a “chimeric vesicle targeting moiety” comprises the “surface-and-transmembrane domain” of one vesicle targeting moiety and the “cytosolic domain” of a second vesicle targeting moiety, wherein the two vesicle targeting moieties are different and distinct proteins and are not isoforms. In an embodiment, the “chimeric vesicle targeting moiety” comprises the “surface-and-transmembrane domain” of one vesicle targeting moiety and the “cytosolic domain” of a second vesicle targeting moiety, wherein the two vesicle targeting moieties are different and distinct proteins and are not isoforms and wherein the “surface-and-transmembrane domain” may have an internal deletion.

An “isoform” of a protein can be, e.g., a protein resulting from alternative splicing of a gene expressing the protein, or a degradation product of the protein. In some embodiments, a skeletal muscle cell targeting moiety is a peptide that can bind to a skeletal muscle specific marker. Examples of such peptides includes the peptides listed in Table 1. In some cases, a skeletal muscle targeting moiety can be a peptide that is a functional fragment of an antibody. In some cases, a skeletal muscle cell targeting moiety can be a peptide having at least 50%, 55%, 60%, 65%, 70%, 80%, 90% or 99% sequence similarity to the peptides listed in Table 1.

“Surface-and-transmembrane domain” is a contiguous polypeptide containing both a domain that is exposed to extracellular or extra-EV solvent and a transmembrane domain as described above.

A “linker” is a peptide or polypeptide with 3 to 1000 amino acids that are generally non-hydrophobic and encode no secondary structural elements such as helices or beta-sheets.

In some embodiments, a composition herein comprises an isolated or enriched set of vesicles that selectively targets a tissue or cell of interest. Preferably, such tissue or cell of interest is a skeletal muscle cell or tissue. Such vesicles can be naturally occurring or non-naturally occurring. Such vesicles can be loaded with a payload to be delivered to the skeletal muscle cell or tissue.

As used herein “isolated” means a state following one or more purifying steps but does not require absolute purity. “Isolated” vesicle, extracellular vesicle, exosome or composition thereof means a vesicle, extracellular vesicle, exosome or composition thereof passed through one or more purifying steps that separate the vesicle, extracellular vesicle, exosome or composition from other molecules, materials or cellular components found in a mixture or outside of the vesicle, extracellular vesicle or exosome or found as part of the composition prior to purification or separation. “Isolated” does not require absolute purity.

In accordance with the practice of the invention, the skeletal muscle targeting moiety may be heterologously expressed on the surface of the exosome.

In one embodiment, the vesicle carrying the skeletal targeting moiety may increase targeting of a payload to the skeletal muscle cell by at least 2 fold compared to a control vesicle that does not comprise a skeletal muscle targeting moiety.

In one embodiment, the vesicle further comprises a vesicle-targeting moiety. Examples of the vesicle-targeting moiety include, but are not limited to, a lysosome-associated membrane protein (LAMP), Lamp2a, Lamp2b, Lamb2c, syndecan, synaptotagmin, ALIX (CHAMP 4) domain, ALIX-syntenin binding domain, ESCRT-proteins, PDGF, syntenin-PDZ, P6- and P9-domain, CD53, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fe receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, Tsg101, LIRB4, FPRP, ITA3, CD166, ITA5, ADAM10, CD81, CD9, CD63, FASN, sodium/potassium-transporting ATPase subunit alpha-1, sodium/potassium-transporting ATPase subunit beta-3, FN1, PTGFRN, ITGA3, IL3RA, SELPL, ITGB1, CSTN1, ITGA4 and IGSF8, or a homologue or fragment thereof.

In another embodiment of the invention, the vesicle targeting moiety is a chimeric vesicle targeting moiety comprising a surface-and-transmembrane domain of a first vesicle targeting moiety and a cytosolic domain of a second vesicle targeting moiety. In a preferred embodiment, the first and second vesicle targeting moieties are distinct/different proteins and not isoforms.

Examples of suitable first vesicle targeting moieties include, but are not limited to, SELPL, IL3RA, LIRB4, FPRP, IGSF8, ITA3, CSTN1, ITA5, ITGB1, ADAM10, CD81, CD9, CD63, FASN, sodium/potassium-transporting ATPase subunit alpha-1, sodium/potassium-transporting ATPase subunit beta-3, and FN1. Further examples of suitable vesicle targeting moieties may include, but are not limited to, a growth factor, Fc receptor, interleukin receptor, immunoglobulin, MHC-I or MHC-II component, CD antigen, and escort protein. Examples of suitable second vesicle targeting moieties may include the same examples as described for the first vesicle targeting moieties.

The vesicle-targeting moiety may further comprise a peptide or protein with a modified amino acid. The modified amino acid may result from an attachment of a hydrophobic group. The attachment of a hydrophobic group may be myristoylation for attachment of myristate, palmitoylation for attachment of palmitate, prenylation for attachment of a prenyl group, farnesylation for attachment of a farnesyl group, geranylgeranylation for attachment of a geranylgeranyl group or glycosylphosphatidylinositol (GPI) anchor formation for attachment of a glycosylphosphatidylinositol comprising a phosphoethanolamine linker, glycan core and phospholipid tail. The attachment of a hydrophobic group may be performed by chemical synthesis in vitro or is performed enzymatically in a post-translational modification reaction.

Examples of the first vesicle targeting moiety include, but are not limited to, a lysosome-associated membrane protein (LAMP), Lamp2a, Lamp2b, Lamb2c, syndecan, synaptotagmin, ALIX (CHAMP 4) domain, ALIX-syntenin binding domain, ESCRT-proteins, PDGF, syntenin-PDZ, P6- and P9-domain, CD53, CD54, LD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, Tsg101, LIRB4, FPRP, ITA3, CD166, ITA5, ADAM10, CD81, CD9, CD63, FASN, sodium/potassium-transporting ATPase subunit alpha-1, sodium/potassium-transporting ATPase subunit beta-3, FN1, PTGFRN, ITGA3, IL3RA, SELPL, ITGB1, ITGA4 and CSTN1, or a homologue thereof.

Examples of the second vesicle targeting moiety may include the same examples described above for the first vesicle targeting moieties.

In one embodiment of the invention, the cytosolic domain of PTGFRN has an amino acid sequence as provided in Figure: 5 or a homologue or portion thereof. Merely by way of example, the homologue or portion may retain at least about 80% of cytosolic domain activity of PTGFRN in accumulating at an extracellular vesicle. Accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

In another embodiment, the cytosolic domain of ITGA3 has an amino acid sequence as provided in Figure: 5 or a homologue or portion thereof. Merely by way of example, the homologue or portion may retain at least 80% of cytosolic domain activity of ITGA3 in accumulating at an extracellular vesicle. Accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

In yet another embodiment, the cytosolic domain of IL3RA has an amino acid sequence as provided in Figure: 6 or a homologue or portion thereof. As an example, the homologue or portion may retain at least about 80% of cytosolic domain activity of IL3RA in accumulating at an extracellular vesicle. Accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

Additionally, in a further embodiment, the cytosolic domain of SELPL has an amino acid sequence as provided in Figure: 6 or a homologue or portion thereof. In an example of the invention, the homologue or portion retains at least about 80% of cytosolic domain activity of SELPL in accumulating at an extracellular vesicle. Merely by way of example, accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

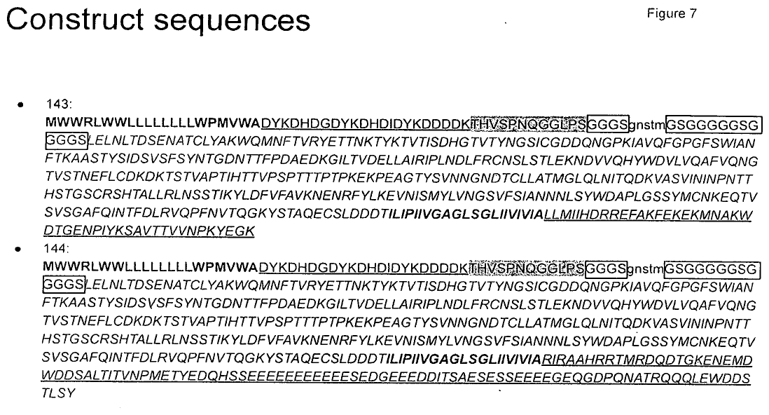

Further, in one embodiment of the invention, the cytosolic domain of ITGB1 may have an amino acid sequence as provided in FIG. 7 or a homologue or portion thereof, wherein the homologue or portion retains at least 80% of cytosolic domain activity of ITGB1 in accumulating at an extracellular vesicle. In one example accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

Further, the cytosolic domain of CSTN1 may have an amino acid sequence as provided in Figure: 7 or a homologue or portion thereof, wherein the homologue or portion retains at least 80% of cytosolic domain activity of CSTN1 in accumulating at an extracellular vesicle. Merely by way of example accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

Further still, in one embodiment, the cytosolic domain of IGSF8 may have an amino acid sequence as provided in Figure: 16 or a homologue or portion thereof. Merely by way of example, the homologue or portion may retain at least 80% of cytosolic domain activity of IGSF8 in accumulating at an extracellular vesicle. Accumulating at an extracellular vesicle may be assessed on the basis of the percent of extracellular vesicle positive for the chimeric vesicle targeting moiety, total number of extracellular vesicle positive for the targeting moiety, and/or the concentration of targeting moiety in an extracellular vesicle.

Merely by way of example, the exosome may have an average diameter in the range of about 50 nm to about 200 nm. In a specific embodiment, the exosome has an average diameter of about 120 nm±20 nm.

In some embodiments of the invention, the vesicle targeting moiety is a chimeric vesicle targeting moiety. The chimeric vesicle targeting moiety may be incorporated into an extracellular vesicle. In a specific embodiment, the extracellular vesicle is an exosome.

In one embodiment of the invention, greater than 40% of the extracellular vesicles are positive for the chimeric protein. In another embodiment, greater than 50% of the extracellular vesicles are positive for the chimeric protein. In yet another embodiment, greater than 60% of the extracellular vesicles are positive for the chimeric protein. Further, in another embodiment, about 50% of the extracellular vesicles are positive for the chimeric protein. Additionally, in another embodiment, about 60% of the extracellular vesicles are positive for the chimeric protein. Further still, in another embodiment, between 40-65% of the extracellular vesicles are positive for the chimeric protein.

In some embodiments of the invention, the cytosolic domain increases the accumulation of the surface and transmembrane domain of the chimeric vesicle targeting moiety in an extracellular vesicle. The increase may be an increase in the amount or concentration or percent of the chimeric vesicle targeting moiety in an extracellular vesicle compared to a parental from which the chimeric vesicle targeting moiety is derived or a parental comprising the surface-and-transmembrane domain but lacking cytosolic domain. Merely by way of example, the increase in the amount or concentration may be at least 1.5-fold. In another embodiment, the increase in the amount or concentration is at least 2.5-fold. Further, in another embodiment, increase in the percent of extracellular vesicle positive for the chimeric vesicle targeting moiety is at least about 1.3-fold over a parental from which the chimeric vesicle targeting moiety is derived or a parental comprising the surface-and-transmembrane domain but lacking cytosolic domain. Further still, in another embodiment, the increase in the percent of extracellular vesicle positive for the chimeric vesicle targeting moiety is at least about 2.5-fold over a parental from which the chimeric vesicle targeting moiety is derived or a parental comprising the surface-and-transmembrane domain but lacking cytosolic domain.

Merely as an example, the increase may be synergistic. In another example, the increase further comprises increased production of exosome.

In an embodiment of the invention, the chimeric vesicle targeting moiety maintains cellular production of extracellular vesicles when the chimeric vesicle targeting moiety is expressed in a cell. In another embodiment, the chimeric vesicle targeting moiety increases cellular production of extracellular vesicles when the chimeric vesicle targeting moiety is expressed in a cell. Additionally, in another embodiment, the chimeric vesicle targeting moiety does not inhibit cellular production of extracellular vesicles when the chimeric vesicle targeting moiety is expressed in a cell.

In one embodiment of the invention, the chimeric vesicle targeting moiety is a fusion protein comprising a domain arrangement from amino-to-carboxyl terminus in the order: surface domain of the surface-and-transmembrane domain, followed by transmembrane domain of the surface-and-transmembrane domain, and followed by the cytosolic domain.

In another embodiment, the chimeric vesicle targeting moiety is any of the chimeric protein as provided in Figures: 5-7 or Table 1 or a homologue or fragment thereof. Merely as an example, the homologue may have between at least about 80% but less than 100% sequence identity and the fragment is a function fragment retaining at least about 80% of a vesicle targeting activity.

As used herein, “sequence identity” or “identity” refers, in the context of two nucleic acid sequences or amino acid sequences, to the residues in the two sequences that are the same when aligned for maximum correspondence over a specified comparison window.

As used herein, “percent sequence identity” means the value determined by comparing two optimally aligned sequences over a comparison window, wherein (the portion of the polynucleotide or polypeptide sequence in the comparison window may comprise additions or deletions (i.e., gaps) compared to the reference sequence which does not comprise additions or deletions comprises) can for optimal alignment of the two sequences. The percentage can be calculated by determining the number of positions at which the identical nucleotide or amino acid, occurs in both sequences to yield the number of matched positions, dividing the number of matched positions by the total number of positions to give in the comparison window and multiplying the result by 100 to determine the percentage of sequence identity is calculated.

A function can be similar to a function of a full-length protein if it retains at least 75%, 80%, 85%, 90%, 95%, 99%, or 100% of that function of the full-length protein. The function can be measured e.g., using an assay, e.g., an in vivo binding assay, a binding assay in a cell, or an in vitro binding assay.

In another embodiment, the vesicle targeting activity is the ability of the chimeric vesicle targeting moiety to accumulate at a vesicle assessed by the percent of vesicles positive for the targeting moiety, total number of vesicles positive for the targeting moiety and/or concentration of targeting moiety at a vesicle.

### Engineered Vesicles

In some instances, a vesicle herein is engineered for enhanced targeting to a cell or tissue of interest. Such engineered vesicle can be non-naturally occurring. Such engineered vesicles can be ‘targeted’ or ‘guided’ via a functionalized moiety (a targeting moiety) for increased affinity to a cell/tissue/organ of interest. An engineered vesicle can be derived from a skeletal muscle cell or from any other cell type (e.g., HEK-293). A vesicle can be engineered to include a heterologous expression of one or more targeting moieties.

Vesicle functionalization can occur by modification of vesicles such as exosomes, to display an exogenous protein or nucleic acid. As used herein, a “targeting moiety” can be a small molecule, glycoprotein, protein, peptide, lipid, carbohydrate, nucleic acid, or other molecules involved in EV trafficking and/or EV interaction with target cells. The targeting moiety may be displayed inside or on the outside of a vesicle membrane or may span the inner membrane, outer membrane, or both inner and other membranes. The targeting moiety may be expressed in exosomes that are ‘emptied’ of natural cargo or expressed in addition to naturally occurring cargo. Functionalization may also be carried out through the incorporation of a payload.

In one instance, an engineered vesicle is one that is functionalized or is engineered to express a targeting moiety (e.g., a protein, peptide or nucleic acid) that selectively target a skeletal muscle cell or tissue. Such engineered vesicle is preferably an exosome, a liposome, a lipid nanoparticle, a microvesicle, an ectosome, a microparticle, or an apoptotic body. In some embodiments, such engineered vesicle is an exosome.

The targeting moiety is a skeletal muscle targeting moiety or a binding partner of a skeletal muscle marker (i.e., a marker that is selectively expressed in or at the surface of a skeletal muscle cell or tissue). Merely by way of example, the binding partner can be a protein, antibody, antibody fragment, a peptide or an aptamer. Examples of skeletal muscle markers include but are not limited to the peptides or proteins as shown in SEQ ID Nos: 2, 6-7, 10-11, 17, 28, 40, 42, 48, 57, 68, 70, 81, 89-90, 101, 104-105, 116-117, and 136-176 in Table 1.

An engineered vesicle herein can have at least 1, 2, 3, 4, 5, 6, 7, 8, 9, or 10 different types of skeletal muscle targeting moieties or binding partners for skeletal muscle markers. In one embodiment of the invention, the skeletal muscle marker is a subunit of an acetylcholine receptor (AChR).

### Target Cells

The vesicles described herein can be used to selectively target a cell, tissue, or organ of interest. In some embodiments, the target cell is a eukaryotic cell or, more preferably, a skeletal muscle cell. A target skeletal muscle cell can be a skeletal muscle cell from an animal such as a mouse, rat, rabbit, hamster, porcine, bovine, feline, or canine. The target cells can be cells of primates, including but not limited to, monkeys, chimpanzees, gorillas, and humans. Preferably, the skeletal muscle cells are skeletal myocytes, myoblasts (progenitors of skeletal myocytes), or myosatellite cells (which active myogenesis and development of muscle fiber upon stimulation). In some examples, skeletal muscle cells are cell types that reside in skeletal muscle tissue, such as connective tissue cells such as mast cells, fibroblasts, adipose cells, plasma cells and lymphocytes, dendritic cells. A target cell can be any skeletal muscle cell expressing any of the targets listed in Table 1, or any isoform, homolog or functional fragment thereof. A target call can be any cell in the microenvironment of a cell expressing any one of the targets listed in Table 1, or any isoform, homolog or functional fragment thereof.

### Skeletal Muscle Targeting Moieties

The present disclosure provides for skeletal muscle targeting moieties. Such targeting moieties can be used to target a payload to skeletal muscles. In some instances, the skeletal muscle targeting moiety is conjugated to the payload via an AAV, antibody, or other carrier. In some embodiments, the skeletal muscle targeting moiety is conjugated to the payload via a vesicle.

Any of the vesicles herein, preferably include one or more skeletal muscle targeting moieties. Such skeletal muscle targeting moieties can be embedded in or displayed on vesicle membranes. Preferably, the vesicle is an exosome, and the skeletal muscle targeting moiety is displayed on the outer surface of the exosome.

Tissue specific targeting moieties can comprise a small molecule, glycoprotein, peptide, protein, lipid, carbohydrate, nucleic acid or other molecules that facilitates the targeting of the vesicle to the skeletal muscle cell or tissue. In one embodiment, a skeletal muscle targeting moiety can be an antibody or a functional epitope thereof that binds to a skeletal muscle cell surface marker, for example, a cell surface receptor. As used herein, the term “antibody” is defined to be a protein or polypeptides functionally defined as a binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

Any of the targeting moieties described herein preferably enhance the selectivity of the vesicles towards the target cell of interest as compared to one or more other tissues or cells. Typically, the selective targeting moieties are expressed on modified vesicles in a way that allows such modified vesicles to bind to intended targets, such as, skeletal muscle markers. More particularly, the targeting moieties expose sufficient amount of amino acids to allow such binding.

The modified vesicles herein comprise targeting moieties that selectively target the vesicles to skeletal muscle cells or tissue by binding or physically interacting with markers expressed on skeletal muscle cells.

The term “selective” or “selectively” as used herein in the context of selective targeting or selective binding or selective interaction refers to a preferential targeting, binding or interaction to a cell, tissue, or organ of interest as compared to at least one other type of cell, tissue or organ.

A skeletal muscle targeting moiety can steer a vesicle to a skeletal muscle cell, e.g., a myocyte, myoblast, myosatellite. In some examples, a skeletal muscle targeting moiety is a moiety that can steer a vesicle to a cell that resides in skeletal muscle tissue, such as connective tissue cells, mast cells, fibroblasts, adipose cells, plasma cells and lymphocytes, dendritic cells.

Examples of skeletal muscle targeting moieties include any binding partner of a skeletal muscle marker including (non-limiting exemplary isoforms provided in parenthesis), CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any other isoforms thereof.

In some embodiments, a binding partner of a skeletal muscle marker is an antibody or antibody fragment that selectively binds to a skeletal muscle marker such as: CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any other isoforms thereof.

In some embodiments, a binding partner of a skeletal muscle marker is a ligand or fragment of a ligand that selectively binds to any one of the skeletal muscle markers such as CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any other isoforms thereof.

In some cases, the skeletal muscle markers may be expressed on the surface of the vesicles.

Examples of skeletal muscle targeting moieties include, but are not limited to, the following proteins and any of their isoforms (exemplary ones illustrated): ENO2 (e.g., SEQ ID NOS:39, 41, 43-46), JSRP1 (e.g., SEQ ID NOS:63), VAPA (e.g., SEQ ID NOS: 113-115, 192), TMOD1 (e.g., SEQ ID NOS: 99-100), or a functional fragment thereof, or a homologue thereof.

In some embodiments, a skeletal muscle cell targeting moiety comprises a non-naturally occurring amino acid sequence having at least 80%, 85%, 90%, 95%, or 99% sequence homology to any of ENO2 (e.g., SEQ ID NOS:39, 41, 43-46), JSRP1 (e.g., SEQ ID NOS:63), VAPA (e.g., SEQ ID NOS: 192, TMOD1 (e.g., SEQ ID NOS: 99-100), or a functional fragment. In some instances, the sequence homology is less than 85%, 90%, 95%, or 99%.

Homology comparisons may be conducted with sequence comparison programs. Computer programs may calculate percent (%) homology between two or more sequences and may also calculate the sequence identity shared by two or more amino acid or nucleic acid sequences. Sequence homologies may be generated by any of a number of computer programs, for example BLAST or FASTA, etc. A suitable computer program for carrying out such an alignment is the GCG Wisconsin Bestfit package (University of Wisconsin, U.S.A.; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Percent homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments can be performed over a relatively short number of residues.

In some embodiments, a skeletal muscle cell targeting moiety is a functional fragment to any isoforms of ENO2 (e.g., SEQ ID NOS:39, 41, 43-46), JSRP1 (e.g., SEQ ID NOS:63), VAPA (e.g., SEQ ID NOS: 192, TMOD1 (e.g., SEQ ID NOS: 99-100).

A “functional fragment” of a protein means a fragment of the protein which results in a targeting or binding function similar to that of the full protein.

In some embodiments, a skeletal muscle cell targeting moiety is a peptide that can bind to the muscle specific markers. Examples of such peptides includes: CLVSGGMAC (e.g., SEQ ID NO: 118), CLVSGCNTC (e.g., SEQ ID NO: 119), CDLVSGYGC (e.g., SEQ ID NO: 120), CLVSTSATC (e.g., SEQ ID NO: 121), CTALVSQTC (e.g., SEQ ID NO: 122), CWLVSGIGC (e.g., SEQ ID NO: 123), CLVSSVFPC (e.g., SEQ ID NO: 124), CPSLVSSVC (e.g., SEQ ID NO: 125), CGVSLVSTC (e.g., SEQ ID NO: 126), CQLVSGEPC (e.g., SEQ ID NO: 127), CNLVSRRLC (e.g., SEQ 1D NO: 128), CLVSWRGSC (e.g., SEQ ID NO: 129), CDHFLVSPC (e.g., SEQ ID NO: 130), CGRGLVSLC (e.g., SEQ ID NO: 131), CFPVALVSC (e.g., SEQ ID NO: 132), CRWSSLVSC (e.g., SEQ ID NO: 133), CWSKSLVSC (e.g., SEQ ID NO: 134), CPGRSLVSC (e.g., SEQ ID NO: 135), THRPPMWSPVWP (e.g., SEQ ID NO: 211) and THVSPNQGGLPS (e.g., SEQ ID NO: 211).

A “homologue” refers to any sequence that has at least about 80%, 85%, 90%, 95%, 96%, 97%, 98%, 99%, or 99.5% sequence homology to another sequence but less than 100% sequence homology. In general, “sequence identity” or “sequence homology”, which can be used interchangeably, refer to an exact nucleotide-to-nucleotide or amino acid-to-amino acid correspondence of two polynucleotides or polypeptide sequences, respectively. Techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_wated, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). High sequence identity generally includes ranges of sequence identity of approximately 80% to 99% and integer values there between.

Thus, a vesicle of the present disclosure is one that comprises (preferably on its surface) one or more binding partner(s) to any of the above skeletal muscle markers. In some embodiments, a vesicle of the present disclosure is one that comprises (preferably on its surface) binding partner(s) to a homologue(s) of the above skeletal muscle markers.

In some embodiments, a vesicle comprises at least 2, 3, 4, 5, 6, 7, 8, 9 or 10 different binding partners. In some embodiments a vesicle comprises a sufficient number of binding partners to selectively target skeletal muscle cells over non-skeletal muscle cells.

Any of the skeletal muscle targeting moieties herein can be used in combination with one another to improve targeting. Examples include a vesicle engineered to express two or more, three or more, or four or more skeletal muscle targeting moieties from Table 1. Any of the skeletal muscle targeting moieties may be used in combination with one another or in combination with other proteins, r subcellular location sequences such as monopartitie or bipartite nuclear localization sequences (NLS), importin alpha (KPNA1), and/or importin beta 1 (KPNB1), or other skeletal proteins or CD47, CD55, and/or CD59 to improve targeting or evade immune response to alter pharmacokinetic properties such as circulation time in blood.

In some embodiments, the skeletal muscle targeting moiety is coupled to the vesicle by the vesicle targeting moiety. For example, said skeletal muscle targeting moiety may be crosslinked to a vesicle targeting moiety. In another embodiment, said skeletal muscle targeting moiety comprises a fusion protein comprising a skeletal muscle targeting moiety and a vesicle targeting moiety.

In some embodiments, the skeletal muscle targeting moiety may be an antibody or a fragment or variant thereof, a peptide, an aptamer, a ligand, or protein or protein fragment. The antibody or a fragment or variant thereof, the peptide, the aptamer, the ligand, or protein or protein fragment may specifically recognize and bind a skeletal muscle marker. Examples of the fragment or variant of the antibody include, but are not limited to, scFv, Fv, Fab, Fab′, and F(ab′)₂ fragments. In one embodiment, the fragment or variant of the antibody is derived from an antibody directed to a subunit of an acetylcholine receptor. In another embodiment, the acetylcholine receptor is nicotinic acetylcholine receptor.

In a specific embodiment of the conjugate, the payload is a drug and the skeletal muscle targeting moiety is an antibody or antibody fragment that binds to any of ART1, CACNA1C, CACNA1D, CACNA1F, CACNA1S, CACNA2D1, CHRNA1, CHRNB1, CHRND, CHRNE, CHRNG or FGF6 or any homologue or fragment thereof. In another embodiment of the conjugate, the skeletal muscle targeting moiety is coupled to an adeno-associated virus (AAV) capsid.

### Fusion Proteins

The skeletal muscle targeting moieties can be fused with a vesicle targeting moiety. Together the two are referred to as a “fusion protein”. The vesicle targeting moiety of the fusion protein targets the skeletal muscle targeting moieties (or other fused molecule) to a vesicle. In some embodiments, a vesicle targeting moiety targets the skeletal muscle targeting moieties (or other fused molecule) to the membrane of a vesicle. In some embodiments, the vesicle targeting moiety itself is the skeletal muscle targeting moiety. Preferably the vesicle targeting moiety targets to the membrane of an exosome. In some embodiments, fusion proteins can be made with a vesicle targeting moiety and a ligand (skeletal muscle targeting moiety) that binds a skeletal muscle cell receptor. The ligand will be surface exposed and will selectively bind to a receptor or receptors on the surface of the target cell. These fusion proteins of skeletal muscle targeting moieties can be loaded into vesicles (e.g., exosomes and EVs) endogenously or exogenously. Alternatively, nucleic acids encoding fusion proteins or skeletal muscle targeting moieties and vesicle targeting moieties separately can be used to express the exosome targeting moiety and skeletal muscle targeting moieties.

Examples of vesicle targeting moieties can comprise any one or more of the following: lysosome-associated membrane protein (LAMP), Lamp2a, Lamp2b, Lamp2c, CD63, syndecan, synaptotagmin, ALIX (CHAMP 4) domain, ALIX-syntenin binding domain, ESCRT-proteins, PDGF, syntenin-PDZ, P6- and P9-domain, CD81, CD9, CD53, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8 (lactadherin—C1C2 domain), SLIT2, STX3, TCRA, TCRB, TCRD, TCRG, VTI1A, VTI1B, Tsg101.glycosylphosphatidylinositol, other exosomal polypeptides, and combinations thereof.

The fusion protein may also include a binding domain of a bacteriophage protein fused to a lysosome membrane protein. The fusion protein may comprise the cytoplasmic part of syndecan because the cytoplasmic part of syndecan has a PDZ-binding domain which binds the syntenin-ALIX complex, and the Syntenin-ALIX complex subsequently forms an extracellular vesicle and/or an exosome. Methods for making such fusion proteins and for targeting fusion proteins to exosomes are known in the art e.g., Limoni S K, et al. Appl Biochem Biotechnol. 2018 Jun. 28. doi: 10.1007/s12010-018-2813-4.

The vesicle targeting moiety in the fusion protein may be an exosome targeting moiety. In one example, the exosome targeting moiety can be C1C2 domain of lactadherin. The vesicle targeting moiety can be a lysosome targeting moiety. The fusion protein may further comprise a linker between the skeletal muscle targeting moiety and the vesicle targeting moiety. The lysosome targeting moiety may be a lysosome-associated membrane protein (LAMP), Lamp2a, Lamp2b, Lamb2c, CD63, syndecan, synaptotagmin, ALIX (CHAMP 4) domain, ALIX-syntenin binding domain, ESCRT-proteins, PDGF, syntenin-PDZ, P6- and P9-domain, CD81, CD9, CD53, CD81, CD54, CD50, FLOT1, FLOT2, CD49d, CD71, CD133, CD138, CD235a, Syntenin-1, Syntenin-2, TSPAN8, syndecan-1, syndecan-2, syndecan-3, syndecan-4, TSPAN14, CD37, CD82, CD151, CD231, CD102, NOTCH 1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1 CAM, LAMB1, LAMC1, LFA-1, LGALS3BP, Mac-1 alpha, Mac-1 beta, MFGE8 (lactadherin—C1C2 domain), SLIT2, STX3, TCRA, TCRB, TCRD, TCRG, VTI1A, or VTI1B, Tsg101, or glycosyiphosphatidylinositol.

In some embodiments, the vesicle targeting moiety comprises a peptide or protein with a modified amino acid. Said modified amino acid may result from an attachment of a hydrophobic group. Said attachment of a hydrophobic group may be myristoylation for attachment of myristate, palmitoylation for attachment of palmitate, prenylation for attachment of a prenyl group, farnesylation for attachment of a farnesyl group, geranylgeranylation for attachment of a geranylgeranyl group or glycosyiphosphatidylinositol (GPI) anchor formation for attachment of a glycosyiphosphatidylinositol comprising a phosphoethanolamine linker, glycan core and phospholipid tail. In one embodiment, the attachment of a hydrophobic group is performed by chemical synthesis in vitro or is performed enzymatically in a post-translational modification reaction.

In some embodiments, the fusion protein may further comprise a linker between the skeletal muscle targeting moiety and the vesicle targeting moiety.

Further provided herein is a fusion protein comprising a chimeric vesicle targeting moiety of the invention as described above. In one embodiment, the fusion protein is expressed on the surface of an exosome. In another embodiment, the chimeric vesicle targeting moiety is an exosome targeting moiety. In yet another embodiment, the fusion protein further comprises a linker. For example, the linker may be a peptide linker.

### Nucleic Acids

The production of engineered vesicles can involve generation of nucleic acids that encode, at least, in part, one or more of the cell-type specific targeting moieties described herein, one or more of the binding partners described herein, one or more of the vesicle targeting moieties described herein, one or more fusion proteins described herein, or a combination thereof. The nucleic acids may be natural, synthetic or a combination thereof. The nucleic acids may be RNA, mRNA, lincRNA, siRNA, DNA or cDNA.

The disclosure includes vectors. In some embodiments, a vector comprises nucleic acids encoding one or more cell-type specific targeting moieties operably linked to nucleic acids that encode one or more vesicle targeting moieties. In some embodiments, a vector comprises nucleic acids encoding one or more skeletal-muscle binding partners operably linked to nucleic acids encoding one or more vesicle targeting moieties. In some embodiments, a vector comprises nucleic acids encoding a vesicle targeting moieties operably linked to a nucleic acid encoding any one or more of: ENO2 (e.g., SEQ ID NOS:39, 41, 43-46), JSRP1 (e.g., SEQ ID NOS:63), VAPA (e.g., SEQ ID NOS: 192, TMOD1 (e.g., SEQ ID NOS: 99-100), or a functional fragment or homologue thereof. In one example, a vector comprises nucleic acids encoding a vesicle targeting moieties operably linked to nucleic acids encoding any one or more of: a binding partner of any one or more of CACNA2D1 (e.g., SEQ ID NOS:23-26), CCDC80 (e.g., SEQ 1D NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ ID NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) and any homologue or functional fragment thereof.

The nucleic acids may be natural, synthetic or a combination thereof. The nucleic acids may be RNA, mRNA, DNA or cDNA. Nucleic acid encoding the protein may be produced using known synthetic techniques, incorporated into a suitable expression vector using well established methods to form a protein-encoding expression vector which is introduced into isolated exosomes using known techniques, Similarly, the selected protein may be produced using recombinant techniques, or may be otherwise obtained, and then may be introduced directly into isolated exosomes by electroporation or transfection e.g. electroporation, transfection using cationic lipid-based transfection reagents, and the like.

The nucleic acids can also include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well known for a variety of cells. The origin of replication from the plasmid pBR322 is suitable for most Gram-negative bacteria. In eukaryotic host cells, e.g., mammalian cells, the expression vector can be integrated into the host cell chromosome and then replicate with the host chromosome.

Expression vectors also generally contain a selection gene, also termed a selectable marker. Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. An exemplary selection scheme can utilize a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art.

An example of a promoter that is capable of expressing a transgene in a mammalian nervous system cells is the EF1a promoter. Another example of a promoter is the immediate early cytomegalovirus (CMV) promoter sequence. Other constitutive promoter sequences may also be used, including, but not limited to the simian virus 40 (SV40) early promoter, mouse mammary tumor virus promoter (MMTV), human immunodeficiency virus (HIV) long terminal repeat (LTR) promoter, MoMuLV promoter, phosphoglycerate kinase (PGK) promoter, MND promoter (a synthetic promoter that contains the U3 region of a modified MoMuLV LTR with myeloproliferative sarcoma virus enhancer, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention is not limited to the use of constitutive promoters.

Inducible or repressible promoters are also contemplated for use in this disclosure. Examples of inducible promoters include, but are not limited to a metallothionein promoter, a glucocorticoid promoter, a progesterone promoter, a tetracycline promoter, a c-fos promoter, the T-REx system of ThermoFisher which places expression from the human cytomegalovirus immediate-early promoter under the control of tetracycline operator(s), and RheoSwitch promoters of Intrexon.

Expression vectors typically have promoter elements, e.g., enhancers, to regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. The expression vector may be a bi-cistronic construct or multiple cistronic construct. The two cistrons may be oriented in opposite directions with the control regions for the cistrons located in between the two cistrons. When the construct has more than two cistrons, the cistrons may be arranged in two groups with the two groups oriented in opposite directions for transcription.

It may be desirable to modify the polypeptides described herein. One of skill will recognize many ways of generating alterations in a given nucleic acid construct to generate variant polypeptides. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see, e.g., Gillam and Smith, Gene 8:81-97, 1979; Roberts et al., Nature 328:731-734, 1987, which is incorporated by reference in its entirety for all purposes). The recombinant nucleic acids encoding the polypeptides described herein can be modified to provide preferred codons which enhance translation of the nucleic acid in a selected organism or cell line.

The polynucleotides can also include nucleotide sequences that are substantially equivalent (homologues) to other polynucleotides described herein. Polynucleotides can have at least about 80%, more typically at least about 90%, and even more typically at least about 95%, sequence identity to another polynucleotide.

The nucleic acids also provide the complement of the polynucleotides including a nucleotide sequence that has at least about 80%, more typically at least about 90%, and even more typically at least about 95%, sequence identity to a polynucleotide encoding a polypeptide recited herein. The polynucleotide can be DNA (genomic, cDNA, amplified, or synthetic) or RNA. Nucleic acids which encode protein analogs or variants (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., Molecular Cloning: A Laboratory Manual, Cold Spring Harbor Laboratory Press, Cold Spring Harbor, N.Y. (1989) and/or Current Protocols in Molecular Biology, Ausubel et al., eds, Green Publishers Inc. and Wiley and Sons, N.Y. (1994), each of which is incorporated by reference in its entirety for all purposes. Chemical synthesis using methods well known in the art, such as that described by Engels et al., Angew Chem Intl Ed. 28:716-34, 1989 (which is incorporated by reference in its entirety for all purposes), may also be used to prepare such nucleic acids.

Amino acid “substitutions” for creating variants are preferably the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

When the nucleic acid is introduced into a cell ex. vivo, the nucleic acid may be combined with a substance that promotes transference of a nucleic acid into a cell, for example, a reagent for introducing a nucleic acid such as a liposome or a cationic lipid, in addition to the aforementioned excipients. Electroporation applying voltages in the range of about 20-1000 V/cm may be used to introduce nucleic acid or protein into exosomes. Transfection using cationic lipid-based transfection reagents such as, but not limited to, Lipofectamine® MessengerMAX™ Transfection Reagent, Lipofectamine® RNAiMAX Transfection Reagent, Lipofectamine® 3000 Transfection Reagent, or Lipofectamine® LTX Reagent with PLUS™ Reagent, may also be used, The amount of transfection reagent used may vary with the reagent, the sample and the cargo to be introduced. Alternatively, a vector carrying the nucleic acid of the present invention is also useful. Particularly, a composition in a form suitable for administration to a living body which contains the nucleic acid of the present invention carried by a suitable vector is suitable for in vivo gene therapy.

Also provided is a vector comprising: a nucleic acid sequence encoding a fusion protein of the invention. In one embodiment, the vector further comprises a promoter sequence and optionally one or more additional regulatory elements.

Production of Vesicles with Skeletal Cell Targeting Moieties

Any of the nucleic acids herein can be used for heterologous expression in a cell of a fusion protein of a vesicle targeting moiety operably linked to a skeletal muscle targeting moiety or a binding partner of a skeletal muscle cell marker.

Common GMP-grade cells used in such heterologous expression include HEK293 (kidney epithelial cell line), variants of HEK293 such as HEK293T, HEK 293-F, HEK 293T, and HEK 293-H, dendritic cells, mesenchymal stem cell (MSCs), HT-1080, PER.C6, HeLa, and any variants thereof. Additional optional cells include skeletal muscle-specific cell lines such as variations of human skeletal myoblasts (HSkM), such as Hs 235.Sk (ATCC® CRL-7201™), Hs 792(C).M (ATCC® CRL-7522™), or Hs 1.Sk/Mu (ATCC® CRL-7001™). Additional optional cells include Animal cells include, for example, fibroblasts, epithelial cells (e.g., renal, mammary, prostate, lung), keratinocytes, hepatocytes, adipocytes, endothelial cells, and hematopoietic cells. The animal cells can be adult cells (e.g., terminally differentiated, dividing or non-dividing) or embryonic cells (e.g., blastocyst cells, etc.) or stem cells. The target cell also can be a cell line derived from an animal or other source. Examples of specific cell lines include HEK293 and variants of HEK293 such as HEK293T, ARPE19, CHO NS0, NS1 (mice cell lines), CHO-K1 (general CHO), GS-CHO, CHO-DG44 (Chinese hamster ovary, HeLa, PER.C6, hTERT, and Sf9 insect cell line.

Any of the polypeptides herein can be produced by a cell (or cell line) generating the vesicles to which it is coupled. Alternatively, the skeletal muscle targeting moiety can be heterologously expressed by the cell producing the vesicle. In a different embodiment, the skeletal muscle targeting moiety is coupled to the vesicle after the vesicles are produced and/or isolated.

Modified vesicles can be obtained from a subject, from primary cell culture cells obtained from a subject, from cell lines (e.g., immortalized cell lines), and other cell sources. One can make modified vesicles with specific markers, such as targeting moieties, targeted to skeletal muscle markers on skeletal muscle cells in several ways. One such method includes engineering cells directly in culture to express targeting moieties that are then incorporated into the modified vesicles harvested as delivery vehicles from these engineered cells. Cells which are used for modified vesicle production are not necessarily related to or derived from the cell targets of interest. Once derived, vesicles may be isolated based on their size, biochemical parameters, or a combination thereof. Another method that can be used in conjunction with or independent of the direct cell engineering is physical isolation of particular subpopulations (subtypes) of modified vesicles with desired targeting moieties from the broad, general set of all vesicles produced by a subject. Another method that can be used in conjunction with the previously described two methods or independently is direct incorporation of desired targeting moieties (e.g., proteins/polypeptides) on the vesicles surface. In this method, a general population of vesicles or a specific population of vesicles are isolated from cell culture. The isolated vesicles are then treated to incorporate desired targeting moieties into the vesicles (e.g., fusion) to generate modified vesicles. The isolated vesicles may also be combined with chemical reagents that couple the targeting moieties to the existing proteins on the surface of the vesicles. It is noted that these methods can be combined in different ways. For example, the process can be direct engineering of cells for modified vesicles production followed by isolating target modified vesicles subpopulation.

Further provided is a method of making a vesicle of the invention as described herein. In one embodiment, the method comprises the steps of: (a) isolating a vesicle secreted into a culture medium by a producer cell; and (b) incorporating a skeletal targeting moiety of the above into the vesicle by incubation in an appropriate buffer. In one embodiment of the method, after step (b), the method further comprises the step of introducing a payload into the vesicle.

Further provided is a method of making a vesicle of the invention as described herein. In one embodiment, the method comprises the steps of: (a) expressing a nucleic acid encoding a protein comprising the skeletal muscle targeting moiety of the above coupled to a vesicle targeting moiety in a producer cell; and (b) isolating skeletal muscle targeting vesicles secreted into a culture medium by the producer cell. In one embodiment of the method, after step (b), the method further comprises the step of introducing a payload into the vesicle.

Further provided is an extracellular vesicle made by any of the production methods of the invention as described herein.

Further provided is a method of manufacturing of a vesicle of the invention, wherein the vesicle targeting moiety is a chimeric vesicle targeting moiety comprising a surface-and-transmembrane domain of a first vesicle targeting moiety and a cytosolic domain of a second vesicle targeting moiety, wherein the two vesicle targeting moieties are distinct proteins and not isoforms. In one embodiment, the method comprises the following steps: (a) expressing a nucleic acid encoding a protein comprising the chimeric vesicle targeting moiety of the invention in a producer cell; and (b) isolating a vesicle secreted into a culture medium by the producer cell expressing the chimeric vesicle targeting moiety.

### Genetically Modified Cell

The invention provides a genetically modified cell; which may comprise a heterologously expressed nucleic acid sequences encoding the one or more skeletal muscle targeting moieties as described herein. Examples of genetically modified cell include but are not limited to HEK293, variants of HEK293 (e.g. HEK293T, HEK 293-F, HEK 293T, and HEK 293-H), HT-1080, PER.C6, HeLa, CHO-K1, variants of CHO (e.g. GS-CHO, and CHO-DG44) Sf9, NS0, NS1, human skeletal myoblasts (HSkM), Hs 235.Sk (ATCC® CRL-7201™), Hs 792(C).M (ATCC® CRL-7522™), or Hs 0.1.Sk/Mu (ATCC® CRL-7001™), or variants of these cells.

Also provided is a genetically modified cell comprising a nucleic acid sequence encoding a vector of the invention as described herein.

### Kits

According to another aspect of the invention, kits are provided. Kits according to the invention include package(s) comprising vesicles or compositions of the invention. In one embodiment, the kit comprises the vesicle of the invention and instructions for use and/or storage. In another embodiment, the kit comprises the fusion protein of the invention and instructions for use and/or storage. In another embodiment, the kit comprises the vector of the invention and instructions for use and/or storage. In another embodiment, the kit comprises the genetically modified cell of the invention and instructions for use and/or storage.

The phrase “package” means any vessel containing compounds or compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

The kit can also contain items that are not contained within the package but are attached to the outside of the package, for example, pipettes.

Kits may optionally contain instructions for administering vesicles or compositions of the present invention to a subject having a condition in need of treatment. Kits may also comprise instructions for approved uses of compounds herein by regulatory agencies, such as the United States Food and Drug Administration. Kits may optionally contain labeling or product inserts for the present vesicles or compositions of the invention. The package(s) and/or any product insert(s) may themselves be approved by regulatory agencies. The kits can include active agents in the solid phase or in a liquid phase (such as buffers provided) in a package. The kits also can include buffers for preparing solutions for conducting the methods, and pipettes for transferring liquids from one container to another.

The kit may optionally also contain one or more other compositions for use in combination therapies as described herein. In certain embodiments, the package(s) is a container for intravenous administration. In other embodiments, compounds are provided in an injectable means.

### Examples

1. Example of Engineering Cells to Produce Desired Modified Vesicles.

Vesicle producing cells can be transfected with nucleic acids such as a plasmid or virus carrying nucleic acids encoding the targeting moiety or moieties. The experimental steps can be as the following:
a. Culture vesicle producing cell line in its optimal growth conditions.
b. Prepare the plasmid or virus vector carrying a nucleic acid encoding the targeting moiety or moieties. The nucleic acid encoding the targeting moiety or moieties can be linked with a nucleic acid encoding an exosome localization moieties such as known exosomal surface protein (such as Lamp2) to make a fusion protein.
c. Transfect the vesicle producing cell lines by the construct made in (b). The transfection can be performed in various ways, such as electroporation or liposome-based nucleic acid transfer. The transfection can be transient or stable transfection. For establishing a stable target protein (targeting moiety) expressing EV producing cell lines, integration of target sequence into the recipient cell genome may be needed.
d. The transfected cell culture is then grown on complete media with exosome-depleted FBS for further exosome collection. Alternatively, the transfected cell culture can be seeded into a bioreactor for exosome production.
e. Collect the conditioned media after a certain period of time (e.g., 1 day, 2 day, 3 day, 4 day) from regular flask or dish culture or bioreactor culture.
f. Isolate modified vesicles from conditioned media. Exosomes may be obtained from the appropriate biological sample using any protocol that yields exosomes useful for therapeutic use, e.g. sufficiently pure, intact exosomes with good stability. The isolation methods can include but are not limited to ultracentrifugation, ultrafiltration, polymer-based pulldown, or immunoaffinity-based pulldown. An antibody, ligand, receptor, and/or aptamer complementary to the desired EV targeting moiety(s) can be linked to immunomagnetic beads or rods for binding to target EV subpopulation and subsequent isolation. Alternatively, other immune enrichment/isolation techniques can be used. Examples of immunoaffinity capture techniques that may be used to capture exosomes using a selected antibody cocktail include, but are not limited to, immunoprecipitation, column affinity chromatography, magnetic-activated cell sorting, fluorescence-activated cell sorting, adhesion-based sorting and microfluidic-based sorting. The antibodies in the antibody cocktail may be utilized together, in a single solution, or two or more solutions that are used simultaneously or consecutively.

2. Example of Engineering Vesicles with Peptide Targeting Moieties on the Surface.
a. Culture vesicle producing cell line in its optimal growth conditions.
b. Prepare the plasmid or virus vector carrying a nucleic acid encoding the appropriate promoter and sequence for antibiotic resistance such as puromycin and fluorescence signal such as green fluorescent protein.
c. Procure source cell line containing a vesicle targeting sequence such as Lamp2b. Design primers for amplification of Lamp-2b comprising appropriate cloning sites for future insertions from the mouse or appropriate animal cell line cDNA. Design separate primers for the EV-targeting signal peptide region and transmembrane region. Examples of such peptide regions are Lamp5F, Lamp5R. Examples of transmembrane regions are Lamp3F and Lamp3R.
d. Obtain nucleotide sequence for the targeting peptide moiety using appropriate software tools. Create a forward and reverse primers, flanked by restriction sites to allow for integration of the targeting sequence.
e. Integrate peptide sequence with the vesicle targeting sequence using standard cloning Methods into the plasmid or virus vector in (b), to generate a construct that can target the peptide to the exosomal surface.
f. Transfect the vesicle producing cell lines by the construct made in (e). The transfection can be performed in various ways, such as electroporation or liposome-based nucleic acid transfer. The transfection can be transient or stable transfection. For establishing a stable target protein (marker) expressing EV producing cell lines, integration of target sequence into the recipient cell genome may be needed.
g. The transfected cell culture is then grown on complete media with exosome-depleted FBS for further exosome collection. Alternatively, the transfected cell culture can be seeded into a bioreactor for exosome production.
h. Collect the conditioned media after a certain period of time (e.g., 1 day, 2 day, 3 day, 4 day) from regular flask or dish culture or bioreactor culture.
i. Isolate modified vesicles from conditioned media using any technique known in the art or described herein.
j. In an otherwise identical pair of sequences, one insertion or deletion may cause the following amino acid or nucleotide residues to be put out of alignment, thus potentially resulting in a large reduction in % homology when a global alignment is performed. Consequently, the sequence comparison method can be designed to produce optimal alignments that take into consideration possible insertions and deletions without unduly penalizing the overall homology or identity score. This can be achieved by inserting “gaps” in the sequence alignment to try to maximize local homology or identity.

3. Example of Physical Isolation of a Specific EV Subpopulation from a General Vesicle Population from a Cell Culture.

This method can be combined with the method above or used as a stand-alone method on a non-engineered cell line. The modified vesicle subpopulation carrying targeting moiety or moieties can be isolated from a parental population. The experimental steps can be the following:
k. Culture a vesicle producing cell line under its growth conditions with exosome-depleted FBS containing media. Alternatively, the vesicle producing cell line can be seeded into a bioreactor for exosome production.
l. Collect the conditioned media after a certain period of time (e.g., 1 day, 2 day, 3 day, 4 day) from regular flask or dish culture or a bioreactor culture.
m. Isolate vesicles from the conditioned media. The isolation methods can include but are not limited to ultracentrifugation, ultrafiltration, polymer-based pulldown, or immunoaffinity-based pulldown.
n. Isolate modified vesicle subpopulations from parental EV populations using immunoaffinity-based pulldown. An antibody, ligand, receptor, and/or aptamer complementary to the desired EV marker(s) can be linked to immunomagnetic beads or rods for binding to target EV subpopulation and subsequent isolation. Alternatively, other immune enrichment/isolation techniques can be used.

4. Example of Direct Incorporation of the Desired Targeting Moiety or Moieties on the Vesicle Surface.

A parental vesicle or vesicle subpopulation produced from regular flask/dish culture or bioreactor culture of transfected cells or non-transfected cells can be directly incorporated with the desired selective markers on the surface. The experimental steps can be as the following:
o. Prepare a vesicle for engineering.
p. The binding of proteins or polypeptides on the vesicle surface can be achieved by:
   i. Electroporation of the vesicle with desired selective targeting moieties. The controlled electric pulse permeabilizes areas on the vesicle surface membrane for insertion/incorporation of desired selective targeting moieties.
   ii. The vesicle can also fuse with a particular liposome (or lipid/protein complex) carrying the desired selective targeting moieties on its surface. Via the fusion, the selective targeting moieties will then effectively be on the surface of the liposome-modified vesicle complex. See Sato et al., Sci. Reports 6:21933, DOI: 10.1038/srep21933 (2016), which is incorporated by reference in its entirety for all purposes. The vesicle can also be fused with an adeno-associated virus (AAV).

The modified vesicles can be incorporated with the targeting moieties directly with or without cholesterol or other phospholipids. The modified vesicle protein mixture can be created via gentle mixing and incubation or several cycles of freezing and thawing.

The modified vesicles can be derived from eukaryotic cells that can be obtained from a subject (autologous) or from allogeneic cell lines. The subject may be any living organisms. Examples of subjects include humans, dogs, cats, mice, rats, and transgenic species thereof. Vesicles can be concentrated and separated from the circulatory cells using centrifugation, filtration, or affinity chromatography columns. In some embodiments, the vesicles are derived from skeletal muscle tissue and can be concentrated and separated using centrifugation, filtration or affinity chromatography columns.

### Payloads

The vesicle delivery systems described herein, for example modified vesicles such as exosomes, can be used to deliver payloads to target cells. In some embodiments, the payload is embedded in the vesicle, e.g., the lipid bilayer. Alternatively, or additionally, the payload can be surrounded by the vesicle or lipid bilayer.

As described above, targeting moieties on the modified vesicles traffic the modified vesicles in the body to target cells, and the targeting moieties are also involved in target cell recognition and interaction. These targeting moieties can also be used with liposomes, nanoparticles and other delivery vehicles to be directed to a target cell. Modified vesicles with these targeting moieties of interest can also be associated with or fused with other delivery vehicles, such as liposomes or adeno-associated viral vectors to enhance delivery to target cell. See György, Bence, et al. Biomaterials 35 (2014) 26:7598-7609. Modified vesicles, liposomes, nanoparticles, ADC conjugates etc. can carry a payload that is to be delivered to the target cell.

A payload can be, for example, a small molecule, polypeptide, nucleic acid, lipid, carbohydrate, ligand, receptor, reporter, drug, or combination of the foregoing (e.g., two or more drugs, or one or more drugs combined with a lipid, etc.). Examples of payloads, include, for example therapeutic biologics (e.g., antibodies, recombinant proteins, or monoclonal antibodies), RNA (siRNA, shRNA, miRNA, antisense RNA, mRNA, noncoding RNA, tRNA, rRNA, other RNAs), reporters, lipids, carbohydrates, nucleic acid constructs (e.g., viral vectors, plasmids, lentivirus, expression constructs, other constructs), oligonucleotides, aptamers, cytotoxic agents, anti-inflammatory agents, antigenic peptides, small molecules, and nucleic acids and polypeptides for gene therapy. Payloads can also be complex molecular structures such as viral nucleic acid constructs (encoding transgenes) with accessory proteins for delivery to target cells where the nucleic acid construct can be (if needed) reverse transcribed, delivered to the nucleus, and integrated (or maintained extrachromosomally). Optionally, the construct with a desired transgene(s) can be specifically targeted to a site in the chromosome of the target cell using CRISPR/CAS (CAS9, CAS13a, CAS13b) and appropriate guide RNAs. Payloads may be loaded into the extracellular vesicle internal membrane space, displayed on, or partially or fully embedded in the lipid bi-layer surface of the extracellular vesicle.

Examples of pharmaceutical and biologic payloads include drugs for treating organ diseases and syndromes, cytotoxic agents, and anti-inflammatory drugs.

Examples of RNA payloads include siRNAs, miRNAs, shRNA, antisense RNAs, small nuclear RNA (snRNA), small nucleolar RNA (snoRNA), long intergenic noncoding RNA (lincRNA), piwi interacting RNA (piRNA), ribosomal RNA (rRNA), tRNA, and rRNA. Examples of noncoding RNA payloads include microRNA (miRNA), long non-coding RNA (lncRNA), small nuclear RNA (snRNA), small nucleolar RNA (snoRNA), long intergenic non-coding RNA (lincRNA), piwi-interacting RNA (piRNA), ribosomal RNA (rRNA), yRNA and transfer RNA (tRNA). miRNAs and lncRNAs in particular are powerful regulators of homeostasis and cell signaling pathways, and delivery of such RNAs by an EV can impact the target cell.

Reporters are moieties capable of being detected indirectly or directly. Reporters include, without limitation, a chromophore, a fluorophore, a fluorescent protein, a luminescent protein, a receptor, a hapten, an enzyme, and a radioisotope.

Examples of reporters include one or more of a fluorescent reporter, a bioluminescent reporter, an enzyme, and an ion channel. Examples of fluorescent reporters include, for example, green fluorescent protein from Aequorea victoria or Renilla reniformis, and active variants thereof (e.g., blue fluorescent protein, yellow fluorescent protein, cyan fluorescent protein, etc.); fluorescent proteins from Hydroid jellyfishes, Copepod, Ctenophora, Anthrozoas, and Entacmaea quadricolor, and active variants thereof; and phycobiliproteins and active variants thereof. Chemiluminescent reporters include, for example, placental alkaline phosphatase (PLAP) and secreted placental alkaline phosphatase (SEAP) based on small molecule substrates such as CPSD (Disodium 3-(4-methoxyspiro {1,2-dioxetane-3,2′-(5′-chloro)tricyclo [3.3.1.13,7]decan}-4-yl)phenyl phosphate, β-galactosidase based on 1,2-dioxetane substrates, neuraminidase based on NA-Star® substrate, all of which are commercially available from ThermoFisher Scientific. Bioluminescent reporters include, for example, aequorin (and other Ca+2 regulated photoproteins), luciferase based on luciferin substrate, luciferase based on Coelenterazine substrate (e.g., Renilla, Gaussia, and Metridina), and luciferase from Cypridina, and active variants thereof. In some embodiments, the bioluminescent reporter include, for example, North American firefly luciferase, Japanese firefly luciferase, Italian firefly luciferase, East European firefly luciferase, Pennsylvania firefly luciferase, Click beetle luciferase, railroad worm luciferase, Renilla luciferase, Gaussia luciferase, Cypridina luciferase, Metrida luciferase, OLuc, and red firefly luciferase, all of which are commercially available from ThermoFisher Scientific and/or Promega. Enzyme reporters include, for example, β-galactosidase, chloramphenicol acetyltransferase, horseradish peroxidase, alkaline phosphatase, acetylcholinesterase, and catalase. Ion channel reporters, include, for example, cAMP activated cation channels. The reporter or reporters may also include a Positron Emission Tomography (PET) reporter, a Single Photon Emission Computed Tomography (SPECT) reporter, a photoacoustic reporter, an X-ray reporter, and an ultrasound reporter.

Nucleic acid payloads can be oligonucleotides, recombinant polynucleotides, DNA, RNA, or otherwise synthetic nucleic acids. The nucleic acids can cause splice switching of RNAs in the target cell, turn off aberrant gene expression in the target cell, replace aberrant (mutated) genes in the chromosome of the target cell with genes encoding a desired sequence. The replacement nucleic acids can be an entire transgene or can be short segments of the mutated/aberrant gene that replaces the mutated sequence with a desired sequence (e.g., a wild-type sequence). Alternatively, the nucleic acid payloads can alter a wild-type gene sequence in the target cell to a desired sequence to produce a desired result. The payload nucleic acids can also introduce a transgene into the target cell that is not normally expressed. The payload nucleic acids can also cause desired deletions of nucleic acids from the genome of the target cell.

Appropriate genome editing systems can be used with the payload nucleic acids such as CRISPR, TALEN, or Zinc-Finger nucleases. The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems. See, e.g., Cong et al. Science 339.6121 (2013): 819-823, Li et al. Nucl. Acids Res (2011): gkr188, Gaj et al. Trends in Biotechnology 31.7 (2013): 397-405, all of which are incorporated by reference in their entirety for all purposes. Payload nucleic acids can be integrated into desired sites in the genome (e.g., to repair or replace nucleic acids in the chromosome of the target cell), or transgenes can be integrated at desired sites in the genome including, for example, genomic safe harbor site, such as, for example, the CCRS, AAVS1, human ROSA26, or PSIP1 loci. Sadelain et al., Nature Rev. 12:51-58 (2012); Fadel et al., J. Virol. 88(17):9704-9717 (2014); Ye et al., PNAS 111(26):9591-9596 (2014), all of which are incorporated by reference in their entirety for all purposes. When a CRISPR system is used, Cas9 in the target cell may be derived from a plasmid encoding Cas9, an exogenous mRNA encoding Cas9, or recombinant Cas9 polypeptide alone or in a ribonucleoprotein complex. Kim et al (2014) Genome 1012-19. doi:10.1101/gr.171322.113.; Wang et al (2013) Cell 153 (4). Elsevier Inc.: 910-18. doi:10.1016/j.ce11.2013.04.025, both of which are incorporated by reference in their entirety for all purposes.

BLAST 2 Sequences is another tool that can be used for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

Homologous sequences can also have deletions, insertions or substitutions of amino acid residues which result in a functionally equivalent substance. and it is therefore useful to group amino acids together in functional groups. Amino acids may be grouped together based on the properties of their side chains alone.

Substantially homologous sequences of the present invention include variants of the disclosed sequences, e.g., those resulting from site-directed mutagenesis, as well as synthetically generated sequences. In some cases, the

### Introducing Payloads

Payloads can be incorporated into vesicles through several methods involving physical manipulation. Physical manipulation methods include but are not limited to, electroporation, sonication, mechanical vibration, extrusion through porous membranes, electric current and combinations thereof, which cause disruption of vesicle membrane. Loading of cargo to vesicles described herein may involve passive loading processes such as mixing, co-incubation, or active loading processes such as electroporation, sonication, mechanical vibration, extrusion through porous membranes, electric current and combinations thereof. In some embodiments, said loading can be done concomitantly with vesicle assembly.

Payloads of interest can be passively loaded into vesicles by incubation with payloads to allow diffusion into the vesicles along the concentration gradient. The hydrophobicity of the drug molecules can affect the loading efficiency. Hydrophobic drugs can interact with the lipid layers of the vesicle membrane and enable stable packaging of the drug in the vesicle's lipid bilayer. In some embodiments, purified exosome solution suspended in buffer solution can be incubated with payload. In some preferred embodiments, the payload is dissolved in a solvent mixture that can include DMSO, to allow passive diffusion into exosomes. Following this, the payload-exosomes mixture is made free from un-encapsulated payload. In preferred embodiments, centrifugation or size-exclusion columns are used to remove precipitates from the supernatant. LC/MS methods can be used for the measurement and characterization of payload in the exosome-payload formulation, following lysis and removal of the exosome fraction.

Nucleic acids of interest can be incubated with purified exosomes to allow transfection of purified exosomes in the presence of a suitable lipid based transfection reagent. Centrifugation can be used to purify the suspension and isolate the transfected exosome population. Transfected exosomes can then be added to target cells or used in vivo.

Payload can be diffused into cells by incubation with cells that then produce exosomes that Carry the payload. For example, cells treated with a drug can secrete exosomes loaded with the drug. In a previous example, Pascucci et. al, have treated SR4987 mesenchymal stroma cells with a low dose of paclitaxel for 24 h, then washed the cells and reseeded them in a new flask with fresh medium. After 48 h of culture, the cell conditioned medium was collected, and exosomes were isolated. The paclitaxel-loaded exosomes from the treated cells had significant, strong anti-proliferative activities against CFPAC-1 human pancreatic cells in vitro, as compared with the exosomes from untreated cells (Pascucci, L. et. al, Journal of Controlled Release, 192 (2014): 262-270.

Extracellular vesicles secreted from cells can be mixed with payloads and subsequently sonicated by using a homogenizer probe. The mechanical shear force from the sonicator probe can compromise the membrane integrity of the exosomes and subsequently allow the drug to diffuse into the exosomes during this membrane deformation.

In another embodiment, extracellular vesicles from cells can be mixed with a payload, and the mixture can be loaded into a syringe-based lipid extruder with 100-400 nm porous membranes under a controlled temperature. The exosome membrane can be disrupted during the extrusion process can allow vigorous mixing with the drug. In some examples, the number of effective extrusions can vary from 1-10 to effectively deliver drugs into exosomes.

Payload of interest can be incubated with exosomes at room temperature for a fixed amount of time. Repeated freeze-thaw cycles are then performed to ensure drug encapsulation. The method can result in a broad distribution of size ranges for the resulting exosomes, and then, the mixture is rapidly frozen at −80° C. or in liquid nitrogen and thawed at room temperature. The number of effective freeze-thaw cycle may vary from 2-7 for effective encapsulation. In another embodiment, membrane fusion between exosomes and liposomes can be initiated through freeze-thaw cycles to create exosome-mimetic particles.

In another cases, small pores can be created in exosomes membrane through application of an electrical field to exosomes suspended in a conductive solution. The phospholipid bilayer of the exosomes can be disturbed by the electrical current. Payloads can subsequently diffuse into the interior of the exosomes via the pores. The integrity of the exosome membrane can then be recovered after the drug loading process. In some examples, siRNA or miRNA can be loaded into exosomes using this method.

In some cases, electroporation can be conducted in an optimized buffer such as trehalose disaccharide to aid in maintaining structural integrity and can inhibit the aggregation of exosomes.

Membrane permeabilization can be initiated through incubation with surfactants such as saponin. In some examples, hydrophilic molecules can be assisted in exosome encapsulation by this process.

Chemistry based approaches can also be used to directly attach molecules to the surfaces of exosomes via covalent bonds. In some examples, copper-catalyzed azide alkyne cycloaddition can be used for the bioconjugation of small molecules and macromolecules to the surfaces of exosomes as shown in Wang et. al, 2015 and Hood et. al, 2016 [Here the references are incorporated in their entirety].

In another embodiment, fluorophores and microbeads conjugated to highly specific antibodies can bind a particular antigen on the cell surface. Specific antigen-conjugated microbeads can be used for exosome isolation and tracking in vivo.

Treatment payloads carried by the modified vesicles can include, for example, miR-133a (downregulate miR-133a targets Smarcd1 and Runx2), other miRNA can include miR-1, miR-133, miR-133b, miR-181a-5p, miR-206, and miR-499. Other payloads include, for example, Eteplirsen (Exondys 51) (muscular dystrophies), Corticosteroids (muscular dystrophies, polymyositis, dermatomyositis, Myasthenia gravis), Lumizyme (acid maltase deficiency), Myozyme (acid maltase deficiency), Rituximab (polymyositis, dermatomyositis), Antimalarial medications (dermatomyositis), Cholinesterase inhibitors (Myasthenia gravis), and Immunosuppressants (Myasthenia gravis).

### Interfering Vesicle/Skeletal Muscle Interaction

This disclosure further contemplates compositions and methods for interfering with an interaction between a skeletal-muscle targeting vesicle and a skeletal muscle cell. Such interference can be to impede, delay, attenuate, or stop a delivery of a payload to a skeletal muscle cell. Such interference can be for research or therapeutic uses. Such interference can be effectuated by contacting a vesicle such as a skeletal-muscle targeting vesicle with one or more skeletal muscle markers or a homologue or functional fragment of a skeletal muscle marker. A skeletal muscle marker contemplated herein include, for example, an isoform of CACNA2D1 (e.g., SEQ ID NOS:23-26), CCDC80 (e.g., SEQ ID NOS:27, 29-30), ART1 (e.g., SEQ ID NO:20), ALDOA (e.g., SEQ ID NOS:1, 3-5, 8-9, 12-16, 18-19), SVIL (e.g., SEQ ID NOS: 95-98), RAPSN (e.g., SEQ 1D NOS:82-85), CHRND (e.g., SEQ ID NOS:31-36), CHRNG (e.g., SEQ ID NOS:37-38), FGF6 (e.g., SEQ ID NOS:58-59), ITIH6 (e.g., SEQ ID NO:60), TRDN (e.g., SEQ ID NOS:107-112), JPH1 (e.g., SEQ ID NOS:61-62), KCNA7 (e.g., SEQ ID NO:64), KLHL41 (e.g., SEQ ID NO:65), TNNI2 (e.g., SEQ ID NOS:102-103, and 106), ENO3 (e.g., SEQ ID NOS:47, 49-56), SH3BGR (e.g., SEQ ID NOS:86-88, 91-94), OBSCN (e.g., SEQ ID NO:66-67, 69, 71-74), CACNA1S (e.g., SEQ ID NOS:21-22), or OSBPL6 (e.g., SEQ ID NOS:75-80) or a homologue or functional fragment thereof. In some embodiments a skeletal muscle marker is an antibody or antibody fragment, ligand or peptide that selectively binds ENO2, JSRP1, VAPA, TMOD1.

### Pharmaceutical Compositions

Pharmaceutical compositions disclosed herein may comprise modified vesicles and/or liposomes with (or without) a payload, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are in one aspect formulated for intravenous administration or intracranial administration or intranasal administration to the central nervous system. Compositions described herein may include lyophilized EVs, exosomes, and/or liposomes.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. The composition can be formulated into a known form suitable for parenteral administration, for example, injection or infusion. The composition may comprise formulation additives such as a suspending agent, a preservative, a stabilizer and/or a dispersant, and a preservation agent for extending a validity term during storage.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, intranasally, intraarterially, intratumorally, into an afferent lymph vessel, by intravenous (i.v.) injection, or intracranially injection, or intraperitoneally. In one aspect, the T cell compositions of the present invention are administered to a patient by intradermal or subcutaneous injection. In one aspect, the Modified vesicles compositions described herein are administered by i.v. injection.

### Uses of Organ/Tissue/Cell Specific EVs

Exosomes or exosome mimetics have many of the desirable features of an ideal drug delivery system, such as a long circulating half-life, the intrinsic ability to target tissues, biocompatibility, and minimal or no inherent toxicity issues. Alternatively, liposomes or polymeric nanoparticles can be modified with the targeting proteins and lipids from specific types of exosomes (or EVs) to make modified liposomes or polymeric nanoparticles that can traffic to desired locations, interact with target cells, and fuse with target cells to deliver a payload.

The modified EVs herein can include a payload, such as, e.g., any of the payloads described herein.

Modified EVs that target skeletal muscle can be used to carry payloads to treat skeletal muscle related diseases. Examples of skeletal muscle diseases or conditions include: obesity, fibrosis, Duchenne muscular dystrophy, Becker muscular dystrophy, Emery-Dreifuss muscular dystrpohy, Limb Girdle muscular dystrophy, Oculopharyngeal muscular dystrophy, congenital Facioscapulohumeral, distal Facioscapulohumeral, Central core disease, Centronuclear myopathies, Congenital fiber type disproportion myopathy, Nemaline myopathy, Multiminicore disease, Myotubular myopathy, autophagic vacuolar myopathy, cap disease, congenital myopathy with arrest of myogenesis, myosin storage (hyaline body) myopathy, zebra body, acid maltase deficiency (AMD, Pompe disease, glycogenosis type 2, lysosomal storage disease), carnitine deficiency, carnitine palmityl transferase deficiency (CPT deficiency), debrancher enzyme deficiency (Con or Forbes disease, glycogenosis type 3), lactate dehydrogenase deficiency (glycogenosis type 11), myoadenylate deaminase deficiency, phosphofructokinase deficiency (Tarui disease, glycogenosis type 7), phosphogylcerate kinase deficiency (glycogenosis type 9), phosphogylcerate mutase deficiency (glycogenosis type 10), phosphorylase deficiency (McArdle disease, myophosphorylase deficiency, glycogenosis type 5), polymyositis, dermatomyositis, inclusion body myositis, necrotizing autoimmune myopathy, Myasthenia gravis, Botulism, Eaton-Lambert syndrome, Isaacs syndrome, Stiff-person syndrome, Spinal Muscular Atrophy, infantile motor neuron disease, etc.

The desired amount of targeting moiety's expression on an EV, exosome, liposome, nanoparticle, or other delivery vehicle (collectively “delivery vehicles”) may consider the target cell concentration, density of markers on the target cell, whether target cells are associated with other target cells, target cells' local microenvironment, the binding affinity (K_{d}) of a targeting moiety for a marker on the target cell, and the concentration of delivery vehicle. These parameters can be used to arrive at a desired density of markers on the delivery vehicle.

TABLE 1
Sequences for cell target proteins and engineered sequences on exosome
surface for targeting

| SEQ | | |
|---|---|---|
| ID | | |
| NO: | Sequence | Source¹ |
| 1 | MPYQYPALTPEQKKELSDIAHRIVAPGKGILAADESTGSIAKRLQSIG | ALDOA protein |
| | TENTEENRRFYRQLLLTADDRVNPCIGGVILFHETLYQKADDGRPFP | (ENSP00000378661) encoded |
| | QVIKSKGGVVGIKVDKGVVPLAGTNGETTTQGLDGLSERCAQYKK | by Transcript ID |
| | DGADFAKWRCVLKIGEHTPSALAIMENANVLARYASICQQVGLQN | ENST00000395240 |
| | GIVPIVEPEILPDGDHDLKRCQYVTEKVLAAVYKALSDHHIYLEGTL | from Gene ID |
| | LKPNMVTPGHACTQKFSHEEIAMATVTALRRTVPPAVTGITFLSGGQ | ENSG00000149925; Homo |
| | SEEEASINLNAINKCPLLKPWALTFSYGRALQASALKAWGGKKENL | sapiens; |
| | KAAQEEYVKRALANSLACQGKYTPSGQAGAAASESLFVSNHAY | ALDOA protein |
| | | (ENSP00000485952) encoded |
| | | by Transcript ID |
| | | ENST00000627059 from Gene |
| | | ID ENSG00000149925; Homo |
| | | sapiens |
| 2 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000266376) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000347598 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494095) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000643038 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKILGNADYVFTSIFTLEIILKMTAYGAFLHKGSFCRNYFNILD | |
| | LLVVSVSLISFGIQSSAINVVKILRVLRVLRPLRAINRAKGLKHVVQC | |
| | VFVAIRTIGNIVIVTTLLQFMFACIGVQLFKGKLYTCSDSSKQTEAEC | |
| | KGNYITYKDGEVDHPIIQPRSWENSKFDFDNVLAAMMALFTVSTFE | |
| | GWPELLYRSIDSHTEDKGPIYNYRVEISIFFIIYIIIIAFFMMNIFVGFVI | |
| | VTFQEQGEQEYKNCELDKNQRQCVEYALKARPLRRYIPKNQHQYK | |
| | VWYVVNSTYFEYLMFVLILLNTICLAMQHYGQSCLFKIAMNILNML | |
| | FTGLFTVEMILKLIAFKPKGYFSDPWNVFDFLIVIGSIIDVILSETNHYF | |
| | CDAWNTFDALIVVGSIVDIAITEVNPAEHTQCSPSMNAEENSRISITFF | |
| | RLFRVMRLVKLLSRGEGIRTLLWTFIKSFQALPYVALLIVMLFFIYAV | |
| | IGMQVFGKIALNDTTEINRNNNFQTFPQAVLLLFRCATGEAWQDIM | |
| | LACMPGKKCAPESEPSNSTEGETPCGSSFAVFYFISFYMLCAFLIINLF | |
| | VAVIMDNFDYLTRDWSILGPHHLDEFKRIWAEYDPEAKGRIKHLDV | |
| | VTLLRRIQPPLGFGKLCPHRVACKRLVSMNMPLNSDGTVMFNATLF | |
| | ALVRTALRIKTEGNLEQANEELRAIIKKIWKRTSMKLLDQVVPPAGD | |
| | DEVTVGKFYATFLIQEYFRKFKKRKEQGLVGKPSQRNALSLQAGLR | |
| | TLHDIGPEIRRAISGDLTAEEELDKAMKEAVSAASEDDIFRRAGGLF | |
| | GNHVSYYQSDGRSAFPQTFTTQRPLHINKAGSSQGDTESPSHEKLVD | |
| | STFTPSSYSSTGSNANINNANNTALGRLPRPAGYPSTVSTVEGHGPPL | |
| | SPAIRVQEVAWKLSSNRCHSRESQAAMAGQEETSQDETYEVKMNH | |
| | DTEACSEPSLLSTEMLSYQDDENRQLTLPEEDKRDIRQSPKRGFLRS | |
| | ASLGRRASFHLECLKRQKDRGGDISQKTVLPLHLVHHQALAVAGLS | |
| | PLLQRSHSPASFPRPFATPPATPGSRGWPPQPVPTLRLEGVESSEKLN | |
| | SSFPSIHCGSWAETTPGGGGSSAARRVRPVSLMVPSQAGAPGRQFH | |
| | GSASSLVEAVLISEGLGQFAQDPKFIEVTTQELADACDMTIEEMESA | |
| | ADNILSGGAPQSPNGALLPFVNCRDAGQDRAGGEEDAGCVRARGR | |
| | PSEEELQDSRVYVSSL | |
| 3 | MDASSSPWNPTPAPVSSPPLLLPIPAIVFIAVGIYLLLLGLVLLTRNCLL | ALDOA protein |
| | AQGCCADGSSPCRKQGSSGPPDCCWTCAEACNFPLPSPAHFLDACCP | (ENSP00000336927) encoded |
| | QPTRADWAPRCPRCCPLCDCACTCQLPDCQSLNCLCFEIKLR | by Transcript ID |
| | | ENST00000338110 |
| | | from Gene ID |
| | | ENSG00000149925; Homo |
| | | sapiens |
| 4 | MDASSSPWNPTPAPVSSPPLLLPIPAIVFIAVGIYLLLLGLVLLTRNCLL | ALDOA protein |
| | AQGCCADGSSPCRKQGSSGPPDCCWTCAEACNFPLPSPAHFLDACCP | (ENSP00000378669) encoded |
| | QPTRAGFHSGDGEREITRPLQQRVRNDVLWSSGCSALHAIRSRTGHLA | by Transcript ID |
| | APAAAHSATVPVRASSPTARASTVSASRSSSDEDPGPLPSGERPAPRA | ENST00000395248 |
| | HVPSSLKSLSPRHWNHRWPAEHPGLGTGSGSAGPGSLQGRTLGRLDG | from Gene ID |
| | SSSGGPEKRGASARVPGQRFISLDNQGPPSGFPRKNFL | ENSG00000149925; Homo |
| | | sapiens |
| 5 | MPYQYPALTPEQKKELSDIAHRIVAPGKGILAADESTGSIAKRLQSIG | ALDOA protein |
| | TENTEENRRFYRQLLLTADDRVNPCIGGVILFHETLYQKADDGRPFP | (ENSP00000400452) encoded |
| | QVIKSKGGVVGIKVDKGVVPLAGTNGETTTQGLDGLSERCAQYKK | by Transcript ID |
| | DGADFAKWRCVLKIGEHTPSALAIMENANVLARYASICQQNGIVPIV | ENST00000412304 from Gene |
| | EPEILPDGDHDLKRCQYVTEKVLAAVYKALSDHHIYLEGTLLKPNM | ID ENSG00000149925; Homo |
| | VTPGHACTQKFSHEEIAMATVTALRRTVPPAVTGITFLSGGQSEEEA | sapiens; |
| | SINLNAINKCPLLKPWALTFSYGRALQASALKAWGGKKENLKAAQE | ALDOA protein |
| | EYVKRALANSLACQGKYTPSGQAGAAASESLFVSNHAY | (ENSP00000455800) encoded |
| | | by Transcript ID |
| | | ENST00000563060 |
| | | from Gene ID |
| | | ENSG00000149925; Homo |
| | | sapiens; |
| | | ALDOA protein |
| | | (ENSP00000455917) encoded |
| | | by Transcript ID |
| | | ENST00000564546 |
| | | from Gene ID |
| | | ENSG00000149925; Homo |
| | | sapiens; |
| | | ALDOA protein |
| | | (ENSP00000455700) encoded |
| | | by Transcript ID |
| | | ENST00000569545 |
| | | from Gene ID |
| | | ENSG00000149925; Homo |
| | | sapiens; |
| | | ALDOA protein |
| | | (ENSP00000494188) encoded |
| | | by Transcript ID |
| | | ENST00000643777 from Gene |
| | | ID ENSG00000149925; Homo |
| | | sapiens |
| 6 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000329877) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000327702 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494109) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000642375 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLIWLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | HYFCDAWNTFDALIVVGSIVDIAITEVNPAEHTQCSPSMNAEENSRIS | |
| | ITFFRLFRVMRLVKLLSRGEGIRTLLWTFIKSFQALPYVALLIVMLFFI | |
| | YAVIGMQVFGKIALNDTTEINRNNNFQTFPQAVLLLFRCATGEAWQ | |
| | DIMLACMPGKKCAPESEPSNSTEGETPCGSSFAVFYFISFYMLCAFLII | |
| | NLFVAVIMDNFDYLTRDWSILGPHHLDEFKRIWAEYDPEAKGRIKH | |
| | LDVVTLLRRIQPPLGFGKLCPHRVACKRLVSMNMPLNSDGTVMFNA | |
| | TLFALVRTALRIKTEGNLEQANEELRAIIKKIWKRTSMKLLDQVVPP | |
| | AGDDEVTVGKFYATFLIQEYFRKFKKRKEQGLVGKPSQRNALSLQA | |
| | GLRTLHDIGPEIRRAISGDLTAEEELDKAMKEAVSAASEDDIFRRAG | |
| | GLFGNHVSYYQSDGRSAFPQTFTTQRPLHINKAGSSQGDTESPSHEK | |
| | LVDSTFTPSSYSSTGSNANINNANNTALGRLPRPAGYPSTVSTVEGH | |
| | GPPLSPAIRVQEVAWKLSSNRERHVPMCEDLELRRDSGSAGTQAHC | |
| | LLLRRANPSRCHSRESQAAMAGQEETSQDETYEVKMNHDTEACSEP | |
| | SLLSTEMLSYQDDENRQLTLPEEDKRDIRQSPKRGFLRSASLGRRAS | |
| | FHLECLKRQKDRGGDISQKTVLPLHLVHHQALAVAGLSPLLQRSHS | |
| | PASFPRPFATPPATPGSRGWPPQPVPTLRLEGVESSEKLNSSFPSIHCG | |
| | SWAETTPGGGGSSAARRVRPVSLMVPSQAGAPGRQFHGSASSLVEA | |
| | VLISEGLGQFAQDPKFIEVTTQELADACDMTIEEMESAADNILSGGA | |
| | PQSPNGALLPFVNCRDAGQDRAGGEEDAGCVRARGRPSEEELQDSR | |
| | VYVSSL | |
| 7 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000336982) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000335762 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000495321) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNRGTP | ENST00000647229 from Gene |
| | AGMLDQKKGKFAWFSHSTETHVSMPTSETESVNTENVAGGDIEGE | ID ENSG00000285479; Homo |
| | NCGARLAHRISKSKFSRYWRRWNRFCRRKCRAAVKSNVFYWLVIF | sapiens |
| | LVFLNTLTIASEHYNQPNWLTEVQDTANKALLALFTAEMLLKMYSL | |
| | GLQAYFVSLFNRFDCFVVCGGILETILVETKIMSPLGISVLRCVRLLRI | |
| | FKITRYWNSLSNLVASLLNSVRSIASLLLLLFLFIIIFSLLGMQLFGGK | |
| | FNFDEMQTRRSTFDNFPQSLLTVFQILTGEDWNSVMYDGIMAYGGP | |
| | SFPGMLVCIYFIILFICGNYILLNVFLAIAVDNLADAESLTSAQKEEEE | |
| | EKERKKLARTASPEKKQELVEKPAVGESKEEKIELKSITADGESPPAT | |
| | KINMDDLQPNENEDKSPYPNPETTGEEDEEEPEMPVGPRPRPLSELH | |
| | LKEKAVPMPEASAFFIFSSNNRFRLQCHRIVNDTIFTNLILFFILLSSIS | |
| | LAAEDPVQHTSFRNHILFYFDIVFTTIFTIEIALKMTAYGAFLHKGSFC | |
| | RNYFNILDLLVVSVSLISFGIQSSAINVVKILRVLRVLRPLRAINRAKG | |
| | LKHVVQCVFVAIRTIGNIVIVTTLLQFMFACIGVQLFKGKLYTCSDSS | |
| | KQTEAECKGNYITYKDGEVDHPIIQPRSWENSKFDFDNVLAAMMAL | |
| | FTVSTFEGWPELLYRSIDSHTEDKGPIYNYRVEISIFFIIYIIIIAFFMMN | |
| | IFVGFVIVTFQEQGEQEYKNCELDKNQRQCVEYALKARPLRRYIPKN | |
| | QHQYKVWYVVNSTYFEYLMFVLILLNTICLAMQHYGQSCLFKIAM | |
| | NILNMLFTGLFTVEMILKLIAFKPKHYFCDAWNTFDALIVVGSIVDIA | |
| | ITEVNPAEHTQCSPSMNAEENSRISITFFRLFRVMRLVKLLSRGEGIRT | |
| | LLWTFIKSFQALPYVALLIVMLFFIYAVIGMQVFGKIALNDTTEINRN | |
| | NNFQTFPQAVLLLFRCATGEAWQDIMLACMPGKKCAPESEPSNSTE | |
| | GETPCGSSFAVFYFISFYMLCAFLIINLFVAVIMDNFDYLTRDWSILG | |
| | PHHLDEFKRIWAEYDPEAKGRIKHLDVVTLLRRIQPPLGFGKLCPHR | |
| | VACKRLVSMNMPLNSDGTVMFNATLFALVRTALRIKTEGNLEQAN | |
| | EELRAIIKKIWKRTSMKLLDQVVPPAGDDEVTVGKFYATFLIQEYFR | |
| | KFKKRKEQGLVGKPSQRNALSLQAGLRTLHDIGPEIRRAISGDLTAE | |
| | EELDKAMKEAVSAASEDDIFRRAGGLFGNHVSYYQSDGRSAFPQTF | |
| | TTQRPLHINKAGSSQGDTESPSHEKLVDSTFTPSSYSSTGSNANINNA | |
| | NNTALGRLPRPAGYPSTVSTVEGHGPPLSPAIRVQEVAWKLSSNRCH | |
| | SRESQAAMAGQEETSQDETYEVKMNHDTEACSEPSLLSTEMLSYQD | |
| | DENRQLTLPEEDKRDIRQSPKRGFLRSASLGRRASFHLECLKRQKDR | |
| | GGDISQKTVLPLHLVHHQALAVAGLSPLLQRSHSPASFPRPFATPPA | |
| | TPGSRGWPPQPVPTLRLEGVESSEKLNSSFPSIHCGSWAETTPGGGGS | |
| | SAARRVRPVSLMVPSQAGAPGRQFHGSASSLVEAVLISEGLGQFAQ | |
| | DPKFIEVTTQELADACDMTIEEMESAADNILSGGAPQSPNGALLPFV | |
| | NCRDAGQDRAGGEEDAGCVRARGRPSEEELQDSRVYVSSL | |
| 8 | MARRKPEGSSFNMTHLSMAMAFSFPPVASGQLHPQLGNTQHQTEL | ALDOA protein |
| | GKELATTSTMPYQYPALTPEQKKELSDIAHRIVAPGKGILAADESTG | (ENSP00000457468) encoded |
| | SIAKRLQSIGTENTEENRRFYRQLLLTADDRVNPCIGGVILFHETLYQ | by Transcript ID |
| | KADDGRPFPQVIKSKGGVVGIKVDKGVVPLAGTNGETTTQGLDGLS | ENST00000564595 |
| | ERCAQYKKDGADFAKWRCVLKIGEHTPSALAIMENANVLARYASIC | from Gene ID |
| | QQNGIVPIVEPEILPDGDHDLKRCQYVTEKVLAAVYKALSDHHIYLE | ENSG00000149925; Homo |
| | GTLLKPNMVTPGHACTQKFSHEEIAMATVTALRRTVPPAVTGITFLS | sapiens; |
| | GGQSEEEASINLNAINKCPLLKPWALTFSYGRALQASALKAWGGKK | ALDOA protein |
| | ENLKAAQEEYVKRALANSLACQGKYTPSGQAGAAASESLFVSNHA | (ENSP00000496166) encoded |
| | Y | by Transcript ID |
| | | ENST00000642816 from Gene |
| | | ID ENSG00000149925; Homo |
| | | sapiens |
| 9 | MDAQGCCADGSSPCRKQGSSGPPDCCWTCAEACNFPLPSPAHFLDA | ALDOA protein |
| | CCPQPTRADWAPRCPRCCPLCDCACTCQLPDCQSLNCLCFEIKLR | (ENSP00000455724) encoded |
| | | by Transcript ID |
| | | ENST00000566897 from Gene |
| | | ID ENSG00000149925; Homo |
| | | sapiens; |
| | | ALDOA protein |
| | | (ENSP00000457683) encoded |
| | | by Transcript ID |
| | | ENST00000568435 from Gene |
| | | ID ENSG00000149925; Homo |
| | | sapiens |
| 10 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000341092) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000344100 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000495576) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000643858 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVONLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKH | |
| | YFCDAWNTFDALIVVGSIVDIAITEVNPAEHTQCSPSMGPSCSHPPLA | |
| | VLTAPPVADGFQNAEENSRISITFFRLFRVMRLVKLLSRGEGIRTLLW | |
| | TFIKSFQALPYVALLIVMLFFIYAVIGMQVFGKIALNDTTEINRNNNF | |
| | QTFPQAVLLLFRCATGEAWQDIMLACMPGKKCAPESEPSNSTEGET | |
| | PCGSSFAVFYFISFYMLCAFLIINLFVAVIMDNFDYLTRDWSILGPHH | |
| | LDEFKRIWAEYDPEAKGRIKHLDVVTLLRRIQPPLGFGKLCPHRVAC | |
| | KRLVSMNMPLNSDGTVMFNATLFALVRTALRIKTEEGPSPSEAHQG | |
| | AEDPFRPAGNLEQANEELRAIIKKIWKRTSMKLLDQVVPPAGDDEV | |
| | TVGKFYATFLIQEYFRKFKKRKEQGLVGKPSQRNALSLQAGLRTLH | |
| | DIGPEIRRAISGDLTAEEELDKAMKEAVSAASEDDIFRRAGGLFGNH | |
| | VSYYQSDGRSAFPQTFTTQRPLHINKAGSSQGDTESPSHEKLVDSTF | |
| | TPSSYSSTGSNANINNANNTALGRLPRPAGYPSTVSTVEGHGPPLSPA | |
| | IRVQEVAWKLSSNRCHSRESQAAMAGQEETSQDETYEVKMNHDTE | |
| | ACSEPSLLSTEMLSYQDDENRQLTLPEEDKRDIRQSPKRGFLRSASL | |
| | GRRASFHLECLKRQKDRGGDISQKTVLPLHLVHHQALAVAGLSPLL | |
| | QRSHSPASFPRPFATPPATPGSRGWPPQPVPTLRLEGVESSEKLNSSF | |
| | PSIHCGSWAETTPGGGGSSAARRVRPVSLMVPSQAGAPGRQFHGSA | |
| | SSLVEAVLISEGLGQFAQDPKFIEVTTQELADACDMTIEEMESAADNI | |
| | LSGGAPQSPNGALLPFVNCRDAGQDRAGGEEDAGCVRARGRPSEEE | |
| | LQDSRVYVSSL | |
| 11 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382500) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399591 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494999) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000642583 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKEDFDNVLAAMMALFTVSTFEGWPELLYRSIDSHTEDKG | |
| | PlYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | HYFCDAWNTFDALIVVGSIVDIAITEVNNAEENSRISITFFRLFRVMR | |
| | LVKLLSRGEGIRTLLWTFIKSFQALPYVALLIVMLFFIYAVIGMQVFG | |
| | KIALNDTTEINRNNNFQTFPQAVLLLFRCATGEAWQDIMLACMPGK | |
| | KCAPESEPSNSTEGETPCGSSFAVFYFISFYMLCAFLIINLFVAVIMDN | |
| | FDYLTRDWSILGPHHLDEFKRIWAEYDPEAKGRIKHLDVVTLLRRIQ | |
| | PPLGFGKLCPHRVACKRLVSMNMPLNSDGTVMFNATLFALVRTAL | |
| | RIKTEEGPSPSEAHQGAEDPFRPAGNLEQANEELRAIIKKIWKRTSM | |
| | KLLDQVVPPAGDDEVTVGKFYATFLIQEYFRKFKKRKEQGLVGKPS | |
| | QRNALSLQAGLRTLHDIGPEIRRAISGDLTAEEELDKAMKEAVSAAS | |
| | EDDIFRRAGGLFGNHVSYYQSDGRSAFPQTFTTQRPLHINKAGSSQG | |
| | DTESPSHEKLVDSTFTPSSYSSTGSNANINNANNTALGRLPRPAGYPS | |
| | TVSTVEGHGPPLSPAIRVQEVAWKLSSNRCHSRESQAAMAGQEETS | |
| | QDETYEVKMNHDTEACSEPSLLSTEMLSYQDDENRQLTLPEEDKRD | |
| | IRQSPKRGFLRSASLGRRASFHLECLKRQKDRGGDISQKTVLPLHLV | |
| | HHQALAVAGLSPLLQRSHSPASFPRPFATPPATPGSRGWPPQPVPTL | |
| | RLEGVESSEKLNSSFPSIHCGSWAETTPGGGGSSAARRVRPVSLMVP | |
| | SQAGAPGRQFHGSASSLVEAVLISEGLGQFAQDPKFIEVTTQELADA | |
| | CDMTIEEMESAADNILSGGAPQSPNGALLPFVNCRDAGQDRAGGEE | |
| | DAGCVRARGRPSEEELQDSRVYVSSL | |
| 12 | XPGHACTQKFSHEEIAMATVTALRRTVPPAVTGITFLSGGQSEEEASI | ALDOA protein |
| | NLNAINKCPLLKPWALTFSYGRALQASALKAWGGKKENLKAAQEE | (ENSP00000456098) encoded |
| | YVKRALANSLACQGKYTPSGQAGAAASESLFVSNHAY | by Transcript ID |
| | | ENST00000565355 |
| | | from Gene ID |
| | | ENSG00000149925; Homo |
| | | sapiens |
| 13 | MPYQYPALTPEQKKELSDIAHRIVAPGSIAKRLQSIGTENTEENRRFY | ALDOA protein |
| | RQLLLTADDRVNPCIGGVILFHETLYQKADDGRPFPQVIKSKGGVVG | (ENSP00000454499) encoded |
| | IKVDKGVVPLAGTNGETTTQGLDGLSERCAQYKKDGADFAKWRCV | by Transcript ID |
| | | ENST00000566846 from Gene |
| | | ID ENSG00000149925; Homo |
| | | sapiens |
| 14 | MPYQYPALTPEQKKELSDIAHRIVAPGKGILAADESTGSIAKRLQSIG | ALDOA protein |
| | TENTEENRRFYRQLLLTADDRVNPCIGGVILFHETLYQKADDGRPFP | (ENSP00000457643) encoded |
| | QVIKSKGGVVGIKVDKGVVPLAGTNGETTTQGLDGLSERCAQYKK | by Transcript ID |
| | DGADFAKWRCVLKIG | ENST00000563987 from Gene |
| | | ID ENSG00000149925; Homo |
| | | sapiens |
| 15 | MPYQYPALTPEQKKELSDIAHRIVAPGSIAKRLQSIGTENTEENRRFY | ALDOA protein |
| | RQLLLTADDRVNPCIGGVILFHETLYQKADDGRPFPQVIKSKGGVVG | (ENSP00000456020) encoded |
| | IKVDKGVVPLAGTNGETTTQGLDGLSERCAQYKKDGADFAKWRCV | by Transcript ID |
| | LKIGEHTPSALAIMENANVLAR | ENST00000562168 |
| | | from Gene ID |
| | | ENSG00000149925; Homo |
| | | sapiens |
| 16 | MARRKPEGSSFNMTHLSMAMAFSFPPVASGQLHPQLGNTQHQTEL | ALDOA protein |
| | GKELATTSTMPYQYPALTPEQKKELSDIAHRIVAPGKGILAADESTG | (ENSP00000455455) encoded |
| | SIAKRLQSIGTENTEENRRFYRQLLLTADDRVNPCIGGVILFHETLYQ | by Transcript ID |
| | KADDGRPFPQVIKSKGGVVGIKVDKGVVPLAGTNGETTTQGLDGLS | ENST00000562679 from Gene |
| | ERCAQYKKDGADFAKWRCVLKIGEHTPSALAIMENANVLARYASIC | ID ENSG00000149925; Homo |
| | QQNGIVPIVEPEILPDGDHDLKRCQYVTEKVLAAVYKALSDHHIYL | sapiens |
| 17 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382504) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399595 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000495678) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000647521 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKH | |
| | YFCDAWNTFDALIVVGSIVDIAITEVNNAEENSRISITFFRLFRVMRL | |
| | VKLLSRGEGIRTLLWTFIKSFQALPYVALLIVMLFFIYAVIGMQVFGK | |
| | IALNDTTEINRNNNFQTFPQAVLLLFRCATGEAWQDIMLACMPGKK | |
| | CAPESEPSNSTEGETPCGSSFAVFYFISFYMLCAFLIINLFVAVIMDNF | |
| | DYLTRDWSILGPHHLDEFKRIWAEYDPEAKGRIKHLDVVTLLRRIQP | |
| | PLGFGKLCPHRVACKRLVSMNMPLNSDGTVMFNATLFALVRTALRI | |
| | KTEEGPSPSEAHQGAEDPFRPAGNLEQANEELRAIIKKIWKRTSMKL | |
| | LDQVVPPAGDDEVTVGKFYATFLIQEYFRKFKKRKEQGLVGKPSQR | |
| | NALSLQAGLRTLHDIGPEIRRAISGDLTAEEELDKAMKEAVSAASED | |
| | DIFRRAGGLFGNHVSYYQSDGRSAFPQTFTTQRPLHINKAGSSQGDT | |
| | ESPSHEKLVDSTFTPSSYSSTGSNANINNANNTALGRLPRPAGYPSTV | |
| | STVEGHGPPLSPAIRVQEVAWKLSSNRCHSRESQAAMAGQEETSQD | |
| | ETYEVKMNHDTEACSEPSLLSTEMLSYQDDENRQLTLPEEDKRDIR | |
| | QSPKRGFLRSASLGRRASFHLECLKRQKDRGGDISQKTVLPLHLVHH | |
| | QALAVAGLSPLLQRSHSPASFPRPFATPPATPGSRGWPPQPVPTLRLE | |
| | GVESSEKLNSSFPSIHCGSWAETTPGGGGSSAARRVRPVSLMVPSQA | |
| | GAPGRQFHGSASSLVEAVLISEGLGQFAQDPKFIEVTTQELADACDM | |
| | TIEEMESAADNILSGGAPQSPNGALLPFVNCRDAGQDRAGGEEDAG | |
| | CVRARGRPSEEELQDSRVYVSSL | |
| 18 | RRFYRQLLLTADDRVNPCIGGVILFHETLYQKADDGRPFPQVIKSKG | ALDOA protein |
| | GVVGIKVDKGVVPLAGTNGETTTQGLDGLSERCAQYKKDGADFAK | (ENSP00000457514) encoded |
| | WRCVLKIGEHTPSALAIMENANVLARYASICQQVLAAVYKALSDHH | by Transcript ID |
| | IYLEGTLLKPNMVTPGHACTQKFSHEEIAMATVTALRRTVPPAVTGI | ENST00000564688 from Gene |
| | TFLSGGQSEEEASINLNAI | ID ENSG00000149925; Homo |
| | | sapiens |
| 19 | MPYQYPALTPEQKKELSDIAHRIVAPGKGILAADESTGSIAKRLQSIG | ALDOA protein |
| | TENTEENRRFYRQLLLTADDRVNPCIGGVILFHETLYQKADDGRPFP | (ENSP00000455857) encoded |
| | QVIKSKGGVVGIKVDKGVVPLAGTNGETTTQGLDGLSERCAQYKK | by Transcript ID |
| | DGADFAKWRCVLKIGEHTPSALAIMENANVLARYASICQQNGIVPIV | ENST00000569798 from Gene |
| | EPEILPDGDHDLKRCQYVTEKVLAAVYKALSDHHIYLEGTLLKPNM | ID ENSG00000149925; Homo |
| | VTPGHACTQKFSHEEIAMATVTALRRTVPPAVTGITFLSGGQSEEEA | sapiens |
| | SINLNAINKCPLLKPWALTFSYGRALQASALKAWGGKKENLKAAQE | |
| | EYVKRALVRIGRRWAGCLGGWDSEKSPSHSTPLPA | |
| 20 | MQMPAMMSLLLVSVGLMEALQAQSHPITRRDLFSQEIQLDMALASF | ART1 protein |
| | DDQYAGCAAAMTAALPDLNHTEFQANQVYADSWTLASSQWQERQ | (ENSP00000250693) encoded |
| | ARWPEWSLSPTRPSPPPLGFRDEHGVALLAYTANSPLHKEFNAAVR | by Transcript ID |
| | EAGRSRAHYLHHFSFKTLHFLLTEALQLLGSGQRPPRCHQVFRGVH | ENST00000250693 from Gene |
| | GLRFRPAGPRATVRLGGFASASLKHVAAQQFGEDTFFGIWTCLGAPI | ID ENSG00000129744; Homo |
| | KGYSFFPGEEEVLIPPFETFQVINASRLAQGPARIYLRALGKHSTYNC | sapiens |
| | EYIKDKKCKSGPCHLDNSAMGQSPLSAVWSLLLLLWFLVVRAFPDG | |
| | PGLL | |
| 21 | MEPSSPQDEGLRKKQPKKPVPEILPRPPRALFCLTLENPLRKACISIVE | CACNA1S protein |
| | WKPFETIILLTIFANCVALAVYLPMPEDDNNSLNLGLEKLEYFFLIVF | (ENSP00000356307) encoded |
| | SIEAAMKIIAYGFLFHQDAYLRSGWNVLDFTIVFLGVFTVILEQVNVI | by Transcript ID |
| | QSHTAPMSSKGAGLDVKALRAFRVLRPLRLVSGVPSLQVVLNSIFK | ENST00000367338 from Gene |
| | AMLPLFHIALLVLFMVIIYAIIGLELFKGKMHKTCYFIGTDIVATVEN | ID ENSG00000081248; Homo |
| | EEPSPCARTGSGRRCTINGSECRGGWPGPNHGITHFDNFGFSMLTVY | sapiens |
| | QCITMEGWTDVLYWVNDAIGNEWPWIYFVTLILLGSFFILNLVLGV | |
| | LSGEFTKEREKAKSRGTFQKLREKQQLDEDLRGYMSWITQGEVMD | |
| | VEDFREGKLSLDEGGSDTESLYEIAGLNKIIQFIRHWRQWNRIFRWK | |
| | CHDIVKSKVFYWLVILIVALNTLSIASEHHNQPLWLTRLQDIANRVL | |
| | LSLFTTEMLMKMYGLGLRQYFMSIFNRFDCFVVCSGILEILLVESGA | |
| | MTPLGISVLRCIRLLRIFKITKYWTSLSNLVASLLNSIRSIASLLLLLFL | |
| | FIVIFALLGMQLFGGRYDFEDTEVRRSNFDNFPQALISVFQVLTGED | |
| | WTSMMYNGIMAYGGPSYPGMLVCIYFIILFVCGNYILLNVFLAIAVD | |
| | NLAEAESLTSAQKAKAEEKKRRKMSKGLPDKSEEEKSTMAKKLEQ | |
| | KPKGEGIPTTAKLKIDEFESNVNEVKDPYPSADFPGDDEEDEPEIPLS | |
| | PRPRPLAELQLKEKAVPIPEASSFFIFSPTNKIRVLCHRIVNATWFTNFI | |
| | LLFILLSSAALAAEDPIRADSMRNQILKHFDIGFTSVFTVEIVLKMTT | |
| | YGAFLHKGSFCRNYFNMLDLLVVAVSLISMGLESSAISVVKILRVLR | |
| | VLRPLRAINRAKGLKHVVQCMFVAISTIGNIVLVTTLLQFMFACIGV | |
| | QLFKGKFFRCTDLSKMTEEECRGYYYVYKDGDPMQIELRHREWVH | |
| | SDFHFDNVLSAMMSLFTVSTFEGWPQLLYKAIDSNAEDVGPIYNNR | |
| | VEMAIFFIIYIILIAFFMMNIFVGFVIVTFQEQGETEYKNCELDKNQRQ | |
| | CVQYALKARPLRCYIPKNPYQYQVWYIVTSSYFEYLMFALIMLNTIC | |
| | LGMQHYNQSEQMNHISDILNVAFTIIFTLEMILKLMAFKARGYFGDP | |
| | WNVFDFLIVIGSIIDVILSEIDDPDESARISSAFFRLFRVMRLIKLLSRA | |
| | EGVRTLLWTFIKSFQALPYVALLIVMLFFIYAVIGMQMFGKIALVDG | |
| | TQINRNNNFQTFPQAVLLLFRCATGEAWQEILLACSYGKLCDPESDY | |
| | APGEEYTCGTNFAYYYFISFYMLCAFLVINLFVAVIMDNFDYLTRD | |
| | WSILGPHHLDEFKAIWAEYDPEAKGRIKHLDVVTLLRRIQPPLGFGK | |
| | FCPHRVACKRLVGMNMPLNSDGTVTFNATLFALVRTALKIKTEGNF | |
| | EQANEELRAIIKKIWKRTSMKLLDQVIPPIGDDEVTVGKFYATFLIQE | |
| | HFRKFMKRQEEYYGYRPKKDIVQIQAGLRTIEEEAAPEICRTVSGDL | |
| | AAEEELERAMVEAAMEEGIFRRTGGLFGQVDNFLERTNSLPPVMAN | |
| | QRPLQFAEIEMEEMESPVFLEDFPQDPRTNPLARANTNNANANVAY | |
| | GNSNHSNSHVFSSVHYEREFPEETETPATRGRALGQPCRVLGPHSKP | |
| | CVEMLKGLLTQRAMPRGQAPPAPCQCPRVESSMPEDRKSSTPGSLH | |
| | EETPHSRSTRENTSRCSAPATALLIQKALVRGGLGTLAADANFIMAT | |
| | GQALADACQMEPEEVEIMATELLKGREAPEGMASSLGCLNLGSSLG | |
| | SLDQHQGSQETLIPPRL | |
| 22 | MEPSSPQDEGLRKKQPKKPVPEILPRPPRALFCLTLENPLRKACISIVE | CACNA1S protein |
| | WKPFETTILLTIFANCVALAVYLPMPEDDNNSLNLGLEKLEYFFLIVF | (ENSP00000355192) encoded |
| | SIEAAMKIIAYGFLFHQDAYLRSGWNVLDFTIVFLGVFTVILEQVNVI | by Transcript ID |
| | QSHTAPMSSKGAGLDVKALRAFRVLRPLRLVSGVPSLQVVLNSIFK | ENST00000362061 from Gene |
| | AMLPLFHIALLVLFMVIIYAIIGLELFKGKMHKTCYFIGTDIVATVEN | ID ENSG00000081248; Homo |
| | EEPSPCARTGSGRRCTINGSECRGGWPGPNHGITHFDNFGFSMLTVY | sapiens |
| | QCITMEGWTDVLYWVNDAIGNEWPWIYFVTLILLGSFFILNLVLGV | |
| | LSGEFTKEREKAKSRGTFQKLREKQQLDEDLRGYMSWITQGEVMD | |
| | VEDFREGKLSLDEGGSDTESLYEIAGLNKIIQFIRHWRQWNRIFRWK | |
| | CHDIVKSKVFYWLVILIVALNTLSIASEHHNQPLWLTRLQDIANRVL | |
| | LSLFTTEMLMKMYGLGLRQYFMSIFNRFDCFVVCSGILEILLVESGA | |
| | MTPLGISVLRCIRLLRIFKITKYWTSLSNLVASLLNSIRSIASLLLLLFL | |
| | FIVIFALLGMQLFGGRYDFEDTEVRRSNFDNFPQALISVFQVLTGED | |
| | WTSMMYNGIMAYGGPSYPGMLVCIYFIILFVCGNYILLNVFLAIAVD | |
| | NLAEAESLTSAQKAKAEEKKRRKMSKGLPDKSEEEKSTMAKKLEQ | |
| | KPKGEGIPTTAKLKIDEFESNVNEVKDPYPSADFPGDDEEDEPEIPLS | |
| | PRPRPLAELQLKEKAVPIPEASSFFIFSPTNKIRVLCHRIVNATWFTNFI | |
| | LLFILLSSAALAAEDPIRADSMRNQILKHFDIGFTSVFTVEIVLKMTT | |
| | YGAFLHKGSFCRNYFNMLDLLVVAVSLISMGLESSAISVVKILRVLR | |
| | VLRPLRAINRAKGLKHVVQCMFVAISTIGNIVLVTTLLQFMFACIGV | |
| | QLFKGKFFRCTDLSKMTEEECRGYYYVYKDGDPMQIELRHREWVH | |
| | SDFHFDNVLSAMMSLFTVSTFEGWPQLLYKAIDSNAEDVGPIYNNR | |
| | VEMAIFFIIYIILIAFFMMNIFVGFVIVTFQEQGETEYKNCELDKNQRQ | |
| | CVQYALKARPLRCYIPKNPYQYQVWYIVTSSYFEYLMFALIMLNTIC | |
| | LGMQHYNQSEQMNHISDILNVAFTIIFTLEMILKLMAFKARGYFGDP | |
| | WNVFDFLIVIGSIIDVILSEIDTFLASSGGLYCLGGGCGNVDPDESARI | |
| | SSAFFRLFRVMRLIKLLSRAEGVRTLLWTFIKSFQALPYVALLIVMLF | |
| | FIYAVIGMQMFGKIALVDGTQINRNNNFQTFPQAVLLLFRCATGEA | |
| | WQEILLACSYGKLCDPESDYAPGEEYTCGTNFAYYYFISFYMLCAFL | |
| | VINLFVAVIMDNFDYLTRDWSILGPHHLDEFKAIWAEYDPEAKGRIK | |
| | HLDVVTLLRRIQPPLGFGKFCPHRVACKRLVGMNMPLNSDGTVTFN | |
| | ATLFALVRIALKIKTEGNFEQANEELRAIIKKIWKRTSMKLLDQVIPP | |
| | IGDDEVTVGKFYATFLIQEHFRKFMKRQEEYYGYRPKKDIVQIQAGL | |
| | RTIEEEAAPEICRTVSGDLAAEEELERAMVEAAMEEGIFRRTGGLFG | |
| | QVDNFLERTNSLPPVMANQRPLQFAEIEMEEMESPVFLEDFPQDPRT | |
| | NPLARANTNNANANVAYGNSNHSNSHVFSSVHYEREFPEETETPAT | |
| | RGRALGQPCRVLGPHSKPCVEMLKGLLTQRAMPRGQAPPAPCQCP | |
| | RVESSMPEDRKSSTPGSLHEETPHSRSTRENTSRCSAPATALLIQKAL | |
| | VRGGLGTLAADANFIMATGQALADACQMEPEEVEIMATELLKGRE | |
| | APEGMASSLGCLNLGSSLGSLDQHQGSQETLIPPRL | |
| 23 | MAAGCLLALTLTLFQSLLIGPSSEEPFPSAVTIKSWVDKMQEDLVTL | CACNA2D1 protein |
| | AKTASGVNQLVDIYEKYQDLYTVEPNNARQLVEIAARDIEKLLSNR | (ENSP00000348589) encoded |
| | SKALVRLALEAEKVQAAHQWREDFASNEVVYYNAKDDLDPEKND | by Transcript ID |
| | SEPGSQRIKPVFIEDANFGRQISYQHAAVHIPTDIYEGSTIVLNELNWT | ENST00000356253 from Gene |
| | SALDEVFKKNREEDPSLLWQVFGSATGLARYYPASPWVDNSRTPNK | ID ENSG00000153956; Homo |
| | IDLYDVRRRPWYIQGAASPKDMLILVDVSGSVSGLTLKLIRTSVSEM | sapiens |
| | LETLSDDDIFVNVASFNSNAQDVSCFQHLVQANVRNKKVLKDAVNN | |
| | ITAKGITDYKKGFSFAFEQLLNYNVSRANCNKIIMLFTDGGEERAQEI | |
| | FNKYNKDKKVRVFTFSVGQHNYDRGPIQWMACENKGYYYEIPSIG | |
| | AIRINTQEYLDVLGRPMVLAGDKAKQVQWTNVYLDALELGLVITG | |
| | TLPVFNITGQFENKTNLKNQLILGVMGVDVSLEDIKRLTPRFTLCPN | |
| | GYYFAIDPNGYVLLHPNLQPKP1GVGIPTINLRKRRPNIQNPKSQEPV | |
| | TLDFLDAELENDIKVEIRNKMIDGESGEKTFRTLVKSQDERYIDKGN | |
| | RTYTWTPVNGTDYSLALVLPTYSFYYIKAKLEETITQARYSETLKPD | |
| | NFEESGYTFIAPRDYCNDLKISDNNTEFLLNFNEFIDRKTPNNPSCNA | |
| | DLINRVLLDAGFTNELVQNYWSKQKNIKGVKARFVVTDGGITRVYP | |
| | KEAGENWQENPETYEDSFYKRSLDNDNYVFTAPYFNKSGPGAYES | |
| | GIMVSKAVEIYIQGKLLKPAVVGIKIDVNSWIENFTKTSIRDPCAGPV | |
| | CDCKRNSDVMDCVILDDGGFLLMANHDDYTNQIGRFFGEIDPSLMR | |
| | HLVNISVYAFNKSYDYQSVCEPGAAPKQGAGHRSAYVPSVADILQI | |
| | GWWATAAAWSILQQFLLSLTFPRLLEAVEMEDDDFTASLSKQSCIT | |
| | EQTQYFFDNDSKSFSGVLDCGNCSRIFHGEKLMNTNLIFIMVESKGT | |
| | CPCDTRLLIQAEQTSDGPNPCDMVKQPRYRKGPDVCFDNNVLEDYT | |
| | DCGGVSGLNPSLWYIIGIQFLLLWLVSGSTHRLL | |
| 24 | MAAGCLLALTLTLFQSLLIGPSSEEPFPSAVTIKSWVDKMQEDLVTL | CACNA2D1 protein |
| | AKTASGVNQLVDIYEKYQDLYTVEPNNARQLVEIAARDIEKLLSNR | (ENSP00000349320) encoded |
| | SKALVRLALEAEKVQAAHQWREDFASNEVVYYNAKDDLDPEKND | by Transcript ID |
| | SEPGSQRIKPVFIEDANFGRQISYQHAAVHIPTDIYEGSTIVLNELNWT | ENST00000356860 from Gene |
| | SALDEVFKKNREEDPSLLWQVFGSATGLARYYPASPWVDNSRTPNK | ID ENSG00000153956; Homo |
| | IDLYDVRRRPWYIQGAASPKDMLILVDVSGSVSGLTLKLIRTSVSEM | sapiens |
| | LETLSDDDFVNVASFNSNAQDVSCFQHLVQANVRNKKVLKDAVNN | |
| | ITAKGITDYKKGFSFAFEQLLNYNVSRANCNKIIMLFTDGGEERAQEI | |
| | FNKYNKDKKVRVFTFSVGQHNYDRGPIQWMACENKGYYYEIPSIG | |
| | AIRINTQEYLDVLGRPMVLAGDKAKQVQWTNVYLDALELGLVITG | |
| | TLPVFNITGQFENKTNLKNQLILGVMGVDVSLEDIKRLTPRFTLCPN | |
| | GYYFAIDPNGYVLLHPNLQPKNPKSQEPVTLDFLDAELENDIKVEIR | |
| | NKMIDGESGEKTFRTLVKSQDERYIDKGNRTYTWTPVNGTDYSLAL | |
| | VLPTYSFYYIKAKLEETITQARSKKGKMKDSETLKPDNFEESGYTFIA | |
| | PRDYCNDLKISDNNTEFLLNFNEFIDRKTPNNPSCNADLINRVLLDA | |
| | GFTNELVQNYWSKQKNIKGVKARFVVTDGGITRVYPKEAGENWQE | |
| | NPETYEDSFYKRSLDNDNYVFTAPYFNKSGPGAYESGIMVSKAVEIY | |
| | IQGKLLKPAVVGIKIDVNSWIENFTKTSIRDPCAGPVCDCKRNSDVM | |
| | DCVILDDGGFLLMANHDDYTNQIGRFFGEIDPSLMRHLVNISVYAFN | |
| | KSYDYQSVCEPGAAPKQGAGHRSAYVPSVADILQIGWWATAAAWS | |
| | ILQQFLLSLTFPRLLEAVEMEDDDFTASLSKQSCITEQTQYFFDNDSK | |
| | SFSGVLDCGNCSRIFHGEKLMNTNLIFIMVESKGTCPCDTRLLIQAEQ | |
| | TSDGPNPCDMVKQPRYRKGPDVCFDNNVLEDYTDCGGVSGLNPSL | |
| | WYIIGIQFLLLWLVSGSTHRLL | |
| 25 | MAAGCLLALTLTLFQSLLIGPSSEEPFPSAVTIKSWVDKMQEDLVTL | CACNA2D1 protein |
| | AKTASGVNQLVDIYEKYQDLYTVEPNNARQLVEIAARDIEKLLSNR | (ENSP00000405395) encoded |
| | SKALVRLALEAEKVQAAHQWREDFASNEVVYYNAKDDLDPEKND | by Transcript ID |
| | SEPGSQRIKPVFIEDANFGRQISYQHAAVHIPTDIYEGSTIVLNELNWT | ENST00000423588 from Gene |
| | SALDEVFKKNREEDPSLLWQVFGSATGLARYYPASPWVDNSRTPNK | ID ENSG00000153956; Homo |
| | IDLYDVRRRPWYIQGAASPKDMLILVDVSGSVSGLTLKLIRTSVSEM | sapiens |
| | LETLSDDDFVNVASDSKEISPSPEEIFNAE | |
| 26 | XKRLTPRFTLCPNGYYFAIDPNGYVLLHPNLQPKPIGVGIPTINLRKR | CACNA2D1 protein |
| | RPNIQEPVTLDFLDAELENDIKVEIRNKMIDGESGEKTFRTLVKSQDE | (ENSP00000409374) encoded |
| | RYIDKGNRTYTWTPVNGTDYSLALVLPTYSFYYIKAKLEETITQARY | by Transcript ID |
| | SETLKPDNFEESGYTFIAPRDYCNDLKISDNNTEFLLNFNEFIDRKTP | ENST00000443883 from Gene |
| | NNPSCNADLINRVLLDAGFTNELVQNYWSKQKNIKGVKARFVVTD | ID ENSG00000153956; Homo |
| | GGITRVYPKEAGENWQENPETYEDSFYKRSLDNDNYVFTAPYFN | sapiens |
| 27 | MTWRMGPRFTMLLAMWLVCGSEPHPHATIRGSHGGRKVPLVSPDS | CCDC80 protein |
| | SRPARFLRHTGRSRGIERSTLEEPNLQPLQRRRSVPVLRLARPTEPPA | (ENSP00000206423) encoded |
| | RSDINGAAVRPEQRPAARGSPREMIRDEGSSARSRMLRFPSGSSSPNI | by Transcript ID |
| | LASFAGKNRVWVISAPHASEGYYRLMMSLLKDDVYCELAERHIQQI | ENST00000206423 from Gene |
| | VLFHQAGEEGGKVRRITSEGQILEQPLDPSLIPKLMSFLKLEKGKFG | ID ENSG00000091986; Homo |
| | MVLLKKTLQVEERYPYPVRLEAMYEVIDQGPIRRIEKIRQKGFVQKC | sapiens; |
| | KASGVEGQVVAEGNDGGGGAGRPSLGSEKKKEDPRRAQVPPTRES | CCDC80 protein |
| | RVKVLRKLAATAPALPQPPSTPRATTLPPAPATTVTRSTSRAVTVAA | (ENSP00000411814) encoded |
| | RPMTTTAFPTTQRPWTPSPSHRPPTTTEVITARRPSVSENLYPPSRKD | by Transcript ID |
| | QHRERPQTTRRPSKATSLESFTNAPPTTISEPSTRAAGPGRFRDNRMD | ENST00000439685 from Gene |
| | RREHGHRDPNVVPGPPKPAKEKPPKKKAQDKILSNEYEEKYDLSRP | ID ENSG00000091986; Homo |
| | TASQLEDELQVGNVPLKKAKESKKHEKLEKPEKEKKKKMKNENAD | sapiens |
| | KLLKSEKQMKKSEKKSKQEKEKSKKKKGGKTEQDGYQKPTNKHFT | |
| | QSPKKSVADLLGSFEGKRRLLLITAPKAENNMYVQQRDEYLESFCK | |
| | MATRKISVITIFGPVNNSTMKIDHFQLDNEKPMRVVDDEDLVDQRLI | |
| | SELRKEYGMTYNDFFMVLTDVDLRVKQYYEVPITMKSVFDLIDTFQ | |
| | SRIKDMEKQKKEGIVCKEDKKQSLENFLSRFRWRRRLLVISAPNDED | |
| | WAYSQQLSALSGQACNFGLRHITILKLLGVGEEVGGVLELFPINGSS | |
| | VVEREDVPAHLVKDIRNYFQVSPEYFSMLLVGKDGNVKSWYPSPM | |
| | WSMVIVYDLIDSMQLRRQEMAIQQSLGMRCPEDEYAGYGYHSYHQ | |
| | GYQDGYQDDYRHHESYHHGYPY | |
| 28 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382506) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399597 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000496049) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000643138 from Gene |
| | PTSETESVNTENVAGGDIEGENCGARLAHRISKSKFSRYWRRWNRF | ID ENS000000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQIIQYKVWYVVNSTYFEYLMFVLIL | |
| | LNTICLAMQHYGQSCLFKIAMNILNMLFTGLFTVEMILKLIAFKPKG | |
| | YFSDPWNVFDFLIVIGSIIDVILSETNPAEHTQCSPSMNAEENSRISITF | |
| | FRLFRVMRLVKLLSRGEGIRTLLWTFIKSFQALPYVALLIVMLFFIYA | |
| | VIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEGNLEQANEELRAIIKKIWKRTSMKLLDQVVPPA | |
| | GDDEVTVGKFYATFLIQEYFRKFKKRKEQGLVGKPSQRNALSLQAG | |
| | LRTLHDIGPEIRRAISGDLTAEEELDKAMKEAVSAASEDDIFRRAGG | |
| | LFGNHVSYYQSDGRSAFPQTFTTQRPLHINKAGSSQGDTESPSHEKL | |
| | VDSTFTPSSYSSTGSNANINNANNTALGRLPRPAGYPSTVSTVEGHG | |
| | PPLSPAIRVQEVAWKLSSNRCHSRESQAAMAGQEETSQDETYEVKM | |
| | NHDTEACSEPSLLSTEMLSYQDDENRQLTLPEEDKRDIRQSPKRGFL | |
| | RSASLGRRASFHLECLKRQKDRGGDISQKTVLPLHLVHHQALAVAG | |
| | LSPLLQRSHSPASFPRPFATPPATPGSRGWPPQPVPTLRLEGVESSEK | |
| | LNSSFPSIHCGSWAETTPGGGGSSAARRVRPVSLMVPSQAGAPGRQF | |
| | HGSASSLVEAVLISEGLGQFAQDPKFIEVTTQELADACDMTIEEMES | |
| | AADNILSGGAPQSPNGALLPFVNCRDAGQDRAGGEEDAGCVRARG | |
| | RPSEEELQDSRVYVSSL | |
| 29 | XFTQSPKKSVADLLGSFEGKRRLLLITAPKAENNMYVQQRDEYLES | CCDC80 protein |
| | FCKMATRKISVITIFGPVNNSTMKIDHFQLDNEKPMRVVDDEDLVD | (ENSP00000420123) encoded |
| | QRLISELRKEYGMTYNDFFMVLTDVDLRVKQYYEVPITMKSVFDLI | by Transcript ID |
| | DTFQSRIKDMEKQKKEGIVCKEDKKQSLENFLSRFRWRRRLLVISAP | ENST00000461431 from Gene |
| | NDEDWAYSQQLSALSGQACNFGSSVVEREDVPAHLVKDIRNYFQV | ID ENSG00000091986; Homo |
| | SPEYFSMLLVGKDGNVKSWYPSPMWSMVIVY | sapiens |
| 30 | MALRSMGQFSVFFFFFFWAQKKIASLSHSGFLHWFLLFEAVSSNGVL | CCDC80 protein |
| | LFFSRFRWRRRLLVISAPNDEDWAYSQQLSALSGQACNFGLRHITIL | (ENSP00000418188) encoded |
| | KLLGVGEEVGGVLELFPINGSSVVEREDVPAHLVKDIRNYFQVSPEY | by Transcript ID |
| | FSMLLVGKDGNVKSWYPSPMWSMVIVYDLIDSMQLRRQEMAIQQS | ENS100000479368 from Gene |
| | LGMRCPEDEYAGYGYHSYHQGYQDGY | ID ENSG00000091986; Homo |
| | | sapiens |
| 31 | MEGPVLTLGLLAALAVCGSWGLNEEERLIRHLFQEKGYNKELRPVA | CHRND protein |
| | HKEESVDVALALTLSNLISLKEVEETLTTNVWIEHGWTDNRLKWNA | (ENSP00000408819) encoded |
| | EEFGNISVLRLPPDMVWLPEIVLENNNDGSFQISYSCNVLVYHYGFV | by Transcript ID |
| | YWLPPAIFRSSCPISVTYFPFDWQNCSLKFRERGVGDSPPAGQGQRG | ENST00000441621 from Gene |
| | PQSPSGQPQPPGHHLLPHHPPQAPLLHHQHPGALRAHLLHGQPGLLP | ID ENS000000135902; Homo |
| | TO | sapiens |
| 32 | MEGPVLTLGLLAALAVCGSWGLNEEERLIRHLFQEKGYNKELRPVA | CHRND protein |
| | HKEESVDVALALTLSNLISLKEVEETLTTNVWIEHGWTDNRLKWNA | (ENSP00000258385) encoded |
| | EEFGNISVLRLPPDMVWLPEIVLENNNDGSFQISYSCNVLVYHYGFV | by Transcript ID |
| | YWLPPAIFRSSCPISVTYFPFDWQNCSLKFSSLKYTAKEITLSLKQDA | ENST00000258385 from Gene |
| | KENRTYPVEWIIIDPEGFTENGEWEIVHRPARVNVDPRAPLDSPSRQ | ID ENSG00000135902; Homo |
| | DITFYLIIRRKPLFYIINILVPCVLISFMVNLVFYLPADSGEKTSVAISV | sapiens |
| | LLAQSVFLLLISKRLPATSMAIPLIGKFLLFGMVLVTMVVVICVIVLNI | |
| | HFRTPSTHVLSEGVKKLFLETLPELLHMSRPAEDGPSPGALVRRSSSL | |
| | GYISKAEEYFLLKSRSDLMFEKQSERHGLARRLTTARRPPASSEQAQ | |
| | QELFNELKPAVDGANFIVNHMRDQNNYNEEKDSWNRVARTVDRLC | |
| | LFVVTPVMVVGTAWIFLQGVYNQPPPQPFPGDPYSYNVQDKRFI | |
| 33 | MEGPVLTLGLLAALAVCGSWGLNEEERLIRHLFQEKGYNKELRPVA | CHRND protein |
| | HKEESVDVALALTLSNLISLGWTDNRLKWNAEEFGNISVLRLPPDM | (ENSP00000438380) encoded |
| | VWLPEIVLENNNDGSFQISYSCNVLVYHYGFVYWLPPAIFRSSCPISV | by Transcript ID |
| | TYFPFDWQNCSLKFSSLKYTAKEITLSLKQDAKENRTYPVEWIIIDPE | ENST00000543200 from Gene |
| | GFTENGEWEIVHRPARVNVDPRAPLDSPSRQDITFYLIIRRKPLFYIIN | ID ENSG00000135902; Homo |
| | ILVPCVLISFMVNLVFYLPADSGEKTSVAISVLLAQSVFLLLISKRLPA | sapiens |
| | TSMAIPLIGKFLLFGMVLVTMVVVICVIVLNIHFRTPSTHVLSEGVKK | |
| | LFLETLPELLHMSRPAEDGPSPGALVRRSSSLGYISKAEEYFLLKSRS | |
| | DLMFEKQSERHGLARRLTTARRPPASSEQAQQELFNELKPAVDGAN | |
| | FIVNHMRDQNNYNEEKDSWNRVARTVDRLCLFVVTPVMVVGTAW | |
| | IFLQGVYNQPPPQPFPGDPYSYNVQDKRFI | |
| 34 | MEGPVLTLGLLAALAVCGSWGLNEEERLIRHLFQEKGYNKELRPVA | CHRND protein |
| | HKEESVDVALALTLSNLISLKEVEETLTTNVWIEHGWTDNRLKWNA | (ENSP00000410801) encoded |
| | EEFGNISVLRLPPDMQ | by Transcript ID |
| | | ENS100000446616 from Gene |
| | | ID ENSG00000135902; Homo |
| | | sapiens |
| 35 | MEGPVLTLGLLAALAVCGSWGLNEEERLIRHLFQEKGYNKELRPVA | CHRND protein |
| | HKEESVDVALALTLSNLISLKEVEETLTTNVWIEHGWTDNRLKWNA | (ENSP00000398143) encoded |
| | EEFGNISVLRLPPDMVWLPEIVLENNNDGSFQISYSCNVLVYHYGFV | by Transcript ID |
| | YWLPPAIFRSSCPISVTYFPFDWQNCSLKFRW | ENST00000412233 from Gene |
| | | ID ENSG00000135902; Homo |
| | | sapiens |
| 36 | MEGPVLTLGLLAALAVCGSWGLNEEERLIRHLFQEKGYNKELRPVA | CHRND protein |
| | HKEESVDVALALTLSNLISLGWTDNRLKWNAEEFGNISVLRLPPDM | (ENSP00000404950) encoded |
| | VWLPEIVLENNNDGSFQISYSCNVLVYHYGFVYWLPPAIFRSSCPISV | by Transcript ID |
| | TYFPFDWQNCSLKFSSLKYTAKEITLSLKQDA | ENST00000449596 from Gene |
| | | ID ENSG00000135902; Homo |
| | | sapiens |
| 37 | MHGGQGPLLLLLLLAVCLGAQGRNQEERLLADLMQNYDPNLRPAE | CHRNG protein |
| | RDSDVVNVSLKLTLTNLISLNEREEALTTNVWIEMQWCDYRLRWDP | (ENSP00000374143) encoded |
| | RDYEGLWVLRVPSTMVWRPDIVLENKSQTYSTNEIDLQLSQEDGQT | by Transcript ID |
| | IEWIFIDPEAFTENGEWAIQHRPAKMLLDPAAPAQEAGHQKVVFYLL | ENST00000389492 from Gene |
| | IQRKPLFYVINIIAPCVLISSVAILIHFLPAKAGGQKCTVAINVLLAQT | ID ENSG00000196811; Homo |
| | VFLFLVAKKVPETSQAVPLISKYLTFLLVVTILIVVNAVVVLNVSLRS | sapiens |
| | PHTHSMARGVRKVFLRLLPQLLRMHVRPLAPAAVQDTQSRLQNGS | |
| | SGWSITTGEEVALCLPRSELLFQQWQRQGLVAAALEKLEKGPELGL | |
| | SQFCGSLKQAAPAIQACVEACNLIACARHQQSHFDNGNEEWFLVGR | |
| | VLDRVCFLAMLSLFICGTAGIFLMAHYNRVPALPFPGDPRPYLPSPD | |
| 38 | MHGGQGPLLLLLLLAVCLGAQGRNQEERLLADLMQNYDPNLRPAE | CHRNG protein |
| | RDSDVVNVSLKLTLTNLISLNEREEALTTNVWIEMQWCDYRLRWDP | (ENSP00000374145) encoded |
| | RDYEGLWVLRVPSTMVWRPDIVLENNVDGVFEVALYCNVLVSPDG | by Transcript ID |
| | CIYWLPPAIFRSACSISVTYFPFDWQNCSLIFQSQTYSTNEIDLQLSQE | ENST00000389494 from Gene |
| | DGQTIEWIFIDPEAFTENGEWAIQHRPAKMLLDPAAPAQEAGHQKV | ID ENSG00000196811; Homo |
| | VFYLLIQRKPLFYVINIIAPCVLISSVAILIHFLPAKAGGQKCTVAINVL | sapiens |
| | LAQTVFLFLVAKKVPETSQAVPLISKYLTFLLVVTILIVVNAVVVLN | |
| | VSLRSPHTHSMARGVRKVFLRLLPQLLRMHVRPLAPAAVQDTQSRL | |
| | QNGSSGWSITTGEEVALCLPRSELLFQQWQRQGLVAAALEKLEKGP | |
| | ELGLSQFCGSLKQAAPAIQACVEACNLIACARHQQSHFDNGNEEWF | |
| | LVGRVLDRVCFLAMLSLFICGTAGIFLMAHYNRVPALPFPGDPRPYL | |
| | PSPD | |
| 39 | MSIEKIWAREILDSRGNPTVEVDLYTAKGLFRAAVPSGASTGIYEAL | ENO2 protein |
| | ELRDGDKQRYLGKGVLKAVDHINSTIAPALISSGLSVVEQEKLDNL | (ENSP00000229277) encoded |
| | MLELDGTENKSKFGANAILGVSLAVCKAGAAERELPLYRHIAQLAG | by Transcript ID |
| | NSDLILPVPAFNVINGGSHAGNKLAMQEFMILPVGAESFRDAMRLG | ENST00000229277 from Gene |
| | AEVYHTLKGVIKDKYGKDATNVGDEGGFAPNILENSEALELVKEAI | ID ENSG00000111674; Homo |
| | DKAGYTEKIVIGMDVAASEFYRDGKYDLDFKSPTDPSRYITGDQLG | sapiens; |
| | ALYQDFVRDYPVVSIEDPFDQDDWAAWSKFTANVGIQIVGDDLTVT | ENO2 protein |
| | NPKRIERAVEEKACNCLLLKVNQIGSVTEAIQACKLAQENGWGVM | (ENSP00000437402) encoded |
| | VSHRSGETEDTFIADLVVGLCTGQIKTGAPCRSERLAKYNQLMRIEE | by Transcript ID |
| | ELGDEARFAGHNFRNPSVL | ENST00000535366 from Gene |
| | | ID ENSG00000111674; Homo |
| | | sapiens; |
| | | ENO2 protein |
| | | (ENSP00000438873) encoded |
| | | by Transcript ID |
| | | ENST00000541477 from Gene |
| | | ID ENSG00000111674; Homo |
| | | sapiens |
| 40 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382510) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399601 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000496749) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000644660 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | HYFCDAWNTFDALIVVGSIVDIAITEVNPAEHTQCSPSMNAEENSRIS | |
| | ITFFRLFRVMRLVKLLSRGEGIRTLLWTFIKSFQALPYVALLIVMLFFI | |
| | YAVIGMQVFGKIALNDTTEINRNNNFQTFPQAVLLLFRCATGEAWQ | |
| | DIMLACMPGKKCAPESEPSNSTEGETPCGSSFAVFYFISFYMLCAFLII | |
| | NLFVAVIMDNFDYLTRDWSILGPHHLDEFKRIWAEYDPEAKGRIKH | |
| | LDVVTLLRRIQPPLGFGKLCPHRVACKRLVSMNMPLNSDGTVMFNA | |
| | TLFALVRTALRIKTEGNLEQANEELRAIIKKIWKRTSMKLLDQVVPP | |
| | AGDDEVTVGKFYATFLIQEYFRKFKKRKEQGLVGKPSQRNALSLQA | |
| | GLRTLHDIGPEIRRAISGDLTAEEELDKAMKEAVSAASEDDIFRRAG | |
| | GLFGNHVSYYQSDGRSAFPQTFTTQRPLHINKAGSSQGDTESPSHEK | |
| | LVDSTFTPSSYSSTGSNANINNANNTALGRLPRPAGYPSTVSTVEGH | |
| | GPPLSPAIRVQEVAWKLSSNRCHSRESQAAMAGQEETSQDETYEVK | |
| | MNHDTEACSEPSLLSTEMLSYQDDENRQLTLPEEDKRDIRQSPKRGF | |
| | LRSASLGRRASFHLECLKRQKDRGGDISQKTVLPLHLVHHQALAVA | |
| | GLSPLLQRSHSPASFPRPFATPPATPGSRGWPPQPVPTLRLEGVESSE | |
| | KLNSSFPSIHCGSWAETTPGGGGSSAARRVRPVSLMVPSQAGAPGR | |
| | QFHGSASSLVEAVLISEGLGQFAQDPKFIEVTTQELADACDMTIEEM | |
| | ESAADNILSGGAPQSPNGALLPFVNCRDAGQDRAGGEEDAGCVRAR | |
| | GRPSEEELQDSRVYVSSL | |
| 41 | MSIEKIWAREILDSRGNPTVEVDLYTAKGLFRAAVPSGASTGIYEAL | ENO2 protein |
| | ELRDGDKQRYLGKAKFGANAILGVSLAVCKAGAAERELPLYRHIAQ | (ENSP00000441490) encoded |
| | LAGNSDLILPVPAFNVINGGSHAGNKLAMQEFMILPVGAESFRDAM | by Transcript ID |
| | RLGAEVYHTLKGVIKDKYGKDATNVGDEGGFAPNILENSEALELVK | ENST00000538763 from Gene |
| | EAIDKAGYTEKIVIGMDVAASEFYRDGKYDLDFKSPTDPSRYITGDQ | ID ENSG00000111674; Homo |
| | LGALYQDFVRDYPVVSIEDPFDQDDWAAWSKFTANVGIQIVGDDLT | sapiens |
| | VTNPKRIERAVEEKACNCLLLKVNQIGSVTEAIQACKLAQENGWGV | |
| | MVSHRSGETEDTFIADLVVGLCTGQIKTGAPCRSERLAKYNQLMRIE | |
| | EELGDEARFAGHNFRNPSVL | |
| 42 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382512) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399603 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWMQD | (ENSP00000493781) encoded |
| | AMGYELPWVYFVSLVIFGSFFVLNLVLGVLSGEFSKEREKAKARGD | by Transcript ID |
| | FQKLREKQQLEEDLKGYLDWITQAEDIDPENEDEGMDEEKPRNMS | ENST00000647327 from Gene |
| | MPTSETESVNTENVAGGDIEGENCGARLAHRISKSKFSRYWRRWNR | ID ENSG00000285479; Homo |
| | FCRRKCRAAVKSNVFYWLVIFLVFLNTLTIASEHYNQPNWLTEVQD | sapiens |
| | TANKALLALFTAEMLLKMYSLGLQAYFVSLFNRFDCFVVCGGILETI | |
| | LVETKIMSPLGISVLRCVRLLRIFKITRYWNSLSNLVASLLNSVRSIAS | |
| | LLLLLFLFIIIFSLLGMQLFGGKFNFDEMQTRRSTFDNFPQSLLTVFQI | |
| | LTGEDWNSVMYDGIMAYGGPSFPGMLVCIYFIILFICGNYILLNVFL | |
| | AIAVDNLADAESLTSAQKEEEEEKERKKLARTASPEKKQELVEKPA | |
| | VGESKEEKIELKSITADGESPPATKINMDDLQPNENEDKSPYPNPETT | |
| | GEEDEEEPEMPVGPRPRPLSELHLKEKAVPMPEASAFFIFSSNNRFRL | |
| | QCHRIVNDTIFTNLILFFILLSSISLAAEDPVQHTSFRNHILFYFDIVFTT | |
| | IFTIEIALKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | GYFSDPWNVFDFLIVIGSIIDVILSETNPAEHTQCSPSMNAEENSRISIT | |
| | FFRLFRVMRLVKLLSRGEGIRTLLWTFIKSFQALPYVALLIVMLFFIY | |
| | AVIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEGNLEQANEELRAIIKKIWKRTSMKLLDQVVPPA | |
| | GDDEVTVGKFYATFLIQEYFRKFKKRKEQGLVGKPSQRNALSLQAG | |
| | LRTLHDIGPEIRRAISGDLTAEEELDKAMKEAVSAASEDDIFRRAGG | |
| | LFGNHVSYYQSDGRSAFPQTFTTQRPLHINKAGSSQGDTESPSHEKL | |
| | VDSTFTPSSYSSTGSNANINNANNTALGRLPRPAGYPSTVSTVEGHG | |
| | PPLSPAIRVQEVAWKLSSNRCHSRESQAAMAGQEETSQDETYEVKM | |
| | NHDTEACSEPSLLSTEMLSYQDDENRQLTLPEEDKRDIRQSPKRGFL | |
| | RSASLGRRASFHLECLKRQKDRGGDISQKTVLPLHLVHHQALAVAG | |
| | LSPLLQRSHSPASFPRPFATPPATPGSRGWPPQPVPTLRLEGVESSEK | |
| | LNSSFPSIHCGSWAETTPGGGGSSAARRVRPVSLMVPSQAGAPGRQF | |
| | HGSASSLVEAVLISEGLGQFAQDPKFIEVTTQELADACDMTIEEMES | |
| | AADNILSGGAPQSPNGALLPFVNCRDAGQDRAGGEEDAGCVRARG | |
| | RPSEEELQDSRVYVSSL | |
| 43 | LVKEAIDKAGYTEKIVIGMDVAASEFYRDGKYDLDFKSPTDPSRYIT | ENO2 protein |
| | GDQLGALYQDFVRDYPGNKGACSCLM | (ENSP00000476096) encoded |
| | | by Transcript ID |
| | | ENST00000535275 from Gene |
| | | ID ENSG00000111674; Homo |
| | | sapiens |
| 44 | MSIEKIWAREILDSRGNPTVEVDLYTAKGLFRAAVPSGASTGIYEAL | ENO2 protein |
| | ELRDGDKQRYLGKGVLKAVDHINSTIAPALISSGLSVVEQEKLDNL | (ENSP00000445788) encoded |
| | MLELDGTENK | by Transcript ID |
| | | ENST00000537688 from Gene |
| | | ID ENSG00000111674; Homo |
| | | sapiens |
| 45 | MSIEKIWAREILDSRGNPTVEVDLYTAKGLFRAAVPSGASTGIYEAL | ENO2 protein |
| | ELRDGDKQRYLGKGVLKAVDHINSTIAPALISSGLSVVEQEKLDNL | (ENSP00000438062) encoded |
| | MLELDGTENKSLELVKEAIDKAGYTEKIVIGMDVAASEFYRDGKYD | by Transcript ID |
| | LDFKSPTDPSRYITGDQLGALYQDFVRDYPVVSIEDPFDQDDWAAW | ENST00000545045 from Gene |
| | SKFTANVGIQIVGDDLTVTNPKRIERAVEEKACNCLLLKVNQIGSVT | ID ENSG00000111674; Homo |
| | EAIQACKLAQENGWGVMVSHRSGETEDTFIADLVVGLCTGQIKTGA | sapiens |
| | PCRSERLAKYNQLMRIEEELGDEARFAGHNFRNPSVL | |
| 46 | XVSIEDPFDQDDWAAWSKFTANVGIQIVGDDLTVTNPKRIERAVEE | ENO2 protein |
| | KACNCLLLKVNQIGSVTEAIQASRLVPRAVLNVWLNTTSS | (ENSP00000476112) encoded |
| | | by Transcript ID |
| | | ENST00000543975 from Gene |
| | | ID ENSG00000111674; Homo |
| | | sapiens |
| 47 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000324105) encoded |
| | KFMIELDGTENKSKFGANAILGVSLAVCKAGAAEKGVPLYRHIADL | by Transcript ID |
| | AGNPDLILPVPAFNVINGGSHAGNKLAMQEFMILPVGASSFKEAMRI | ENST00000323997 from Gene |
| | GAEVYHHLKGVIKAKYGKDATNVGDEGGFAPNILENNEALELLKT | ID ENSG00000108515; Homo |
| | AIQAAGYPDKVVIGMDVAASEFYRNGKYDLDFKSPDDPARHITGEK | sapiens; |
| | LGELYKSFIKNYPVVSIEDPFDQDDWATWTSFLSGVNIQIVGDDLTV | ENO3 protein |
| | TNPKRIAQAVEKKACNCLLLKVNQIGSVTESIQACKLAQSNGWGVM | (ENSP00000431087) encoded |
| | VSHRSGETEDTFIADLVVGLCTGQIKTGAPCRSERLAKYNQLMRIEE | by Transcript ID |
| | ALGDKAIFAGRKFRNPKAK | ENST00000518175 from Gene |
| | | ID ENSG00000108515; Homo |
| | | sapiens |
| 48 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382515) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399606 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494420) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000644691 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKILGNADYVFTSIFTLEIILKMTAYGAFLHKGSFCRNYFNILD | |
| | LLVVSVSLISFGIQSSAINVVKILRVLRVLRPLRAINRAKGLKHVVQC | |
| | VFVAIRTIGNIVIVTTLLQFMFACIGVQLFKGKLYTCSDSSKQTEAEC | |
| | KGNYITYKDGEVDHPIIQPRSWENSKFDFDNVLAAMMALFTVSTFE | |
| | GWPELLYRSIDSHTEDKGPIYNYRVEISIFFIIYIIIIAFFMMNIFVGFVI | |
| | VTFQEQGEQEYKNCELDKNQRQCVEYALKARPLRRYIPKNQHQYK | |
| | VWYVVNSTYFEYLMFVLILLNTICLAMQHYGQSCLFKIAMNILNML | |
| | FTGLFTVEMILKLIAFKPKHYFCDAWNTFDALIVVGSIVDIAITEVNP | |
| | AEHTQCSPSMNAEENSRISITFFRLFRVMRLVKLLSRGEGIRTLLWTF | |
| | IKSFQALPYVALLIVMLFFIYAVIGMQVFGKIALNDTTEINRNNNFQT | |
| | FPQAVLLLFRCATGEAWQDIMLACMPGKKCAPESEPSNSTEGETPC | |
| | GSSFAVFYFISFYMLCAFLIINLFVAVIMDNFDYLTRDWSILGPHHLD | |
| | EFKRIWAEYDPEAKGRIKHLDVVTLLRRIQPPLGFGKLCPHRVACKR | |
| | LVSMNMPLNSDGTVMFNATLFALVRTALRIKTEGNLEQANEELRAII | |
| | KKIWKRTSMKLLDQVVPPAGDDEVTVGKFYATFLIQEYFRKFKKRK | |
| | EQGLVGKPSQRNALSLQAGLRTLHDIGPEIRRAISGDLTAEEELDKA | |
| | MKEAVSAASEDDIFRRAGGLFGNHVSYYQSDGRSAFPQTFTTQRPL | |
| | H1NKAGSSQGDTESPSHEKLVDSTFTPSSYSSTGSNANINNANNTAL | |
| | GRLPRPAGYPSTVSTVEGHGPPLSPAIRVQEVAWKLSSNRCHSRESQ | |
| | AAMAGQEETSQDETYEVKMNHDTEACSEPSLLSTEMLSYQDDENR | |
| | QLTLPEEDKRDIRQSPKRGFLRSASLGRRASFHLECLKRQKDRGGDI | |
| | SQKTVLPLHLVHHQALAVAGLSPLLQRSHSPASFPRPFATPPATPGS | |
| | RGWPPQPVPTLRLEGVESSEKLNSSFPSIHCGSWAETTPGGGGSSAA | |
| | RRVRPVSLMVPSQAGAPGRQFHGSASSLVEAVLISEGLGQFAQDPKF | |
| | IEVTTQELADACDMTIEEMESAADNILSGGAPQSPNGALLPFVNCRD | |
| | AGQDRAGGEEDAGCVRARGRPSEEELQDSRVYVSSL | |
| 49 | MAMQKIFAREILDSRGNPTVEVDLHTAKGRFRAAVPSGASTGIYEA | ENO3 protein |
| | LELRDGDKGRYLGKAKFGANAILGVSLAVCKAGAAEKGVPLYRHI | (ENSP00000430636) encoded |
| | ADLAGNPDLILPVPAFNVINGGSHAGNKLAMQEFMILPVGASSFKEA | by Transcript ID |
| | MRIGAEVYHHLKGVIKAKYGKDATNVGDEGGFAPNILENNEALELL | ENST00000519584 from Gene |
| | KTAIQAAGYPDKVVIGMDVAASEFYRNGKYDLDFKSPDDPARHITG | ID ENSG00000108515; Homo |
| | EKLGELYKSFIKNYINVSIEDPFDQDDWATWTSFLSGVNIQIVGDDL | sapiens |
| | TVTNPKRIAQAVEKKACNCLLLKVNQIGSVTESIQACKLAQSNGWG | |
| | VMVSHRSGETEDTFIADLVVGLCTGQIKTGAPCRSERLAKYNQLMR | |
| | IEEALGDKAIFAGRKFRNPKAK | |
| 50 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000428811) encoded |
| | KFMIELDGTENKSKFGANAILGVSLAVCKAGAAEKGVPLYRHIADL | by Transcript ID |
| | AGNPDLILPVPAFNVIN | ENST00000522249 from Gene |
| | | ID ENSG00000108515; Homo |
| | | sapiens |
| 51 | MAMQKIFAREILDSRGNPTVEVDLHTAKDFFPSPLLPTWNQSWKLG | ENO3 protein |
| | EAGVSLCTASCP | (ENSP00000430554) encoded |
| | | by Transcript ID |
| | | ENST00000521659 from Gene |
| | | ID ENSG00000108515; Homo |
| | | sapiens |
| 52 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000467444) encoded |
| | KFMIELDGTENKSKFGANAILGVSLAVCKAGAAEKGVPLYRHIADL | by Transcript ID |
| | AGNPDLILPVPAFNVINGGSHAGNKLAMQEFMILPVGASSFKEAMRI | ENST00000520221 from Gene |
| | GAEVYHHLKGVI | ID ENSG00000108515; Homo |
| | | sapiens |
| 53 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000430055) encoded |
| | KFMIELDGTENKSKFGANAILGVSLAVCKAGAAEKGVPLYRHIADL | by Transcript ID |
| | AGNPDLILPVPAFNVINGGSHAGNKLAMQEFMILPVGASSFKEAMRI | ENST00000519602 from Gene |
| | GAEVYHHLKGVIKAKYGKDATNVGDEGGFAPNILENNEALELLKT | ID ENSG00000108515; Homo |
| | AIQAAGYPDKVVIGMDVAASEFYRNGKYDLDFKSPDDPARHITGEK | sapiens |
| | LGELYKSF | |
| 54 | MAMQKIFAREILDSRGNPTVEVDLHTAK | ENO3 protein |
| | | (ENSP00000467270) encoded |
| | | by Transcript ID |
| | | ENST00000519266 from Gene |
| | | ID ENSG00000108515; Homo |
| | | sapiens |
| 55 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000465697) encoded |
| | KFMIELDGTENKSKFGANAILGVSLAVCKAGAAEKGVPLYRHIADL | by Transcript ID |
| | AGNPDLILPVPAFNVINGGSHAGNKLAMQEFMILPVGASSFKEAMRI | ENST00000522301 from Gene |
| | GAEVYHHLKGV1KAKYGKDATNVGDEGG | ID ENSG00000108515; Homo |
| | | sapiens |
| 56 | MAMQKIFAREILDSRGNPTVEVDLHTAKGRFRAAVPSGASTGIYEA | ENO3 protein |
| | LELRDGDKGRYLGKGVLKAVENINNTLGPALLQKKLSVVDQEKVD | (ENSP00000464874) encoded |
| | KFMIELDGTENKSKFGANAILGVSLAVCKAGAAEKGVPLYRHIADL | by Transcript ID |
| | AGNPDLILPVP | ENST00000521811 from Gene |
| | | ID ENSG00000108515; Homo |
| | | sapiens; |
| | | ENO3 protein |
| | | (ENSP00000428502) encoded |
| | | by Transcript ID |
| | | ENST00000522798 from Gene |
| | | ID ENSG00000108515; Homo |
| | | sapiens |
| 57 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382526) encoded |
| | KQGSTTATRPPRALLCLTLKNP1RRACISIVEWKPFEIIILLTIFANCVA | by Transcript ID |
| | LAIYIPFPEDDSNATNSNLERVEYLFLIIFTVEAFLKVIAYGLLFHPNA | ENST00000399617 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWMQD | (ENSP00000494149) encoded |
| | AMGYELPWVYFVSLVIFGSFFVLNLVLGVLSGEFSKEREKAKARGD | by Transcript ID |
| | FQKLREKQQLEEDLKGYLDWITQAEDIDPENEDEGMDEEKPRNMS | ENST00000646280 from Gene |
| | MPTSETESVNTENVAGGDIEGENCGARLAHRISKSKFSRYWRRWNR | ID ENSG00000285479; Homo |
| | FCRRKCRAAVKSNVFYWLVIFLVFLNTLTIASEHYNQPNWLTEVQD | sapiens |
| | TANKALLALFTAEMLLKMYSLGLQAYFVSLFNRFDCFVVCGGILETI | |
| | LVETKIMSPLGISVLRCVRLLRIFKITRYWNSLSNLVASLLNSVRSIAS | |
| | LLLLLFLFIIIFSLLGMQLFGGKFNFDEMQTRRSTFDNFPQSLLTVFQI | |
| | LTGEDWNSVMYDGIMAYGGPSFPGMLVCIYFIILFICGNYILLNVFL | |
| | AIAVDNLADAESLTSAQKEEEEEKERKKLARTASPEKKQELVEKPA | |
| | VGESKEEKIELKSITADGESPPATKINMDDLQPNENEDKSPYPNPETT | |
| | GEEDEEEPEMPVGPRPRPLSELHLKEKAVPMPEASAFFIFSSNNRFRL | |
| | QCHRIVNDTIFTNLILFFILLSSISLAAEDPVQHTSFRNHILFYFDIVFTT | |
| | IFTIEIALKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | GYFSDPWNVFDFLIVIGSIIDVILSETNPAEHTQCSPSMNAEENSRISIT | |
| | FFRLFRVMRLVKLLSRGEGIRTLLWTFIKSFQALPYVALLIVMLFFIY | |
| | AVIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEGNLEQANEELRAIIKKIWKRTSMKLLDQVVPPA | |
| | GDDEVTVGKFYATFLIQEYFRKFKKRKEQGLVGKPSQRNALSLQAG | |
| | LRILHDIGPEIRRAISGDLTAEEELDKAMKEAVSAASEDDIFRRAGG | |
| | LFGNHVSYYQSDGRSAFPQTFTTQRPLHINKAGSSQGDTESPSHEKL | |
| | VDSTFTPSSYSSTGSNANINNANNTALGRLPRPAGYPSTVSTVEGHG | |
| | PPLSPAIRVQEVAWKLSSNRERHVPMCEDLELRRDSGSAGTQAHCL | |
| | LLRRANPSRCHSRESQAAMAGQEETSQDETYEVKMNHDTEACSEPS | |
| | LLSTEMLSYQDDENRQLTLPEEDKRDIRQSPKRGFLRSASLGRRASF | |
| | HLECLKRQKDRGGDISQKTVLPLHLVHHQALAVAGLSPLLQRSHSP | |
| | ASFPRPFATPPATPGSRGWPPQPVPTLRLEGVESSEKLNSSFPSIHCGS | |
| | WAETTPGGGGSSAARRVRPVSLMVPSQAGAPGRQFHGSASSLVEA | |
| | VLISEGLGQFAQDPKFIEVTTQELADACDMTIEEMESAADNILSGGA | |
| | PQSPNGALLPFVNCRDAGQDRAGGEEDAGCVRARGRPSEEELQDSR | |
| | VYVSSL | |
| 58 | MALGQKLFITMSRGAGRLQGTLWALVFLGILVGMVVPSPAGTRAN | FGF6 protein |
| | NTLLDSRGWGTLLSRSRAGLAGEIAGVNWESGYLVGIKRQRRLYCN | (ENSP00000228837) encoded |
| | VGIGFHLQVLPDGRISGTHEENPYSLLEISTVERGVVSLFGVRSALFV | by Transcript ID |
| | AMNSKGRLYATPSFQEECKFRETLLPNNYNAYESDLYQGTYIALSK | ENST00000228837 from Gene |
| | YGRVKRGSKVSPIMTVTHFLPRI | ID ENSG00000111241; Homo |
| | | sapiens |
| 59 | TVERGVVSLFGVRSALFVAMNSKGRLYATVK | FGF6 protein |
| | | (ENSP00000445479) encoded |
| | | by Transcript ID |
| | | ENST00000543077 from Gene |
| | | ID ENSG00000111241; Homo |
| | | sapiens |
| 60 | MSGWRYLICVSFLLTILLELTYQGPPVPASSSTKLLMTSYSMRSTVV | ITIH6 protein |
| | SRYAHTLVTSVLFNPHAEAHEAIFDLDLPHLAFISNFTMTINNKVYIA | (ENSP00000218436) encoded |
| | EVKEKFIQAKKIYEEAHQQGKTAAHVGIRDRESEKFRISTSLAAGTE | by Transcript ID |
| | VTFSLAYEELLQRHQGQYQLVVSLRPGQLVKRLSIEVTVSERTGISY | ENST00000218436 from Gene |
| | VHIPPLRTGRLRTNAHASEVDSPPSTRIERGETCVRITYCPTLQDQSSI | ID ENSG00000 102313; Homo |
| | SGSGIMADFLVQYDVVMEDIIGDVQIYDDYFIHYFAPRGLPPMEKNV | sapiens |
| | VFVIDVSSSMFGTKMEQTKTAMNVILSDLQANDYFNIISFSDTVNVW | |
| | KAGGSIQATIQNVHSAKDYLHCMEADGWTDVNSALLAAASVLNHS | |
| | NQEPGRGPSVGRIPLIIFLTDGEPTAGVTTPSVILSNVRQALGHRVSLF | |
| | SLAFGDDADFTLLRRLSLENRGIARRIYEDTDAALQLKGLYEEISMP | |
| | LLADVRLNYLGGLVGASPWAVFPNYFGGSELVVAGQVQPGKQELG | |
| | IHLAARGPKDQLLVAHHSEGATNNSQKAFGCPGEPAPNVAHFIRRL | |
| | WAYVTIGELLDAHFQARDTTTRHLLAAKVLNLSLEYNFVTPLTSLV | |
| | MVQPKQASEETRRQTSTSAGPDTIMPSSSSRHGLGVSTAQPALVPKV | |
| | ISPKSRPVKPKFYLSSTTTASTKKMLSSKELEPLGESPHTLSMPTYPK | |
| | AKIPAQQDSGTLAQPTLRTKPTILVPSNSGTLLPLKPGSLSHQNPDILP | |
| | TNSRTQVPPVKPGIPASPKADTVKCVTPLHSKPGAPSHPQLGALTSQ | |
| | APKGLPQSRPGVSTLQVPKYPLHTRPRVPAPKTRNNMPHLGPGILLS | |
| | KTPKILLSLKPSAPPHQISTSISLSKPETPNPHMPQTPLPPRPDRPRPPL | |
| | PESLSTFPNTISSSTGPSSTTTTSVLGEPLPMPFTPTLPPGRFWHQYDL | |
| | LPGPQRTRQVLGPSRPGVPTMSLLNSSRPTPEGSPPNLPILLPSSILPEA | |
| | ISLLLLPEELELLSESMVESKFVESLNPPAFYTFLTPDEDGSPNWDGN | |
| | SEEILGGAGGSMESQGSSVGLAKGTLPSIFTFSSSVDGDPHFVIQIPHS | |
| | EEKICFTLNGHPGDLLQLIEDPKAGLHVSGKLLGAPPRPGHKDQTRT | |
| | YFQIITVTTDKPRAYTITISRSSISLRGEGTLRLSWDQPALLKRPQLEL | |
| | YVAAAARLTLRLGPYLEFLVLRHRYRHPSTLQLPHLGFYVANGSGL | |
| | SPSARGLIGQFQHADIRLVTGPMGPCLRRHHGPDVPVILGKRLLKDS | |
| | PRLLPRWASCWLVKRSHVELLLGHPYLSYVL | |
| 61 | MTGGRFDFDDGGTYCGGWEEGKAHGHGICTGPKGQGEYSGSWSH | JPH1 protein |
| | GFEVVGGYTWPSGNTYQGYWAQGKRHGLGVETKGKWMYRGEWS | (ENSP00000344488) encoded |
| | HGFKGRYGVRQSLCTPARYEGTWSNGLQDGYGVETYGDGGTYQG | by Transcript ID |
| | QWAGGMRHGYGVRQSVPYGMATVIRSPLRTSLASLRSEQSNGSVL | ENST00000342232 from Gene |
| | HDAAAAADSPAGTRGGFVLNFHADAELAGKKKGGLFRRGSLLGSM | ID ENSG00000104369; Homo |
| | KLRKSESKSSISSKRSSVRSDAAMSRISSSDANSTISFGDVDCDFCPVE | sapiens |
| | DHVDATTTETYMGEWKNDKRNGFGVSERSNGMKYEGEWANNKR | |
| | HGYGCTVFPDGSKEEGKYKNNILVRGIRKQLIPIRHTKTREKVDRAI | |
| | EGAQRAAAMARTKVEIANSRTAHARAKADAADQAALAARQECDI | |
| | ARAVARELSPDFYQPGPDYVKQRFQEGVDAKENPEEKVPEKPPTPK | |
| | ESPHFYRKGTTPPRSPEASPKHSHSPASSPKPLKKQNPSSGARLNQDK | |
| | RSVADEQVTAIVNKPLMSKAPTKEAGAVVPQSKYSGRHHIPNPSNG | |
| | ELHSQYHGYYVKLNAPQHPPVDVEDGDGSSQSSSALVHKPSANKW | |
| | SPSKSVTKPVAKESKAEPKAKKSELAIPKNPASNDSCPALEKEANSG | |
| | PNSIMIVLVMLLNIGLAILFVHFLT | |
| 62 | MTGGRYLPGPVGRRHAAWLRRAPERALRHGHGDPLTAAYLAGLA | JPH1 protein |
| | AQRAEQWQRAPRRRSRRRQPGRHPRRFRAQLPRRR | (ENSP00000429652) encoded |
| | | by Transcript ID |
| | | ENST00000519947 from Gene |
| | | ID ENSG00000104369; Homo |
| | | sapiens |
| 63 | MSMTTRAWEELDGGLGSCQALEDHSALAETQEDRASATPRLADSG | JSRP1 protein |
| | SVPHDSQVAEGPSVDTRPKKMEKEPAARGTPGTGKERLKAGASPRS | (ENSP00000300961) encoded |
| | VPARKKAQTAPPLQPPPPPPALSEELPWGDLSLNKCLVLASLVALLG | by Transcript ID |
| | SAFQLCRDAVPGEAALQARVPEPWVPPSSAPREPSSPLPKFEAQAPP | ENST00000300961 from Gene |
| | SAPPAPRAEAEVRPKIPGSREAAENDEEEPGEATGEAVREDRVTLAD | ID ENSG00000167476; Homo |
| | RGPKERPRREGKPRKEKPRKEERPKKERPRKEERPRAAREPREALPQ | sapiens |
| | RWESREGGHRPWARDSRDAEPRKKQAWVSPRRPDEEQRPGSRQKL | |
| | RAGKGRD | |
| 64 | MEPRCPPPCGCCERLVLNVAGLRFETRARTLGRFPDTLLGDPARRG | KCNA7 protein |
| | RFYDDARREYFFDRHRPSFDAVLYYYQSGGRLRRPAHVPLDVFLEE | (ENSP00000221444) encoded |
| | VAFYGLGAAALARLREDEGCPVPPERPLPRRAFARQLWLLFEFPESS | by Transcript ID |
| | QAARVLAVVSVLVILVSIVVFCLETLPDFRDDRDGTGLAAAAAAGP | ENST00000221444 from Gene |
| | FPAPLNGSSQMPGNPPRLPFNDPFFVVETLCICWFSFELLVRLLVCPS | ID ENSG00000104848; Homo |
| | KAIFFKNVMNLIDFVAILPYFVALGTELARQRGVGQQAMSLAILRVI | sapiens |
| | RLVRVFRIFKLSRHSKGLQILGQTLRASMRELGLLIFFLFIGVVLFSSA | |
| | VYFAEVDRVDSHFTSIPESFWWAVVTMTTVGYGDMAPVTVGGKIV | |
| | GSLCAIAGVLTISLPVPVIVSNFSYFYHRETEGEEAGMFSHVDMQPC | |
| | GPLEGKANGGLVDGEVPELPPPLWAPPGKHLVTEV | |
| 65 | MDSQRELAEELRLYQSTLLQDGLKDLLDEKKFIDCTLKAGDKSLPC | KLHL41 protein |
| | HRLILSACSPYFREYFLSEIDEAKKKEVVLDNVDPAILDLIIKYLYSAS | (ENSP00000284669) encoded |
| | IDLNDGNVQDIFALASRFQIPSVFTVCVSYLQKRLAPGNCLAILRLGL | by Transcript ID |
| | LLDCPRLAISAREFVSDRFVQICKEEDFMQLSPQELISVISNDSLNVEK | ENST00000284669 from Gene |
| | EEAVFEAVMKWVRTDKENRVKNLSEVFDCIRFRLMTEKYFKDHVE | ID ENSG00000239474; Homo |
| | KDDIIKSNPDLQKKIKVLKDAFAGKLPEPSKNAAKTGAGEVNGDVG | sapiens |
| | DEDLLPGYLNDIPRHGMFVKDLILLVNDTAAVAYDPTENECYLTAL | |
| | AEQIPRNHSSIVTQQNQIYVVGGLYVDEENKDQPLQSYFFQLDSIASE | |
| | WVGLPPLPSARCLFGLGEVDDKIYVVAGKDLQTEASLDSVLCYDPV | |
| | AAKWNEVKIKLPIKVYGHNVISHKGMIYCLGGKTDDKKCTNRVFIFN | |
| | PKKGDWKDLAPMKIPRSMFGVAVHKGKIVIAGGVTEDGLSASVEAF | |
| | DLTTNKWDVMTEFPQERSSISLVSLAGSLYAIGGFAMIQLESKEFAP | |
| | TEVNDIWKYEDDKKEWAGMLKEIRYASGASCLATRLNLFKLSKL | |
| 66 | MDQPQFSGAPRFLTRPKAFVVSVGKDATLSCQIVGNPTPQVSWEKD | OBSCN protein |
| | QQPVAAGARFRLAQDGDLYRLTILDLALGDSGQYVCRARNAIGEAF | (ENSP00000284548) encoded |
| | AAVGLQVDAEAACAEQAPHFLLRPTSIRVREGSEATFRCRVGGSPRP | by Transcript ID |
| | AVSWSKDGRRLGEPDGPRVRVEELGEASALRIRAARPRDGGTYEVR | ENST00000284548 from Gene |
| | AENPLGAASAAAALVVDSDAADTASRPGTSTAALLAHLQRRREAM | ID ENSG00000154358; Homo |
| | RAEGAPASPPSTGTRTCTVTEGKHARLSCYVTGEPKPETVWKKDGQ | sapiens |
| | LVTEGRRHVVYEDAQENFVLKILFCKQSDRGLYTCTASNLVGQTYS | |
| | SVLVVVREPAVPFKKRLQDLEVREKESATFLCEVPQPSTEAAWFKE | |
| | ETRLWASAKYGIEEEGTERRLTVRNVSADDDAVYICETPEGSRTVA | |
| | ELAVQGNLLRKLPRKTAVRVGDTAMFCVELAVPVGPVHWLRNQEE | |
| | VVAGGRVAISAEGTRHTLTISQCCLEDVGQVAFMAGDCQTSTQFCV | |
| | SAPRKPPLQPPVDPVVKARMESSVILSWSPPPHGERPVTIDGYLVEK | |
| | KKLGTYTWIRCHEAEWVATPELTVADVAEEGNFQFRVSALNSFGQS | |
| | PYLEFPGTVHLAPKLAVRTPLKAVQAVEGGEVTFSVDLTVASAGEW | |
| | FLDGQALKASSVYEIHCDRTRHTLTIREVPASLHGAQLKFVANGIES | |
| | SIRMEVRAAPGLTANKPPAAAAREVLARLHEEAQLLAELSDQAAAV | |
| | TWLKDGRTLSPGPKYEVQASAGRRVLLVRDVARDDAGLYECVSRG | |
| | GRIAYQLSVQGLARFLHKDMAGSCVDAVAGGPAQFECETSEAHVH | |
| | VHWYKDGMELGHSGERFLQEDVGTRHRLVAATVTRQDEGTYSCR | |
| | VGEDSVDFRLRVSEPKVVFAKEQLARRKLQAEAGASATLSCEVAQA | |
| | QTEVTWYKDGKKLSSSSKVCMEATGCTRRLVVQQAGQADAGEYS | |
| | CEAGGQRLSFHLDVKEPKVVFAKDQVAHSEVQAEAGASATLSCEV | |
| | AQAQTEVMWYKDGKKLSSSLKVHVEAKGCRRRLVVQQAGKTDAG | |
| | DYSCEARGQRVSFRLHITEPKMMFAKEQSVHNEVQAEAGASAMLS | |
| | CEVAQAQTEVTWYKDGKKLSSSSKVGMEVKGCTRRLVLPQAGKA | |
| | DAGEYSCEAGGQRVSFHLHITEPKGVFAKEQSVHNEVQAEAGTTA | |
| | MLSCEVAQPQTEVTWYKDGKKLSSSSKVRMEVKGCTRRLVVQQV | |
| | GKADAGEYSCEAGGQRVSFQLHITEPKAVFAKEQLVHNEVRTEAG | |
| | ASATLSCEVAQAQTEVTWYKDGKKLSSSSKVRIEAAGCMRQLVVQ | |
| | QAGQADAGEYTCEAGGQRLSFHLDVSEPKAVFAKEQLAHRKVQAE | |
| | AGAIATLSCEVAQAQTEVTWYKDGKKLSSSSKVRMEAVGCTRRLV | |
| | VQQACQADTGEYSCEAGGQRLSFSLDVAEPKVVFAKEQPVHREVQ | |
| | AQAGASTTLSCEVAQAQTEVMWYKDGKKLSFSSKVRMEAVGCTR | |
| | RLVVQQAGQAVAGEYSCEAGSQRLSFHLHVAEPKAVFAKEQPASR | |
| | EVQAEAGTSATLSCEVAQAQTEVTWYKDGKKLSSSSKVRMEAVGC | |
| | TRRLVVQEAGQADAGEYSCKAGDQRLSFHLHVAEPKVVFAKEQPA | |
| | HREVQAEAGASATLSCEVAQAQTEVTWYKDGKKLSSSSKVRVEAV | |
| | GCTRRLVVQQAGQAEAGEYSCEAGGQQLSFRLQVAELEPQISERPC | |
| | RREPLVVKEHEDIILTATLATPSAATVTWLKDGVEIRRSKRHETASQ | |
| | GDTHTLTVHGAQVLDSAIYSCRVGAEGQDFPVQVEEVAAKFCRLLE | |
| | PVCGELGGTVTLACELSPACAEVVWRCGNTQLRVGKRFQMVAEGP | |
| | VRSLTVLGLRAEDAGEYVCESRDDHTSAQLTVSVPRVVKFMSGLST | |
| | VVAEEGGEATFQCVVSPSDVAVVWFRDGALLQPSEKFAISQSGASH | |
| | SLTISDLVLEDAGQITVEAEGASSSAALRVREAPVLFKKKLEPQTVEE | |
| | RSSVTLEVELTRPWPELRWTRNATALAPGKNVEIHAEGARHRLVLH | |
| | NVGFADRGFFGCETPDDKTQAKLTVEMRQVRLVRGLQAVEAREQG | |
| | TATMEVQLSHADDVDGSWTRDGLRFQQGPTCHLAVRGPMHTLTLSG | |
| | LRPEDSGLMVFKAEGVHTSARLVVTELPVSFSRPLQDVVTTEKEKV | |
| | TLECELSRPNVDVRWLKDGVELRAGKTMAIAAQGACRSLTIYRCEF | |
| | ADQGVYVCDAHDAQSSASVKVQGRTYTLIYRRVLAEDAGEIQFVA | |
| | ENAESRAQLRVKELPVTLVRPLRDKIAMEKHRGVLECQVSRASAQV | |
| | RWFKGSQELQPGPKYELVSDGLYRKLIISDVHAEDEDTYTCDAGDV | |
| | KTSAQFFVEEQSITIVRGLQDVTVMEPAPAWFECETSIPSVRPPKWLL | |
| | GKTVLQAGGNVGLEQEGTVHRLMLRRTCSTMTGPVHFTVGKSRSS | |
| | ARLVVSDIPVVLTRPLEPKTGRELQSVVLSCDFRPAPKAVQWYKDD | |
| | TPLSPSEKFKMSLEGQMAELRILRLMPADAGVYRCQAGSAHSSTEV | |
| | TVEAREVTVTGPLQDAEATEEGWASFSCELSHEDEEVEWSLNGMPL | |
| | YNDSFHEISHKGRRHTLVLKSIQRADAGIVRASSLKVSTSARLEVRV | |
| | KPVVFLKALDDLSAEERGTLALQCEVSDPEAHVVWRKDGVQLGPS | |
| | DKYDFLHTAGTRGLVVHDVSPEDAGLYTCHVGSEETRARVRVHDL | |
| | HVGITKRLKTMEVLEGESCSFECVLSHESASDPAMWTVGGKTVGSS | |
| | SRFQATRQGRKYILVVREAAPSDAGEVVFSVRGLTSKASLIVRERPA | |
| | AIIKPLEDQWVAPGEDVELRCELSRAGTPVHWLKDRKAIRKSQKYD | |
| | VVCEGTMAMLVIRGASLKDAGEYTCEVEASKSTASLHVEEKANCFT | |
| | EELTNLQVEEKGTAVFTCKTEHPAATVTWRKGLLELRASGKHQPSQ | |
| | EGLTLRLTISALEKADSDTYTCDIGQAQSRAQLLVQGRRVHIIEDLED | |
| | VDVQEGSSATFRCRISPANYEPVHWFLDKTPLHANELNEIDAQPGG | |
| | YHVLTLRQLALKDSGTIYFEAGDQRASAALRVTEKPSVFSRELTDAT | |
| | ITEGEDLTLVCETSTCDIPVCWTKDGKTLRGSARCQLSHEGHRAQLL | |
| | ITGATLQDSGRYKCEAGGACSSSIVRVHARPVRFQEALKDLEVLEG | |
| | GAATLRCVLSSVAAPVKWCYGNNVLRPGDKYSLRQEGAMLELVV | |
| | RNLRPQDSGRYSCSFGDQTTSATLTVTALPAQFIGKLRNKEATEGAT | |
| | ATLRCELSKAAPVEWRKGSETLRDGDRYCLRQDGAMCELQIRGLA | |
| | MVDAAEYSCVCGEERTSASLTIRPMPAHFIGRLRHQESIEGATATLR | |
| | CELSKAAPVEWRKGRESLRDGDRHSLRQDGAVCELQICGLAVADA | |
| | GEYSCVCGEERTSATLTVKALPAKFTEGLRNEEAVEGATAMLWCEL | |
| | SKVAPVEWRKGPENLRDGDRYILRQEGTRCELQICGLAMADAGEY | |
| | LCVCGQERTSATLTIRALPARFIEDVKNQEAREGATAVLQCELNSAA | |
| | PVEWRKGSETLRDGDRYSLRQDGTKCELQIRGLAMADTGEYSCVC | |
| | GQERTSAMLTVRALPIKFTEGLRNEEATEGATAVLRCELSKMAPVE | |
| | WWKGHETLRDGDRHSLRQDGARCELQIRGLVAEDAGEYLCMCGK | |
| | ERTSAMLTVRAMPSKFIEGLRNEEATEGDTATLWCELSKAAPVEWR | |
| | KGHETLRDGDRHSLRQDGSRCELQIRGLAVVDAGEYSCVCGQERTS | |
| | ATLTVRALPARFIEDVKNQEAREGATAVLQCELSKAAPVEWRKGSE | |
| | TLRGGDRYSLRQDGTRCELQIHGLSVADTGEYSCVCGQERTSATLT | |
| | VRAPQPVEREPLQSLQAEEGSTATLQCELSEPTATVVWSKGGLQLQ | |
| | ANGRREPRLQGCTAELVLQDLQREDTGEYTCTCGSQATSATLTVTA | |
| | APVRFLRELQHQEVDEGGTAHLCCELSRAGASVEWRKGSLQLFPCA | |
| | KYQMVQDGAAAELLVRGVEQEDAGDYTCDTGHTQSMASLSVRVP | |
| | RPKFKTRLQSLEQETGDIARLCCQLSDAESGAVVQWLKEGVELHAG | |
| | PKYEMRSQGATRELLIHQLEAKDTGEYACVTGGQKTAASLRVTEPE | |
| | VTIVRGLVDAEVTADEDVEFSCEVSRAGATGVQWCLQGLPLQSNEV | |
| | TEVAVRDGRIHTLRLKGVTPEDAGTVSFHLGNHASSAQLTVRAPEV | |
| | TILEPLQDVQLSEGQDASFQCRLSRASGQEARWALGGVPLQANEMN | |
| | DITVEQGTLHLLTLHKVTLEDAGTVSFHVGTCSSEAQLKVTAKNTV | |
| | VRGLENVEALEGGEALFECQLSQPEVAAHTWLLDDEPVHTSENAEV | |
| | VFFENGLRHLLLLKNLRPQDSCRVTFLAGDMVTSAFLTVRGWRLEI | |
| | LEPLKNAAVRAGAQACFTCTLSEAVPVGEASWYINGAAVQPDDSD | |
| | WTVTADGSHHALLLRSAQPHHAGEVTFACRDAVASARLTVLGLPD | |
| | PPEDAEVVARSSHTVTLSWAAPMSDGGGGLCGYRVEVKEGATGQ | |
| | WRLCHELVPGPECVVDGLAPGETYRFRVAAVGPVGAGEPVHLPQT | |
| | VRLAEPPKPVPPQPSAPESRQVAAGEDVSLELEVVAEAGEVIWHKG | |
| | MERIQPGGRFEVVSQGRQQMLVIKGFTAEDQGEYHCGLAQGSICPA | |
| | AATFQVALSPASVDEAPQPSLPPEAAQEGDLHLLWEALARKRRMSR | |
| | EPTLDSISELPEEDGRSQRLPQEAEEVAPDLSEGYSTADELARTGDA | |
| | DLSHTSSDDESRAGTPSLVTYLKKAGRPGTSPLASKVGAPAAPSVKP | |
| | QQQQEPLAAVRPPLGDLSTKDLGDPSMDKAAVKIQAAFKGYKVRK | |
| | EMKQQEGPMFSHTFGDTEAQVGDALRLECVVASKADVRARWLKD | |
| | GVELTDGRHHHIDQLGDGTCSLLITGLDRADAGCYTCQVSNKFGQV | |
| | THSACVVVSGSESEAESSSGGELDDAFRRAARRLHRLFRTKSPAEVS | |
| | DEELFLSADEGPAEPEEPADWQTYREDEHFICIRFEALTEARQAVTR | |
| | FQEMFATLGIGVEIKLVEQGPRRVEMCISKETPAPVVPPEPLPSLLTS | |
| | DAAPVFLTELQNQEVQDGYPVSFDCVVTGQPMPSVRWFKDGKLLE | |
| | EDDHYMINEDQQGGHQLIITAVVPADMGVYRCLAENSMGVSSTKA | |
| | ELRVDLTSTDYDTAADATESSSYFSAQGYLSSREQEGTESTTDEGQL | |
| | PQVVEELRDLQVAPGTRLAKFQLKVKGYPAPRLYWFKDGQPLTAS | |
| | AHIRMTDKKILHTLEIISVTREDSGQYAAYISNAMGAAYSSARLLVR | |
| | GPDEPEEKPASDVHEQLVPPRMLERFTPKKVKKGSSITFSVKVEGRP | |
| | VPTVHWLREEAERGVLWIGPDTPGYTVASSAQQHSLVLLDVGRQH | |
| | QGTYTCIASNAAGQALCSASLHVSGLPKVEEQEKVKEALISTFLQGT | |
| | TQAISAQGLETASFADLGGQRKEEPLAAKEALGHLSLAEVGTEEFLQ | |
| | KLTSQITEMVSAKITQAKLQVPGGDSDEDSKTPSASPRHGRSRPSSSI | |
| | QESSSESEDGDARGEIFDIYVVTADYLPLGAEQDAITLREGQYVEVL | |
| | DAAHPLRWLVRTKPTKSSPSRQGWVSPAYLDRRLKLSPEWGAAEA | |
| | PEFPGEAVSEDEYKARLSSVIQELLSSEQAFVEELQFLQSHHLQHLER | |
| | CPHVPIAVAGQKAVIFRNVRDIGRFHSSFLQELQQCDTDDDVAMCFI | |
| | KNQAAFEQYLEFLVGRVQAESVVVSTAIQEFYKKYAEEALLAGDPS | |
| | QPPPPPLQHYLEQPVERVQRYQALLKELIRNKARNRQNCALLEQAY | |
| | AVVSALPQRAENKLHVSLMENYPGTLQALGEPIRQGHFIVWEGAPG | |
| | ARMPWKGHNRHVFLFRNHLVICKPRRDSRTDTVSYVFRNMMKLSSI | |
| | DLNDQVEGDDRAFEVWQEREDSVRKYLLQARTAIIKSSWVKEICGI | |
| | QQRLALPVWRPPDFEEELADCTAELGETVKLACRVTGTPKPVISWY | |
| | KDGKAVQVDPHHILIEDPDGSCALILDSLTGVDSGQYMCFAASAAG | |
| | NCSTLGKILVQVPPRFVNKVRASPFVEGEDAQFTCTIEGAPYPQIRW | |
| | YKDGALLTTGNKFQTLSEPRSGLLVLVIRAASKEDLGLYECELVNRL | |
| | GSARASAELRIQSPMLQAQEQCHREQLVAAVEVTEQETKVPKKTVII | |
| | EETITTVVKSPRGQRRSPSKSPSRSPSRCSASPLRPGLLAPDLLYLPGA | |
| | GQPRRPEAEPGQKPVVPTLYVTEAEAHSPALPGLSGPQPKWVEVEE | |
| | TIEVRVKKMGPQGVSPTTEVPRSSSGHLFTLPGATPGGDPNSNNSNN | |
| | KLLAQEAWAQGTAMVGVREPLVFRVDARGSVDWAASGMGSLEEE | |
| | GTMEEAGEEEGEDGDAFVTEESQDTHSLGDRDPKILTHNGRMLTLA | |
| | DLEDYVPGEGETFHCGGPGPGAPDDPPCEVSVIQREIGEPTVGQPVL | |
| | LSVGHALGPRGPLGLFRPEPRGASPPGPQVRSLEGTSFLLREAPARPV | |
| | GSAPWTQSFCTRIRRSADSGQSSFTTELSTQTVNFGTVGETVTLHICP | |
| | DRDGDEAAQP | |
| 67 | MDQPQFSGAPRFLTRPKAFVVSVGKDATLSCQIVGNPTPQVSWEKD | OBSCN protein |
| | QQPVAAGARFRLAQDGDLYRLTILDLALGDSGQYVCRARNAIGEAF | (ENSP00000409493) encoded |
| | AAVGLQVDAEAACAEQAPHFLLRPTSIRVREGSEATFRCRVGGSPRP | by Transcript ID |
| | AVSWSKDGRRLGEPDGPRVRVEELGEASALRIRAARPRDGGTYEVR | ENST00000422127 from Gene |
| | AENPLGAASAAAALVVDSDAADTASRPGTSTAALLAHLQRRREAM | ID ENSG00000154358; Homo |
| | RAEGAPASPPSTGTRTCTVTEGKHARLSCYVTGEPKPETVWKKDGQ | sapiens; |
| | LVTEGRRHVVYEDAQENFVLKILFCKQSDRGLYTCTASNLVGQTYS | OBSCN protein |
| | SVLVVVREPAVPFKKRLQDLEVREKESATFLCEVPQPSTEAAWFKE | (ENSP00000490900) encoded |
| | ETRLWASAKYGIEEEGTERRLTVRNVSADDDAVYICETPEGSRTVA | by Transcript ID |
| | ELAVQGNLLRKLPRKTAVRVGDTAMFCVELAVPVGPVHWLRNQEE | ENST00000636875 from Gene |
| | VVAGGRVAISAEGTRHTLTISQCCLEDVGQVAFMAGDCQTSTQFCV | ID ENSG00000154358; Homo |
| | SAPRKPPLQPPVDPVVKARMESSVILSWSPPPHGERPVTIDGYLVEK | sapiens |
| | KKLGTYTWIRCHEAEWVATPELTVADVAEEGNFQFRVSALNSFGQS | |
| | PYLEFPGTVHLAPKLAVRTPLKAVQAVEGGEVTFSVDLTVASAGEW | |
| | FLDGQALKASSVYEIHCDRTRHTLTIREVPASLHGAQLKFVANGIES | |
| | SIRMEVRAAPGLTANKPPAAAAREVLARLHEEAQLLAELSDQAAAV | |
| | TWLKDGRTLSPGPKYEVQASAGRRVLLVRDVARDDAGLYECVSRG | |
| | GRIAYQLSVQGLARFLHKDMAGSCVDAVAGGPAQFECETSEAHVH | |
| | VHWYKDGMELGHSGERFLQEDVGTRHRLVAATVTRQDEGTYSCR | |
| | VGEDSVDFRLRVSEPKVVFAKEQLARRKLQAEAGASATLSCEVAQA | |
| | QTEVTWYKDGKKLSSSSKVCMEATGCTRRLVVQQAGQADAGEYS | |
| | CEAGGQRLSFHLDVKEPKVVFAKDQVAHSEVQAEAGASATLSCEV | |
| | AQAQTEVMWYKDGKKLSSSLKVHVEAKGCRRRLVVQQAGKTDAG | |
| | DYSCEARGQRVSFRLHITEPKMMFAKEQSVHNEVQAEAGASAMLS | |
| | CEVAQAQTEVTWYKDGKKLSSSSKVGMEVKGCTRRLVLPQAGKA | |
| | DAGEYSCEAGGQRVSFHLHITEPKGVFAKEQSVHNEVQAEAGTTA | |
| | MLSCEVAQPQTEVTWYKDGKKLSSSSKVRMEVKGCTRRLVVQQV | |
| | GKADAGEYSCEAGGQRVSFQLHITEPKAVFAKEQLVHNEVRTEAG | |
| | ASATLSCEVAQAQTEVTWYKDGKKLSSSSKVRIEAAGCMRQLVVQ | |
| | QAGQADAGEYTCEAGGQRLSFHLDVSEPKAVFAKEQLAHRKVQAE | |
| | AGAIATLSCEVAQAQTEVTWYKDGKKLSSSSKVRMEAVGCTRRLV | |
| | VQQACQADTGEYSCEAGGQRLSFSLDVAEPKVVFAKEQPVHREVQ | |
| | AQAGASTTLSCEVAQAQTEVMWYKDGKKLSFSSKVRMEAVGCTR | |
| | RLVVQQAGQAVAGEYSCEAGSQRLSFHLHVAEPKAVFAKEQPASR | |
| | EVQAEAGTSATLSCEVAQAQTEVTWYKDGKKLSSSSKVRMEAVGC | |
| | TRRLVVQEAGQADAGEYSCKAGDQRLSFHLHVAEPKVVFAKEQPA | |
| | HREVQAEAGASATLSCEVAQAQTEVTWYKDGKKLSSSSKVRVEAV | |
| | GCTRRLVVQQAGQAEAGEYSCEAGGQQLSFRLQVAELEPQISERPC | |
| | RREPLVVKEHEDIILTATLATPSAATVTWLKDGVEIRRSKRHETASQ | |
| | GDTHTLTVHGAQVLDSAIYSCRVGAEGQDFPVQVEEVAAKFCRLLE | |
| | PVCGELGGTVTLACELSPACAEVVWRCGNTQLRVGKRFQMVAEGP | |
| | VRSLTVLGLRAEDAGEYVCESRDDHTSAQLTVSVPRVVKFMSGLST | |
| | VVAEEGGEATFQCVVSPSDVAVVWFRDGALLQPSEKFAISQSGASH | |
| | SLTISDLVLEDAGQITVEAEGASSSAALRVREAPVLFKKKLEPQTVEE | |
| | RSSVTLEVELTRPWPELRWTRNATALAPGKNVEIHAEGARHRLVLH | |
| | NVGFADRGFFGCETPDDKTQAKLTVEMRQVRLVRGLQAVEAREQG | |
| | TATMEVQLSHADVDGSWTRDGLRFQQGPTCHLAVRGPMHTLTLSG | |
| | LRPEDSGLMVFKAEGVHTSARLVVTELPVSFSRPLQDVVTTEKEKV | |
| | TLECELSRPNVDVRWLKDGVELRAGKTMAIAAQGACRSLTIYRCEF | |
| | ADQGVYVCDAHDAQSSASVKVQGRTYTLIYRRVLAEDAGEIQFVA | |
| | ENAESRAQLRVKELPVTLVRPLRDKIAMEKHRGVLECQVSRASAQV | |
| | RWFKGSQELQPGPKYELVSDGLYRKLIISDVHAEDEDTYTCDAGDV | |
| | KTSAQFFVEEQSITIVRGLQDVTVMEPAPAWFECETSIPSVRPPKWLL | |
| | GKTVLQAGGNVGLEQEGTVHRLMLRRTCSTMTGPVHFTVGKSRSS | |
| | ARLVVSDIPVVLTRPLEPKTGRELQSVVLSCDFRPAPKAVQWYKDD | |
| | TPLSPSEKFKMSLEGQMAELRILRLMPADAGVYRCQAGSAHSSTEV | |
| | TVEAREVTVTGPLQDAEATEEGWASFSCELSHEDEEVEWSLNGMPL | |
| | YNDSFHEISHKGRRHTLVLKSIQRADAGIVRASSLKVSTSARLEVRV | |
| | KPVVFLKALDDLSAEERGTLALQCEVSDPEAHVVWRKDGVQLGPS | |
| | DKYDFLHTAGTRGLVVHDVSPEDAGLYTCHVGSEETRARVRVHDL | |
| | HVGITKRLKTMEVLEGESCSFECVLSHESASDPAMWTVGGKTVGSS | |
| | SRFQATRQGRKYILVVREAAPSDAGEVVFSVRGLTSKASLIVRERPA | |
| | AIIKPLEDQWVAPGEDVELRCELSRAGTPVHWLKDRKAIRKSQKYD | |
| | VVCEGTMAMLVIRGASLKDAGEYTCEVEASKSTASLHVEEKANCFT | |
| | EELTNLQVEEKGTAVFTCKTEHPAATVTWRKGLLELRASGKHQPSQ | |
| | EGLTLRLTISALEKADSDTYTCDIGQAQSRAQLLVQGRRVHIIEDLED | |
| | VDVQEGSSATFRCRISPANYEPVHWFLDKTPLHANELNEIDAQPGG | |
| | YHVLTLRQLALKDSGTIYFEAGDQRASAALRVTEKPSVFSRELTDAT | |
| | ITEGEDLTLVCETSTCDIPVCWTKDGKTLRGSARCQLSHEGHRAQLL | |
| | ITGATLQDSGRYKCEAGGACSSSIVRVHARPVRFQEALKDLEVLEG | |
| | GAATLRCVLSSVAAPVKWCYGNNVLRPGDKYSLRQEGAMLELVV | |
| | RNLRPQDSGRYSCSFGDQTTSATLTVTALPAQFIGKLRNKEATEGAT | |
| | ATLRCELSKAAPVEWRKGSETLRDGDRYCLRQDGAMCELQIRGLA | |
| | MVDAAEYSCVCGEERTSASLTIRPMPAHFIGRLRHQESIEGATATLR | |
| | CELSKAAPVEWRKGRESLRDGDRHSLRQDGAVCELQICGLAVADA | |
| | GEYSCVCGEERTSATLTVKALPAKFTEGLRNEEAVEGATAMLWCEL | |
| | SKVAPVEWRKGPENLRDGDRYILRQEGTRCELQICGLAMADAGEY | |
| | LCVCGQERTSATLTIRALPARFIEDVKNQEAREGATAVLQCELNSAA | |
| | PVEWRKGSETLRDGDRYSLRQDGTKCELQIRGLAMADTGEYSCVC | |
| | GQERTSAMLTVRALPIKFTEGLRNEEATEGATAVLRCELSKMAPVE | |
| | WWKGHETLRDGDRHSLRQDGARCELQIRGLVAEDAGEYLCMCGK | |
| | ERTSAMLTVRAMPSKFIEGLRNEEATEGDTATLWCELSKAAPVEWR | |
| | KGHETLRDGDRHSLRQDGSRCELQIRGLAVVDAGEYSCVCGQERTS | |
| | AILTVRALPARFIEDVKNQEAREGATAVLQCELSKAAPVEWRKGSE | |
| | TLRGGDRYSLRQDGTRCELQIHGLSVADTGEYSCVCGQERTSATLT | |
| | VRAPQPVFREPLQSLQAEEGSTATLQCELSEPTATVVWSKGGLQLQ | |
| | ANGRREPRLQGCTAELVLQDLQREDTGEYTCTCGSQATSATLTVTA | |
| | APVRFLRELQHQEVDEGGTAHLCCELSRAGASVEWRKGSLQLFPCA | |
| | KYQMVQDGAAAELLVRGVEQEDAGDYTCDTGHTQSMASLSVRVP | |
| | RPKFKTRLQSLEQETGDIARLCCQLSDAESGAVVQWLKEGVELHAG | |
| | PKYEMRSQGATRELLIHQLEAKDTGEYACVTGGQKTAASLRVTEPE | |
| | VTIVRGLVDAEVTADEDVEFSCEVSRAGATGVQWCLQGLPLQSNEV | |
| | TEVAVRDGRIHTLRLKGVTPEDAGTVSFHLGNHASSAQLTVRAPEV | |
| | TILEPLQDVQLSEGQDASFQCRLSRASGQEARWALGGVPLQANEMN | |
| | DITVEQGTLHLLTLHKVTLEDAGTVSFHVGTCSSEAQLKVTAKNTV | |
| | VRGLENVEALEGGEALFECQLSQPEVAAHTWLLDDEPVHTSENAEV | |
| | VFFENGLRHLLLLKNLRPQDSCRVTFLAGDMVTSAFLTVRGWRLEI | |
| | LEPLKNAAVRAGAQACFTCTLSEAVPVGEASWYINGAAVQPDDSD | |
| | WTVTADGSHHALLLRSAQPHHAGEVTFACRDAVASARLTVLGLPD | |
| | PPEDAEVVARSSHTVTLSWAAPMSDGGGGLCGYRVEVKEGATGQ | |
| | WRLCHELVPGPECVVDGLAPGETYRFRVAAVGPVGAGEPVHLPQT | |
| | VRLAEPPKPVPPQPSAPESRQVAAGEDVSLELEVVAEAGEVIWHKG | |
| | MERIQPGGRFEVVSQGRQQMLVIKGFTAEDQGEYHCGLAQGSICPA | |
| | AATFQVALSPASVDEAPQPSLPPEAAQEGDLHLLWEALARKRRMSR | |
| | EPTLDSISELPEEDGRSQRLPQEAEEVAPDLSEGYSTADELARTGDA | |
| | DLSHTSSDDESRAGTPSLVTYLKKAGRPGTSPLASKVGAPAAPSVKP | |
| | QQQQEPLAAVRPPLGDLSTKDLGDPSMDKAAVKIQAAFKGYKVRK | |
| | EMKQQEGPMFSHTFGDTEAQVGDALRLECVVASKADVRARWLKD | |
| | GVELTDGRHHHIDQLGDGTCSLLITGLDRADAGCYTCQVSNKFGQV | |
| | THSACVVVSGSESEAESSSGGELDDAFRRAARRLHRLFRTKSPAEVS | |
| | DEELFLSADEGPAEPEEPADWQTYREDEHFICIRFEALTEARQAVTR | |
| | FQEMFATLGIGVEIKLVEQGPRRVEMCISKETPAPVVPPEPLPSLLTS | |
| | DAAPVFLTELQNQEVQDGYPVSFDCVVTGQPMPSVRWFKDGKLLE | |
| | EDDHYMINEDQQGGHQLIITAVVPADMGVYRCLAENSMGVSSTKA | |
| | ELRVDLTSTDYDTAADATESSSYFSAQGYLSSREQEGTESTTDEGQL | |
| | PQVVEELRDLQVAPGTRLAKFQLKVKGYPAPRLYWFKDGQPLTAS | |
| | AHIRMTDKKILHTLEIISVTREDSGQYAAYISNAMGAAYSSARLLVR | |
| | GPDEPEEKPASDVHEQLVPPRMLERFTPKKVKKGSSITFSVKVEGRP | |
| | VPTVHWLREEAERGVLWIGPDTPGYTVASSAQQHSLVLLDVGRQH | |
| | QGTYTCIASNAAGQALCSASLHVSGLPKVEEQEKVKEALISTFLQGT | |
| | TQAISAQGLETASFADLGGQRKEEPLAAKEALGHLSLAEVGTEEFLQ | |
| | KLTSQITEMVSAKITQAKLQVPGGDSDEDSKTPSASPRHGRSRPSSSI | |
| | QESSSESEDGDARGEIFDIYVVTADYLPLGAEQDAITLREGQYVEVL | |
| | DAAHPLRWLVRTKPTKSSPSRQGWVSPAYLDRRLKLSPEWGAAEA | |
| | PEFPGEAVSEDEYKARLSSVIQELLSSEQAFVEELQFLQSHHLQHLER | |
| | CPHVPIAVAGQKAVIFRNVRDIGRFHSSFLQELQQCDTDDDVAMCFI | |
| | KNQAAFEQYLEFLVGRVQAESVVVSTAIQEFYKKYAEEALLAGDPS | |
| | QPPPPPLQHYLEQPVERVQRYQALLKELIRNKARNRQNCALLEQAY | |
| | AVVSALPQRAENKLHVSLMENYPGTLQALGEPIRQGHFIVWEGAPG | |
| | ARMPWKGHNRHVFLFRNHLVICKPRRDSRTDTVSYVFRNMMKLSSI | |
| | DLNDQVEGDDRAFEVWQEREDSVRKYLLQARTAIIKSSWVKEICGI | |
| | QQRLALPVWRPPDFEEELADCTAELGETVKLACRVTGTPKPVISWY | |
| | KDGKAVQVDPHHILIEDPDGSCALILDSLTGVDSGQYMCFAASAAG | |
| | NCSTLGKILVQVPPRFVNKVRASPFVEGEDAQFTCTIEGAPYPQIRW | |
| | YKDGALLTTGNKFQTLSEPRSGLLVLVIRAASKEDLGLYECELVNRL | |
| | GSARASAELRIQSPMLQAQEQCHREQLVAAVEDTTLERADQEVTSV | |
| | LKRLLGPKAPGPSTGDLTGPGPCPRGAPALQETGSQPPVTGTSEAPA | |
| | VPPRVPQPLLHEGPEQEPEAIARAQEWTVPIRMEGAAWPGAGTGEL | |
| | LWDVHSHVVRETTQRTYTYQAIDTHTARPPSMQVTIEDVQAQTGGT | |
| | AQFEAIIEGDPQPSVTWYKDSVQLVDSTRLSQQQEGTTYSLVLRHV | |
| | ASKDAGVYTCLAQNTGGQVLCKAELLVLGGDNEPDSEKQSHRRKL | |
| | HSFYEVKEEIGRGVFGFVKRVQHKGNKILCAAKFIPLRSRTRAQAYR | |
| | ERDILAALSHPLVTGLLDQFETRKTLILILELCSSEELLDRLYRKGVV | |
| | TEAEVKVYIQQLVEGLHYLHSHGVLHLDIKPSNILMVHPAREDIKIC | |
| | DFGFAQNITPAELQFSQYGSPEFVSPEIIQQNPVSEASDIWAMGVISY | |
| | LSLTCSSPFAGESDRATLLNVLEGRVSWSSPMAAHLSEDAKDFIKAT | |
| | LQRAPQARPSAAQCLSHPWFLKSMPAEEAHFINTKQLKFLLARSRW | |
| | QRSLMSYKSILVMRSIPELLRGPPDSPSLGVARHLCRDTGGSSSSSSS | |
| | SDNELAPFARAKSLPPSPVTHSPLLHPRGFLRPSASLPEEAEASERSTE | |
| | APAPPASPEGAGPPAAQGCVPRHSVIRSLFYHQAGESPEHGALAPGS | |
| | RRHPARRRHLLKGGYIAGALPGLREPLMEHRVLEEEAAREEQATLL | |
| | AKAPSPETALRLPASGTHLAPGHSHSLEHDSPSTPRPSSEACGEAQRL | |
| | PSAPSGGAPIRDMGHPQGSKQLPSTGGHPGTAQPERPSPDSPWGQPA | |
| | PFCHPKQGSAPQEGCSPHPAVAPCPPGSFPPGSCKEAPLVPSSPFLGQ | |
| | PQAPPAPAKASPPLDSKMGPGDISLPGRPKPGPCSSPGSASQASSSQV | |
| | SSLRVGSSQVGTEPGPSLDAEGWTQEAEDLSDSTPTLQRPQEQATM | |
| | RKFSLGGRGGYAGVAGYGTFAFGGDAGGMLGQGPMWARIAWAVS | |
| | QSEEEEQEEARAESQSEEQQEARAESPLPQVSARPVPEVGRAPTRSSP | |
| | EPTPWEDIGQVSLVQIRDLSGDAEAADTISLDISEVDPAYLNLSDLYD | |
| | IKYLPFEFMIFRKVPKSAQPEPPSPMAEEELAEFPEPTWPWPGELGPH | |
| | AGLEITEESEDVDALLAEAAVGRKRKWSSPSRSLFHFPGRHLPLDEP | |
| | AELGLRERVKASVEHISRILKGRPEGLEKEGPPRKKPGLASFRLSGLK | |
| | SWDRAPTFLRELSDETVVLGQSVTLACQVSAQPAAQATWSKDGAP | |
| | LESSSRVLISATLKNFQLLTILVVVAEDLGVYTCSVSNALGTVTTTG | |
| | VLRKAERPSSSPCPDIGEVYADGVLLVWKPVESYGPVTYIVQCSLEG | |
| | GSWTTLASDIFDCCYLTSKLSRGGTYTFRTACVSKAGMGPYSSPSEQ | |
| | VLLGGPSHLASEEESQGRSAQPLPSTKTAFQTQIQRGRFSVVRQCW | |
| | EKASGRALAAKIIPYHPKDKTAVLREYEALKGLRHPHLAQLHAAYL | |
| | SPRHLVLILELCSGPELLPCLAERASYSESEVKDYLWQMLSATQYLH | |
| | NQHILHLDLRSENMIITEYNLLKVVDLGNAQSLSQEKVLPSDKFKDY | |
| | LETMAPELLEGQGAVPQTDIWAIGVTAFIMLSAEYPVSSEGARDLQR | |
| | GLRKGLVRLSRCYAGLSGGAVAFLRSTLCAQPWGRPCASSCLQCP | |
| | WLTEEGPACSRPAPVTFPTARLRVFVRNREKRRALLYKRHNLAQVR | |
| 68 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382530) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399621 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494018) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000645584 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKH | |
| | YKDAWNTFDALIVVGSIVDIAITEVNPAEHTQCSPSMNAEENSRISI | |
| | TFFRLFRVMRLVKLLSRGEGIRTLLWTFIKSFQALPYVALLIVMLFFI | |
| | YAVIGMQVFGKIALNDTTEINRNNNFQTFPQAVLLLFRCATGEAWQ | |
| | DIMLACMPGKKCAPESEPSNSTEGETPCGSSFAVFYFISFYMLCAFLII | |
| | NLFVAVIMDNFDYLTRDWSILGPHHLDEFKRIWAEYDPEAKGRIKH | |
| | LDVVTLLRRIQPPLGFGKLCPHRVACKRLVSMNMPLNSDGTVMFNA | |
| | TLFALVRTALRIKTEEGPSPSEAHQGAEDPFRPAGNLEQANEELRAII | |
| | KKIWKRTSMKLLDQVVPPAGDDEVTVGKFYATFLIQEYFRKFKKRK | |
| | EQGLVGKPSQRNALSLQAGLRTLHDIGPEIRRAISGDLTAEEELDKA | |
| | MKEAVSAASEDDIFRRAGGLFGNHVSYYQSDGRSAFPQTFTTQRPL | |
| | HINKAGSSQGDTESPSHEKLVDSTFTPSSYSSTGSNANINNANNTAL | |
| | GRLPRPAGYPSTVSTVEGHGPPLSPAIRVQEVAWKLSSNRCHSRESQ | |
| | AAMAGQEETSQDETYEVKMNHDTEACSEPSLLSTEMLSYQDDENR | |
| | QLTLPEEDKRDIRQSPKRGFLRSASLGRRASFHLECLKRQKDRGGDI | |
| | SQKTVLPLHLVHHQALAVAGLSPLLQRSHSPASFPRPFATPPATPGS | |
| | RGWPPQPVPTLRLEGVESSEKLNSSFPSIHCGSWAETTPGGGGSSAA | |
| | RRVRPVSLMVPSQAGAPGRQFHGSASSLVEAVLISEGLGQFAQDPKF | |
| | IEVTTQELADACDMTIEEMESAADNILSGGAPQSPNGALLPFVNCRD | |
| | AGQDRAGGEEDAGCVRARGRPSEEELQDSRVYVSSL | |
| 69 | MDQPQFSGAPRFLTRPKAFVVSVGKDATLSCQIVGNPTPQVSWEKD | OBSCN protein |
| | QQPVAAGARFRLAQDGDLYRLTILDLALGDSGQYVCRARNAIGEAF | (ENSP00000355668) encoded |
| | AAVGLQVDAEAACAEQAPHFLLRPTSIRVREGSEATFRCRVGGSPRP | by Transcript ID |
| | AVSWSKDGRRLGEPDGPRVRVEELGEASALRIRAARPRDGGTYEVR | ENST00000366707 from Gene |
| | AENPLGAASAAAALVVDSDAADTASRPGTSTAALLAHLQRRREAM | ID ENSG00000154358; Homo |
| | RAEGAPASPPSTGTRTCTVTEGKHARLSCYVTGEPKPETVWKKDGQ | sapiens; |
| | LVTEGRRHVVYEDAQENFVLKILFCKQSDRGLYTCTASNLVGQTYS | OBSCN protein |
| | SVLVVVREPAVPFKKRLQDLEVREKESATFLCEVPQPSTEAAWFKE | (ENSP00000455507) encoded |
| | ETRLWASAKYGIEEEGTERRLTVRNVSADDDAVYICETPEGSRTVA | by Transcript ID |
| | ELAVQGNLLRKLPRKTAVRVGDTAMFCVELAVPVGPVHWLRNQEE | ENST00000570156 from Gene |
| | VVAGGRVAISAEGTRHTLTISQCCLEDVGQVAFMAGDCQTSTQFCV | ID ENSG00000154358; Homo |
| | SAPRKPPLQPPVDPVVKARMESSVILSWSPPPHGERPVTIDGYLVEK | sapiens |
| | KKLGTYTWIRCHEAEWVATPELTVADVAEEGNFQFRVSALNSFGQS | |
| | PYLEFPGTVHLAPKLAVRTPLKAVQAVEGGEVTFSVDLTVASAGEW | |
| | FLDGQALKASSVYEIHCDRTRHTLTIREVPASLHGAQLKFVANGIES | |
| | SIRMEVRAAPGLTANKPPAAAAREVLARLHEEAQLLAELSDQAAAV | |
| | TWLKDGRTLSPGPKYEVQASAGRRVLLVRDVARDDAGLYECVSRG | |
| | GRIAYQLSVQGLARFLHKDMAGSCVDAVAGGPAQFECETSEAHVH | |
| | VHWYKDGMELGHSGERFLQEDVGTRHRLVAATVTRQDEGTYSCR | |
| | VGEDSVDFRLRVSEPKAVFAKEQPACREVQAEVGASATLSCEVAQD | |
| | QMEVTWYKDGKKLSSSSKVHVEAVGCMRRLVVQQVGQADSGEYS | |
| | CEARGQRVSFRLDVAEPKVVFAKEQLARRKLQAEAGASATLSCEV | |
| | AQAQTEVTWYKDGKKLSSSSKVCMEATGCTRRLVVQQAGQADAG | |
| | EYSCEAGGQRLSFHLDVKEPKVVFAKDQVAHSEVQAEAGASATLS | |
| | CEVAQAQTEVMWYKDGKKLSSSLKVHVEAKGCRRRLVVQQAGKT | |
| | DAGDYSCEARGQRVSFRLHITEPKMMFAKEQSVHNEVQAEAGASA | |
| | MLSCEVAQAQTEVTWYKDGKKLSSSSKVGMEVKGCTRRLVLPQA | |
| | GKADAGEYSCEAGGQRVSFHLHITEPKGVFAKEQSVHNEVQAEAG | |
| | TTAMLSCEVAQPQTEVTWYKDGKKLSSSSKVRMEVKGCTRRLVVQ | |
| | QVGKADAGEYSCEAGGQRVSFQLHITEPKAVFAKEQLVHNEVRTE | |
| | AGASATLSCEVAQAQTEVTWYKDGKKLSSSSKVRIEAAGCMRQLV | |
| | VQQAGQADAGEYTCEAGGQRLSFHLDVSEPKAVFAKEQLAHRKVQ | |
| | AEAGAIATLSCEVAQAQTEVTWYKDGKKLSSSSKVRMEAVGCTRR | |
| | LVVQQACQADTGEYSCEAGGQRLSFSLDVAEPKVVFAKEQPVHRE | |
| | VQAQAGASTTLSCEVAQAQTEVMWYKDGKKLSFSSKVRMEAVGC | |
| | TRRLVVQQAGQAVAGEYSCEAGSQRLSFHLHVAEPKAVFAKEQPA | |
| | HREVQAEAGASATLSCEVAQAQTEVTWYKDGKKLSSSLKVHVEAA | |
| | GCTRRLVVQQAGQADTGEYSCEAGGQQLSFRLQVAEPKAVFAKEQ | |
| | PASREVQAEAGTSATLSCEVAQAQTEVTWYKDGKKLSSSSKVRME | |
| | AVGCTRRLVVQEAGQADAGEYSCKAGDQRLSFHLHVAEPKVVFAK | |
| | EQPAHREVQAEAGASATLSCEVAQAQTEVTWYKDGKKLSSSSKVR | |
| | VEAVGCTRRLVVQQAGQAEAGEYSCEAGGQQLSFRLQVAEPKAVF | |
| | AKEQVVFAKDQPVHREVQAEAGTSTMLSCEVAQAQTEVMWYKDG | |
| | KKLSSSSKMRVEAVGCTRRLVVQEAGQADAGEYSCEAGGQRLSFH | |
| | LHVAEPKVVFAKEQPACREVQAEAGASATLSCEVAQGQMEVTWY | |
| | KDGKKLSSSSKVHMEASGYTRRLVVQQAGQADAGEYSCEAGGQRL | |
| | SFRLHVAELEPQISERPCRREPLVVKEHEDIILTATLATPSAATVTWL | |
| | KDGVEIRRSKRHETASQGDTHTLTVHGAQVLDSAIYSCRVGAEGQD | |
| | FPVQVEEVAAKFCRLLEPVCGELGGTVTLACELSPACAEVVWRCGN | |
| | TQLRVGKRFQMVAEGPVRSLTVLGLRAEDAGEYVCESRDDHTSAQ | |
| | LTVSVPRVVKFMSGLSTVVAEEGGEATFQCVVSPSDVAVVWFRDG | |
| | ALLQPSEKFAISQSGASHSLTISDLVLEDAGQITVEAEGASSSAALRV | |
| | REAPVLFKKKLEPQTVEERSSVTLEVELTRPWPELRWTRNATALAP | |
| | GKNVEIHAEGARHRLVLHNVGFADRGFFGCETPDDKTQAKLTVEM | |
| | RQVRLVRGLQAVEAREQGTATMEVQLSHADVDGSWTRDGLRFQQ | |
| | GPTCHLAVRGPMHTLTLSGLRPEDSGLMVFKAEGVHTSARLVVTEL | |
| | PVSFSRPLQDVVTTEKEKVTLECELSRPNVDVRWLKDGVELRAGKT | |
| | MAIAAQGACRSLTIYRCEFADQGVYVCDAHDAQSSASVKVQGRNI | |
| | QIVRPLEDVEVMEKDGATFSCEVSHDEVPGQWFWEGSKLRPTDNV | |
| | RIRQEGRTYTLIYRRVLAEDAGEIQFVAENAESRAQLRVKELPVTLV | |
| | RPLRDKIAMEKHRGVLECQVSRASAQVRWFKGSQELQPGPKYELVS | |
| | DGLYRKLIISDVHAEDEDTYTCDAGDVKTSAQFFVEEQSITIVRGLQ | |
| | DVTVMEPAPAWFECETSIPSVRPPKWLLGKTVLQAGGNVGLEQEGT | |
| | VHRLMLRRTCSTMTGPVIIFTVGKSRSSARLVVSDIPVVLTRPLEPKT | |
| | GRELQSVVLSCDFRPAPKAVQWYKDDTPLSPSEKFKMSLEGQMAEL | |
| | RILRLMPADAGVYRCQAGSAHSSTEVTVEAREVTVTGPLQDAEATE | |
| | EGWASFSCELSHEDEEVEWSLNGMPLYNDSFHEISHKGRRHTLVLK | |
| | SIQRADAGIVRASSLKVSTSARLEVRVKPVVFLKALDDLSAEERGTL | |
| | ALQCEVSDPEAHVVWRKDGVQLGPSDKYDFLHTAGTRGLVVHDVS | |
| | PEDAGLYTCHVGSEETRARVRVHDLHVGITKRLKTMEVLEGESCSF | |
| | ECVLSHESASDPAMWTVGGKTVGSSSRFQATRQGRKYILVVREAAP | |
| | SDAGEVVFSVRGLTSKASLIVRERPAAIIKPLEDQWVAPGEDVELRC | |
| | ELSRAGTPVHWLKDRKAIRKSQKYDVVCEGTMAMLVIRGASLKDA | |
| | GEYTCEVEASKSTASLHVEEKANCFTEELTNLQVEEKGTAVFTCKT | |
| | EHPAATVTWRKGLLELRASGKHQPSQEGLTLRLTISALEKADSDTY | |
| | TCDIGQAQSRAQLLVQGRRVHIIEDLEDVDVQEGSSATFRCRISPAN | |
| | YEPVHWFLDKTPLHANELNEIDAQPGGYHVLTLRQLALKDSGTIYF | |
| | EAGDQRASAALRVTEKPSVFSRELTDATITEGEDLTLVCETSTCDIPV | |
| | CWTKDGKTLRGSARCQLSHEGHRAQLLITGATLQDSGRYKCEAGG | |
| | ACSSSIVRVHARPVRFQEALKDLEVLEGGAATLRCVLSSVAAPVKW | |
| | CYGNNVLRPGDKYSLRQEGAMLELVVRNLRPQDSGRYSCSFGDQT | |
| | TSATLTVTALPAQFIGKLRNKEATEGATATLRCELSKAAPVEWRKG | |
| | SETLRDGDRYCLRQDGAMCELQIRGLAMVDAAEYSCVCGEERTSA | |
| | SLTIRPMPAHFIGRLRHQESIEGATATLRCELSKAAPVEWRKGRESLR | |
| | DGDRHSLRQDGAVCELQICGLAVADAGEYSCVCGEERTSATLTVK | |
| | ALPAKFTEGLRNEEAVEGATAMLWCELSKVAPVEWRKGPENLRDG | |
| | DRYILRQEGTRCELQICGLAMADAGEYLCVCGQERTSATLTIRALPA | |
| | RFIEDVKNQEAREGATAVLQCELNSAAPVEWRKGSETLRDGDRYSL | |
| | RQDGTKCELQIRGLAMADTGEYSCVCGQERTSAMLTVRALPIKFTE | |
| | GLRNEEATEGATAVLRCELSKMAPVEWWKGHETLRDGDRHSLRQ | |
| | DGARCELQIRGLVAEDAGEYLCMCGKERTSAMLTVRAMPSKFIEGL | |
| | RNEEATEGDTATLWCELSKAAPVEWRKGHETLRDGDRHSLRQDGS | |
| | RCELQIRGLAVVDAGEYSCVCGQERTSATLTVRALPARFIEDVKNQ | |
| | EAREGATAVLQCELSKAAPVEWRKGSETLRGGDRYSLRQDGTRCE | |
| | LQIHGLSVADTGEYSCVCGQERTSATLTVRALPARFTQDLKTKEASE | |
| | GATATLQCELSKVAPVEWKKGPETLRDGGRYSLKQDGTRCELQIHD | |
| | LSVADAGEYSCMCGQERTSATLTVRALPARFTEGLRNEEAMEGAT | |
| | ATLQCELSKAAPVEWRKGLEALRDGDKYSLRQDGAVCELQIHGLA | |
| | MADNGVYSCVCGQERTSATLTVRALPARFIEDMRNQKATEGATVT | |
| | LQCKLRKAAPVEWRKGPNTLKDGDRYSLKQDGTSCELQIRGLVIAD | |
| | AGEYSCICEQERTSATLTVRALPARFIEDVRNHEATEGATAVLQCEL | |
| | SKAAPVEWRKGSETLRDGDRYSLRQDGTRCELQIRGLAVEDTGEYL | |
| | CVCGQERTSATLTVRALPARFIDNMTNQEAREGATATLHCELSKVA | |
| | PVEWRKGPETLRDGDRHSLRQDGTRCELQIRGLSVADAGEYSCVCG | |
| | QERTSATLTIRALPAKFTKGLRNEEATEGATAMLQCELSKVAPVEW | |
| | RKGPETLRDGDRYNLRQDGTRCELQIHGLSVADTGEYSCVCGQEKT | |
| | SATLTVKAPQPVFREPLQSLQAEEGSTATLQCELSEPTATVVWSKGG | |
| | LQLQANGRREPRLQGCTAELVLQDLQREDTGEYTCTCGSQATSATL | |
| | TVTAAPVRFLRELQHQEVDEGGTAHLCCELSRAGASVEWRKGSLQ | |
| | LFPCAKYQMVQDGAAAELLVRGVEQEDAGDYTCDTGHTQSMASL | |
| | SVRVPRPKFKTRLQSLEQETGDIARLCCQLSDAESGAVVQWLKEGV | |
| | ELHAGPKYEMRSQGATRELLIHQLEAKDTGEYACVTGGQKTAASL | |
| | RVTEPEVTIVRGLVDAEVTADEDVEFSCEVSRAGATGVQWCLQGLP | |
| | LQSNEVTEVAVRDGRIHTLRLKGVTPEDAGTVSFHLGNHASSAQLT | |
| | VRAPEVTILEPLQDVQLSEGQDASFQCRLSRASGQEARWALGGVPL | |
| | QANEMNDITVEQGTLHLLTLHKVTLEDAGTVSFHVGTCSSEAQLKV | |
| | TAKNTVVRGLENVEALEGGEALFECQLSQPEVAAHTWLLDDEPVH | |
| | TSENAEVVFFENGLRHLLLLKNLRPQDSCRVTFLAGDMVTSAFLTV | |
| | RGWRLEILEPLKNAAVRAGAQACFTCTLSEAVPVGEASWYINGAAV | |
| | QPDDSDWTVTADGSHIIALLLRSAQPHHAGEVTFACRDAVASARLT | |
| | VLGLPDPPEDAEVVARSSHTVTLSWAAPMSDGGGGLCGYRVEVKE | |
| | GATGQWRLCHELVPGPECVVDGLAPGETYRFRVAAVGPVGAGEPV | |
| | HLPQTVRLAEPPKPVPPQPSAPESRQVAAGEDVSLELEVVAEAGEVI | |
| | WHKGMERIQPGGRFEVVSQGRQQMLVIKGFTAEDQGEYHCGLAQG | |
| | SICPAAATFQVALSPASVDEAPQPSLPPEAAQEGDLHLLWEALARKR | |
| | RMSREPTLDSISELPEEDGRSQRLPQEAEEVAPDLSEGYSTADELART | |
| | GDADLSHTSSDDESRAGTPSLVTYLKKAGRPGTSPLASKVGAPAAPS | |
| | VKPQQQQEPLAAVRPPLGDLSTKDLGDPSMDKAAVKIQAAFKGYK | |
| | VRKEMKQQEGPMFSHTFGDTEAQVGDALRLECVVASKADVRARW | |
| | LKDGVELTDGRHHHIDQLGDGTCSLLITGLDRADAGCYTCQVSNKF | |
| | GQVTHSACVVVSGSESEAESSSGGELDDAFRRAARRLHRLFRTKSP | |
| | AEVSDEELFLSADEGPAEPEEPADWQTYREDEHFICIRFEALTEARQ | |
| | AVTRFQEMFATLGIGVEIKLVEQGPRRVEMCISKETPAPVVPPEPLPS | |
| | LLTSDAAPVFLTELQNQEVQDGYPVSFDCVVTGQPMPSVRWFKDG | |
| | KLLEEDDHYMINEDQQGGHQLIITAVVPADMGVYRCLAENSMGVS | |
| | STKAELRVDLTSTDYDTAADATESSSYFSAQGYLSSREQEGTESTTD | |
| | EGQLPQVVEELRDLQVAPGTRLAKFQLKVKGYPAPRLYWFKDGQP | |
| | LTASAHIRMTDKKILHTLEIISVTREDSGQYAAYISNAMGAAYSSAR | |
| | LLVRGPDEPEEKPASDVHEQLVPPRMLERFTPKKVKKGSSITFSVKV | |
| | EGRPVPTVHWLREEAERGVLWIGPDTPGYTVASSAQQHSLVLLDVG | |
| | RQHQGTYTCIASNAAGQALCSASLHVSGLPKVEEQEKVKEALISTFL | |
| | QGTTQAISAQGLETASFADLGGQRKEEPLAAKEALGHLSLAEVGTE | |
| | EFLQKLTSQITEMVSAKITQAKLQVPGGDSDEDSKTPSASPRHGRSR | |
| | PSSSIQESSSESEDGDARGEIFDIYVVTADYLPLGAEQDAITLREGQY | |
| | VEVLDAAHPLRWLVRTKPTKSSPSRQGWVSPAYLDRRLKLSPEWG | |
| | AAEAPEFPGEAVSEDEYKARLSSVIQELLSSEQAFVEELQFLQSHHL | |
| | QHLERCPHVPIAVAGQKAVIFRNVRDIGRFHSSFLQELQQCDTDDDV | |
| | AMCFIKNQAAFEQYLEFLVGRVQAESVVVSTAIQEFYKKYAEEALL | |
| | AGDPSQPPPPPLQHYLEQPVERVQRYQALLKELIRNKARNRQNCAL | |
| | LEQAYAVVSALPQRAENKLHVSLMENYPGTLQALGEPIRQGHFIVW | |
| | EGAPGARMPWKGHNRHVFLFRNHLVICKPRRDSRTDTVSYVFRNM | |
| | MKLSSIDLNDQVEGDDRAFEVWQEREDSVRKYLLQARTAIIKSSWV | |
| | KEICGIQQRLALPVWRPPDFEEELADCTAELGETVKLACRVTGTPKP | |
| | VISWYKDGKAVQVDPHHILIEDPDGSCALILDSLTGVDSGQYMCFA | |
| | ASAAGNCSTLGKILVQVPPRFVNKVRASPFVEGEDAQFTCTIEGAPY | |
| | PQIRWYKDGALLTTGNKFQTLSEPRSGLLVLVIRAASKEDLGLYECE | |
| | LVNRLGSARASAELRIQSPMLQAQEQCHREQLVAAVEDTTLERADQ | |
| | EVTSVLKRLLGPKAPGPSTGDLTGPGPCPRGAPALQETGSQPPVTGT | |
| | SEAPAVPPRVPQPLLHEGPEQEPEAIARAQEWTVPIRMEGAAWPGA | |
| | GTGELLWDVHSHVVRETTQRTYTYQAIDTHTARPPSMQVTIEDVQA | |
| | QTGGTAQFEAIIEGDPQPSVTWYKDSVQLVDSTRLSQQQEGTTYSLV | |
| | LRHVASKDAGVYTCLAQNTGGQVLCKAELLVLGGDNEPDSEKQSH | |
| | RRKLHSFYEVKEEIGRGVFGFVKRVQHKGNKILCAAKFIPLRSRTRA | |
| | QAYRERDILAALSHPLVTGLLDQFETRKTLILILELCSSEELLDRLYR | |
| | KGVVTEAEVKVYIQQLVEGLHYLHSHGVLHLDIKPSNILMVHPARE | |
| | DIKICDFGFAQNITPAELQFSQYGSPEFVSPEIIQQNPVSEASDIWAMG | |
| | VISYLSLTCSSPFAGESDRATLLNVLEGRVSWSSPMAAHLSEDAKDF | |
| | IKATLQRAPQARPSAAQCLSHPWFLKSMPAEEAHFINTKQLKFLLAR | |
| | SRWQRSLMSYKSILVMRSIPELLRGPPDSPSLGVARHLCRDTGGSSSS | |
| | SSSSDNELAPFARAKSLPPSPVTHSPLLHPRGFLRPSASLPEEAEASER | |
| | STEAPAPPASPEGAGPPAAQGCVPRHSVIRSLFYHQAGESPEHGALA | |
| | PGSRRHPARRRHLLKGGYIAGALPGLREPLMEHRVLEEEAAREEQA | |
| | TLLAKAPSFETALRLPASGTHLAPGHSHSLEHDSPSTPRPSSEACGEA | |
| | QRLPSAPSGGAPIRDMGHPQGSKQLPSTGGHPGTAQPERPSPDSPWG | |
| | QPAPFCHPKQGSAPQEGCSPHPAVAPCPPGSFPPGSCKEAPLVPSSPF | |
| | LGQPQAPPAPAKASPPLDSKMGPGDISLPGRPKPGPCSSPGSASQASS | |
| | SQVSSLRVGSSQVGTEPGPSLDAEGWTQEAEDLSDSTPTLQRPQEQA | |
| | TMRKFSLGGRGGYAGVAGYGTFAFGGDAGGMLGQGPMWARIAW | |
| | AVSQSEEEEQEEARAESQSEEQQEARAESPLPQVSARPVPEVGRAPT | |
| | RSSPEPTPWEDIGQVSLVQIRDLSGDAEAADTISLDISEVDPAYLNLS | |
| | DLYDIKYLPFEFMIFRKVPKSAQPEPPSPMAEEELAEFPEPTWPWPGE | |
| | LGPHAGLEITEESEDVDALLAEAAVGRKRKWSSPSRSLFHFPGRHLP | |
| | LDEPAELGLRERVKASVEHISRILKGRPEGLEKEGPPRKKPGLASFRL | |
| | SGLKSWDRAPTFLRELSDETVVLGQSVTLACQVSAQPAAQATWSK | |
| | DGAPLESSSRVLISATLKNFQLLTILVVVAEDLGVYTCSVSNALGTV | |
| | TTTGVLRKAERPSSSPCPDIGEVYADGVLLVWKPVESYGPVTYIVQC | |
| | SLEGGSWTTLASDIFDCCYLTSKLSRGGTYTFRTACVSKAGMGPYSS | |
| | PSEQVLLGGPSHLASEEESQGRSAQPLPSTKTFAFQTQIQRGRFSVVR | |
| | QCWEKASGRALAAKIIPYHPKDKTAVLREYEALKGLRHPHLAQLHA | |
| | AYLSPRHLVLILELCSGPELLPCLAERASYSESEVKDYLWQMLSATQ | |
| | YLHNQHILHLDLRSENMIITEYNLLKVVDLGNAQSLSQEKVLPSDKF | |
| | KDYLETMELLEGQGAVPQTDIWAIGVTAFIMLSAEYPVSSEGARDL | |
| | QRGLRKGLVRLSRCYAGLSGGAVAFLRSTLCAQPWGRPCASSCLQC | |
| | PWLTEEGPACSRPAPVTFPTARLRVFVRNREKRRALLYKRHNLAQV | |
| | R | |
| 70 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382537) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399629 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000496681) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000644891 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNEDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTILLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | GYFSDPWNVFDFLIVIGSIIDVILSETNHYFCDAWNTFDALIVVGSIV | |
| | DIAITEVNNAEENSRISITFFRLFRVMRLVKLLSRGEGIRTLLWTFIKS | |
| | FQALPYVALLIVMLFFIYAVIGMQVFGKIALNDTTEINRNNNFQTFPQ | |
| | AVLLLFRCATGEAWQDIMLACMPGKKCAPESEPSNSTEGETPCGSSF | |
| | AVFYFISFYMLCAFLIINLFVAVIMDNFDYLTRDWSILGPHHLDEFKR | |
| | IWAEYDPEAKGRIKHLDVVTLLRRIQPPLGFGKLCPHRVACKRLVS | |
| | MNMPLNSDGTVMFNATLFALVRTALRIKTEGNLEQANEELRAIIKKI | |
| | WKRTSMKLLDQVVPPAGDDEVTVGKFYATFLIQEYFRKFKKRKEQ | |
| | GLVGKPSQRNALSLQAGLRTLHDIGPEIRRAISGDLTAEEELDKAMK | |
| | EAVSAASEDDIFRRAGGLFGNHVSYYQSDGRSAFPQTFTTQRPLHIN | |
| | KAGSSQGDTESPSHEKLVDSTFTPSSYSSTGSNANINNANNTALGRL | |
| | PRPAGYPSTVSTVEGHGPPLSPAIRVQEVAWKLSSNRCHSRESQAA | |
| | MAGQEETSQDETYEVKMNHDTEACSEPSLLSTEMLSYQDDENRQL | |
| | TLPEEDKRDIRQSPKRGFLRSASLGRRASFHLECLKRQKDRGGDISQ | |
| | KTVLPLHLVHHQALAVAGLSPLLQRSHSPASFPRPFATPPATPGSRG | |
| | WPPQPVPTLRLEGVESSEKLNSSFPSIHCGSWAETTPGGGGSSAARR | |
| | VRPVSLMVPSQAGAPGRQFHGSASSLVEAVLISEGLGQFAQDPKFIE | |
| | VTTQELADACDMTIEEMESAADNILSGGAPQSPNGALLPFVNCRDA | |
| | GQDRAGGEEDAGCVRARGRPSEEELQDSRVYVSSL | |
| 71 | AGKTMAIAAQGACRSLTIYRCEFADQGVYVCDAHDAQSSASVKVQ | OBSCN protein |
| | GRNIQIVRPLEDVEVMEKDGATFSCEVSHDEVPGQWFWEGSKLRPT | (ENSP00000355667) encoded |
| | DNVRIKQEGRTYTLIYRRVLAEDAGEIQFVAENAESRAQLRVKELPV | by Transcript ID |
| | TLVRPLRDKIAMEKHRGVLECQVSRASAQVRWFKGSQELQPGPKY | ENST00000366706 from Gene |
| | ELVSDGLYRKLIISDVHAEDEDTYTCDAGDVKTSAQFFVEEQSITIVR | ID ENSG00000154358; Homo |
| | GLQDVTVMEPAPAWFECETSIPSVRPPKWLLGKTVLQAGGNVGLEQ | sapiens |
| | EGTVHRLMLRRTCSTMTGPVHFTVGKSRSSARLVVSDIPVVLTRPLE | |
| | PKTGRELQSVVLSCDFRPAPKAVQWYKDDTPLSPSEKFKMSLEGQM | |
| | AELRILRLMPADAGVYRCQAGSAHSSTEVTVEAREVTVTGPLQDAE | |
| | ATEEGWASFSCELSHEDEEVEWSLNGMPLYNDSFHEISHKGRRHTL | |
| | VLKSIQRADAGIVRASSLKVSTSARLEVRVKPVVFLKALDDLSAEER | |
| | GTLALQCEVSDPEAHVVWRKDGVQLGPSDKYDFLHTAGTRGLVVH | |
| | DVSPEDAGLYTCHVGSEETRARVRVHDLHVGITKRLKTMEVLEGES | |
| | CSFECVLSHESASDPAMWTVGGKTVGSSSRFQATRQGRKYILVVRE | |
| | AAPSDAGEVVFSVRGLTSKASLIVRERPAAIIKPLEDQWVAPGEDVE | |
| | LRCELSRAGTPVHWLKDRKAIRKSQKYDVVCEGTMAMLVIRGASL | |
| | KDAGEYTCEVEASKSTASLHVEEKANCFTEELTNLQVEEKGTAVFT | |
| | CKTEHPAATVTWRKGLLELRASGKHQPSQEGLTLRLTISALEKADS | |
| | DTYTCDIGQAQSRAQLLVQGEAAKCARA | |
| 72 | XRARVRVHDLHVGITKRLKTMEVLEGESCSFECVLSHESASDPAMW | OBSCN protein |
| | TVGGKTVGSSSRFQATRQGRKYILVVREAAPSDAGEVVFSVRGLTS | (ENSP00000455688) encoded |
| | KASLIVRERPAAIIKPLEDQWVAPGEDVELRCELSRAGTPVHWLKDR | by Transcript ID |
| | KAIRKSQKYDVVCEGTMAMLVIRGASLKDAGEYTCEVEASKSTASL | ENST00000483539 from Gene |
| | HVEEKANCFTEELTNLQVEEKGTAVFTCKTEHPAATVTWRKGLLEL | ID ENSG00000154358; Homo |
| | RASGKHQPSQEGLTLRLTISALEKADSDTYTCDIGQAQSRAQLLVQG | sapiens |
| | RRVHIIEDLEDVDVQEGSSATFRCRISPANYEPVHWFLDKTPLHANE | |
| | LNEIDAQPGGYHVLTLRQLALKDSGTIYFEAGDQRASAALRVTEKPS | |
| | VFSRELTDATITEGEDLTLVCETSTCDIPVCWTKDGKTLRGSARCQL | |
| | SHEGHRAQLLITGATLQDSGRYKCEAGGACSSSIVRVHARPVRFQE | |
| | ALKDLEVLEGGAATLRCVLSSVAAPVKWCYGNNVLRPGDKYSLRQ | |
| | EGAMLELVVRNLRPQDSGRYSCSFGDQTTSATLTVTALPAQFIGKLR | |
| | NKEATEGATATLRCELSKAAPVEWRKGSETLRDGDRYCLRQDGAM | |
| | CELQIRGLAMVDAAEYSCVCGEERTSASLTIRPMPAHFIGRLRHQESI | |
| | EGATATLRCELSKAAPVEWRKGRESLRDGDRHSLRQDGAVCELQIC | |
| | GLAVADAGEYSCVCGEERTSATLTVKALPAKFTEGLRNEEAVEGAT | |
| | AMLWCELSKVAPVEWRKGPENLRDGDRYILRQEGTRCELQICGLA | |
| | MADAGEYLCVCGQERTSATLTIRALPARFIEDVKNQEAREGATAVL | |
| | QCELNSAAPVEWRKGSETLRDGDRYSLRQDGTKCELQIRGLAMAD | |
| | TGEYSCVCGQERTSAMLTVRALPIKFTEGLRNEEATEGATAVLRCEL | |
| | SKMAPVEWWKGHETLRDGDRHSLRQDGARCELQIRGLVAEDAGE | |
| | YLCMCGKERTSAMLTVRAMPSKFIEGLRNEEATEGDTATLWCELSK | |
| | AAPVEWRKGHETLRDGDRHSLRQDGSRCELQIRGLAVVDAGEYSC | |
| | VCGQERTSATLTVRALPARFIEDVKNQEAREGATAVLQCELSKAAP | |
| | VEWRKGSETLRGGDRYSLRQDGTRCELQIHGLSVADTGEYSCVCG | |
| | QERTSATLTVRALPARFTQDLKTKEASEGATATLQCELSKVAPVEW | |
| | KKGPETLRDGGRYSLKQDGTRCELQIHDLSVADAGEYSCMCGQER | |
| | TSATLTVRALPARFTEGLRNEEAMEGATATLQCELSKAAPVEWRKG | |
| | LEALRDGDKYSLRQDGAVCELQIHGLAMADNGVYSCVCGQERTSA | |
| | TLTVRALPARFIEDMRNQKATEGATVTLQCKLRKAAPVEWRKGPN | |
| | TLKDGDRYSLKQDGTSCELQIRGLVIADAGEYSCICEQERTSATLTV | |
| | RALPARFIEDVRNHEATEGATAVLQCELSKAAPVEWRICGSETLRDG | |
| | DRYSLRQDGTRCELQIRGLAVEDTGEYLCVCGQERTSATLTVRALP | |
| | ARFIDNMTNQEAREGATATLHCELSKVAPVEWRKGPETLRDGDRH | |
| | SLRQENCLNPGGRGCSEPRSCHCTPAWVIEQ | |
| 73 | XGSSSWLQWKSLSKRLKSPRKPSSCKCRRVPNLPSDCSASCMPLTCL | OBSCN protein |
| | DHACVAQPPACCLSAHITCTL | (ENSP00000455500) encoded |
| | | by Transcript ID |
| | | ENST00000474237 from Gene |
| | | ID ENSG00000154358; Homo |
| | | sapiens |
| 74 | XSSITFSVKVEGRPVPTVHWLREEAERGVLWIGPDTPGYTVASSAQQ | OBSCN protein |
| | HSLVLLDVGRQHQGTYTCIASNAAGQALCSASLHVSGLPKVEEQEK | (ENSP00000489816) encoded |
| | VKEALISTFLQGTTQAISAQGLETASFADLGGQRKEEPLAAKEALGH | by Transcript ID |
| | LSLAEVGTEEFLQKLTSQITEMVSAKITQAKLQVPGGDSDEDSKTPS | ENST00000636476 from Gene |
| | ASPRHGRSRPSSSIQESSSESEDGDARGEIFDIYVVTADYLPLGAEQD | ID ENSG00000154358; Homo |
| | AITLREGQYVEVLDAAHPLRWLVRTKPTKSSPSRQGWVSPAYLDRR | sapiens |
| | LKLSPEWGAAEAPEFPGEAVSEDEYKARLSSVIQELLSSEQAFVEEL | |
| | QFLQSHHLQHLERCPHVPIAVAGQKAVIFRNVRDIGRFHSSSFLQEL | |
| | QQCDTDDDVAMCFIKNQAAFEQYLEFLVGRVQAESVVVSTAIQEFY | |
| | KKYAEEALLAGDPSQPPPPPLQHYLEQPVERVQRYQALLKELIRNK | |
| | ARNRQNCALLEQAYAVVSALPQRAENKLHVSLMENYPGTLQALGE | |
| | PIRQGHFIVWEGAPGARMPWKGHNRHVFLFRNHLVICKPRRDSRTD | |
| | TVSYVFRNMMKLSSIDLNDQVEGDDRAFEVWQEREDSVRKYLLQA | |
| | RTAIIKSSWVKEICGIQQRLALPVWRPPDFEEELADCTAELGETVKL | |
| | ACRVTGTPKPVISWYKDGKAVQVDPHHILIEDPDGSCALILDSLTGV | |
| | DSGQYMCFAASAAGNCSTLGKILVQVPPRFVNKVRASPFVEGEDAQ | |
| | FTCTIEGAPYPQIRWYKDGALLTTGNKFQTLSEPRSGLLVLVIRAAS | |
| | KEDLGLYECELVNRLGSARASAELRIQSPMLQAQEQCHREQLVAAV | |
| | EDTTLERADQEVTSVLKRLLGPKAPGPSTGDLTGPGPCPRGAPALQE | |
| | TGSQPPVTGTSEAPAVPPRVPQPLLHEGPEQEPEAIARAQEWTVPIR | |
| | MEGAAWPGAGTGELLWDVHSHVVRETTQRTYTYQAIDTHTARPPS | |
| | MQVTIEDVQAQTGGTAQFEAIIEGDPQPSVTWYICDSVQLVDSTRLS | |
| | QQQEGTTYSLVLRHVASKDAGVYTCLAQNTGGQVLCKAELLVLGG | |
| | DNEPDSEKQSHRRKLHSFYEVKEEIGRGVFGFVKRVQHKGNKILCA | |
| | AKFIPLRSRTRAQAYRERDILAALSHPLVTGLLDQFETRKTLILILELC | |
| | SSEELLDRLYRKGVVTEAEVKVYIQQLVEGLHYLHSHGVLHLDIKPS | |
| | NILMVHPAREDIKICDFGFAQNITPAELQFSQYGSPEFVSPEIIQQNPV | |
| | SEASDIWAMGVISYLSLTCSSPFAGESDRATLLNVLEGRVSWSSPMA | |
| | AHLSEDAKDFIKATLQRAPQARPSAAQCLSHPWFLKSMPAEEAHFIN | |
| | TKQLKFLLARSRWQRSLMSYKSILVMRSIPELLRGPPDSPSLGVARH | |
| | LCRDTGGSSSSSSSSDNELAPFARAKSLPPSPVTHSPLLHPRGFLRPSA | |
| | SLPEEAEASERSTEAPAPPASPEGAGPPAAQGCVPRHSVIRSLFYHQA | |
| | GESPEHGALAPGSRRHPARRRHLLKGGYIAGALPGLREPLMEHRVL | |
| | EEEAAREEQATLLAKAPSFETALRLPASGTHLAPGHSHSLEHDSPSTP | |
| | RPSSEACGEAQRLPSAPSGGAPIRDMGHPQGSKQLPSTGGHPGTAQP | |
| | ERPSPDSPWGQPAPFCHPKQGSAPQEGCSPHPAVAPCPPGSFPPGSC | |
| | KEAPLVPSSPFLGQPQAPPAPAKASPPLDSKMGPGDISLPGRPKPGPC | |
| | SSPGSASQASSSQVSSLRVGSSQVGTEPGPSLDAEGWTQEAEDLSDS | |
| | TPTLQRPQEQATMRKFSLGGRGGYAGVAGYGTFAFGGDAGGMLG | |
| | QGPMWARIAWAVSQSEEEEQEEARAESQSEEQQEARAESPLPQVSA | |
| | RPVPEVGRAPTRSSPEPTPWEDIGQVSLVQIRDLSGDAEAADTISLDI | |
| | SEVDPAYLNLSDLYDIKYLPFEFMIFRKVPKSAQPEPPSPMAEEELAE | |
| | FPEPTWPWPGELGPHAGLEITEESEDVDALLAEAAVGRKRKWSSPS | |
| | RSLFHFPGRHLPLDEPAELGLRERVKASVEHISRILKGRPEGLEKEGP | |
| | PRKKPGLASFRLSGLKSWDRAPTFLRELSDETVVLGQSVTLACQVS | |
| | AQPAAQATWSKDGAPLESSSRVLISATLKNFQLLTILVVVAEDLGVY | |
| | TCSVSNALGTVTTTGVLRKAERPSSSPCPDIGEVYADGVLLVWKPV | |
| | ESYGPVTYIVQCSLEGGSWTTLASDIFDCCYLTSKLSRGGTYTFRTA | |
| | CVSKAGMGPYSSPSEQVLLGGPSHLASEEESQGRSAQPLPSTKTFAF | |
| | QTQIQRGRFSVVRQCWEKASGRALAAKIIPYHPKDKTAVLREYEAL | |
| | KGLRHPHLAQLHAAYLSPRHLVLILELCSGPELLPCLAERASYSESE | |
| | VKDYLWQMLSATQYLHNQHILHLDLRSENMIITEYNLLKVVDLGN | |
| | AQSLSQEKVLPSDKFKDYLETMAPELLEGQGAVPQTDIWAIGVTAFI | |
| | MLSAEYPVSSEGARDLQRGLRKGLVRLSRCYAGLSGGAVAFLRSTL | |
| | CAQPWGRPCASSCLQCPWLTEEGPACSRPAPVTFPTARLRVFVRNR | |
| | EKRRALLYKRHNLAQVR | |
| 75 | MSSDEKGISPAHKTSTPTHRSASSSTSSQRDSRQSIHILERTASSSTEPSV | OSBPL6 protein |
| | SRQLLEPEPVPLSKEADSWEIIEGLKIGQTNVQKPDKHEGFMLKKRKW | (ENSP00000190611) encoded |
| | PLKGWHKRFFVLDNGMLKYSKAPLDIQKGKVHGSIDVGLSVMSIKKK | by Transcript ID |
| | ARRIDLDTEEHIYHLKVKSQDWFDAWVSKLRHHRLYRQNEIVRSPRD | ENST00000190611 from Gene |
| | ASFHIFPSTSTAESSPAANVSVMDGKMQPNSFPWQSPLPCSNSLPATCT | ID ENSG00000079156; Homo |
| | TGQSKVAAWLQDSEEMDRCAEDLAHCQSNLVELSKLLQNLEILQRTQ | sapiens |
| | SAPNFTDMQANCVDISKKDKRVTRRWRTKSVSKDTKIQLQVPFSATM | |
| | SPVRLHSSNPNLCADIEFQTPPSHLTDPLESSTDYTKLQEEFCLIAQKVH | |
| | SLLKSAFNSIAIEKEKLKQMVSEQDHSKGHSTQMARLRQSLSQALNQ | |
| | NAELRSRLNRIHSESIICDQVVSVNIIPSPDEAGEQIHVSLPLSQQVANES | |
| | RLSMSESVSEFFDAQEVLLSASSSENEASDDESYISDVSDNISEDNTSV | |
| | ADNISRQILNGELTGGAFRNGRRACLPAPCPDTSNINLWNILRNNIGKD | |
| | LSKVSMPVELNEPLNTLQHLCEEMEYSELLDKASETDDPYERMVLVA | |
| | AFAVSGYCSTYFRAGSKPFNPVLGETYECIREDKGFRFFSEQVSHHPPI | |
| | SACHCESKNFVFWQDIRWKNKFWGKSMEILPVGTLNVMLPKYGDYY | |
| | VWNKVTTCIHNILSGRRWIEHYGEVTIRNTKSSVCICKLTFVKVNYWN | |
| | SNMNEVQGVVIDQEGKAVYRLFGKWHEGLYCGVAPSAKCIWRPGSM | |
| | PTNYELYYGFTRFAIELNELDPVLKDLLPPTDARFRPDQRFLEEGNLEA | |
| | AASEKQRVEELQRSRRRYMEENNLEHIPKFFKKVIDANQREAWVSND | |
| | TYWELRKDPGFSKVDSPVLW | |
| 76 | MHQLSLIRGNRGRSIHILERTASSSTEPSVSRQLLEPEPVPLSKEADSWE | OSBPL6 protein |
| | IIEGLKIGQTNVQKPDKHEGFMLKKRKWPLKGWHKRFFVLDNGMLK | (ENSP00000318723) encoded |
| | YSKAPLDIQKGKVHGSIDVGLSVMSIKKKARRIDLDTEEHIYHLKVKS | by Transcript ID |
| | QDWFDAWVSKLRHHRLYRQNEIVRSPRDASFHIFPSTSTAESSPAANV | ENST00000315022 from Gene |
| | SVMDGKMQPNSFPWQSPLPCSNSLPATCTTGQSKVAAWLQDSEEMD | ID ENSG00000079156; Homo |
| | RCAEDLAHCQSNLVELSKLLQNLEILQRTQSAPNFTDMQANCVDISKK | sapiens |
| | DKRVTRRWRTKSVSKDTKIQLQEGPPAKGQFSTTRRRQRLAAAVATT | |
| | VPFSATMSPVRLHSSNPNLCADIEFQTPPSHLTDPLESSTDYTKLQEEF | |
| | CLIAQKVHSLLKSAFNSIAIEKEKLKQMVSEQDHSKGHSTQMARLRQS | |
| | LSQALNQNAELRSRLNRIHSESIICDQVVSVNIIPSPDEAGEQIHVSLPLS | |
| | QQVANESRLSMSESVSEFFDAQEVLLSASSSENEASDDESYISDVSDNI | |
| | SEDNTSVADNISRQILNGELTGGAFRNGRRACLPAPCPDTSNINLWNIL | |
| | RNNIGKDLSKVSMPVELNEPLNTLQHLCEEMEYSELLDKASETDDPYE | |
| | RMVLVAAFAVSGYCSTYFRAGSKPFNPVLGETYECIREDKGFRFFSEQ | |
| | VSHHPPISACHCESKNFVFWQDIRWKNKFWGKSMEILPVGTLNVMLP | |
| | KYGDYYVWNKVTTCIHNILSGRRWIEHYGEVTIRNTKSSVCICKLTFV | |
| | KVNYWNSNMNEVQGVVIDQEGKAVYRLFGKWHEGLYCGVAPSAKC | |
| | IWRPGSMPTNYELYYGFTRFAIELNELDPVLKDLLPPTDARFRPDQRFL | |
| | EEGNLEAAASEKQRVEELQRSRRRYMEENNLEHIPKFFKKVIDANQRE | |
| | AWVSNDTYWELRKDPGFSKVDSPVLW | |
| 77 | MSSDEKGISPAHKTSTPTHRSASSSTSSQRDSRQSIHILERTASSSTEPSV | OSBPL6 protein |
| | SRQLLEPEPVPLSKEADSWEIIEGLKIGQTNVQKPDKHEGFMLKKRKW | (ENSP00000349591) encoded |
| | PLKGWHKRFFVLDNGMLKYSKAPLDIQKGKVHGSIDVGLSVMSIKKK | by Transcript ID |
| | ARRIDLDTEEHIYHLKVKSQDWFDAWVSKLRHHRLYRQNEIVRSPRD | ENST00000357080 from Gene |
| | ASFHIFPSTSTAESSPAANVSVMDGKMQPNSFPWQSPLPCSNSLPATCT | ID ENSG00000079156; Homo |
| | TGQSKVAAWLQDSEEMDRCAEDLAHCQSNLVELSKLLQNLEILQRTQ | sapiens |
| | SAPNFTDMQVPFSATMSPVRLHSSNPNLCADIEFQTPPSHLTDPLESST | |
| | DYTKLQEEFCLIAQKVHSLLKSAFNSIAIEKEKLKQMVSEQDHSKGHS | |
| | TQMARLRQSLSQAGEQIHVSLPLSQQVANESRLSMSESVSEFFDAQEV | |
| | LLSASSSENEASDDESYISDVSDNISEDNTSVADNISRQSMHHLSFQVV | |
| | LSTCQIATRRLNEQAFPLPRHPHPC | |
| 78 | MSSDEKGISPAHKTSTPTHRSASSSTSSQRDSRQSIHILERTASSSTEPSV | OSBPL6 protein |
| | SRQLLEPEPVPLSKEADSWEIIEGLKIGQTNVQKPDKHEGFMLKKRKW | (ENSP00000352713) encoded |
| | PLKGWHKRFFVLDNGMLKYSKAPLDIQKGKVHGSIDVGLSVMSIKKK | by Transcript 1D |
| | ARRIDLDTEEHIYHLKVKSQDWFDAWVSKLRHHRLYRQNEIVRSPRD | ENSt00000359685 from Gene |
| | ASFHIFPSTSTAESSPAANVSVMDGKMQPNSFPWQSPLPCSNSLPATCT | ID ENSG00000079156; Homo |
| | TGQSKVAAWLQDSEEMDRCAEDLAHCQSNLVELSKLLQNLEILQRTQ | sapiens; |
| | SAPNFTDMQANCVDISKKDKRVTRRWRTKSVSKDTKIQLQVPFSATM | OSBPL6 protein |
| | SPVRLHSSNPNLCADIEFQTPPSHLTDPLESSTDYTKLQEEFCLIAQKVH | (ENSP00000386885) encoded |
| | SLLKSAFNSIAIEKEKLKQMVSEQDHSKGHSTQMARLRQSLSQAGEQI | by Transcript ID |
| | HVSLPLSQQVANESRLSMSESVSEFFDAQEVLLSASSSENEASDDESYI | ENSt00000409631 from Gene |
| | SDVSDNISEDNTSVADNISRQILNGELTGGAFRNGRRACLPAPCPDTSN | ID ENSG00000079156; Homo |
| | INLWNILRNNIGKDLSKVSMPVELNEPLNTLQHLCEEMEYSELLDKAS | sapiens |
| | ETDDPYERMVLVAAFAVSGYCSTYFRAGSKPFNPVLGETYECIREDK | |
| | GFRFFSEQVSHHPPISACHCESKNFVFWQDIRWKNKFWGKSMEILPVG | |
| | TLNVMLPKYGDYYVWNKVTTCIHNILSGRRWIEHYGEVTIRNTKSSV | |
| | CICKLTFVKVNYWNSNMNEVQGVVIDQEGKAVYRLFGKWHEGLYC | |
| | GVAPSAKCIWRPGSMPTNYELYYGFTRFAIELNELDPVLKDLLPPTDA | |
| | RFRPDQRFLEEGNLEAAASEKQRVEELQRSRRRYMEENNLEHIPKFFK | |
| | KVIDANQREAWVSNDTYWELRKDPGFSKVDSPVLW | |
| 79 | MSSDEKGISPAHKTSTPTHRSASSSTSSQRDSRQSIHILERTASSSTEPSV | OSBPL6 protein |
| | SRQLLEPEPVPLSKEADSWEIIEGLKIGQTNVQKPDKHEGFMLKKRKW | (ENSP00000376293) encoded |
| | PLKGWHKRFFVLDNGMLKYSKAPLDIQKGKVHGSIDVGLSVMSIKKK | by Transcript ID |
| | ARRIDLDTEEHIYHLKVKSQDWFDAWVSKLRHHRLYRQNEIVRSPRD | ENST00000392505 from Gene |
| | ASFHIFPSTSTAESSPAANVSVMDGKMQPNSFPWQSPLPCSNSLPATCT | ID ENSG00000079156; Homo |
| | TGQSKVAAWLQDSEEMDRCAEDLAHCQSNLVELSKLLQNLEILQRTQ | sapiens |
| | SAPNFTDMQANCVDISKKDKRVTRRWRTKSVSKDTKIQLQEGPPAKG | |
| | QFSTTRRRQRLAAAVATTVPFSATMSPVRLHSSNPNLCADIEFQTPPSH | |
| | LTDPLESSTDYTKLQEEFCLIAQKVHSLLKSAFNSIAIEKEKLKQMVSE | |
| | QDHSKGHSTQMARLRQSLSQALNQNAELRSRLNRIHSESIICDQVVSV | |
| | NIIPSPDEAGEQIHVSLPLSQQVANESRLSMSESVSEFFDAQEVLLSASS | |
| | SENEASDDESYISDVSDNISEDNTSVADNISRQILNGELTGGAFRNGRR | |
| | ACLPAPCPDTSNINLWNILRNNIGKDLSKVSMPVELNEPLNTLQHLCE | |
| | EMEYSELLDKASETDDPYERMVLVAAFAVSGYCSTYFRAGSKPFNPV | |
| | LGETYECIREDKGFRFFSEQVSHHPPISACHCESKNFVFWQDIRWKNK | |
| | FWGKSMEILPVGTLNVMLPKYGDYYVWNKVTTCIHNILSGRRWIEHY | |
| | GEVTIRNTKSSVCICKLTFVKVNYWNSNMNEVQGVVIDQEGKAVYRL | |
| | FGKWHEGLYCGVAPSAKCIWRPGSMPTNYELYYGFTRFAIELNELDP | |
| | VLKDLLPPTDARFRPDQRFLEEGNLEAAASEKQRVEELQRSRRRYME | |
| | ENNLEHIPKFFKKVIDANQREAWVSNDTYWELRKDPGFSKVDSPVLW | |
| 80 | MSSDEKGISPAHKTSTPTHRSASSSTSSQRDSRQSIHILERTASSSTEPSV | OSBPL6 protein |
| | SRQLLEPEPVPLSKEADSWEIIEGLKIGQTNVQKPDKHEGFMLKKRKW | (ENSP00000387248) encoded |
| | PLKGWHKRFFVLDNGMLKYSKAPLDIQKGKVHGSIDVGLSVMSIKKK | by Transcript ID |
| | ARRIDLDTEEHIYHLKVKSQDWFDAWVSKLRHHRLYRQNEIVRSPRD | ENST00000409045 from Gene |
| | ASFHIFPSTSTAESSPAANVSVMDGKMQPNSFPWQSPLPCSNSLPATCT | ID ENSG00000079156; Homo |
| | TGQSKVAAWLQDSEEMDRCAEDLAHCQSNLVELSKLLQNLEILQRTQ | sapiens |
| | SAPNFTDMQVPFSATMSPVRLHSSNPNLCADIEFQTPPSHLTDPLESST | |
| | DYTKLQEEFCLIAQKVHSLLKSAFNSIAIEKEKLKQMVSEQDHSKGHS | |
| | TQMARLRQSLSQALNQNAELRSRLNRIHSESIICDQVVSVNIIPSPDEA | |
| | GEQIHVSLPLSQQVANESRLSMSESVSEFFDAQEVLLSASSSENEASDD | |
| | ESYISDVSDNISEDNTSVADNISRQILNGELTGGAFRNGRRACLPAPCP | |
| | DTSNINLWNILRNNIGKDLSKVSMPVELNEPLNTLQHLCEEMEYSELL | |
| | DKASETDDPYERMVLVAAFAVSGYCSTYFRAGSKPFNPVLGETYECI | |
| | REDKGFRFFSEQVSHHPPISACHCESKNFVFWQDIRWKNKFWGKSMEI | |
| | LPVGTLNVMLPKYGDYYVWNKVTTCIHNILSGRRWIEHYGEVTIRNT | |
| | KSSVCICKLTFVKVNYWNSNMNEVQGVVIDQEGKAVYRLFGKWHEG | |
| | LYCGVAPSAKCIWRPGSMPTNYELYYGFTRFAIELNELDPVLKDLLPP | |
| | TDARFRPDQRFLEEGNLEAAASEKQRVEELQRSRRRYMEENNLEHIPK | |
| | FFKKVIDANQREAWVSNDTYWELRKDPGFSKVDSPVLW | |
| 81 | MVNENTRMYIPEENHQGSNYGSPRPAHANMNANAAAGLAPEHIPTPG | CACNA1C protein |
| | AALSWQAAIDAARQAKLMGSAGNATISTVSSTQRKRQQYGKPKKQG | (ENSP00000382542) encoded |
| | STTATRPPRALLCLTLKNPIRRACISIVEWKPFEIIILLTIFANCVALAIYI | by Transcript ID |
| | PFPEDDSNATNSNLERVEYLFLIIFTVEAFLKVIAYGLLFHPNAYLRNG | ENST00000399634 from Gene |
| | WNLLDFIIVVVGLFSAILEQATKADGANALGGKGAGFDVKALRAFRV | ID ENSG00000151067; Homo |
| | LRPLRLVSGVPSLQVVLNSIIKAMVPLLHIALLVLFVIIIYAIIGLELFMG | sapiens |
| | KMHKTCYNQEGIADVPAEDDPSPCALETGHGRQCQNGTVCKPGWDG | CACNA1C protein |
| | PKHGITNFDNFAFAMLTVFQCITMEGWTDVLYWMQDAMGYELPWV | (ENSP00000494461) encoded |
| | YFVSLVIFGSFFVLNLVLGVLSGEFSKEREKAKARGDFQKLREKQQLE | by Transcript ID |
| | EDLKGYLDWITQAEDIDPENEDEGMDEEKPRNMSMPTSETESVNTEN | ENST00000643528 from Gene |
| | VAGGDIEGENCGARLAHRISKSKFSRYWRRWNRFCRRKCRAAVKSN | ID ENSG00000285479; Homo |
| | VFYWLVIFLVFLNTLTIASEHYNQPNWLTEVQDTANKALLALFTAEM | sapiens |
| | LLKMYSLGLQAYFVSLFNRFDCFVVCGGILETILVETKIMSPLGISVLR | |
| | CVRLLRIFKITRYWNSLSNLVASLLNSVRSIASLLLLLFLFIIIFSLLGMQ | |
| | LFGGKFNFDEMQTRRSTFDNFPQSLLTVFQILTGEDWNSVMYDGIMA | |
| | YGGPSFPGMLVCIYFIILFICGNYILLNVFLAIAVDNLADAESLTSAQKE | |
| | EEEEKERKKLARTASPEKKQELVEKPAVGESKEEKIELKSITADGESPP | |
| | ATKINMDDLQPNENEDKSPYPNPETTGEEDEEEPEMPVGPRPRPLSEL | |
| | HLKEKAVPMPEASAFFIFSSNNRFRLQCHRIVNDTIFTNLILFFILLSSISL | |
| | AAEDPVQHTSFRNHILFYFDIVFTTIFTIETALKMTAYGAFLHKGSFCRN | |
| | YFNILDLLVVSVSLISFGIQSSAINVVKILRVLRVLRPLRAINRAKGLKH | |
| | VVQCVFVAIRTIGNIVIVTTLLQFMFACIGVQLFKGKLYTCSDSSKQTE | |
| | AECKGNYITYKDGEVDHPIIQPRSWENSKFDFDNVLAAMMALFTVST | |
| | FEGWPELLYRSIDSHTEDKGPIYNYRVEISIFFIIYIIIIAFFMMNIFVGFVI | |
| | VTFQEQGEQEYKNCELDKNQRQCVEYALKARPLRRYIPKNQHQYKV | |
| | WYVVNSTYFEYLMFVLILLNTICLAMQHYGQSCLFKIAMNILNMLFT | |
| | GLFTVEMILKLIAFKPKHYFCDAWNTFDALIVVGSIVDIAITEVNPAEH | |
| | TQCSPSMNAEENSRISITFFRLFRVMRLVKLLSRGEGIRTLLWTFIKSFQ | |
| | ALPYVALLIVMLFFIYAVIGMQVFGKIALNDTTEINRNNNFQTFPQAVL | |
| | LLFRCATGEAWQDIMLACMPGKKCAPESEPSNSTEGETPCGSSFAVFY | |
| | FISFYMLCAFLIINLFVAVIMDNFDYLTRDWSILGPHHLDEFKRIWAEY | |
| | DPEAKGRIKHLDVVTLLRRIQPPLGFGKLCPHRVACKRLVSMNMPLN | |
| | SDGTVMFNATLFALVRTALRIKTEGNLEQANEELRAIIKKIWKRTSMK | |
| | LLDQVVPPAGDDEVTVGKFYATFLIQEYFRKFKKRKEQGLVGKPSQR | |
| | NALSLQAGLRTLHDIGPEIRRAISGDLTAEEELDKAMKEAVSAASEDDI | |
| | FRRAGGLFGNHVSYYQSDGRSAFPQTFTTQRPLHINKAGSSQGDTESP | |
| | SHEKLVDSTFTPSSYSSTGSNANINNANNTALGRLPRPAGYPSTVSTVE | |
| | GHGPPLSPAIRVQEVAWKLSSNRMHCCDMLDGGTFPPALGPRRAPPC | |
| | LHQQLQGSLAGLREDTPCIVPGHASLCCSSRVGEWLPAGCTAPQHAR | |
| | CHSRESQAAMAGQEETSQDETYEVKMNHDTEACSEPSLLSTEMLSYQ | |
| | DDENRQLTLPEEDKRDIRQSPKRGFLRSASLGRRASFHLECLKRQKDR | |
| | GGDISQKTVLPLHLVHHQALAVAGLSPLLQRSHSPASFPRPFATPPATP | |
| | GSRGWPPQPVPTLRLEGVESSEKLNSSFPSIHCGSWAETTPGGGGSSA | |
| | ARRVRPVSLMVPSQAGAPGRQFHGSASSLVEAVLISEGLGQFAQDPKF | |
| | IEVTTQELADACDMTIEEMESAADNILSGGAPQSPNGALLPFVNCRDA | |
| | GQDRAGGEEDAGCVRARGRPSEEELQDSRVYVSSL | |
| 82 | MGQDQTKQQIEKGLQLYQSNQTEKALQVWTKVLEKSSDLMGRFRVL | RAPSN protein |
| | GCLVTAHSEMGRYKEMLKFAVVQIDTARELEDADFLLESYLNLARSN | (ENSP00000298854) encoded |
| | EKLCEFHKTISYCKTCLGLPGTRAGAQLGGQVSLSMGNAFLGLSVFQ | by Transcript ID |
| | KALESFEKALRYAHNNDDAMLECRVCCSLGSFYAQVKDYEKALFFPC | ENST00000298854 from Gene |
| | KAAELVNNYGKGWSLKYRAMSQYHMAVAYRLLGRLGSAMECCEES | ID ENSG00000165917; Homo |
| | MKIALQHGDRPLQALCLLCFADIHRSRGDLETAFPRYDSAMSIMTEIG | sapiens |
| | NRLGQVQALLGVAKCWVARKALDKALDAIERAQDLAEEVGNKLSQL | |
| | KLHCLSESIYRSKGLQRELRAHVVRFHECVEETELYCGLCGESIGEKN | |
| | SRLQALPCSHIFHLRCLQNNGTRSCPNCRRSSMKPGFV | |
| 83 | MGQDQTKQQIEKGLQLYQSNQTEKALQVWTKVLEKSSDLMGRFRVL | RAPSN protein |
| | GCLVTAHSEMGRYKEMLKFAVVQIDTARELEDADFLLESYLNLARSN | (ENSP00000298853) encoded |
| | EKLCEFHKTISYCKTCLGLPGTRAGAQLGGQVSLSMGNAFLGLSVFQ | by Transcript ID |
| | KALESFEKALRYAHNNDDAMLECRVCCSLGSFYAQVKDYEKALFFPC | ENST00000352508 from Gene |
| | KAAELYNNYCKGWSLKYRAMSQYHMAVAYRLLGRLGSAMECCEES | ID ENSG00000165917; Homo |
| | MKIALQHGDRPLQALCLLCFADIHRSRGDLELSQLKLHCLSESIYRSK | sapiens |
| | GLQRELRAHVVRFHECVEETELYCGLCGESIGEKNSRLQALPCSHIFH | |
| | LRCLQNNGTRSCPNCRRSSMKPGFV | |
| 84 | MGQDQTKQQIEKGLQLYQSNQTEKALQVWTKVLEKSSDLMGRFRVL | RAPSN protein |
| | GCLVTAHSEMGRYKEMLKFAVVQIDTARELEDADFLLESYLNLARSN | (ENSP00000435551) encoded |
| | EKLCEFHKTISYCKTCLGLPGTRAGAQLGGQVSLSMGNAFLGLSVFQ | by Transcript ID |
| | KALESFEKALRYAHNNDDAMLECRVCCSLGSFYAQVKESMKIALQH | ENST00000524487 from Gene |
| | GDRPLQALCLLCFADIHRSRGDLETAFPRYDSAMSIMTEIGNRLGQVQ | ID ENSG00000165917; Homo |
| | ALLGVAKCWVARKALDKALDAIERAQDLAEEVGNKLSQLKLHCLSE | sapiens |
| | SIYRSKGLQRELRAHVVRFHECVEETELYCGLCGESIGEKNSRLQALP | |
| | CSHIFHLRCLQNNGTRSCPNCRRSSMKPGFV | |
| 85 | MGQDQTKQQIEKGLQLYQSNQTEKALQVWTKVLEKSSDLMGRFRVL | RAPSN protein |
| | GCLVTAHSEMGRYKEMLKFAVVQIDTARELEDADFLLESYLNLARSN | (ENSP00000431732) encoded |
| | EKLCEFHKTISYCKTCLGLPGTRAGAQLGGQVSLSMGNAFLGLSVFQ | by Transcript ID |
| | KALESFEKALRYAHNNDDAMLECRVCCSLGSFYAQVKDYEKALFFPC | EN ST00000529341 from Gene |
| | KAAELVNNYGKGWSLKYRAMSQYHMAVAYRLLGRLGSAMECCEES | ID ENSG00000165917; Homo |
| | MKIALQHGDRPLQALCLLCFADIHRSRGDLELSQLKLHCLSESIYRSK | sapiens |
| | GLQRELRAHVVRFHECVEETELYCGLCGESIGEKNSRLQALPCSHIFH | |
| | LRWGLLGGWTGVGRISSGHSRAQLA | |
| 86 | MRENVPGEKKPQNGIPLPPQIFNEEQYCGDFDSFFSAKEENIIYSFLGLA | SH3BGR protein |
| | PPPDSKEEEGETATEETEEIAMEGAEGEAEEEEETAEGEEPGEDEDS | (ENSP00000404980) encoded |
| | | by Transcript ID |
| | | ENST00000458295 from Gene |
| | | ID ENSG00000185437; Homo |
| | | sapiens |
| 87 | MPLLLLGETEPLKLERDCRSPVDPWAAASPDLALACLCHCQDLSSGA | SH3BGR protein |
| | FPDRGVLGGVLFPTVEMVIKVFVATSSGSIAIRKKQQEVVGFLEANKI | (ENSP00000332513) encoded |
| | DFKELDIAGDEDNRRWMRENVPGEKKPQNGIPLPPQIFNEEQYCGDFD | by Transcript ID |
| | SITSAKEENIIYSFLGLAPPPDSKGSEKAEEGGETEAQKEGSEDVGNLP | ENST00000333634 from Gene |
| | EAQEKNEEEGETATEETEEIAMEGAEGEAEEEEETAEGEEPGEDEDS | ID ENSG00000185437; Homo |
| | | sapiens |
| 88 | MRENVPGEKKPQNGIPLPPQIFNEEQYCGDFDSFFSAKEENIIYSFLGLA | SH3BGR protein |
| | PPPDSKGSEKAEEGGETEAQKEGSEDVGNLPEAQEKNEEEGETATEET | (ENSP00000370005) encoded |
| | EEIAMEGAEGEAEEEEETAEGEEPGEDEDS | by Transcript ID |
| | | ENST00000380631 from Gene |
| | | ID ENSG00000185437; Homo |
| | | sapiens; |
| | | SH3BGR protein |
| | | (ENSP00000370008) encoded |
| | | by Transcript ID |
| | | ENST00000380634 from Gene |
| | | ID ENSG00000185437; Homo |
| | | sapiens; |
| | | SH3 BGR protein |
| | | (ENSP00000370011) encoded |
| | | by Transcript ID |
| | | ENST00000380637 from Gene |
| | | ID ENSG00000185437; Homo |
| | | sapiens |
| 89 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382546) encoded |
| | KQGSTTATRPPRALLCLTLKNPIRRACISIVEWKPFEIIILLTIFANCVA | by Transcript ID |
| | LAIYIFFPEDDSNATNSNLERVEYLFLIIFTVEAFLKVIAYGLLFHPNA | ENST00000399637 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000493890) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000645965 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | HYFCDAWNTFDALIVVGSIVDIAITEVNPAEHTQCSPSMNAEENSRIS | |
| | ITFFRLFRVMRLVKLLSRGEGIRTLLWTFIKSFQALPYVALLIVMLFFI | |
| | YAVIGMQVFGKIALNDTTEINRNNNFQTFPQAVLLLFRCATGEAWQ | |
| | DIMLACMPGKKCAPESEPSNSTEGETPCGSSFAVFYFISFYMLCAFLII | |
| | NLFVAVIMDNFDYLTRDWSILGPHHLDEFKRIWAEYDPEAKGRIKH | |
| | LDVVTLLRRIQPPLGFGKLCPHRVACKRLVSMNMPLNSDGTVMFNA | |
| | TLFALVRTALRIKTEEGPSPSEAHQGAEDPFRPAGNLEQANEELRAII | |
| | KKIWKRTSMKLLDQVVPPAGDDEVTVGKFYATFLIQEYFRKFKKRK | |
| | EQGLVGKPSQRNALSLQAGLRTLHDIGPEIRRAISGDLTAEEELDKA | |
| | MKEAVSAASEDDIFRRAGGLFGNHVSYYQSDGRSAFPQTFTTQRPL | |
| | HINKAGSSQGDTESPSHEKLVDSTFTPSSYSSTGSNANINNANNTAL | |
| | GRLPRPAGYPSTVSTVEGHGPPLSPAIRVQEVAWKLSSNRCHSRESQ | |
| | AAMAGQEETSQDETYEVKMNHDTEACSEPSLLSTEMLSYQDDENR | |
| | QLTLPEEDKRDIRQSPKRGFLRSASLGRRASFHLECLKRQKDRGGDI | |
| | SQKTVLPLHLVHHQALAVAGLSPLLQRSHSPASFPRPFATPPATPGS | |
| | RGWPPQPVPTLRLEGVESSEKLNSSFPSIHCGSWAETTPGGGGSSAA | |
| | RRVRPVSLMVPSQAGAPGRQFHGSASSLVEAVLISEGLGQFAQDPKF | |
| | IEVTTQELADACDMTIEEMESAADNILSGGAPQSPNGALLPFVNCRD | |
| | AGQDRAGGEEDAGCVRARGRPSEEELQDSRVYVSSL | |
| 90 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382547) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399638 from Gene |
| | YLRNGWNLLDFIIVVVGLFSAILEQATKADGANALGGKGAGEDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494782) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000644048 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLIWLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKG | |
| | YFSDPWNVFDFLIVIGSIIDVILSETNHYFCDAWNTFDALIVVGSIVDI | |
| | AITEVNPAEHTQCSPSMNAEENSRISITFFRLFRVMRLVKLLSRGEGI | |
| | RTLLWTFIKSFQALPYVALLIVMLFFIYAVIGMQVFGKIALNDTTEIN | |
| | RNNNFQTFPQAVLLLFRCATGEAWQDIMLACMPGKKCAPESEPSNS | |
| | TEGETPCGSSFAVFYFISFYMLCAFLIINLFVAVIMDNFDYLTRDWSI | |
| | LGPHHLDEFKRIWAEYDPEAKGRIKHLDVVTLLRRIQPPLGFGKLCP | |
| | HRVACKRLVSMNMPLNSDGTVMFNATLFALVRTALRIKTEGNLEQ | |
| | ANEELRAIIKKIWKRTSMKLLDQVVPPAGDDEVTVGKFYATFLIQEY | |
| | FRKFKKRKEQGLVGKPSQRNALSLQAGLRTLHDIGPEIRRAISGDLT | |
| | AEEELDKAMKEAVSAASEDDIFRRAGGLFGNHVSYYQSDGRSAFPQ | |
| | TFTTQRPLHINKAGSSQGDTESPSHEKLVDSTFTPSSYSSTGSNANIN | |
| | NANNTALGRLPRPAGYPSTVSTVEGHGPPLSPAIRVQEVAWKLSSN | |
| | RCHSRESQAAMAGQEETSQDETYEVKMNHDTEACSEPSLLSTEMLS | |
| | YQDDENRQLTLPEEDKRDIRQSPKRGFLRSASLGRRASFHLECLKRQ | |
| | KDRGGDISQKTVLPLHLVHHQALAVAGLSPLLQRSHSPASFPRPFAT | |
| | PPATPGSRGWPPQPVPTLRLEGVESSEKLNSSFPSIHCGSWAETTPGG | |
| | GGSSAARRVRPVSLMVPSQAGAPGRQFHGSASSLVEAVLISEGLGQF | |
| | AQDPKFIEVTTQELADACDMTIEEMESAADNILSGGAPQSPNGALLP | |
| | FVNCRDAGQDRAGGEEDAGCVRARGRPSEEELQDSRVYVSSL | |
| 91 | GSEKAEEGGETEAQKEGSEDVGNLPEAQEKNKMEAMKQHFKCLST | SH3BGR protein |
| | NQTQQNTLA | (ENSP00000403115) encoded |
| | | by Transcript ID |
| | | ENST00000447939 from Gene |
| | | ID ENSG00000185437; Homo |
| | | sapiens |
| 92 | MRENVPGEKKPQNGIPLPPQIFNEEQYCGDFDSFFSAKEENIIYSFLG | SH3BGR protein |
| | LAPPPDSKGSEKAEEGGETEAQKEGSEDVGNLPEAQEKNEEEGETA | (ENSP00000401572) encoded |
| | TEETEEIAMEGA | by Transcript ID |
| | | ENST00000440288 from Gene |
| | | ID ENSG00000185437; Homo |
| | | sapiens |
| 93 | IFNEEQYCGGSEKAEEGGETEAQKEGSEDVGNLPEAQEKNEEEGET | SH3BGR protein |
| | ATEETEEIAMEGAEGEAEEEEETAEGEEPGEDEDS | (ENSP00000413981) encoded |
| | | by Transcript ID |
| | | ENST00000423596 from Gene |
| | | ID ENSG00000185437; Homo |
| | | sapiens |
| 94 | XSSGSIAIRKKQQEVVGFLEANKIDFKELDIAGDEDNRRWMRENVP | SH3BGR protein |
| | GEKKPQNGIPLPPQIFNEEQYCGDFDSFFSAKEENIIYSFLGLAPPPDS | (ENSP00000405675) encoded |
| | KEEEGETATEETEEIAMEGAEGEAEEEEETAEGEEPGEDEDS | by Transcript ID |
| | | ENST00000452550 from Gene |
| | | ID ENSG00000185437; Homo |
| | | sapiens |
| 95 | MKRKERIARRLEGIENDTQPILLQSCTGLVTHRLLEEDTPRYMRASD | SVIL protein |
| | PASPHIGRSNEEEETSDSSLEKQTRSKYCTETSGVHGDSPYGSGTMD | (ENSP00000348128) encoded |
| | THSLESKAERIARYKAERRRQLAEKYGLTLDPEADSEYLSRYTKSRK | by Transcript ID |
| | EPDAVEKRGGKSDKQEESSRDASSLYPGTETMGLRTCAGESKDYAL | ENST00000355867 from Gene |
| | HVGDGSSDPEVLLNIENQRRGQELSATRQAHDLSPAAESSSTFSFSG | ID ENSG00000197321; HOMO |
| | RDSSFTEVPRSPKHAHSSSLQQAASRSPSFGDPQLSPEARPSTGKPKH | sapiens |
| | EWFLQKDSEGDTPSLINWPSRVKVREKLVKEESARNSPELASESVTQ | |
| | RRHQPAPVHYVSFQSEHSAFDRVPSKAAGSTRQPIRGYVQPADTGH | |
| | TAKLVTPETPENASECSWVASATQNVPKPPSLTVLEGDGRDSPVLH | |
| | VCESKAEEEEGEGEGEEKEEDVCFTEALEQSKKTLLALEGDGLVRSP | |
| | EDPSRNEDFGKPAVSTVTLEHQKELENVAQPPQAPHQPTERTGRSE | |
| | MVLYIQSEPVSQDAKPTGHNREASKKRKVRTRSLSDFTGPPQLQAL | |
| | KYKDPASRRELELPSSKTEGPYGEISMLDTKVSVAQLRSAFLASANA | |
| | CRRPELKSRVERSAEGPGLPTGVERERGSRKPRRYFSPGESRKTSERF | |
| | RTQPITSAERKESDRCTSHSETPTVDDEEKVDERAKLSVAAKRLLFR | |
| | EMEKSFDEQNVPKRRSRNTAVEQRLRRLQDRSLTQPITTEEVVIAAT | |
| | EPIPASCSGGTHPVMARLPSPTVARSAVQPARLQASAHQKALAKDQ | |
| | TNEGKELAEQGEPDSSTLSLAEKLALFNKLSQPVSKAISTRNRIDTRQ | |
| | RRMNARYQTQPVTLGEVEQVQSGKLIPFSPAVNTSVSTVASTVAPM | |
| | YAGDLRTKPPLDHNASATDYKFSSSIENSDSPVRSILKSQAWQPLVE | |
| | GSENKGMLREYGETESKRALTGRDSGMEKYGSFEEAEASYPILNRA | |
| | REGDSHKESKYAVPRRGSLERANPPITHLGDEPKEFSMAKMNAQGN | |
| | LDLRDRLPFEEKVEVENVMKRKFSLRAAEFGEPTSEQTGTAAGKTIA | |
| | QTTAPVSWKPQDSSEQPQEKLCKNPCAMFAAGEIKTPTGEGLLDSPS | |
| | KTMSIKERLALLKKSGEEDWRNRLSRRQEGGKAPASSLHTQEAGRS | |
| | LIKKRVTESRESQMTIEERKQLITVREEAWKTRGRGAANDSTQFTVA | |
| | GRMVKKGLASPTAITPVASPICGKTRGTTPVSKPLEDIEARPDMQLE | |
| | SDLKLDRLETFLRRLNNKVGGMHETVLTVTGKSVKEVMKPDDDET | |
| | FAKFYRSVDYNMPRSPVEMDEDFDVIFDPVAPKLTSSVAEHKRAVR | |
| | PKRRVQASKNPLKMLAAREDLLQEYTEQRLNVAFMESKRMKVEK | |
| | MSSNSNFSEVTLAGLASKENFSNVSLRSVNLTEQNSNNSAVPYKRL | |
| | MLLQIKGRRHVQTRLVEPRASALNSGDCFLLLSPHCCFLWVGEFAN | |
| | VIEKAKASELATLIQTKRELGCRATYIQTIEEGINTHTHAAKDFWKLL | |
| | GGQTSYQSAGDPKEDELYEAAIIETNCIYRLMDDKLVPDDDYWGKI | |
| | PKCSLLQPKEVLVFDFGSEVYVWHGKEVTLAQRKIAFQLAKHLWN | |
| | GTFDYENCDINPLDPGECNPLIPRKGQGRPDWAIFGRLTEHNETILFK | |
| | EKFLDWTELKRSNEKNPGELAQHKEDPRTDVKAYDVTRMVSMPQT | |
| | TAGTILDGVNVGRGYGLVEGHDRRQFEITSVSVDVWHILEFDYSRLP | |
| | KQSIGQFHEGDAYVVKWKFMVSTAVGSRQKGEHSVRAAGKEKCV | |
| | YFFWQGRHSTVSEKGTSALMTVELDEERGAQVQVLQGKEPPCFLQ | |
| | CFQGGMVVHSGRREEEEENVQSEWRLYCVRGEVPVEGNLLEVACH | |
| | CSSLRSRTSMVVLNVNKALIYLWHGCKAQAHTKEVGRTAANKIKE | |
| | QCPLEAGLHSSSKVTIHECDEGSEPLGFWDALGRRDRKAYDCMLQD | |
| | PGSFNFAPRLFILSSSSGDFAATEFVYPARAPSVVSSMPFLQEDLYSA | |
| | PQPALFLVDNHHEVYLWQGWWPIENKITGSARIRWASDRKSAMET | |
| | VLQYCKGKNLKKPAPKSYLIHAGLEPLTFTNMFPSWEHREDIAEITE | |
| | MDTEVSNQITLVEDVLAKLCKTIYPLADLLARPLPEGVDPLKLEIYL | |
| | TDEDFEFALDMTRDEYNALPAWKQVNLKKAKGLF | |
| 96 | MKRKERIARRLEGIENDTQPILLQSCTGLVTHRLLEEDTPRYMRASD | SVIL protein |
| | PASPHIGRSNEEEETSDSSLEKQTRSKYCTETSGVHGDSPYGSGTMD | (ENSP00000364547) encoded |
| | THSLESKAERIARYKAERRRQLAEKYGLTLDPEADSEYLSRYTKSRK | by Transcript ID |
| | EPDAVEKRGGKSDKQEESSRDASSLYPGTETMGLRTCAGESKDYAL | ENST00000375398 from Gene |
| | HVGDGSSDPEVLLNIENQRRGQELSATRQAHDLSPAAESSSTFSFSG | ID ENSG00000197321; Homo |
| | RDSSFTEVPRSPKHAHSSSLQQAASRSPSFGDPQLSPEARPSTGKPKH | sapiens |
| | EWFLQKDSEGDTPSLINWPSRVKVREKLVKEESARNSPELASESVTQ | |
| | RRHQPAPVHYVSFQSEHSAFDRVPSKAAGSTRQPIRGYVQPADTGH | |
| | TAKLVTPETPENASECSWVASATQNVPKPPSLTVLEGDGRDSPVLH | |
| | VCESKAEEEEGEGEGEEKEEDVCFTEALEQSKKTLLALEGDGLVRSP | |
| | EDPSRNEDFGKPAVSTVTLEHQKELENVAQPPQAPHQPTERTGRSE | |
| | MVLYIQSEPVSQDAKPTGHNREASKKRKVRTRSLSDFTGPPQLQAL | |
| | KYKDPASRRELELPSSKTEGPYGEISMLDTKVSVAQLRSAFLASANA | |
| | CRRPELKSRVERSAEGPGLPTGVERERGSRKPRRYFSPGESRKTSERF | |
| | RTQPITSAERKESDRCTSHSETPTVDDEEKVDERAKLSVAAKRLLFR | |
| | EMEKSFDEQNVPKRRSRNTAVEQRLRRLQDRSLTQPITTEEVVIAAT | |
| | LQASAHQKALAKDQTNEGKELAEQGEPDSSTLSLAEKLALFNKLSQ | |
| | PVSKAISTRNRIDTRQRRMNARYQTQPVTLGEVEQVQSGKLIPFSPA | |
| | VNTSVSTVASTVAPMYAGDLRTKPPLDHNASATDYKFSSSIENSDSP | |
| | VRSILKSQAWQPLVEGSENKGMLREYGETESKRALTGRDSGMEKY | |
| | GSFEEAEASYPILNRAREGDSHKESKYAVPRRGSLERANPPITHLGD | |
| | EPKEFSMAKMNAQGNLDLRDRLPFEEKVEVENVMKRKFSLRAAEF | |
| | GEPTSEQTGTAAGKTIAQTTAPVSWKPQDSSEQPQEKLCKNPCAMF | |
| | AAGEIKTPTGEGLLDSPSKTMSIKERLALLKKSGEEDWRNRLSRRQE | |
| | GGKAPASSLHTQEAGRSLIKKRVTESRESQMTIEERKQLITVREEAW | |
| | KTRGRGAANDSTQFTVAGRMVKKGLASPTAITPVASPICGKTRGTT | |
| | PVSKPLEDIEARPDMQLESDLKLDRLETFLRRLNNKVGGMHETVLT | |
| | VTGKSVKEVMKPDDDETFAKFYRSVDYNMPRSPVEMDEDFDVIFD | |
| | PYAPKLTSSVAEHKRAVRPKRRVQASKNPLKMLAAREDLLQEYTE | |
| | QRLNVAFMESKRMKVEKMSSNSNFSEVTLAGLASKENFSNVSLRSV | |
| | NLTEQNSNNSAVPYKRLMLLQIKGRRHVQTRLVEPRASALNSGDCF | |
| | LLLSPHCCFLWVGEFANVIEKAKASELATLIQTKRELGCRATYIQTIE | |
| | EGINTHTHAAKDFWKLLGGQTSYQSAGDPKEDELYEAAIIETNCIYR | |
| | LMDDKLVPDDDYWGKIPKCSLLQPKEVLVFDFGSEVYVWHGKEVT | |
| | LAQRKIAFQLAKHLWNGTFDYENCDINPLDPGECNPLIPRKGQGRPD | |
| | WAIFGRLTEHNETILFKEKFLDWTELKRSNEKNPGELAQHKEDPRTD | |
| | VKAYDVTRMVSMPQTTAGTILDGVNVGRGYGLVEGHDRRQFEITS | |
| | VSVDVWHILEFDYSRLPKQSIGQFHEGDAYVVKWKFMVSTAVGSR | |
| | QKGEHSVRAAGKEKCVYFFWQGRHSTVSEKGTSALMTVELDEERG | |
| | AQVQVLQGKEPPCFLQCFQGGMVVHSGRREEEEENVQSEWRLYCV | |
| | RGEVPVEGNLLEVACHCSSLRSRTSMVVLNVNKALIYLWHGCKAQ | |
| | AHTKEVGRTAANKIKEQCPLEAGLHSSSKVTIHECDEGSEPLGFWD | |
| | ALGRRDRKAYDCMLQDPGSFNFAPRLFILSSSSGDFAATEFVYPARA | |
| | PSVVSSMPFLQEDLYSAPQPALFLVDNHHEVYLWQGWWPIENKITG | |
| | SARIRWASDRKSAMETVLQYCKGKNLKKPAPKSYLIHAGLEPLTFT | |
| | NMFPSWEHREDIAEITEMDTEVSNQITLVEDVLAKLCKTIYPLADLL | |
| | ARPLPEGVDPLKLEIYLTDEDFEFALDMTRDEYNALPAWKQVNLKK | |
| | AKGLF | |
| 97 | MKRKERIARRLEGIENDTQPILLQSCTGLVTHRLLEEDTPRYMRASD | SVIL protein |
| | PASPHIGRSNEEEETSDSSLEKQTRSKYCTETSGVHGDSPYGSGTMD | (ENSP00000364549) encoded |
| | THSLESKAERIARYKAERRRQLAEKYGLTLDPEADSEYLSRYTKSRK | by Transcript ID |
| | EPDAVEKRGGKSDKQEESSRDASSLYPGTETMGLRTCAGESKDYAL | ENST00000375400 from Gene |
| | HVGDGSSDPEVLLNIENQRRGQELSATRQAHDLSPAAESSSTFSFSG | ID ENSG00000197321; Homo |
| | RDSSFTEVPRSPKHAHSSSLQQAASRSPSFGDPQLSPEARPRCTSHSE | sapiens |
| | TPTVDDEEKVDERAKLSVAAKRLLFREMEKSFDEQNVPKRRSRNTA | |
| | VEQRLRRLQDRSLTQPITTEEVVIAATLQASAHQKALAKDQTNEGK | |
| | ELAEQGEPDSSTLSLAEKLALFNKLSQPVSKAISTRNRIDTRQRRMN | |
| | ARYQTQPVTLGEVEQVQSGKLIPFSPAVNTSVSTVASTVAPMYAGD | |
| | LRTKPPLDHNASATDYKFSSSIENSDSPVRSILKSQAWQPLVEGSENK | |
| | GMLREYGETESKRALTGRDSGMEKYGSFEEAEASYPILNRAREGDS | |
| | HKESKYAVPRRGSLERANPPITHLGDEPKEFSMAKMNAQGNLDLRD | |
| | RLPFEEKVEVENVMKRKFSLRAAEFGEPTSEQTGTAAGKTIAQTTAP | |
| | VSWKPQDSSEQPQEKLCKNPCAMFAAGEIKTPTGEGLLDSPSKTMSI | |
| | KERLALLKKSGEEDWRNRLSRRQEGGKAPASSLHTQEAGRSLIKKR | |
| | VTESRESQMTIEERKQLITVREEAWKTRGRGAANDSTQFTVAGRMV | |
| | KKGLASPTAITPVASPICGKTRGTTPVSKPLEDIEARPDMQLESDLKL | |
| | DRLETFLRRLNNKVGGMHETVLTVTGKSVKEVMKPDDDETFAKFY | |
| | RSVDYNMPRSPVEMDEDFDVIFDPYAPKLTSSVAEHKRAVRPKRRV | |
| | QASKNPLKMLAAREDLLQEYTEQRLNVAFMESKRMKVEKMSSNSN | |
| | FSEVTLAGLASKENFSNVSLRSVNLTEQNSNNSAVPYKRLMLLQIKG | |
| | RRHVQTRLVEPRASALNSGDCFLLLSPHCCFLWVGEFANVIEKAKA | |
| | SELATLIQTKRELGCRATYIQTIEEGINTHTHAAKDFWKLLGGQTSY | |
| | QSAGDPKEDELYEAAIIETNCIYRLMDDKLVPDDDYWGKIPKCSLLQ | |
| | PKEVLVEDFGSEVYVWHGKEVTLAQRKIAFQLAKHLWNGTFDYEN | |
| | CDINPLDPGECNPLIPRKGQGRPDWAIFGRLTEHNETILFKEKFLDWT | |
| | ELKRSNEKNPGELAQHKEDPRTDVKAYDVTRMVSMPQTTAGTILD | |
| | GVNVGRGYGLVEGHDRRQFEITSVSVDVWHILEFDYSRLPKQSIGQF | |
| | HEGDAYVVKWKFMVSTAVGSRQKGEHSVRAAGKEKCVYFFWQG | |
| | RHSTVSEKGTSALMTVELDEERGAQVQVLQGKEPPCFLQCFQGGM | |
| | VVHSGRREEEEENVQSEWRLYCVRGEVPVEGNLLEVACHCSSLRSR | |
| | TSMVVLNVNKALIYLWHGCKAQAHTKEVGRTAANKIKEQCPLEAG | |
| | LHSSSKVTIHECDEGSEPLGFWDALGRRDRKAYDCMLQDPGSFNFA | |
| | PRLFILSSSSGDFAATEFVYPARAPSVVSSMPFLQEDLYSAPQPALFL | |
| | VDNHHEVYLWQGWWPIENKITGSARIRWASDRKSAMETVLQYCK | |
| | GKNLKKPAPKSYLIHAGLEPLTFTNMFPSWEHREDIAEITEMDTEVS | |
| | NQITLVEDVLAKLCKTIYPLADLLARPLPEGVDPLKLEIYLTDEDFEF | |
| | ALDMTRDEYNALPAWKQVNLKKAKGLF | |
| 98 | XPQEKLCKNPCAMFAAGEIKTPTGEGLLDSPSKTMSIKERLALLKKS | SVIL protein |
| | GEEDWRNRLSRRQEGGKAPASSLHTQEAGRSLIKKEEGGVADDSAI | (ENSP00000488171) encoded |
| | SNLLWRVTESRESQMTIEERKQLITVREEAWKTRGRGAANDSTQFT | by Transcript ID |
| | VAGRMVKKGLASPTAITPVASPICGKTRGTTPVSKPLEDIEARPDMQ | ENST00000632315 from Gene |
| | LESDLKLDRLETFLRRLNNKVGGMHETVLTVTGKSVKEVMKPDDD | ID ENSG00000197321; Homo |
| | ETFAKFYRSVDYNMPRSPVEMDEDFDVIFDPYAPKLTSSVAEHKRA | sapiens |
| | VRPKRRVQASKNPLKMLAAREDLLQEYTEQRLNVAFMESKRMKVE | |
| | KMSSNSNFSEVTLAGLASKENFSNVSLRSVNLTEQNSNNSAVPYKR | |
| | LMLLQIKGRRHVQTRLVEPRASALNSGDCFLLLSPHCCFLWVGEFA | |
| | NVIEKAKASELATLIQTKRELGCRATYIQTIEEGINTHTHAAKDFWK | |
| | LLGGQTSYQSAGDPKEDELYEAAIIETNCIYRLMDDKLVPDDDYWG | |
| | KIPKCSLLQPKEVLVFDFGSEVYVWHGKEVTLAQRKIAFQLAKHLW | |
| | NGTFDYENCDINPLDPGECNPLIPRKGQGRPDWAIFGRLTEHNETILF | |
| | KEKFLDWTELKRSNEKNPGELAQHKEDPRTDVKAYDVTRMVSMPQ | |
| | TTAGTILDGVNVGRGYGLVEGHDRRQFEITSVSVDVWHILEFDYSR | |
| | LPKQSIGQFHEGDAYVVKWKFMVSTAVGSRQKGEHSVRAAGKEKC | |
| | VYFFWQGRHSTVSEKGTSALMTVELDEERGAQVQVLQGKEPPCFL | |
| | QCFQGGMVVHSGRREEEEENVQSEWRLYCVRGEVPVEGNLLEVAC | |
| | HCSSLRSRTSMVVLNVNKALIYLWHGCKAQAHTKEVGRTAANKIK | |
| | EQCPLEAGLHSSSKVTIHECDEGSEPLGFWDALGRRDRKAYDCMLQ | |
| | DPGSFNFAPRLFILSSSSGDFAATEFVYPARAPSVVSSMPFLQED | |
| 99 | MSYRRELEKYRDLDEDEILGALTEEELRTLENELDELDPDNALLPAG | TMOD1 protein |
| | LRQKDQTTKAPTGPFKREELLDHLEKQAKEFKDREDLVPYTGEKRG | (ENSP00000259365) encoded |
| | KVWVPKQKPLDPVLESVTLEPELEEALANASDAELCDIAAILGMHT | by Transcript ID |
| | LMSNQQYYQALSSSSIMNKEGLNSVIKPTQYKPVPDEEPNSTDVEET | ENST00000259365 from Gene |
| | LERIKNNDPKLEEVNLNNIRNIPIPTLKAYAEALKENSYVKKFSIVGT | ID ENSG00000136842; Homo |
| | RSNDPVAYALAEMLKENKVLKTLNVESNFISGAGILRLVEALPYNTS | sapiens; |
| | LVEMKIDNQSQPLGNKVEMEIVSMLEKNATLLKFGYHFTQQGPRLR | TMOD1 protein |
| | ASNAMMNNNDLVRKRRLADLTGPIIPKCRSGV | (ENSP00000378637) encoded |
| | | by Transcript ID |
| | | ENST00000395211 from Gene |
| | | ID ENSG00000136842; Homo |
| | | sapiens |
| 100 | MSQVLSILGMHTLMSNQQYYQALSSSSIMNKEGLNSVIKPTQYKPV | TMOD1 protein |
| | PDEEPNSTDVEETLERIKNNDPKLEEVNLNNIRNIPIPTLKAYAEALK | (ENSP00000364318) encoded |
| | ENSYVKKFSIVGTRSNDPVAYALAEMLKENKVLKTLNVESNFISGA | by Transcript ID |
| | GILRLVEALPYNTSLVEMKIDNQSQPLGNKVEMEIVSMLEKNATLL | ENST00000375175 from Gene |
| | KFGYHFTQQGPRLRASNAMMNNNDLVRKRRLADLTGPIIPKCRSGV | ID ENSG00000136842; Homo |
| | | sapiens |
| 101 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382549) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399641 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWMQD | (ENSP00000494765) encoded |
| | AMGYELPWVYFVSLVIFGSFFVLNLVLGVLSGEFSKEREKAKARGD | by Transcript ID |
| | FQKLREKQQLEEDLKGYLDWITQAEDIDPENEDEGMDEEKPRNMS | ENST00000644369 from Gene |
| | MPTSETESVNTENVAGGDIEGENCGARLAHRISKSKFSRYWRRWNR | ID ENSG00000285479; Homo |
| | FCRRKCRAAVKSNVFYWLVIFLVFLNTLTIASEHYNQPNWLTEVQD | sapiens |
| | TANKALLALFTAEMLLKMYSLGLQAYFVSLFNRFDCFVVCGGILETI | |
| | LVETKIMSPLGISVLRCVRLLRIFKITRYWNSLSNLVASLLNSVRSIAS | |
| | LLLLLFLFIIIFSLLGMQLFGGKFNFDEMQTRRSTFDNFPQSLLTVFQI | |
| | LTGEDWNSVMYDGIMAYGGPSFPGMLVCIYFIILFICGNYILLNVFL | |
| | AIAVDNLADAESLTSAQKEEEEEKERKKLARTASPEKKQELVEKPA | |
| | VGESKEEKIELKSITADGESPPATKINMDDLQPNENEDKSPYPNPETT | |
| | GEEDEEEPEMPVGPRPRPLSELHLKEKAVPMPEASAFFIFSSNNRFRL | |
| | QCHRIVNDTIFTNLILFFILLSSISLAAEDPVQHTSFRNHILGNADYVF | |
| | TSIFTLEIILKMTAYGAFLHKGSFCRNYFNILDLLVVSVSLISFGIQSSA | |
| | INVVKILRYLRVLRPLRAINRAKGLKHVVQCVFVAIRTIGNIVIVTTL | |
| | LQFMFACIGVQLFKGKLYTCSDSSKQTEAECKGNYITYKDGEVDHPI | |
| | IQPRSWENSKFDFDNVLAAMMALFTVSTFEGWAELLYRSIDSHTED | |
| | KGPIYNYRVEISIFFIIYIIIIAFFMMNIFVGFVIVTFQEQGEQEYKNCEL | |
| | DKNQRQCVEYALKARPLRRYIPKNQHQYKVWYVVNSTYFEYLMF | |
| | VLILLNTICLAMQHYGQSCLFKIAMNILNMLFTGLFTVEMILKLIAFK | |
| | PKHYFCDAWNTFDALIVVGSIVDIAITEVNPAEHTQCSPSMNAEENS | |
| | RISITFFRLFRVMRLVKLLSRGEGIRTLLWTFIKSFQALPYVALLIVML | |
| | FFIYAVIGMQVFGKIALNDTTEINRNNNFQTFPQAVLLLFRCATGEA | |
| | WQDIMLACMPGKKCAPESEPSNSTEGETPCGSSFAVFYFISFYMLCA | |
| | FLIINLFVAVIMDNFDYLTRDWSILGPHHLDEFKRIWAEYDPEAKGRI | |
| | KHLDVVTLLRRIQPPLGFGKLCPHRVACKRLVSMNMPLNSDGTVMF | |
| | NATLFALVRTALRIKTEGNLEQANEELRAIIKKIWKRTSMKLLDQVV | |
| | PPAGDDEVTVGKFYATFLIQEYFRKFKKRKEQGLVGKPSQRNALSL | |
| | QAGLRTLHDIGPEIRRAISGDLTAEEELDKAMKEAVSAASEDDIFRR | |
| | AGGLFGNHVSYYQSDGRSAFPQTFTTQRPLHINKAGSSQGDTESPSH | |
| | EKLVDSTFTPSSYSSTGSNANINNANNTALGRLPRPAGYPSTVSTVE | |
| | GHGPPLSPAIRVQEVAWKLSSNRCHSRESQAAMAGQEETSQDETYE | |
| | VKMNHDTEACSEPSLLSTEMLSYQDDENRQLTLPEEDKRDIRQSPK | |
| | RGFLRSASLORRASFHLECLKRQKDRGGDISQKTVLPLHLVHHQAL | |
| | AVAGLSPLLQRSHSPASFPRPFATPPATPGSRGWPPQPVPTLRLEGVE | |
| | SSEKLNSSFPSIHCGSWAETTPGGGGSSAARRVRPVSLMVPSQAGAP | |
| | GRQFHGSASSLVEAVIASEGLGQFAQDPKFIEVTTQELADACDMTIE | |
| | EMESAADNILSGGAPQSPNGALLPFVNCRDAGQDRAGGEEDAGCV | |
| | RARGRPSEEELQDSRVYVSSL | |
| 102 | MGDEEKRNRAITARRQHLKSVMLQIAATELEKEESRREAEKQNYLA | TNNI2 protein |
| | EHCPPLHIPGSMSEVQELCKQLHAKIDAAEEEKYDMEVRVQKTSKE | (ENSP00000252898) encoded |
| | LEDMNQKLFDLRGKFKRPPLRRVRMSADAMLKALLGSKHKVCMD | by Transcript ID |
| | LRANLKQVKKEDTEKERDLRDVGDWRKNIEEKSGMEGRKKMFESE | ENST00000252898 from Gene |
| | S | ID ENSG00000130598; Homo |
| | | sapiens; |
| | | TNNI2 protein |
| | | (ENSP00000371331) encoded |
| | | by Transcript ID |
| | | ENST00000381906 from Gene |
| | | ID ENSG00000130598; Homo |
| | | sapiens; |
| | | TNNI2 protein |
| | | (ENSP00000371336) encoded |
| | | by Transcript ID |
| | | ENST00000381911 from Gene |
| | | ID ENSG00000130598; Homo |
| | | sapiens |
| 103 | MSQCKKRNRAITARRQHLKSVMLQIAATELEKEESRREAEKQNYLA | TNNI2 protein |
| | EHCPPLHIPGSMSEVQELCKQLHAKIDAAEEEKYDMEVRVQKTSKE | (ENSP00000371330) encoded |
| | LEDMNQKLFDLRGKFKRPPLRRVRMSADAMLKALLGSKHKVCMD | by Transcript ID |
| | LRANLKQVKKEDTEKERDLRDVGDWRKNIEEKSGMEGRKKMFESE | ENST00000381905 from Gene |
| | S | ID ENSG00000130598; Homo |
| | | sapiens |
| 104 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382552) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399644 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALEIGHGRQCQNGT | CACNA1C protein |
| | VCKPGWDGPKHGITNFDNFAFAMLTVFQCITMEGWTDVLYWVND | (ENSP00000493573) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000646257 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | GYFSDPWNVFDFLIVIGSIIDVILSETNPAEHTQCSPSMNAEENSRISIT | |
| | FFRLFRVMRLVKLLSRGEGIRTLLWTFIKSFQALPYVALLIVMLFFIY | |
| | AVIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEGNLEQANEELRAIIKKIWKRTSMKLLDQVVPPA | |
| | GDDEVTVGKFYATFLIQEYFRKFKKRKEQGLVGKPSQRNALSLQAG | |
| | LRTLHDIGPEIRRAISGDLTAEEELDKAMKEAVSAASEDDIFRRAGG | |
| | LFGNHVSYYQSDGRSAFPQTFTTQRPLHINKAGSSQGDTESPSHEKL | |
| | VDSTFTPSSYSSTGSNANINNANNTALGRLPRPAGYPSTVSTVEGHG | |
| | PPLSPAIRVQEVAWKLSSNRCHSRESQAAMAGQEETSQDETYEVKM | |
| | NHDTEACSEPSLLSTEMLSYQDDENRQLTLPEEDKRDIRQSPKRGFL | |
| | RSASLGRRASFHLECLKRQKDRGGDISQKTVLPLHLVHHQALAVAG | |
| | LSPLLQRSHSPASFPRPFATPPATPGSRGWPPQPVPTLRLEGVESSEK | |
| | LNSSFPSIHCGSWAETTPGGGGSSAARRVRPVSLMVPSQAGAPGRQF | |
| | HGSASSLVEAVLISEGLGQFAQDPKFIEVTTQELADACDMTIEEMES | |
| | AADNILSGGAPQSPNGALLPFVNCRDAGQDRAGGEEDAGCVRARG | |
| | RPSEEELQDSRVYVSSL | |
| 105 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382557) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399649 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000496458) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPTNMSM | ENST00000643701 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKH | |
| | YFCDAWNTFDALIVVGSIVDIAITENAEENSRISITFFRLFRVMRLVK | |
| | LLSRGEGIRTLLWTFIKSFQALPYVALLIVMLFFIYAVIGMQVFGKIA | |
| | LNDTTEINRNNNFQTFPQAVLLLFRCATGEAWQDIMLACMPGKKCA | |
| | PESEPSNSTEGETPCGSSFAVFYFISFYMLCAFLIINLFVAVIMDNFDY | |
| | LTRDWSILGPHHLDEFKRIWAEYDPEAKGRIKHLDVVTLLRRIQPPL | |
| | GFGKLCPHRVACKRLVSMNMPLNSDGTVMFNATLFALVRTALRIK | |
| | TEEGPSPSEAHQGAEDPFRPAGNLEQANEELRAIIKKIWKRTSMKLL | |
| | DQVVPPAGDDEVTVGKFYATFLIQEYFRKFKKRKEQGLVGKPSQRN | |
| | ALSLQAGLRTLHDIGPEIRRAISGDLTAEEELDKAMKEAVSAASEDD | |
| | IFRRAGGLFGNHVSYYQSDGRSAFPQTFTTQRPLHINKAGSSQGDTE | |
| | SPSHEKLVDSTFTPSSYSSTGSNANINNANNTALGRLPRPAGYPSTVS | |
| | TVEGHGPPLSPAIRVQEVAWKLSSNRCHSRESQAAMAGQEETSQDE | |
| | TYEVKMNHDTEACSEPSLLSTEMLSYQDDENRQLTLPEEDKRDIRQ | |
| | SPKRGFLRSASLGRRASFHLECLKRQKDRGGDISQKTVLPLHLVHHQ | |
| | ALAVAGLSPLLQRSHSPASFPRPFATPPATPGSRGWPPQPVPTLRLEG | |
| | VESSEKLNSSFPSIHCGSWAETTPGGGGSSAARRVRPVSLMVPSQAG | |
| | APGRQFHGSASSLVEAVLISEGLGQFAQDPKFIEVTTQELADACDMT | |
| | IEEMESAADNILSGGAPQSPNGALLPFVNCRDAGQDRAGGEEDAGC | |
| | VRARGRPSEEELQDSRVYVSSL | |
| 106 | MGDLQKRNRAITARRQHLKSVMLQIAATELEKEESRREAEKQNYLA | TNNI2 protein |
| | EHCPPLHIPGSMSEVQELCKQLHAKIDAAEEEKYDMEVRVQKTSKE | (ENSP00000481242) encoded |
| | LEDMNQKLFDLRGKFKRPPLRRVRMSADAMLKALLGSKHKVCMD | by Transcript ID |
| | LRANLKQVKKEDTEKERDLRDVGDWRKNIEEKSGMEGRKKMFESE | ENST00000617947 from Gene |
| | S | ID ENSG00000130598; Homo |
| | | sapiens |
| 107 | MTEITAEGNASTTTTVIDSKNGSVPKSPGKVLKRTVTEDIVTTFSSPA | TRDN protein |
| | AWLLVIALIITWSAVAIVMFDLVDYKNFSASSIAKIGSDPLKLVRDA | (ENSP00000439281) encoded |
| | MEETTDWIYGFFSLLSDIISSEDEEDDDGDEDTDKGEIDEPPLRKKEI | by Transcript ID |
| | HKDKTEKQEKPERKIQTKVTHKEKEKGKEKVREKEKPEKKATHKE | ENST00000546248 from Gene |
| | KIEKKEKPETKTLAKEQKKAKTAEKSEEKTKKEVKGGKQEKVKQT | ID ENSG00000186439; Homo |
| | AAKVKEVQKTPSKPKEKEDKEKAAVSKHEQKGKHSEQEAAGGSKR | sapiens |
| | ILGKKHMQ | |
| 108 | MTEITAEGNASTTTTVIDSKNGSVPKSPGKVLKRTVTEDIVTTFSSPA | TRDN protein |
| | AWLLVIALIITWSAVAIVMFDLVDYKNFSASSIAKIGSDPLKLVRDA | (ENSP00000333984) encoded |
| | MEETTDWIYGFFSLLSDIISSEDEEDDDGDEDTDKGEIDEPPLRKKEI | by Transcript ID |
| | HKDKTEKQEKPERKIQTKVTHKEKEKGKEKVREKEKPEKKATHKE | ENST00000334268 from Gene |
| | KIEKKEKPETKTLAKEQKKAKTAEKSEEKTKKEVKGGKQEKVKQT | ID ENSG00000186439; Homo |
| | AAKVKEVQKTPSKPKEKEDKEKAAVSKHEQKDQYAFCRYMIDIFV | sapiens |
| | HGDLKPGQSPAIPPPLPTEQASRPTPASPALEEKEGEKKKAEKKVTSE | |
| | TKKKEKEDIKKKSEKETAIDVEKKEPGKASETKQGTVKIAAQAAAK | |
| | KDEKKEDSKKTKKPAEVEQPKGKKQEKKEKHVEPAKSPKKEHSVP | |
| | SDKQVKAKTERAKEEIGAVSIKKAVPGKKEEKTTKTVEQEIRKEKSG | |
| | KTSSILKDKEPIKGKEEKVPASLKEKEPETKKDEKMSKAGKEVKPKP | |
| | PQLQGKKEEKPEPQIKKEAKPAISEKVQIHKQDIVKPEKTVSHGKPEE | |
| | KVLKQVKAVTIEKTAKPKPTKKAEHREREPPSIKTDKPKPTPKGTSE | |
| | VTESGKKKTEISEKESKEKADMKHLREEKVSTRKESLQLHNVTKAE | |
| | KPARVSKDVEDVPASKKAKEGTEDVSPTKQKSPISFFQCVYLDGYN | |
| | GYGFQFPFTPADRPGESSGQANSPGQKQQGQ | |
| 109 | MTEITAEGNASTTTTVIDSKNGSVPKSPGKVLKRTVTEDIVTTFSSPA | TRDN protein |
| | AWLLVIALIITWSAVAIVMFDLVDYKNFSASSIAKIGSDPLKLVRDA | (ENSP00000437684) encoded |
| | MEETTDWIYGFFSLLSDIISSEDEEDDDGDEDTDKGEIDEPPLRKKEI | by Transcript ID |
| | HKDKTEKQEKPERKIQTKEVGHSS | ENST00000542443 from Gene |
| | | ID ENSG00000186439; Homo |
| | | sapiens |
| 110 | MTEITAEGNASTTTTVIDSKNGSVPKSPGKVLKRTVTEDIVTTFSSPA | TRDN protein |
| | AWLLVIALIITWSAVAIVMFDLVDYKNFSASSIAKIGSDPLKLVRDA | (ENSP00000486095) encoded |
| | MEETTDWIYGFFSLLSDIISSEDEEDDDGDEDTDKGEIDEPPLRKKEI | by Transcript ID |
| | HKDKTEKQEKPERKIQTKVTHKEKEKGKEKVREKEKPEKKATHKE | ENST00000628709 from Gene |
| | KIEKKEKPETKTLAKEQKKAKTAEKSEEKTKKEVKGGKQEKVKQT | ID ENSG00000186439; Homo |
| | AAKVKEVQKTPSKPKEKEDKEKAAVSKHEQKGQSPAIPPPLPTEQA | sapiens |
| | SRPTPASPALEGKYFFFS | |
| 111 | XTHKEKEKGKEKVREKEKPEKKATHKEKIEKKEKPETKTLAKEQKK | TRDN protein |
| | AKTAEKSEEKTKKEVKGGKQEKVKQTAAKVKEVQKTPSKPKEKED | (ENSP00000354307) encoded |
| | KEKAAVSKHEQKGFPGPGIRNSSYYCQYIKGQINAWNDLRTSLLPIP | by Transcript ID |
| | YLSCGKMCSNFNN | ENST00000361029 from Gene |
| | | ID ENSG00000186439; Homo |
| | | sapiens |
| 112 | XIHKDKTEKQEKPERKIQTKDWSANSSTIRTLKLTQLKPIYICT | TRDN protein |
| | | (ENSP00000406768) encoded |
| | | by Transcript ID |
| | | ENST00000422596 from Gene |
| | | ID ENSG00000186439; Homo |
| | | sapiens |
| 113 | MASASGAMAKHEQILVLDPPTDLKFKGPFTDVVTTNLKLRNPSDRK | VAPA protein |
| | VCFKVKTTAPRRYCVRANSGIIDPGSTVTVSVMLQPFDYDPNEKSKH | (ENSP00000345656) encoded |
| | KFMVQTIFAPPNTSDMEAVWKEAKPDELMDSKLRCVFEMPNENDK | by Transcript ID |
| | LGITIVGNAPTVTSMSSINNTVATPASYHTKDDPRGLSVLKQEKQKN | ENST00000340541 from Gene |
| | DMEPSKAVPLNASKQDGPMPKPHSVSLNDTETRKLMEECKRLQGE | ID ENSG00000101558; Homo |
| | MMKLSEENRHLRDEGLRLRKVAHSDKPGSTSTASFRDNVTSPLPSL | sapiens |
| | LVVIAAIFIGFFLGKFIL | |
| 114 | MASASGAMAKHEQILVLDPPTDLKFKGPFTDVVTTNLKLRNPSDRK | VAPA protein |
| | VCFKVKTTAPRRYCVRPNSGIIDPGSTVTVSVMLQPFDYDPNKSKH | (ENSP00000382880) encoded |
| | KFMVQTIFAPPNTSDMEAVWKEAKPDELMDSKLRCVFEMPNENDK | by Transcript ID |
| | LNDMEPSKAVPLNASKQDGPMPKPHSVSLNDTETRKLMEECKRLQ | ENST00000400000 fi.om Gene |
| | GEMMKLSEENRHLRDEGLRLRKVAFISDKPGSTSTASFRDNVTSPLP | ID ENSG00000101558; Homo |
| | SLLVVIAAIFIGFFLGKFIL | sapiens |
| 115 | MASASGAMAKHEQILVLDPPTDLKFKVIV | VAPA protein |
| | | (ENSP00000463155) encoded |
| | | by Transcript ID |
| | | ENST00000585042 from Gene |
| | | ID ENSG00000101558; Homo |
| | | sapiens |
| 116 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000382563) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000399655 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000494058) encoded |
| | AVGRDWPWINFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000644235 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKFDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKH | |
| | YFCDAWNTFDALIVVGSIVDIAITEVNPAEHTQCSPSIVINAEENSRISI | |
| | TFFRLFRVMRLVKLLSRGEGIRTLLWTFIKSFQALPYVALLIVMLFFI | |
| | YAVIGMQVFGKIALNDTTEINRNNNFQTFPQAVLLLFRCATGEAWQ | |
| | DIMLACMPGKKCAPESEPSNSTEGETPCGSSFAVFYFISFYMLCAFLII | |
| | NLEVAVIMDNEDYLTRDWSILGPHHLDEFKRIWAEYDPEAKGRIKH | |
| | LDVVTLLRRIQPPLGEGKLCPHRVACKRLVSMNMPLNSDGTVMFNA | |
| | TLFALVRTALRIKTEGNLEQANEELRAIIKKIWKRTSMKLLDQVVPP | |
| | AGDDEVTVGKFYATFLIQEYFRKFKKRKEQGLVGKPSQRNALSLQA | |
| | GLRTLHDIGPEIRRAISGDLTAEEELDKAMKEAVSAASEDDIFRRAG | |
| | GLFGNHVSYYQSDGRSAFPQTFTTQRPLHINKAGSSQGDTESPSHEK | |
| | LVDSTFTPSSYSSTGSNANINNANNTALGRLPRPAGYPSTVSTVEGH | |
| | GPPLSPAIRVQEVAWKLSSNRCHSRESQAAMAGQEETSQDETYEVK | |
| | MNHDTEACSEPSLLSTEMLSYQDDENRQLTLPEEDKRDIRQSPKRGF | |
| | LRSASLGRRASFHLECLKRQKDRGGDISQKTVLPLHLVHHQALAVA | |
| | GLSPLLQRSHSPASFPRPFATPPATPGSRGWPPQPVPTLRLEGVESSE | |
| | KLNSSFPSIHCGSWAETTPGGGGSSAARRVRPVSLMVPSQAGAPGR | |
| | QFHGSASSLVEAVLISEGLGQFAQDPKFIEVTMELADACDMTIEEM | |
| | ESAADNILSGGAPQSPNGALLPFVNCRDAGQDRAGGEEDAGCVRAR | |
| | GRPSEEELQDSRVYVSSL | |
| 117 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000385724) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000402845 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000495080) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000647062 from Gene |
| | PTSETESVNTENVAGGDIEGENCGARLAHRISKSKFSRYWRRWNRF | ID ENSG00000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILFYFDIVFTTIF | |
| | TIEIALKMTAYGAFLHKGSFCRNYFNILDLLVVSVSLISFGIQSSAINV | |
| | VKILRVLRVLRPLRAINRAKGLKHVVQCVFVAIRTIGNIVIVTTLLQF | |
| | MFACIGVQLFKGKLYTCSDSSKQTEAECKGNYITYKDGEVDHPIIQP | |
| | RSWENSKEDFDNVLAAMMALFTVSTFEGWPELLYRSIDSHTEDKGP | |
| | IYNYRVEISIFFIIYIIIIAFFMMNIFVGFVIVTFQEQGEQEYKNCELDKN | |
| | QRQCVEYALKARPLRRYIPKNQHQYKVWYVVNSTYFEYLMFVLIL | |
| | LNTICLAMQHYGQSCLFKIAMNILNMLFTGLFTVEMILKLIAFKPKG | |
| | YESDPWNVFDFLIVIGSIIDVILSETNPAEHTQCSPSMNAEENSRISITF | |
| | FRLFRVMRLVKLLSRGEGIRTLLWTFIKSFQALPYVALLIVMLFFIYA | |
| | VIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEEGPSPSEAHQGAEDPFRPAGNLEQANEELRAIIK | |
| | KIWKRTSMKLLDQVVPPAGDDEVTVGKFYATFLIQEYFRKFKKRKE | |
| | QGLVGKPSQRNALSLQAGLRTLHDIGPEIRRAISGDLTAEEELDKAM | |
| | KEAVSAASEDDIFRRAGGLFGNHVSYYQSDGRSAFPQTFTTQRPLHI | |
| | NKAGSSQGDTESPSHEKLVDSTFTPSSYSSTGSNANINNANNTALGR | |
| | LPRPAGYPSTVSTVEGHGPPLSPAIRVQEVAWKLSSNRCHSRESQAA | |
| | MAGQEETSQDETYEVKMNHDTEACSEPSLLSTEMLSYQDDENRQL | |
| | TLPEEDKRDIRQSPKRGFLRSASLGRRASFHLECLKRQKDRGGDISQ | |
| | KTVLPLHLVHHQALAVAGLSPLLQRSHSPASFPRPFATPPATPGSRG | |
| | WPPQPVPTLRLEGVESSEKLNSSFPSIHCGSWAETTPGGGGSSAARR | |
| | VRPVSLMVPSQAGAPGRQFHGSASSLVEAVLISEGLGQFAQDPKFIE | |
| | VTTQELADACDMTIEEMESAADNILSGGAPQSPNGALLPFVNCRDA | |
| | GQDRAGGEEDAGCVRARGRPSEEELQDSRVYVSSL | |
| 118 | CLVSGGMAC | Artificial Sequence; |
| | | Peptide selected from a random |
| | | peptide library |
| 119 | CLVSGCNTC | Artificial Sequence; |
| | | Peptide selected from a random |
| | | peptide library |
| 120 | CDLVSGYGC | Artificial Sequence; |
| | | Peptide selected from a random |
| | | peptide library |
| 121 | CLVSTSATC | Artificial Sequence; |
| | | Peptide selected from a random |
| | | peptide library |
| 122 | CTALVSQTC | Artificial Sequence; |
| | | Peptide selected from a random |
| | | peptide library |
| 123 | CWLVSGIGC | Artificial Sequence; |
| | | Peptide selected from a random |
| | | peptide library |
| 124 | CLVSSVFPC | Artificial Sequence; |
| | | Peptide selected from a random |
| | | peptide library |
| 125 | CPSLVSSVC | Artificial Sequence; |
| | | Peptide selected from a random |
| | | peptide library |
| 126 | CGVSLVSTC | Artificial Sequence; |
| | | Peptide selected from a random |
| | | peptide library |
| 127 | CQLVSGEPC | Artificial Sequence; |
| | | Peptide selected from a random |
| | | peptide library |
| 128 | CNLVSRRLC | Artificial Sequence; |
| | | Peptide selected from a random |
| | | peptide library |
| 129 | CLVSWRGSC | Artificial Sequence; |
| | | Peptide selected from a random |
| | | peptide library |
| 130 | CDHFLVSPC | Artificial Sequence; |
| | | Peptide selected from a random |
| | | peptide library |
| 131 | CGRGLVSLC | Artificial Sequence; |
| | | Peptide selected from a random |
| | | peptide library |
| 132 | CFPVALVSC | Artificial Sequence; |
| | | Peptide selected from a random |
| | | peptide library |
| 133 | CRWSSLVSC | Artificial Sequence; |
| | | Peptide selected from a random |
| | | peptide library |
| 134 | CWSKSLVSC | Artificial Sequence; |
| | | Peptide selected from a random |
| | | peptide library |
| 135 | CPGRSLVSC | Artificial Sequence; |
| | | Peptide selected from a random |
| | | peptide library |
| 136 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000385896) encoded |
| | KQGSTTATRPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000406454 from Gene |
| | YLRNGWNLLDFIIVVVGLFSAILEQATKADGANALGGKGAGFDVKA | ID ENSG00000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWMQD | (ENSP00000493743) encoded |
| | AMGYELPWVYFVSLVIFGSFFVLNLVLGVLSGEFSKEREKAKARGD | by Transcript ID |
| | FQKLREKQQLEEDLKGYLDWITQAEDIDPENEDEGMDEEKPRNMS | ENST00000643730 from Gene |
| | MPTSETESVNTENVAGGDIEGENCGARLAHRISKSKFSRYWRRWNR | ID ENSG00000285479; Homo |
| | FCRRKCRAAVKSNVFYWLVIFLVFLNTLTIASEHYNQPNWLTEVQD | sapiens |
| | TANKALLALFTAEMLLKMYSLGLQAYFVSLFNRFDCFVVCGGILETI | |
| | LVETKIMSPLGISVLRCVRLLRIFKITRYWNSLSNLVASLLNSVRSIAS | |
| | LLLLLFLFIIIFSLLGMQLFGGKFNFDEMQTRRSTFDNFPQSLLTVFQI | |
| | LTGEDWNSVMYDGIMAYGGPSFPGMLVCIYFIILFICGNYILLNVFL | |
| | AIAVDNLADAESLTSAQKEEEEEKERKKLARTASPEKKQELVEKPA | |
| | VGESKEEKIELKSITADGESPPATKINMDDLQPNENEDKSPYPNPETT | |
| | GEEDEEEPEMPVGPRPRPLSELHLKEKAVPMPEASAFFIFSSNNRFRL | |
| | QCHRIVNDTIFTNLILFFILLSSISLAAEDPVQHTSFRNHILFYFDIVFTT | |
| | IFTIEIALKMTAYGAFLHKGSFCRNYFNILDLLVVSVSLISFGIQSSAIN | |
| | VVKILRVLRVLRPLRAINRAKGLKHVVQCVFVAIRTIGNIVIVYTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PlYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | GYFSDPWNVFDFLIVIGSIIDVILSETNPAEHTQCSPSMNAEENSRISIT | |
| | FFRLFRVMRLVKLLSRGEGIRTLLWTFIKSFQALPYVALLIVMLFFIY | |
| | AVIGMQVFGKIALNDTTEINRNNNFQTFPQAVLLLFRCATGEAWQDI | |
| | MLACMPGKKCAPESEPSNSTEGETPCGSSFAVFYFISFYMLCAFLIIN | |
| | LFVAVIMDNFDYLTRDWSILGPHHLDEFKRIWAEYDPEAKGRIKHL | |
| | DVVTLLRRIQPPLGFGKLCPHRVACKRLVSMNMPLNSDGTVMFNAT | |
| | LFALVRTALRIKTEGNLEQANEELRAIIKKIWKRTSMKLLDQVVPPA | |
| | GDDEVTVGKFYATFLIQEYFRKFKKRKEQGLVGKPSQRNALSLQAG | |
| | LRTLHDIGPEIRRAISGDLTAEEELDKAMKEAVSAASEDDIFRRAGG | |
| | LFGNHVSYYQSDGRSAFPQTFTTQRPLHINKAGSSQGDTESPSHEKL | |
| | VDSTFTPSSYSSTGSNANINNANNTALGRLPRPAGYPSTVSTVEGHG | |
| | PPLSPAIRVQEVAWKLSSNRMHCCDMLDGGTFPPALGPRRAPPCLH | |
| | QQLQGSLAGLREDTPCIVPGHASLCCSSRVGEWLPAGCTAPQHARC | |
| | HSRESQAAMAGQEETSQDETYEVKMNHDTEACSEPSLLSTEMLSYQ | |
| | DDENRQLTLPEEDKRDIRQSPKRGFLRSASLGRRASFHLECLKRQKD | |
| | RGGDISQKTVLPLHLVHHQALAVAGLSPLLQRSHSPASFPRPFATPP | |
| | ATPGSRGWPPQPVPTLRLEGVESSEKLNSSFPSIHCGSWAETTPGGG | |
| | GSSAARRVRPVSLMVPSQAGAPGRQFHGSASSLVEAVLISEGLGQFA | |
| | QDPKFIEVTTQELADACDMTIEEMESAADNILSGGAPQSPNGALLPF | |
| | VNCRDAGQDRAGGEEDAGCVRARGRPSEEELQDSRVYVSSL | |
| 137 | MVNENTRMYIPEENHQGSNYGSPRPAHANMNANAAAGLAPEHIPT | CACNA1C protein |
| | PGAALSWQAAIDAARQAKLMGSAGNATISTVSSTQRKRQQYGKPK | (ENSP00000437936) encoded |
| | KQGSTTATIIPPRALLCLTLKNPIRRACISIVEWKPFEIIILLTIFANCVA | by Transcript ID |
| | LAIYIPFPEDDSNATNSNLERVEYLFLIIFTVEAFLKVIAYGLLFHPNA | ENST00000480911 from Gene |
| | YLRNGWNLLDFIIVVVGLFSAILEQATKADGANALGGKGAGFDVKA | ID ENS000000151067; Homo |
| | LRAFRVLRPLRLVSGVPSLQVVLNSIIKAMVPLLHIALLVLFVIIIYAII | sapiens |
| | GLELFMGKMHKTCYNQEGIADVPAEDDPSPCALETGHGRQCQNGT | CACNA1C protein |
| | VCKPGWDGPKHGITNFDNFAFAMLTVFQCITMEGWTDVLYWVND | (ENSP00000496400) encoded |
| | AVGRDWPWIYFVTLIIIGSFFVLNLVLGVLSGEFSKEREKAKARGDF | by Transcript ID |
| | QKLREKQQLEEDLKGYLDWITQAEDIDPENEDEGMDEEKPRNMSM | ENST00000646676 from Gene |
| | PTSETESVNTENVAGGDIEGENCGARLAHRISKSKFSRYWRRWNRF | ID ENS000000285479; Homo |
| | CRRKCRAAVKSNVFYWLVIFLVFLNTLTIASEHYNQPNWLTEVQDT | sapiens |
| | ANKALLALFTAEMLLKMYSLGLQAYFVSLFNRFDCFVVCGGILETIL | |
| | VETKIMSPLGISVLRCVRLLRIFKITRYWNSLSNLVASLLNSVRSIASL | |
| | LLLLFLFIIIFSLLGMQLFGGKFNFDEMQTRRSTFDNFPQSLLTVFQIL | |
| | TGEDWNSVMYDGIMAYGGPSFPGMLVCIYFIILFICGNYILLNVFLAI | |
| | AVDNLADAESLTSAQKEEEEEKERKKLARTASPEKKQELVEKPAVG | |
| | ESKEEKIELKSITADGESPPATKINMDDLQPNENEDKSPYPNPETTGE | |
| | EDEEEPEMPVGPRPRPLSELHLKEKAVPMPEASAFFIFSSNNRFRLQC | |
| | HRIVNDTIFTNLILFFILLSSISLAAEDPVQHTSFRNHILGNADYVFTSI | |
| | FTLEIILKMTAYGAFLHKGSFCRNYFNILDLLVVSVSLISFGIQSSAIN | |
| | VVKILRVLRVLRPLRAINRAKGLKHVVQCVFVAIRTIGNIVIVTTLLQ | |
| | FMFACIGVQLFKGKLYTCSDSSKQTEAECKGNYITYKDGEVDHPIIQ | |
| | PRSWENSKFDFDNVLAAMMALFTVSTFEGWPELLYRSIDSHTEDKG | |
| | PIYNYRVEISIFFIIYIIIIAFFMMNIFVGFVIVTFQEQGEQEYKNCELDK | |
| | NQRQCVEYALKARPLRRYIPKNQHQYKVWYVVNSTYFEYLMFVLI | |
| | LLNTICLAMQHYGQSCLFKIAMNILNMLFTGLFTVEMILKLIAFKPK | |
| | VGL | |
| 138 | MLRAFVQPGTPAYQPLPSHLSANTEVKFKGTLVHEAQLNYFYISPG | CACNA1C protein |
| | GSNYGSPRPAHANMNANAAAGLAPEHIPTPGAALSWQAAIDAARQ | (ENSP00000445849) encoded |
| | AKLMGSAGN | by Transcript ID |
| | | ENST00000543114 from Gene |
| | | ID ENSG00000151067; Homo |
| | | sapiens |
| | | CACNA1C protein |
| | | (ENSP00000493628) encoded |
| | | by Transcript ID |
| | | ENST00000642774 from Gene |
| | | ID ENS000000285479; Homo |
| | | sapiens |
| 139 | XSFPGMLVCIYFIILFICGNYILLNVFLAIAVDNLADAESLTSAQKEEE | CACNA1C protein |
| | EEKERKKLARTASPEKKQELVEKPAVGESKEEKIELKSITADGESPPA | (ENSP00000478996) encoded |
| | TKINMDDLQPNENEDKSPYPNPETTGEEDEEEPEMPVGPRPRPLSEL | by Transcript ID |
| | HLKEKAVPMPEASAFFIFSSNNRFRLQCHRIVNDTIFTNLILFFILLSSI | ENST00000465278 from Gene |
| | SLAAEDPVQHTSFRNHVCIACVFCTPSPWGCARPTASVADIIVQSGA | ID ENSG00000151067; Homo |
| | QF | sapiens |
| | | CACNA1C protein |
| | | (ENSP00000495307) encoded |
| | | by Transcript ID |
| | | ENST00000647024 from Gene |
| | | ID ENSG00000285479; Homo |
| | | sapiens |
| 140 | MAYVCKLLMSKTRPLVMIGVAWVMLLYRSIDSHTEDKGPIYNYRV | CACNA1C protein |
| | EISIFFIIYIIIIAFFMMNIFVGFVIVTFQEQGEQEYKNCELDKNQRQCV | (ENSP00000479274) encoded |
| | EYALKARPLRRYIPKNQHQYKVWYVVNSTYFEYLMFVLILLNTICL | by Transcript ID |
| | AMQ | ENST00000496818 from Gene |
| | | ID ENSG00000151067; Homo |
| | | sapiens |
| | | CACNA1C protein |
| | | (ENSP00000494843) encoded |
| | | by Transcript ID |
| | | ENST00000642396 from Gene |
| | | ID ENSG00000285479; Homo |
| | | sapiens |
| 141 | MFIVSQNAEENSRISITFFRLFRVMRLVKLLSRGEGIRTLLWTFIKSFQ | CACNA1C protein |
| | ALPYVALLIVMLFFIYAVIGMQVFGKIALNDTTEINRNNNFQTFPQA | (ENSP00000482910) encoded |
| | VLLLFRCATGEAWQDIMLACMPGKKCAPESEPSNSTEGETPCGSSFA | by Transcript ID |
| | VFYFISFYMLCAFLIINLFVAVIMDNFDYLTRDWSILGPHHLDEFKRI | ENST00000616390 from Gene |
| | WAEYDPEAKGRIKHLDVVTLLRRIQPPLGFGKLCPHRVACKRLVSM | ID ENSG00000151067; Homo |
| | NMPLNSDGTVMFNATLFALVRTALRIKTEGNLEQANEELRAIIKKIW | sapiens |
| | KRTSMKLLDQVVPPAGDDEVTVGKFYATFLIQEYFRKFKKRKEQGL | CACNA1C protein |
| | VGKPSQRNALSLQAGLRTLHDIGPEIRRAISGDLTAEEELDKAMKEA | (ENSP00000493529) encoded |
| | VSAASEDDIFRRAGGLFGNHVSYYQSDGRSAFPQTFTTQRPLHINKA | by Transcript ID |
| | GSSQGDTESPSHEKLVDSTFTPSSYSSTGSNANINNANNTALGRLPRP | ENST00000644343 from Gene |
| | AGYPSTVSTVEGHGPPLSPAIRVQEVAWKLSSNRCHSRESQAAMAG | ID ENSG00000285479; Homo |
| | QEETSQDETYEVKMNHDTEACSEPSLLSTEMLSYQDDENRQLTLPE | sapiens |
| | EDKRDIRQSPKRGFLRSASLGRRASFHLECLKRQKDRGGDISQKTVL | |
| | PLHLVHHQALAVAGLSPLLQRSHSPASFPRPFATPPATPGSRGWPPQ | |
| | PVPTLRLEGVESSEKLNSSFPSIHCGSWAETTPGGGGSSAARRVRPVS | |
| | LMVPSQAGAPGRQFHGSASSLVEAVLISEGLGQFAQDPKFIEVTIQE | |
| | LADACDMTIEEMESAADNILSGGAPQSPNGALLPFVNCRDAGQDRA | |
| | GGEEDAGCVRARGRPSEEELQDSRVYVSSL | |
| 142 | MMMMMMMKKMQHQRQQQADHANEANYARGTRLPLSGEGPTSQ | 55 |
| | PNSSKQTVLSWQAAIDAARQAKAAQTMSTSAPPPVGSLSQRKRQQY | |
| | AKSKKQGNSSNSRPARALFCLSLNNPIRRACISIVEWKPFDIFILLAIF | |
| | ANCVALAIYIPFPEDDSNSTNHNLEKVEYAFLIIFTVETFLKIIAYGLL | |
| | LHPNAYVRNGWNLLDFVIVIVGLFSVILEQLTKETEGGNHSSGKSGG | |
| | FDVKALRAFRVLRPLRLVSGVPSLQVVLNSIIKAMVPLLHIALLVLF | |
| | VIIIYAIIGLELFIGKMHKTCFFADSDIVAEEDPAPCAFSGNGRQCTAN | |
| | GTECRSGWVGPNGGITNFDNFAFAMLTVFQCITMEGWTDVLYWM | |
| | NDAMGFELPWVYFVSLVIFGSFFVLNLVLGVLSGEFSKEREKAKAR | |
| | GDFQKLREKQQLEEDLKGYLDWITQAEDIDPENEEEGGEEGKRNTS | |
| | MPTSETESVNTENVSGEGENRGCCGSLCQAISKSKLSRRWRRWNRF | |
| | NRRRCRAAVKSVTFYWLVIVLVFLNTLTISSEHYNQPDWLTQIQDIA | |
| | NKVLLALFTCEMLVKMYSLGLQAYFVSLFNRFDCFVVCGGITETILV | |
| | ELEIMSPLGISVFRCVRLLRIFKVTRHWTSLSNLVASLLNSMKSIASL | |
| | LLLLFLFIIIFSLLGMQLFGGKFNFDETQTKRSTFDNFPQALLTVFQ1L | |
| | TGEDWNAVMYDGIMAYGGPSSSGMIVCIYFIILFICGNYILLNVFLAI | |
| | AVDNLADAESLNTAQKEEAEEKERKKIARKESLENKKNNKPEVNQI | |
| | ANSDNKVTIDDYREEDEDKDPYPPCDVPVGEEEEEEEEDEPEVPAGP | |
| | RPRRISELNMKEKIAPIPEGSAFFILSKTNPIRVGCHKLINHHIFTNLIL | |
| | VFIMLSSAALAAEDPIRSHSFRNTILGYFDYAFTAIFTVEILLKMTTFG | |
| | AFLHKGAFCRNYFNLLDMLVVGVSLVSFGIQSSAISVVKILRVLRVL | |
| | RPLRAINRAKGLKHVVQCVFVAIRTIGNIMIVTTLLQFMFACIGVQLF | |
| | KGKFYRCTDEAKSNPEECRGLFILYKDGDVDSPVVRERIWQNSDFN | |
| | FDNVLSAMMALFTVSTFEGWPALLYKAIDSNGENIGPIYNHRVEISIF | |
| | FIIYIIIVAFFMMNIFVGFVIVTFQEQGEKEYKNCELDKNQRQCVEYA | |
| | LKARPLRRYIPKNPYQYKFWYVVNSSPFEYMMFVLIMLNTLCLAM | |
| | QHYEQSKMFNDAMDILNMVFTGVFTVEMVLKVIAFKPKGYFSDAW | |
| | NTFDSLIVIGSIIDVALSEADPTESENVPVPTATPGNSEESNRISITFFRL | |
| | FRVMRLVKLLSRGEGIRTLLWTFIKSFQALPYVALLIAMLFFIYAVIG | |
| | MQMFGKVAMRDNNQINRNNNFQTFPQAVLLLFRCATGEAWQEIML | |
| | ACLPGKLCDPESDYNPGEEYTCGSNFAIVYFISFYMLCAFLIINLFVA | |
| | VIMDNFDYLTRDWSILGPHHLDEFKRIWSEYDPEAKGRIKHLDVVT | |
| | LLRRIQPPLGFGKLCPHRVACKRLVAMNMPLNSDGTVMFNATLFAL | |
| | VRTALKIKTEGNLEQANEELRAVIKKIWKKTSMKLLDQVVPPAGDD | |
| | EVTVGKFYATFLIQDYFRKFKKRKEQGLVGKYPAKNTTIALQAGLR | |
| | TLHDIGPEIRRAISCDLQDDEPEETKREEEDDVFKRNGALLGNHVNH | |
| | VNSDRRDSLQQTNTTHRPLHVQRPSIPPASDTEKPLFPPAGNSVCHN | |
| | HHNHNSIGKQVPTSTNANLNNANMSKAAHGKRPSIGNLEHVSENG | |
| | HHSSHKHDREPQRRSSVKRTRYYETYIRSDSGDEQLPTICREDPEIHG | |
| | YFRDPHCLGEQEYFSSEECYEDDSSPTWSRQNYGYYSRYPGRNIDSE | |
| | RPRGYHHPQGFLEDDDSPVCYDSRRSPRRRLLPPTPASHRRSSFNFE | |
| | CLRRQSSQEEVPSSPIFPHRTALPLHLMQQQIMAVAGLDSSKAQKYS | |
| | PSHSTRSWATPPATPPYRDWTPCYTPLIQVEQSEALDQVNGSLPSLH | |
| | RSSWYTDEPDISYRTFTPASLTVPSSFRNKNSDKQRSADSLVEAVLIS | |
| | EGLGRYARDPKFVSATKHEIADACDLTIDEMESAASTLLNGNVRPR | |
| | ANGDVGPLSHRQDYELQDFGPGYSDEEPDPGRDEEDLADEMICTTTL | |
| 143 | MMMMMMMKKMQHQRQQQADHANEANYARGTRLPLSGEGPTSQ | CACNA1D protein |
| | PNSSKQTVLSWQAAIDAARQAKAAQTMSTSAPPPVGSLSQRKRQQY | (ENSP00000288139) encoded |
| | AKSKKQGNSSNSRPARALFCLSLNNPIRRACISIVEWKPFDIFILLAIF | by Transcript ID |
| | ANCVALAIYIPFPEDDSNSTNHNLEKVEYAFLIIFTVETFLKIIAYGLL | ENST00000288139 from Gene |
| | LHPNAYVRNGWNLLDFVIVIVGLFSVILEQLTKETEGGNHSSGKSGG | ID ENSG00000157388; Homo |
| | FDVKALRAFRVLRPLRLVSGVPSLQVVLNSIIKAMVPLLHIALLVLF | sapiens |
| | VIIIYAIIGLELFIGKMHKTCFFADSDIVAEEDPAPCAFSGNGRQCTAN | CACNA1D protein |
| | GTECRSGWVGPNGGITNFDNFAFAMLTVFQCITMEGWTDVLYWVN | (ENSP00000494413) encoded |
| | DAIGWEWPWVYFVSLIILGSFFVLNLVLGVLSGEFSKEREKAKARG | by Transcript ID |
| | DFQKLREKQQLEEDLKGYLDWITQAEDIDPENEEEGGEEGKRNTSM | ENST00000645528 from Gene |
| | PTSETESVNTENVSGEGENRGCCGSLWCWWRRRGAAKAGPSGCRR | ID ENSG00000157388; Homo |
| | WGQAISKSKLSRRWRRWNRFNRRRCRAAVKSVTFYWLVIVLVFLN | sapiens |
| | TLTISSEHYNQPDWLTQIQDIANKVLLALFTCEMLVKMYSLGLQAY | |
| | FVSLFNRFDCFVVCGGITETILVELEIMSPLGISVFRCVRLLRIFKVTR | |
| | HWTSLSNLVASLLNSMKSIASLLLLLFLFIIIFSLLGMQLFGGKFNFDE | |
| | TQTKRSTFDNFPQALLTVFQILTGEDWNAVMYDGIMAYGGPSSSGM | |
| | IVCIYFIILFICGNYILLNVFLAIAVDNLADAESLNTAQKEEAEEKERK | |
| | KIARKESLENKKNNKPEVNQIANSDNKVTIDDYREEDEDKDPYPPC | |
| | DVPVGEEEEEEEEDEPEVPAGPRPRRISELNMKEKIAPIPEGSAFFILS | |
| | KTNPIRVGCHKLINHHIFTNLILVFIMLSSAALAAEDPIRSHSFRNTIL | |
| | GYFDYAFTAIFTVEILLKMTTFGAFLHKGAFCRNYFNLLDMLVVGV | |
| | SLVSFGIQSSAISVVKILRVLRVLRPLRAINRAKGLKHVVQCVFVAIR | |
| | TIGNIMIVTTLLQFMFACIGVQLFKGKFYRCTDEAKSNPEECRGLFIL | |
| | YKDGDVDSPVVRERIWQNSDFNFDNVLSAMMALFTVSTFEGWPAL | |
| | LYKAIDSNGENIGPIYNHRVEISIFFIIYIIIVAFFMMNIFVGFVIVTFQE | |
| | QGEKEYKNCELDKNQRQCVEYALKARPLRRYIPKNPYQYKFWYVV | |
| | NSSPFEYMMFVLIMLNTLCLAMQHYEQSKMFNDAMDILNMVFTGV | |
| | FTVEMVLKVIAFKPKGYFSDAWNTFDSLIVIGSIIDVALSEADPTESE | |
| | NVPVPTATPGNSEESNRISITFFRLFRVMRLVKLLSRGEGIRTLLWTFI | |
| | KSFQALPYVALLIAMLFFIYAVIGMQMFGKVAMRDNNQINRNNNFQ | |
| | TFPQAVLLLFRCATGEAWQEIMLACLPGKLCDPESDYNPGEEYTCG | |
| | SNFAIVYFISFYMLCAFLIINLFVAVIMDNFDYLTRDWSILGPHHLDE | |
| | FKRIWSEYDPEAKGRIKHLDVVTLLRRIQPPLGFGKLCPHRVACKRL | |
| | VAMNMPLNSDGTVMFNATLFALVRTALKIKTEGNLEQANEELRAVI | |
| | KKIWKKTSMKLLDQVVPPAGDDEVTVGKFYATFLIQDYFRKFKKR | |
| | KEQGLVGKYPAKNTTIALQAGLRTLHDIGPEIRRAISCDLQDDEPEET | |
| | KREEEDDVFKRNGALLGNHVNHVNSDRRDSLQQTNTTHRPLHVQR | |
| | PSIPPASDTEKPLFPPAGNSVCHNHHNHNSIGKQVPTSTNANLNNAN | |
| | MSKAAHGKRPSIGNLEHVSENGHHSSHKHDREPQRRSSVKRTRYYE | |
| | TYIRSDSGDEQLPTICREDPEIHGYFRDPHCLGEQEYFSSEECYEDDS | |
| | SPTWSRQNYGYYSRYPGRNIDSERPRGYHHPQGFLEDDDSPVCYDS | |
| | RRSPRRRLLPPTPASHRRSSFNFECLRRQSSQEEVPSSPIFPHRTALPL | |
| | HLMQQQIMAVAGLDSSKAQKYSPSHSTRSWATPPATPPYRDWTPC | |
| | YTPLIQVEQSEALDQVNGSLPSLHRSSWYTDEPDISYRTFTPASLTVP | |
| | SSFRNKNSDKQRSADSLVEAVLISEGLGRYARDPKFVSATKHEIADA | |
| | CDLTIDEMESAASTLLNGNVRPRANGDVGPLSHRQDYELQDFGPGY | |
| | SDEEPDPGRDEEDLADEMICITTL | |
| 144 | MMMMMMMKKMQHQRQQQADHANEANYARGTRLPLSGEGPTSQ | CACNA1D protein |
| | PNSSKQTVLSWQAAIDAARQAKAAQTMSTSAPPPVGSLSQRKRQQY | (ENSP00000409174) encoded |
| | AKSKKQGNSSNSRPARALFCLSLNNPIRRACISIVEWKPFDIFILLAIF | by Transcript ID |
| | ANCVALAIYIPFPEDDSNSTNHNLEKVEYAFLIIFTVETFLKIIAYGLL | ENST00000422281 from Gene |
| | LHPNAYVRNGWNLLDFVIVIVGLFSVILEQLTKETEGGNHSSGKSGG | ID ENSG00000157388; Homo |
| | FDVKALRAFRVLRPLRLVSGVPSLQVVLNSIIKAMVPLLHIALLVLF | sapiens |
| | VIIIYAIIGLELFIGKMHKTCFFADSDIVAEEDPAPCAFSGNGRQCTAN | |
| | GTECRSGWVGPNGGITNFDNFAFAMLTVFQCITMEGWTDVLYWM | |
| | NDAMGFELPWVYFVSLVIFGSFFVLNLVLGVLSGEFSKEREKAKAR | |
| | GDFQKLREKQQLEEDLKGYLDWITQAEDIDPENEEEGGEEGKRNTS | |
| | MPTSETESVNTENVSGEGENRGCCGSLCQAISKSKLSRRWRRWNRF | |
| | NRRRCRAAVKSVTFYWLVIVLVFLNTLTISSEHYNQPDWLTQIQDIA | |
| | NKVLLALFTCEMLVKMYSLGLQAYFVSLFNREDCFVVCGGITETILV | |
| | ELEIMSPLGISVFRCVRLLRIFKVTRHWTSLSNLVASLLNSMKSIASL | |
| | LLLLFLFIIIFSLLGMQLFGGKFNFDETQTKRSTFDNFPQALLTVFQIL | |
| | TGEDWNAVMYDGIMAYGGPSSSGMIVCIYFIILFICGNYILLNVFLAI | |
| | AVDNLADAESLNTAQKEEAEEKERKKIARKESLENKKNNKPEVNQI | |
| | ANSDNKVTIDDYREEDEDKDPYPPCDVPVGEEEEEEEEDEPEVPAGP | |
| | RPRRISELNMKEKIAPIPEGSAFFILSKTNPIRVGCHKLINHHIFTNLIL | |
| | VFIMLSSAALAAEDPIRSHSFRNTILGYFDYAFTAIFTVEILLKMTTFG | |
| | AFLHKGAFCRNYFNLLDMLVVGVSLVSFGIQSSAISVVKILRVLRVL | |
| | RPLRAINRAKGLKHVVQCVFVAIRTIGNIMIVTTLLQFMFACIGVQLF | |
| | KGKFYRCTDEAKSNPEECRGLFILYKDGDVDSPVVRERIWQNSDFN | |
| | FDNVLSAMMALFTVSTFEGWPALLYKAIDSNGENIGPIYNFIRVEISIF | |
| | FIIYIIIVAFFMMNIFVGFVIVTFQEQGEKEYKNCELDKNQRQCVEYA | |
| | LKARPLRRYIPKNPYQYKFWYVVNSSPFEYMMFVLIMLNTLCLAM | |
| | QHYEQSKMFNDAMDILNMVFTGVFTVEMVLKVIAFKPKGYFSDAW | |
| | NTFDSLIVIGSIIDVALSEADNSEESNRISITFFRLFRVMRLVKLLSRGE | |
| | GIRTLLWTFIKSFQALPYVALLIAMLFFIYAVIGMQMFGKVAMRDN | |
| | NQINRNNNFQTFPQAVLLLERCATGEAWQEIMLACLPGKLCDPESD | |
| | YNPGEEYTCGSNFAIVYFISFYMLCAFLIINLEVAVIMDNFDYLTRD | |
| | WSILGPHHLDEFKRIWSEYDPEAKGRIKHLDVVTLLRRIQPPLGFGK | |
| | LCPHRVACKRLVAMNMPLNSDGTVMFNATLFALVRTALKIKTEGN | |
| | LEQANEELRAVIKKIWKKTSMKLLDQVVPPAGDDEVTVGKEYATFL | |
| | IQDYFRKFKKRKEQGLVGKYPAKNTTIALQAGLRTLHDIGPEIRRAIS | |
| | CDLQDDEPEETKREEEDDVFKRNGALLGNHVNHVNSDRRDSLQQT | |
| | NTTHRPLHVQRPSIPPASDTEKPLFPPAGNSVCHNHHNHNSIGKQVP | |
| | TSTNANLNNANMSKAAHGKRPSIGNLEHVSENGHHSSHKHDREPQ | |
| | RRSSVKRSDSGDEQLPTICREDPEIHGYFRDPHCLGEQEYFSSEECYE | |
| | DDSSPTWSRQNYGYYSRYPGRNIDSERPRGYHHPQGFLEDDDSPVC | |
| | YDSRRSPRRRLLPPTPASHRRSSFNFECLRRQSSQEEVPSSPIFPHRTA | |
| | LPLHLMQQQIMAVAGLDSSKAQKYSPSHSTRSWATPPATPPYRDWT | |
| | PCYTPLIQVEQSEALDQVNGSLPSLHRSSWYTDEPDISYRTFTPASLT | |
| | VPSSFRNKNSDKQRSADSLVEAVLISEGLGRYARDPKFVSATKHEIA | |
| | DACDLTIDEMESAASTLLNGNVRPRANGDVGPLSHRQDYELQDFGP | |
| | GYSDEEPDPGRDEEDLADEMICITTL | |
| 145 | ANGTECRSGWVGPNGGITNFDNFAFAMLTVFQCITMEGWTDVLYW | CACNA1D protein |
| | MNDAMGFELPWVYFVSLVIFGSFFVLNLVLGVLSGEFSKEREKAKA | (ENSP00000418014) encoded |
| | RGDFQKLREKQQLEEDLKGYLDWITQAEDIDPENEEEGGEEGKRNT | by Transcript ID |
| | SMPTSETESVNTENVSGEGENRGCCGSLWCWWRRRGAAKAGPSGC | ENST00000481478 from Gene |
| | RRWGQAISKSKLSRRWRRWNRFNRRRCRAAVKSVTFYWLVIVLVF | ID ENSG00000157388; Homo |
| | LNTLTISSEHYNQPDWLTQIQDIANKVLLALFTCEMLVKMYSLGLQ | sapiens |
| | AYFVSLFNRFDCFVVCGGITETILVELEIMSPLGISVFRCVRLLRIFKV | |
| | TRHWTSLSNLVASLLNSMKSIASLLLLLFLFIIIFSLLGMQLFGGKFNF | |
| | DETQTKRSTFDNFPQALLTVFQILTGEDWNAVMYDGIMAYGGPSSS | |
| | GMIVCIYFIILFICGNYILLNVFLAIAVDNLADAESLNTAQKEEAEEKE | |
| | RKKIARKESLENKKNNKPEVNQIANSDNKVTIDDYREEDEDKDPYP | |
| | PCDVPVGEEEEEEEEDEPEVPAGPRPRRISELNMKEKIAPIPEGSAFFI | |
| | LSKTNPIRVGCHKLINHHIFTNLILVFIMLSSAALAAEDPIRSHSFRNTI | |
| | LGYFDYAFTAIFTVEILLKMTTFGAFLHKGAFCRNYFNLLDMLVVG | |
| | VSLVSFGIQSSAISVVKILRVLRVLRPLRAINRAKGLKHVVQCVFVAI | |
| | RTIGNIMIVTTLLQFMFACIGVQLFKGKEYRCTDEAKSNPEECRGLFI | |
| | LYKDGDVDSPVVRERIWQNSDFNFDNVLSAMMALFTVSTFEGWPA | |
| | LLYKAIDSNGENIGPIYNHRVEISIFFIIYIIIVAFFMMNIFVGFVIVTFQ | |
| | EQGEKEYKNCELDKNQRQCVEYALKARPLRRYIPKNPYQYKFWYV | |
| | VNSSPFEYMMFVLIMLNTLCLAMQHYEQSKMFNDAMDILNMVFTG | |
| | VFTVEMVLKVIAFKPKHYFTDAWNTFDALIVVGSVVDIAITEVNPTE | |
| | SENVPVPTATPGNSEESNRISITFFRLFRVMRLVKLLSRGEGIRTLLW | |
| | TFIKSFQALPYVALLIAMLFFIYAVIGMQMFGKVAMRDNNQINRNN | |
| | NFQTFPQAVLLLFRCATGEAWQEIMLACLPGKLCDPESDYNPGEEY | |
| | TCGSNFAIVYFISFYMLCAFLIINLFVAVIMDNFDYLTRDWSILGPHH | |
| | LDEFKRIWSEYDPEAKGRIKHLDVVTLLRRIQPPLGFGKLCPHRVAC | |
| | KRLVAMNMPLNSDGTVMFNATLFALVRTALKIKTEGNLEQANEEL | |
| | RAVIKKIWKKTSMKLLDQVVPPAGDDEVTVGKFYATFLIQDYFRKF | |
| | KKRKEQGLVGKYPAKNTTIALQAGLRTLHDIGPEIRRAISCDLQDDE | |
| | PEETKREEEDDVFKRNGALLGNHVNHVNSDRRDSLQQTNTTHRPLH | |
| | VQRPSIPPASDTEKPLFPPAGNSVCHNHHNHNSIGKQVPTSTNANLN | |
| | NANMSKAAHGKRPSIGNLEHVSENGHHSSHKHDREPQRRSSVKRTR | |
| | YYETYIRSDSGDEQLPTICREDPEIHGYFRDPHCLGEQEYFSSEECYE | |
| | DDSSPTWSRQNYGYYSRYPGRNIDSERPRGYHHPQGFLEDDDSPVC | |
| | YDSRRSPRRRLLPPTPASHRRSSFNFECLRRQSSQEEVPSSPIFPHRTA | |
| | LPLHLMQQQIMAVAGLDSSKAQKYSPSHSTRSWATPPATPPYRDWT | |
| | PCYTPLIQVEQSEALDQVNGSLPSLHRSSWYTDEPDISYRTFTPASLT | |
| | VPSSFRNKNSDKQRSADSLVEAVLISEGLGRYARDPKFVSATKHEIA | |
| | DACDLTIDEMESAASTLLNGNVRPRANGDVGPLSHRQDYELQDFGP | |
| | GYSDEEPDPGRDEEDLADEMICITTL | |
| 146 | XPCAFSGNGRQCTANGTECRSGWVGPNGGITNFDNFAFAMLTVFQ | CACNA1D protein |
| | CITMEGWTDVLYWVNDAIGWEWPWVYFVSLIILGSFFVLNLVLGVL | (ENSP00000418045) encoded |
| | SGEFSKEREKAKARGDFQKLREKQQLEEDLKGYLDWITQAEDIDPE | by Transcript ID |
| | NEEEGGEEGKRNRVTVADLLKEDKKKKKFCCFRQRRAKDHASMPT | ENST00000481085 from Gene |
| | SETESVNTENVSGEGENRGCCGSLCQAISKSKLSRRWRRWNRFNRR | ID ENSG00000157388; Homo |
| | RCRAAVKSVTFYWLVIVLVFLNTLIISSEHYNQPDW | sapiens |
| 147 | MEMNLPTFSSDLIFIKSTMYQEIDARYKGKVISEAETKGYFSLAFGEA | CACNA1D protein |
| | NYARGTRLPLSGEGPTSQPNSSKQTVLSWQAAIDAARQAKAAQTMS | (ENSP00000489769) encoded |
| | TSAPPPVGSLSQRKRQQYAKSKKQGNSSNSRPARALFCLSLNNPIRR | by Transcript ID |
| | ACISIVEWKPFDIFILLAIFANCVALAIYIPFPEDDSNSTNHNLEKVEY | ENST00000637424 from Gene |
| | AFLIIFTVETFLKIIAYGLLLHPNAYVRNGWNLLDFVIVIVGLFSVILE | ID ENSG00000157388; Homo |
| | QLTKETEGGNHSSGKSGGFDVKALRAFRVLRPLRLVSGVPSLQVVL | sapiens |
| | NSIIKAMVPLLHIALLVLFVIIIYAIIGLELFIGKMHKTCFFADSDIVAE | |
| | EDPAPCAFSGNGRQCTANGTECRSGWVGPNGGITNFDNFAFAMLTV | |
| | FQCITMEGWTDVLYWVNDAIGWEWPWVYFVSLIILGSFFVLNLVLG | |
| | VLSGEFSKEREKAKARGDFQKLREKQQLEEDLKGYLDWITQAEDID | |
| | PENEEEGGEEGKRNTSMPTSETESVNTENVSGEGENRGCCGSLCQAI | |
| | SKSKLSRRWRRWNRFNRRRCRAAVKSVTFYWLVIVLVFLNTLTISS | |
| | EHYNQPDWLTQIQDIANKVLLALFTCEMLVKMYSLGLQAYFVSLFN | |
| | RFDCFVVCGGITETILVELEIMSPLGISVFRCVRLLRIFKVTRHWTSLS | |
| | NLVASLLNSMKSIASLLLLLFLFIIIFSLLGMQLFGGKFNFDETQTKRS | |
| | TFDNFPQALLTVFQILTGEDWNAVMYDGIMAYGGPSSSGMIVCIYFI | |
| | ILFICGNYILLNVFLAIAVDNLADAESLNTAQKEEAEEKERKKIARKE | |
| | SLENKKNNKPEVNQIANSDNKVTIDDYREEDEDKDPYPPCDVPVGE | |
| | EEEEEEEDEPEVPAGPRPRRISELNMKEKIAPIPEGSAFFILSKTNPIRV | |
| | GCHKLINHHIFTNLILVFIMLSSAALAAEDPIRSHSFRNTILGYFDYAF | |
| | TAIFTVEILLKMTTFGAFLHKGAFCRNYFNLLDMLVVGVSLVSFGIQ | |
| | SSAISVVKILRVLRVLRPLRAINRAKGLKHVVQCVFVAIRTIGNIMIV | |
| | TTLLQFMFACIGVQLFKGKFYRCTDEAKSNPEECRGLFILYKDGDVD | |
| | SPVVRERIWQNSDFNFDNVLSAMMALFTVSTFEGWPALLYKAIDSN | |
| | GENIGPIYNHRVEISIFFIIYIIIVAFFMMNIFVGFVIVTFQEQGEKEYKN | |
| | CELDKNQRQCVEYALKARPLRRYIPKNPYQYKFWYVVNSSPFEYM | |
| | MFVLIMLNTLCLAMQHYEQSKMFNDAMDILNMVFTGVFTVEMVL | |
| | KVIAFKPKGYFSDAWNTFDSLIVIGSIIDVALSEADNSEESNRISITFFR | |
| | LFRVMRLVKLLSRGEGIRTLLWTFIKSFQALPYVALLIAMLFFIYAVI | |
| | GMQMFGKVAMRDNNQINRNNNFQTFPQAVLLLFRCATGEAWQEI | |
| | MLACLPGKLCDPESDYNPGEEYTCGSNFAIVYFISFYMLCAFLIINLF | |
| | VAVIMDNFDYLTRDWSILGPHHLDEFKRIWSEYDPEAKGRIKHLDV | |
| | VTLLRRIQPPLGFGKLCPHRVACKRLVAMNMPLNSDGTVMFNATLF | |
| | ALVRTALKIKTEGNLEQANEELRAVIKKIWKKTSMKLLDQVVPPAG | |
| | DDEVTVGKFYATFLIQDYFRKFKKRKEQGLVGKYPAKNTTIALQAG | |
| | LRTLHDIGPEIRRAISCDLQDDEPEETKREEEDDVFKRNGALLGNHV | |
| | NHVNSDRRDSLQQTNTTHRPLHVQRPSIPPASDTEKPLFPPAGNSVC | |
| | HNHHNHNSIGKQVPTSTNANLNNANMSKAAHGKRPSIGNLEHVSE | |
| | NGHHSSHKHDREPQRRSSVKRTRYYETYIRSDSGDEQLPTICREDPEI | |
| | HGYFRDPHCLGEQEYFSSEECYEDDSSPTWSRQNYGYYSRYPGRNI | |
| | DSERPRGYHHPQGFLEDDDSPVCYDSRRSPRRRLLPPTPASHRRSSF | |
| | NFECLRRQSSQEEVPSSPIFPHRTALPLHLMQQQIMAVAGLDSSKAQ | |
| | KYSPSHSTRSWATPPATPPYRDWTPCYTPLIQVEQSEALDQVNGSLP | |
| | SLHRSSWYTDEPDISYRTFTPASLTVPSSFRNKNSDKQRSADSLVEA | |
| | VLISEGLGRYARDPKFVSATKHEIADACDLTIDEMESAASTLLNGNV | |
| | RPRANGDVGPLSHRQDYELQDFGPGYSDEEPDPGRDEEDLADEMIC | |
| | ITTL | |
| 148 | MMNIFVGFVIVTFQEQGEKEYKNCELDKNQRQCVEYALKARPLRR | CACNA1D protein |
| | YIPKNPYQYKFWYVVNSSPFEYMMFVLIMLNTLCLAMQHYEQSKM | (ENSP00000489790) encoded |
| | FNDAMDILNMVFTGVFTVEMVLKVIAFKPKGYFSDAWNTFDSLIVI | by Transcript ID |
| | GSIIDVALSEADNSEESNRISITFFRLFRVMRLVKLLSRGEGIRTLLWT | ENST00000638120 from Gene |
| | FIKSFQALPYVALLIAMLFFIYAVIGMQMFGKVAMRDNNQINRNNN | ID ENSG00000157388; Homo |
| | FQTFPQAVLLLFRCATGEAWQEIMLACLPGKLCDPESDYNPGEEYT | sapiens |
| | CGSNFAIVYFISFYMLCAFLIINLFVAVIMDNFDYLTRDWSILGPHHL | |
| | DEFKRIWSEYDPEAKGRIKHLDVVTLLRRIQPPLGFGKLCPHRVACK | |
| | RLVAMNMPLNSDGTVMFNATLFALVRTALKIKTEGNLEQANEELR | |
| | AVIKKIWKKTSMKLLDQVVPPAGDDEVTVGKFYATFLIQDYFRKFK | |
| | KRKEQGLVGKYPAKNTTIALQMLERML | |
| 149 | EEEGGEEGKRNRVTVADLLKEDKKKKKFCCFRQRRAKDHASMPTS | CACNA1D protein |
| | ETESVNTENVSGEGENRGCCGSLWCWW | (ENSP00000489962) encoded |
| | | by Transcript ID |
| | | ENS100000637301 from Gene |
| | | ID ENSG00000157388; Homo |
| | | sapiens |
| 150 | MMMMMMMKKMQHQRQQQADHANEANYARGTRLPLSGEGPTSQ | CACNA1D protein |
| | PNSSKQTVLSWQAAIDAARQAKAAQTMSTSAPPPVGSLSQRKRQQY | (ENSP00000490039) encoded |
| | AKSKKQGNSSNSRPARALFCLSLNNPIRRACISIVEWKPFDIFILLAIF | by Transcript ID |
| | ANCVALAIYIPFPEDDSNSTNHNLEKVEYAFLIIFTVETFLKIIAYGLL | ENST00000636938 from Gene |
| | LHPNAYVRNGWNLLDFVIVIVGLFSVILEQLTKETEGGNHSSGKSGG | ID ENSG00000157388; Homo |
| | FDVKALRAFRVLRPLRLVSGVPSLQVVLNSIIKAMVPLLHIALLVLF | sapiens |
| | VIIIYAIIGLELFIGKMHKTCFFADSDIVAEEDPAPCAFSGNGRQCTAN | |
| | GTECRSGWVGPNGGITNFDNFAFAMLTVFQCITMEGWTDVLYWVN | |
| | DAIGWEWPWVYFVSLIILGSFFVLNLVLGVLSGEFSKEREKAKARG | |
| | DFQKLREKQQLEEDLKGYLDWITQAEDIDPENEEEGGEEGKRNTSM | |
| | PTSETESVNTENVSGEGENRGCCGSLCQAISKSKLSRRWRRWNRFN | |
| | RRRCRAAVKSVTFYWLVIVLVFLNTLTISSEHYNQPDWLTQIQDIAN | |
| | KVLLALFTCEMLVKMYSLGLQAYFVSLFNRFDCFVVCGGITETILVE | |
| | LEIMSPLGISVFRCVRLLRIFKVTRHWTSLSNLVASLLNSMKSIASLL | |
| | LLLFLFIIIFSLLGMQLFGGKFNFDETQTKRSTFDNFPQALLTVFQILT | |
| | GEDWNAVMYDGIMAYGGPSSSGMIVCIYFIILFICGNYILLNVFLAIA | |
| | VDNLADAESLNTAQKEEAEEKERKKIARKESLENKKNNKPEVNQIA | |
| | NSDNKVTIDDYREEDEDKDPYPPCDVPVGEEEEEEEEDEPEVPAGPR | |
| | PRRISELNMKEKIAPIPEGSAFFILSKTNPIRVGCHKLINHHIFTNLILV | |
| | FIMLSSAALAAEDPIRSHSFRNTILGYFDYAFTAIFTVEILLKMTTFGA | |
| | FLHKGAFCRNYFNLLDMLVVGVSLVSFGIQSSAISVVKILRVLRVLR | |
| | PLRAINRAKGLKHVVQCVFVAIRTIGNIMIVTTLLQFMFACIGVQLFK | |
| | GKFYRCTDEAKSNPEECRGLFILYKDGDVDSPVVRERIWQNSDFNF | |
| | DNVLSAMMALFTVSTFEGWPALLYKAIDSNGENIGPIYNHRVEISIFF | |
| | IIYIIIVAFFMMNIFVGFVIVTFQEQGEKEYKNCELDKNQRQCVEYAL | |
| | KARPLRRYIPKNPYQYKFWYVVNSSPFEYMMFVLIMLNTLCLAMQ | |
| | HYEQSKMFNDAMDILNMVFTGVFTVEMVLKVIAFKPKGYFSDAWN | |
| | TFDSLIVIGSIIDVALSEADPTESENVPVPTATPGNSEESNRISITFFRLF | |
| | RVMRLVKLLSRGEGIRTLLWTFIKSFQALPYVALLIAMLFFIYAVIG | |
| | MQMFGKVAMRDNNQINRNNNFQTFPQAVLLLFRCATGEAWQEIML | |
| | ACLPGKLCDPESDYNPGEEYTCGSNFAIVYFISFYMLCAFLIINLFVA | |
| | VIMDNFDYLTRDWSILGPHHLDEFKRIWSEYDPEAKGRIKHLDVVT | |
| | LLRRIQPPLGFGKLCPHRVACKRLVAMNMPLNSDGTVMFNATLFAL | |
| | VRTALKIKTEGNLEQANEELRAVIKKIWKKTSMKLLDQVVPPAGDD | |
| | EVTVGKFYATFLIQDYFRKFKKRKEQGLVGKYPAKNTTIALQMLER | |
| | ML | |
| 151 | XADAESLNTAQKEEAEEKERKKIARKESLENKKNNKPEVNQIANSD | CACNA1D protein |
| | NKVTIDDYREEDEDKDPYPPCDVPGEEEEEEEEDEPEVPAGPRPRRIS | (ENSP00000490108) encoded |
| | ELNMKEKIAPIPEGSAFFILSKTNPIRVGCHKLINHHIFTNLILVFIMLS | by Transcript ID |
| | SAALAAEDPIRSHSFRNTMLGYADYVFTGTFAFEIILKMTTFGAFLH | ENST00000637714 from Gene |
| | KGAFCRNYFNLLDMLVVGVSLVSFGIQSSAISVVKILRVLRVLRPLR | ID ENSG00000157388; Homo |
| | AINRAKGLKHVVQCVFVAIRTIGNIMIVTTLLQFMFACIGVQLFKGK | sapiens |
| | FYRCTDEAKSNP | |
| 152 | MMMMMMMKKMQHQRQQQADHANEANYARGTRLPLSGEGPTSQ | CACNA1D protein |
| | PNSSKQTVLSWQAAIDAARQAKAAQTMSTSAPPPVGSLSQRKRQQY | (ENSP00000490183) encoded |
| | AKSKKQGNSSNSRPARALFCLSLNNPIRRACISIVEWKPFDIFILLAIF | by Transcript ID |
| | ANCVALAIYIPFPEDDSNSTNHNLEKVEYAFLIIFTVETFLKIIAYGLL | ENST00000636570 from Gene |
| | LHPNAYVRNGWNLLDFVIVIVGLFSVILEQLTKETEGGNHSSGKSGG | ID ENSG00000157388; Homo |
| | FDVKALRAFRVLRPLRLVSGVPSLQVVLNSIIKAMVPLLHIALLVLF | sapiens |
| | VIIIYAIIGLELFIGKMHKTCFFADSDIVAEEDPAPCAFSGNGRQCTAN | |
| | GTECRSGWVGPNGGITNFDNFAFAMLTVFQCITMEGWTDVLYWVN | |
| | DAIGWEWPWVYFVSLIILGSFFVLNLVLGVLSGEFSKEREKAKARG | |
| | DFQKLREKQQLEEDLKGYLDWITQAEDIDPENEEEGGEEGKRNTSM | |
| | PTSETESVNTENVSGEGENRGCCGSLCQAISKSKLSRRWRRWNRFN | |
| | RRRCRAAVKSVTFYWLVIVLVFLNTLTISSEHYNQPDWLTQIQDIAN | |
| | KVLLALFTCEMLVKMYSLGLQAYFVSLFNRFDCFVVCGGITETILVE | |
| | LEIMSPLGISVFRCVRLLRIFKVTRHWTSLSNLVASLLNSMKSIASLL | |
| | LLLFLFIIIFSLLGMQLFGGKFNFDETQTKRSTFDNFPQALLTVFQILT | |
| | GEDWNAVMYDGIMAYGGPSSSGMIVCIYFIILFICGNYILLNVFLAIA | |
| | VDNLADAESLNTAQKEEAEEKERKKIARKESLENKKNNKPEVNQIA | |
| | NSDNKVTIDDYREEDEDKDPYPPCDVPVGEEEEEEEEDEPEVPAGPR | |
| | PRRISELNMKEKIAPIPEGSAFFILSKTNPIRVGCHKLINHHIFTNLILV | |
| | FIMLSSAALAAEDPIRSHSFRNTILGYFDYAFTAIFTVEILLKMTTFGA | |
| | FLHKGAFCRNYFNLLDMLVVGVSLVSFGIQSSAISVVKILRVLRVLR | |
| | PLRAINRAKGLKHVVQCVFVAIRTIGNIMIVTTLLQFMFACIGVQLFK | |
| | GKFYRCTDEAKSNPEECRGLFILYKDGDVDSPVVRERIWQNSDFNF | |
| | DNVLSAMMALFTVSTFEGWPALLYKAIDSNGENIGPIYNHRVEISIFF | |
| | IIYIIIVAFFMMNIFVGFVIVTFQEQGEKEYKNCELDKNQRQCVEYAL | |
| | KARPLRRYIPKNPYQYKFWYVVNSSPFEYMMFVLIMLNTLCLAMQ | |
| | HYEQSKMFNDAMDILNMVFTGVFTVEMVLKVIAFKPKGYFSDAWN | |
| | TFDSLIVIGSIIDVALSEADNSEESNRISITFFRLFRVMRLVKLLSRGEG | |
| | IRTLLWTFIKSFQALPYVALLIAMLFFIYAVIGMQMFGKVAMRDNN | |
| | QINRNNNFQTFPQAVLLLFRCATGEAWQEIMLACLPGKLCDPESDY | |
| | NPGEEYTCGSNFAIVYFISFYMLCAFLIINLFVAVIMDNFDYLTRDWS | |
| | ILGPHHLDEFKRIWSEYDPEAKGRIKHLDVVTLLRRIQPPLGFGKLCP | |
| | HRVACKRLVAMNMPLNSDGTVMFNATLFALVRTALKIKTEGNLEQ | |
| | ANEELRAVIKKIWKKTSMKLLDQVVPPAGDDEVTVGKFYATFLIQD | |
| | YFRKFKKRKEQGLVGKYPAKNTTIALQAGLRTLHDIGPEIRRAISCD | |
| | LQDDEPEETKREEEDDVFKRNGALLGNHVNHVNSDRRDSLQQTNT | |
| | THRPLHVQRPSIPPASDTEKPLFPPAGNSVCHNHHNHNSIGKQVPTST | |
| | NANLNNANMSKAAHGKRPSIGNLEHVSENGHHSSHKHDREPQRRS | |
| | SVKRTRYYETYIRSDSGDEQLPTICREDPEIHGYFRDPHCLGEQEYFS | |
| | SEECYEDDSSPTWSRQNYGYYSRYPGRNIDSERPRGYHHPQGFLED | |
| | DDSPVCYDSRRSPRRRLLPPTPASHRRSSFNFECLRRQSSQEEVPSSPI | |
| | FPHRTALPLHLMQQQIMAVAGLDSSKAQKYSPSHSTRSWATPPATP | |
| | PYRDWTPCYTPLIQVEQSEALDQVNGSLPSLHRSSWYTDEPDISYRT | |
| | FTPASLTVPSSFRNKNSDKQRSADSLVEAVLISEGLGRYARDPKFVS | |
| | ATKHEIADACDLTIDEMESAASTLLNGNVRPRANGDVGPLSHRQDY | |
| | ELQDFGPGYSDEEPDPGRDEEDLADEMICITTL | |
| 153 | FFMMNIFVGFVIVTFQEQGEKEYKNCELDKNQRQCVEYALKARPLR | CACNA1D protein |
| | RYIPKNPYQYKFWYVVNSSPFEYMMFVLIMLNTLCLAMQHYEQSK | (ENSP00000490523) encoded |
| | MFNDAMDILNMVFTGVFTVEMVLKVIAFKPKALPYVALLIAMLFFI | by Transcript ID |
| | YAVIGMQMFGKVAMRDNNQINRNNNFQTFPQAVLLLFRCATGEA | ENST00000638129 from Gene |
| | WQEIMLACLPGKLCDPESDYNPGEEYTCGSNFAWYFISFYM | ID ENSG00000157388; Homo |
| | | sapiens |
| 154 | XVFTGVFTVEMVLKVIAFKPKHYFTDAWNTFDALIVVGSVVDIAITE | CACNA1D protein |
| | VNPTESENVPVPTATPGNSEESNRISITFFRLFRVMRLVKLLSRGEGIR | (ENSP00000490839) encoded |
| | TLLWTFIKSFQALPYVALLIAMLFFIYAVIGMQMFGKVAMRDNNQI | by Transcript ID |
| 152 | MMMMMMMKKMQHQRQQQADHANEANYARGTRLPLSGEGPTSQ | CACNA1D protein |
| | PNSSKQTVLSWQAAIDAARQAKAAQTMSTSAPPPVGSLSQRKRQQY | (ENSP00000490183) encoded |
| | AKSKKQGNSSNSRPARALFCLSLNNPIRRACISIVEWKPFDIFILLAIF | by Transcript ID |
| | ANCVALAIYIPFPEDDSNSTNIINLEKVEYAFLIIFTVETFLKIIAYGLL | ENST00000636570 from Gene |
| | LHPNAYVRNGWNLLDFVIVIVGLFSVILEQLTKETEGGNHSSGKSGG | ID ENSG00000157388; Homo |
| | FDVKALRAFRVLRPLRLVSGVPSLQVVLNSIIKAMVPLLHIALLVLF | sapiens |
| | VIIIYAIIGLELFIGKMHKTCFFADSDIVAEEDPAPCAFSGNGRQCTAN | |
| | GTECRSGWVGPNGGITNFDNFAFAMLTVFQCITMEGWTDVLYWVN | |
| | DAIGWEWPWVYFVSLIILGSFFVLNLVLGVLSGEFSKEREKAKARG | |
| | DFQKLREKQQLEEDLKGYLDWITQAEDIDPENEEEGGEEGKRNTSM | |
| | PTSETESVNTENVSGEGENRGCCGSLCQAISKSKLSRRWRRWNRFN | |
| | RRRCRAAVKSVTFYWLVIVLVFLNTLTISSEHYNQPDWLTQIQDIAN | |
| | KVLLALFTCEMLVKMYSLGLQAYFVSLFNRFDCFVVCGGITETILVE | |
| | LEIMSPLGISVFRCVRLLRIFKVTRHWTSLSNLVASLLNSMKSIASLL | |
| | LLLFLFIIIFSLLGMQLFGGKFNFDETQTKRSTFDNFPQALLTVFQILT | |
| | GEDWNAVMYDGIMAYGGPSSSGMIVCIYFIILFICGNYILLNVFLAIA | |
| | VDNLADAESLNTAQKEEAEEKERKKIARKESLENKKNNKPEVNQIA | |
| | NSDNKVTIDDYREEDEDKDPYPPCDVPVGEEEEEEEEDEPEVPAGPR | |
| | PRRISELNMKEKIAPIPEGSAFFILSKTNPIRVGCHKLINHHIFTNLILV | |
| | FIMLSSAALAAEDPIRSHSFRNTILGYFDYAFTAIFTVEILLKMTTFGA | |
| | FLHKGAFCRNYFNLLDMLVVGVSLVSFGIQSSAISVVKILRVLRVLR | |
| | PLRAINRAKGLKHVVQCVFVAIRTIGNIMIVTTLLQFMFACIGVQLFK | |
| | GKFYRCTDEAKSNPEECRGLFILYKDGDVDSPVVRERIWQNSDFNF | |
| | DNVLSAMMALFTVSTFEGWPALLYKAIDSNGENIGPIYNHRVEISIFF | |
| | IIYIIIVAFFMNINIFVGFVIVTFQEQGEKEYKNCELDKNQRQCVEYAL | |
| | KARPLRRYIPKNPYQYKFWYVVNSSPFEYMMFVLIMLNTLCLAMQ | |
| | HYEQSKMFNDAMDILNMVFTGVFTVEMVLKVIAFKPKGYFSDAWN | |
| | TFDSLIVIGSIIDVALSEADNSEESNRISITFFRLFRVMRLVKLLSRGEG | |
| | IRTLLWTFIKSFQALPYVALLIAMLFFIYAVIGMQMFGKVAMRDNN | |
| | QINRNNNFQTFPQAVLLLFRCATGEAWQEIMLACLPGKLCDPESDY | |
| | NPGEEYTCGSNFAIVYFISFYMLCAFLIINLFVAVIMDNFDYLTRDWS | |
| | ILGPHHLDEFKRIWSEYDPEAKGRIKHLDVVTLLRRIQPPLGFGKLCP | |
| | HRVACKRLVAMNMPLNSDGTVMFNATLFALVRTALKIKTEGNLEQ | |
| | ANEELRAVIKKIWKKTSMKLLDQVVPPAGDDEVTVGKFYATFLIQD | |
| | YFRKFKKRKEQGLVGKYPAKNTTIALQAGLRTLHDIGPEIRRAISCD | |
| | LQDDEPEETKREEEDDVFKRNGALLGNHVNHVNSDRRDSLQQTNT | |
| | THRPLHVQRPSIPPASDTEKPLFPPAGNSVCHNHHNHNSIGKQVPTST | |
| | NANLNNANMSKAAHGKRPSIGNLEHVSENGHHSSHKHDREPQRRS | |
| | SVKRTRYYETYIRSDSGDEQLPTICREDPEIHGYFRDPHCLGEQEYFS | |
| | SEECYEDDSSPTWSRQNYGYYSRYPGRNIDSERPRGYHHPQGFLED | |
| | DDSPVCYDSRRSPRRRLLPPTPASHRRSSFNFECLRRQSSQEEVPSSPI | |
| | FPHRTALPLHLMQQQIMAVAGLDSSKAQKYSPSHSTRSWATPPATP | |
| | PYRDWTPCYTPLIQVEQSEALDQVNGSLPSLHRSSWYTDEPDISYRT | |
| | FTPASLTVPSSFRNKNSDKQRSADSLVEAVLISEGLGRYARDPKFVS | |
| | ATKHEIADACDLTIDEMESAASTLLNGNVRPRANGDVGPLSHRQDY | |
| | ELQDFGPGYSDEEPDPGRDEEDLADEMICITTL | |
| 153 | FFMMNIFVGFVIVTFQEQGEKEYKNCELDKNQRQCVEYALKARPLR | CACNA1D protein |
| | RYIPKNPYQYKFWYVVNSSPFEYMMFVLIMLNTLCLAMQHYEQSK | (ENSP00000490523) encoded |
| | MFNDAMDILNMVFTGVFTVEMVLKVIAFKPKALPYVALLIAMLFFI | by Transcript ID |
| | YAVIGMQMFGKVAMRDNNQINRNNNFQTFPQAVLLLFRCATGEA | ENST00000638129 from Gene |
| | WQEIMLACLPGKLCDPESDYNPGEEYTCGSNFAIVYFISFYM | ID ENSG00000157388; Homo |
| | | sapiens |
| 154 | XVFTGVFTVEMVLKVIAFKPKHYFTDAWNTFDALIVVGSVVDIAITE | CACNA1D protein |
| | VNPTESENVPVPTATPGNSEESNRISITFFRLFRVMRLVKLLSRGEGIR | (ENSP00000490839) encoded |
| | TLLWTFIKSFQALPYVALLIAMLFFIYAVIGMQMFGKVAMRDNNQI | by Transcript ID |
| | NRNNNFQTFPQAVLLLFRCATGEAWQEIMLACLPGKLCDPESDYNP | ENST00000636448 from Gene |
| | GEEYTCGSNFAIVYFISFYMLCAFLIINLFVAVIMDNFDYLTRDWSIL | ID ENSG00000157388; Homo |
| | GPHHLDEFKRIWSEYDPEAKGRIKHLDVVTLLRRIQPPLGFGKLCPH | sapiens |
| | RVACKRLVAMNMPLNSDGTVMFNATLFALVRTALKIKTEGNLEQA | |
| | NEELRAVIKKIWKKTSMKLLDQVVPPAGDDEVTVGKFYATFLIQDY | |
| | FRKFKKRKEQGLVGKYPAKNTTIALQAGLRTLHDIGPEIRRAISCDL | |
| | QDDEPEETKREEEDDVFKQEIRCVITIITIIP | |
| 155 | XPSLQVVLNSIIKAMVPLLHIALLVLFVIIIYAIIGLELFIGKMHKTCFF | CACNA1D protein |
| | ADSDIVAEEDPAPCAFSGNGRQCTANGTECRSGWVGPNGGITNFDN | (ENSP00000490889) encoded |
| | FAFAMLTVFQCITMEGWTDVLYWVNDAIGWEWPWVYFVSLIILGS | by Transcript ID |
| | FFVLNLVLGVLSGEFSKEREKAKARGDFQKLREKQQLEEDLKGYLD | ENST00000636627 from Gene |
| | WITQAEDIDPENEEEGGEEGKRNTSMPTSETESVNTENVSGEGENRG | ID ENSG00000157388; Homo |
| | CCGSLWCWWRRRGAAKAGPSGCRRWGQAISKSKLSRRWRRWNRF | sapiens |
| | NRRRCRAAVKSVTFYWLVIVLVFLNTLTISSEHYNQPDWLTQIQDIA | |
| | NKVLLALFTCEMLVKMYSLGLQAYFVSLFNRFDCFVVCGGITETILV | |
| | ELEIMSPLGISVFRCVRLLRIFKVTRHWTSLSNLVASLLNSMKSIASL | |
| | LLLLFLFIIIFSLLGMQLFGGKFNFDETQTKRSTFDNFPQALLTVFQIL | |
| | TGEDWNAVMYDGIMAYGGPSSSGMIVCIYFIILFICGNYILLNVFLAI | |
| | AVDNLADAESLNTAQKEEAEEKERKKIARKESLENKKNNKPEVNQI | |
| | ANSDNKVTIDDYREEDEDKDPYPPCDVPVGEEEEEEEEDEPEVPAGP | |
| | RPRRISELNMKEKIAPIPEGSAFFILSKTNPIRVGCHKLINHHIFTNLIL | |
| | VFIMLSSAALAAEDPIRSHSFRNTILGYFDYAFTAIFTVEILLKMTTFG | |
| | AFLHKGAFCRNYFNLLDMLVVGVSLVSFGIQSSAISVVKILRVLRVL | |
| | RPLRAINRAKGLKHVVQCVFVAIRTIGNIMIVTTLLQFMFACIGVQLF | |
| | KGKFYRCTDEAKSNPEECRGLFILYKDGDVDSPVVRERIWQNSDFN | |
| | FDNVLSAMMALFTVSTFEGWPALLYKAIDSNGENIGPIYNHRVEISIF | |
| | FIIYIIIVAFFMMNIFVGFVIVTFQEQGEKEYKNCELDKNQRQCVEYA | |
| | LKARPLRRYIPKNPYQYKFWYVVNSSPFEYMMFVLIMLNTLCLAM | |
| | QHYEQSKMFNDAMDILNMVFTGVFTVEMVLKVIAFKPKHYFTDAW | |
| | NTFDALIVVGSVVDIAITEVNPTESENVPVPTATPGNSEESNRISITFF | |
| | RLFRVMRLVKLLSRGEGIRTLLWTFIKSFQALPYVALLIAMLFFIYAV | |
| | IGMQMFGKVAMRDNNQINRNNNFQTFPQAVLLLFRCATGEAWQEI | |
| | MLACLPGKLCDPESDYNPGEEYTCGSNFAIVYFISFYMLCAFLIINLF | |
| | VAVIMDNFDYLTRDWSILGPHHLDEFKRIWSEYDPEAKGRIKHLDV | |
| | VTLLRRIQPPLGFGKLCPHRVACKRLVAMNMPLNSDGTVMFNATLF | |
| | ALVRTALKIKTEGNLEQANEELRAVIKKIWKKTSMKLLDQVVPPAG | |
| | GQCSLFPK | |
| 156 | XFYRCTDEAKSNPEECRGLFILYKDGDVDSPVVRERIWQNSDFNFD | |
| | NVLSAMMALFTVSTFEGWPALLYKAIDSNGENIGPIYNHRVEISIFFII | CACNA1D protein |
| | YIIIVAFFMMNIFVGFVIVTFQEQGEKEYKNCELDKNQRQCVEYALK | (ENSP00000490908) encoded |
| | ARPLRRYIPKNPYQYKFWYVVNSSPFEYMMFVLIMLNTLCLAMQH | by Transcript ID |
| | YEQSKMFNDAMDILNMVFTGVFTVEMVLKVIAFKPKNSEESNRISIT | ENST00000636723 from Gene |
| | FFRLFRVMRLVKLLSRGEGIRTLLWTFIKSFQALPYVALLIAMLFFIY | ID ENSG00000157388; Homo |
| | AV | sapiens |
| 157 | MQHQRQQQADHANEANYARGTRLPLSGEGPTSQPNSSKQTVLSWQ | CACNA1D protein |
| | AAIDAARQAKAAQTMSTSAPPPVGSLSQRKRQQYAKSKKQGNSSNS | (ENSP00000491921) encoded |
| | RPARALFCLSLNNPIRRACISIVEWKPFDIFILLAIFANCVALAIYIPFPE | by Transcript ID |
| | DDSNSTNHNLEKVEYAFLIIFTVETFLKIIAYGLLLHPNAYVRNGWN | ENST00000640483 from Gene |
| | LLDFVIVIVGLFSVILEQLTKETEGGNHSSGKSGGFDVKALRAFRVLR | ID ENSG00000157388; Homo |
| | PLRLVSGVPSLQVVLNSIIKAMVPLLHIALLVLFVIIIYAIIGLELFIGK | sapiens |
| | MHKTCFFADSDIVAEEDPAPCAFSGNGRQCTANGTECRSGWVGPN | |
| | GGITNFDNFAFAMLTVFQCITMEGWTDVLYWVNDAIGWEWPWVY | |
| | FVSLIILGSFFVLNLVLGVLSGEFSKEREKAKARGDFQKLREKQQLEE | |
| | DLKGYLDWITQAEDIDPENEEEGGEEGKRNTSMPTSETESVNTENVS | |
| | GEGENRGCCGSLWCWWRRRGAAKAGPSGCRRWGQAISKSKLSRR | |
| | WRRWNRFNRRRCRAAVKSVTFYWLVIVLVFLNTLTISSEHYNQPD | |
| | WLTQIQDIANKVLLALFTCEMLVKMYSLGLQAYFVSLFNRFDCFVV | |
| | CGGITETILVELEIMSPLGISVFRCVRLLRIFKVTRHWTSLSNLVASLL | |
| | NSMKSIASLLLLLFLFIIIFSLLGMQLFGGKFNFDETQTKRSTFDNFPQ | |
| | ALLTVFQILTGEDWNAVMYDGIMAYGGPSSSGMIVCIYFIILFICGNY | |
| | ILLNVFLAIAVDNLADAESLNTAQKEEAEEKERKKIARKESLENKKN | |
| | NKPEVNQIANSDNKVTIDDYREEDEDKDPYPPCDVPVGEEEEEEEED | |
| | EPEVPAGPRPRRISELNMKEKIAPIPEGSAFFILSKTNPIRVGCHKLINH | |
| | HIFTNLILVFIMLSSAALAAEDPIRSHSFRNTILGYFDYAFTAIFTVEIL | |
| | LKMTTFGAFLHKGAFCRNYFNLLDMLVVGVSLVSFGIQSSAISVVKI | |
| | LRVLRVLRPLRAINRAKGLKHVVQCVFVAIRTIGNIMIVTTLLQFMF | |
| | ACIGVQLFKGKFYRCTDEAKSNPEECRGLFILYKDGDVDSPVVRERI | |
| | WQNSDFNFDNVLSAMMALFTVSTFEGWPALLYKAIDSNGENIGPIY | |
| | NHRVEISIFFIIYIIIVAFFMMNIFVGFVIVTFQEQGEKEYKNCELDKN | |
| | QRQCVEYALKARPLRRYIPKNPYQYKFWYVVNSSPFEYMMFVLIM | |
| | LNTLCLAMQHYEQSKMFNDAMDILNMVFTGVFTVEMVLKVIAFKP | |
| | KHYFTDAWNTFDALIVVGSVVDIAITEVNNSEESNRISITFFRLFRVM | |
| | RLVKLLSRGEGIRTLLWTFIKSFQALPYVALLIAMLFFIYAVIGMQMF | |
| | GKVAMRDNNQINRNNNFQTFPQAVLLLFRCATGEAWQEIMLACLP | |
| | GKLCDPESDYNPGEEYTCGSNFAIVYFISFYMLCAFLIINLFVAVIMD | |
| | NFDYLTRDWSILGPHHLDEFKRIWSEYDPEAKGRIKHLDVVTLLRRI | |
| | QPPLGFGKLCPHRVACKRLVAMNMPLNSDGTVMFNATLFALVRTA | |
| | LKIKTEGNLEQANEELRAVIKKIWKKTSMKLLDQVVPPAGDDEVTV | |
| | GKFYATFLIQDYFRKFKKRKEQGLVGKYPAKNTTIALQMLERML | |
| 158 | MSESEGGKDTTPEPSPANGAGPGPEWGLCPGPPAVEGESSGASGLG | CACNA1F protein |
| | TPKRRNQHSKHKTVAVASAQRSPRALFCLTLANPLRRSCISIVEWKP | (ENSP00000321618) encoded |
| | FDILILLTIFANCVALGVYIPFPEDDSNTANHNLEQVEYVFLVIFTVET | by Transcript ID |
| | VLKIVAYGLVLHPSAYIRNGWNLLDFIIVVVGLFSVLLEQGPGRPGD | ENST00000323022 from Gene |
| | APHTGGKPGGFDVKALRAFRVLRPLRLVSGVPSLHIVLNSIMKALVP | ID ENSG00000102001; 'Homo |
| | LLHIALLVLFVIIIYAIIGLELFLGRMHKTCYFLGSDMEAEEDPSPCAS | sapiens |
| | SGSGRACTLNQTECRGRWPGPNGGITNFDNFFFAMLTVFQCVTMEG | |
| | WTDVLYWMQDAMGYELPWVYFVSLVIFGSFFVLNLVLGVLSGEFS | |
| | KEREKAKARGDFQKQREKQQMEEDLRGYLDWITQAEELDMEDPSA | |
| | DDNLGPQLAELTNRRRGRLRWFSHSTRSTHSTSSHASLPASDTGSMT | |
| | ETQGDEDEEEGALASCTRCLNKIMKTRVCRRLRRANRVLRARCRRA | |
| | VKSNACYWAVLLLVFLNTLTIASEHHGQPVWLTQIQEYANKVLLCL | |
| | FTVEMLLKLYGLGPSAYVSSFFNREDCFVVCGGILETTLVEVGAMQ | |
| | PLGISVLRCVRLLRIFKVTRHWASLSNLVASLLNSMKSIASLLLLLFL | |
| | FIIIFSLLGMQLFGGKFNFDQTHTKRSTFDTFPQALLTVFQILTGEDW | |
| | NVVMYDGIMAYGGPFFPGMLVCIYFIILFICGNYILLNVFLAIAVDNL | |
| | ASGDAGTAKDKGGEKSNEKDLPQENEGLVPGVEKEEEEGARREGA | |
| | DMEEEEEEEEEEEEEEEEEGAGGVELLQEVVPKEKVVPIPEGSAFFC | |
| | LSQTNPLRKGCHTLIHHHVFTNLILVFIILSSVSLAAEDPIRAHSFRNHI | |
| | LGYFDYAFTSIFTVEILLKMTVFGAFLHRGSFCRSWFNMLDLLVVSV | |
| | SLISFGIHSSAISVVKILRVLRVLRPLRAINRAKGLKHVVQCVFVAIRT | |
| | IGNIMIVTTLLQFMFACIGVQLFKGKFYTCTDEAKHTPQECKGSFLV | |
| | YPDGDVSRPLVRERLWVNSDFNFDNVLSAMMALFTVSTFEGWPAL | |
| | LYKAIDAYAEDHGPIYNYRVEISVFFIVYIIIIAFFMMNIFVGFVIITFR | |
| | AQGEQEYQNCELDKNQRQCVEYALKAQPLRRYIPKNPHQYRVWAT | |
| | VNSAAFEYLMFLLILLNTVALAMQHYEQTAPFNYAMDILNMVFTGL | |
| | FTIEMVLKIIAFKPKHYFTDAWNTFDALIVVGSIVDIAVTEVNNGGH | |
| | LGESSEDSSRISITFFRLFRVMRLVKLLSKGEGIRTLLWTFIKSFQALP | |
| | YVALLIAMIFFIYAVIGMQMFGKVALQDGTQINRNNNFQTFPQAVLL | |
| | LFRCATGEAWQEIMLASLPGNRCDPESDFGPGEEFTCGSNFAIAYFIS | |
| | FFMLCAFLIINLFVAVIMDNFDYLTRDWSILGPHHLDEFKRIWSEYDP | |
| | GAKGRIKHLDVVALLRRIQPPLGFGKLCPHRVACKRLVAMNMPLNS | |
| | DGTVTFNATLFALVRTSLKIKTEGNLEQANQELRIVIKKIWKRMKQK | |
| | LLDEVIPPPDEEEVTVGKFYATFLIQDYFRKFRRRKEKGLLGNDAAP | |
| | STSSALQAGLRSLQDLGPEMRQALTCDTEEEEEEGQEGVEEEDEKD | |
| | LETNKATMVSQPSARRGSGISVSLPVGDRLPDSLSFGPSDDDRGTPT | |
| | SSQPSVPQAGSNTHRRGSGALIFTIPEEGNSQPKGTKGQNKQDEDEE | |
| | VPDRLSYLDEQAGTPPCSVLLPPHRAQRYMDGHLVPRRRLLPPTPA | |
| | GRKPSFTIQCLQRQGSCEDLPIPGTYHRGRNSGPNRAQGSWATPPQR | |
| | GRLLYAPLLLVEEGAAGEGYLGRSSGPLRTFTCLHVPGTHSDFSHGK | |
| | RGSADSLVEAVLISEGLGLFARDPRFVALAKQEIADACRLTLDEMD | |
| | NAASDLLAQGTSSLYSDEESILSRFDEEDLGDEMACVHAL | |
| 159 | MSESEGGKGERILPSLQTLGASIVEWKPFDILILLTIFANCVALGVYIP | CACNA1F protein |
| | FPEDDSNTANHNLEQVEYVFLVIFTVETVLKIVAYGLVLHPSAYIRN | (ENSP00000365427) encoded |
| | GWNLLDFIIVVVGLFSVLLEQGPGRPGDAPHTGGKPGGFDVKALRA | by Transcript ID |
| | FRVLRPLRLVSGVPSLHIVLNSIMKALVPLLHIALLVLFVIIIYAIIGLE | ENST00000376251 from Gene |
| | LFLGRMFIKTCYFLGSDMEAEEDPSPCASSGSGRACTLNQTECRGRW | ID ENSG00000102001; Homo |
| | PGPNGGITNFDNFFFAMLTVFQCVTMEGWTDVLYWMQDAMGYEL | sapiens |
| | PWVYFVSLVIFGSFFVLNLVLGVLSGEFSKEREKAKARGDFQKQRE | |
| | KQQMEEDLRGYLDWITQAEELDMEDPSADDNLGSMAEEGRAGHRP | |
| | QLAELTNRRRGRLRWFSHSTRSTHSTSSHASLPASDTGSMTETQGDE | |
| | DEEEGALASCTRCLNKIMKTRVCRRLRRANRVLRARCRRAVKSNA | |
| | CYWAVLLLVFLNTLTIASEHHGQPVWLTQIQEYANKVLLCLFTVEM | |
| | LLKLYGLGPSAYVSSFFNRFDCFVVCGGILETTLVEVGAMQPLGISV | |
| | LRCVRLLRIFKVTRHWASLSNLVASLLNSMKSIASLLLLLFLFIIIFSLL | |
| | GMQLFGGKFNFDQTHTKRSTFDTFPQALLTVFQILTGEDWNVVMY | |
| | DGIMAYGGPFFPGMLVCIYFIILFICGNYILLNVFLAIAVDNLASGDA | |
| | GTAKDKGGEKSNEKDLPQENEGLVPGVEKEEEEGARREGADMEEE | |
| | EEEEEEEEEEEEEEGAGGVELLQEVVPKEKVVPIPEGSAFFCLSQTNP | |
| | LRKGCHTLIHHHVFTNLILVFIILSSVSLAAEDPIRAHSFRNHILGYFD | |
| | YAFTSIFTVEILLKMTVFGAFLHRGSFCRSWFNMLDLLVVSVSLISFG | |
| | IHSSAISVVKILRVLRVLRPLRAINRAKGLKHVVQCVFVAIRTIGNIMI | |
| | VTTLLQFMFACIGVQLFKGKFYTCTDEAKHTPQECKGSFLVYPDGD | |
| | VSRPLVRERLWVNSDFNFDNVLSAMMALFTVSTFEGWPALLYKAID | |
| | AYAEDHGPIYNYRVEISVFFIVYIIIIAFFMMNIFVGFVIITFRAQGEQE | |
| | YQNCELDKNQRQCVEYALKAQPLRRYIPKNPHQYRVWATVNSAAF | |
| | EYLMFLLILLNTVALAMQHYEQTAPFNYAMDILNMVFTGLFTIEMV | |
| | LKIIAFKPKHYFTDAWNTFDALIVVGSIVDIAVTEVNNGGHLGESSE | |
| | DSSRISITFFRLFRVMRLVKLLSKGEGIRTLLWTFIKSFQALPYVALLI | |
| | AMIFFIYAVIGMQMFGKVALQDGTQINRNNNFQTFPQAVLLLFRCA | |
| | TGEAWQEIMLASLPGNRCDPESDFGPGEEFTCGSNFAIAYFISFFMLC | |
| | AFLIINLFVAVIMDNFDYLTRDWSILGPHHLDEFKRIWSEYDPGAKG | |
| | RIKHLDVVALLRRIQPPLGFGKLCPHRVACKRLVAMNMPLNSDGTV | |
| | TFNATLFALVRTSLKIKTEGNLEQANQELRIVIKKIWKRMKQKLLDE | |
| | VIPPPDEEEVTVGKFYATFLIQDYFRKFRRRKEKGLLGNDAAPSTSS | |
| | ALQAGLRSLQDLGPEMRQALTCDTEEEEEEGQEGVEEEDEKDLETN | |
| | KATMVSQPSARRGSGISVSLPVGDRLPDSLSFGPSDDDRGTPTSSQPS | |
| | VPQAGSNTHRRGSGALIFTIPEEGNSQPKGTKGQNKQDEDEEVPDRL | |
| | SYLDEQAGTPPCSVLLPPHRAQRYMDGHLVPRRRLLPPTPAGRKPSF | |
| | TIQCLQRQGSCEDLPIPGTYHRGRNSGPNRAQGSWATPPQRGRLLY | |
| | APLLLVEEGAAGEGYLGRSSGPLRTFTCLHVPGTHSDPSHGKRGSA | |
| | DSLVEAVLISEGLGLFARDPRFVALAKQEIADACRLTLDEMDNAAS | |
| | DLLAQGTSSLYSDEESILSRFDEEDLGDEMACVHAL | |
| 160 | MSESEGGKDTTPEPSPANGAGPGPEWGLCPGPPAVEGESSGASGLG | CACNA1F protein |
| | TPKRRNQHSKHKTVAVASAQRSPRALFCLTLANPLRRSCISIVEWKP | (ENSP00000365441) encoded |
| | FDILILLTIFANCVALGVYIPFPEDDSNTANHNLEQVEYVFLVIFTVET | by Transcript ID |
| | VLKIVAYGLVLHPSAYIRNGWNLLDFIIVVVGLFSVLLEQGPGRPGD | ENST00000376265 from Gene |
| | APHTGGKPGGFDVKALRAFRVLRPLRLVSGVPSLHIVLNSIMKALVP | ID ENSG00000102001; Homo |
| | LLHIALLVLFVIIIYAIIGLELFLGRMHKTCYFLGSDMEAEEDPSPCAS | sapiens |
| | SGSGRACTLNQTECRGRWPGPNGGITNFDNFFFAMLTVFQCVTMEG | |
| | WTDVLYWMQDAMGYELPWVYFVSLVIFGSFFVLNLVLGVLSGEFS | |
| | KEREKAKARGDFQKQREKQQMEEDLRGYLDWITQAEELDMEDPSA | |
| | DDNLGSMAEEGRAGHRPQLAELTNRRRGRLRWFSHSTRSTHSTSSH | |
| | ASLPASDTGSMTETQGDEDEEEGALASCTRCLNKIMKTRVCRRLRR | |
| | ANRVLRARCRRAVKSNACYWAVLLLVFLNTLTIASEHHGQPVWLT | |
| | QIQEYANKVLLCLFTVEMLLKLYGLGPSAYVSSFFNRFDCFVVCGGI | |
| | LETTLVEVGAMQPLGISVLRCVRLLRIFKVTRHWASLSNLVASLLNS | |
| | MKSIASLLLLLFLFIIIFSLLGMQLFGGKFNFDQTHTKRSTFDTFPQAL | |
| | LIVFQILTGEDWNVVMYDGIMAYGGPFFPGMLVCIYFIILFICGNYIL | |
| | LNVFLAIAVDNLASGDAGTAKDKGGEKSNEKDLPQENEGLVPGVE | |
| | KEEEEGARREGADMEEEEEEEEEEEEEEEEEGAGGVELLQEVVPKE | |
| | KVVPIPEGSAFFCLSQTNPLRKGCHTLIHHHVFTNLILVFIILSSVSLA | |
| | AEDPIRAHSFRNHILGYFDYAFTSIFTVEILLKMTVFGAFLHRGSFCR | |
| | SWFNMLDLLVVSVSLISFGIHSSAISVVKILRVLRVLRPLRAINRAKG | |
| | LKHVVQCVFVAIRTIGNIMIVTTLLQFMFACIGVQLFKGKFYTCTDE | |
| | AKHTPQECKGSFLVYPDGDVSRPLVRERLWVNSDFNFDNVLSAMM | |
| | ALFTVSTFEGWPALLYKAIDAYAEDHGPIYNYRVEISVFFIVYIIIIAFF | |
| | MMNIFVGFVIITFRAQGEQEYQNCELDKNQRQCVEYALKAQPLRRY | |
| | IPKNPHQYRVWATVNSAAFEYLMFLLILLNTVALAMQHYEQTAPFN | |
| | YAMDILNMVFTGLFTIEMVLKIIAFKPKHYFTDAWNTFDALIVVGSI | |
| | VDIAVTEVNNGGHLGESSEDSSRISITFFRLFRVMRLVKLLSKGEGIR | |
| | TLLWTFIKSFQALPYVALLIAMIFFIYAVIGMQMFGKVALQDGTQIN | |
| | RNNNFQTFPQAVLLLFRCATGEAWQEIMLASLPGNRCDPESDFGPG | |
| | EEFTCGSNFAIAYFISFFMLCAFLIINLFVAVIMDNFDYLTRDWSILGP | |
| | HHLDEFKRIWSEYDPGAKGRIKHLDVVALLRRIQPPLGFGKLCPHRV | |
| | ACKRLVAMNMPLNSDGTVTFNATLFALVRTSLKIKTEGNLEQANQE | |
| | LRIVIKKIWKRMKQKLLDEVIPPPDEEEVTVGKFYATFLIQDYFRKFR | |
| | RRKEKGLLGNDAAPSTSSALQAGLRSLQDLGPEMRQALTCDTEEEE | |
| | EEGQEGVEEEDEKDLETNKATMVSQPSARRGSGISVSLPVGDRLPDS | |
| | LSFGPSDDDRGTPTSSQPSVPQAGSNTHRRGSGALIFTIPEEGNSQPK | |
| | GTKGQNKQDEDEEVPDRLSYLDEQAGTPPCSVLLPPHRAQRYMDG | |
| | HLVPRRRLLPPTPAGRKPSFTIQCLQRQGSCEDLPIPGTYHRGRNSGP | |
| | NRAQGSWATPPQRGRLLYAPLLLVEEGAAGEGYLGRSSGPLRTFTC | |
| | LHVPGTHSDPSHGKRGSADSLVEAVLISEGLGLFARDPRFVALAKQE | |
| | IADACRLTLDEMDNAASDLLAQGTSSLYSDEESILSRFDEEDLGDEM | |
| | ACVHAL | |
| 161 | DYFRKFRKRKEKGLLGNDAAPSTSSALQAGLRSLQDLGPEMRQALT | CACNA1F protein |
| | CDTEEEEEEGQEGVEEEDEKDLETNKVGTSFHSPRNLIVKYFVNPLS | (ENSP00000418961) encoded |
| | DFDTASGKKLPQRSPLCNRKIFI | by Transcript ID |
| | | ENST00000486943 from Gene |
| | | ID ENSG00000102001; Homo |
| | | sapiens |
| 162 | MEMPLLLLFSLCSAGLVLGSEHETRLVAKLFKDYSSVVRPVEDHR | CHRNA1 protein |
| | QVVEVTVGLQLIQLINVDEVNQIVTTNVRLKQGDMVDLPRPSCVTL | (ENSP00000261007) encoded |
| | GVPLFSHLQNEQWVDYNLKWNPDDYGGVKKIHIPSEKIWRPDLVL | by Transcript ID |
| | YNNADGDFAIVKFTKVLLQYTGHITWTPPAIFKSYCEIIVTHFPFDEQ | ENST00000261007 from Gene |
| | NCSMKLGTWTYDGSVVAINPESDQPDLSNFMESGEWVIKESRGWK | ID ENSG00000138435; Homo |
| | HSVTYSCCPDTPYLDITYHFVMQRLPLYFIVNVIIPCLLFSFLTGLVFY | sapiens |
| | LPTDSGEKMTLSISVLLSLTVFLLVIVELIPSTSSAVPLIGKYMLFTMV | |
| | FVIASIIITVIVINTHHRSPSTHVMPNWVRKVFIDTIPNIMFFSTMKRPS | |
| | REKQDKKIFTEDIDISDISGKPGPPPMQFHSPLIKHPEVKSAIEGIKYIA | |
| | ETMKSDQESNNAAAEWKYVAMVMDHILLGVFMLVCIIGTLAVFAG | |
| | RLIELNQQG | |
| 163 | MEPWPLLLLFSLCSAGLVLGSEHETRLVAKLFKDYSSVVRPVEDHR | CHRNA1 protein |
| | QVVEVTVGLQLIQLINVDEVNQIVTTNVRLKQQWVDYNLKWNPDD | (ENSP00000261008) encoded |
| | YGGVKKIHIPSEKIWRPDLVLYNNADGDFAIVKFTKVLLQYTGHITW | by Transcript ID |
| | TPPAIFKSYCEIIVTHFPFDEQNCSMKLGTWTYDGSVVAINPESDQPD | ENST00000348749 from Gene |
| | LSNFMESGEWVIKESRGWKHSVTYSCCPDTPYLDITYHFVMQRLPL | ID ENSG00000138435; Homo |
| | YFIVNVIIPCLLFSFLTGLVFYLPTDSGEKMTLSISVLLSLTVFLLVIVE | sapiens |
| | LIPSTSSAVPLIGKYMLFTMVFVIASIIITVIVINTHHRSPSTHVMPNW | |
| | VRKVFIDTIPNIMFFSTMKRPSREKQDKKIFTEDIDISDISGKPGPPPM | |
| | GFHSPLIKHPEVKSAIEGIKYIAETMKSDQESNNAAAEWKYVAMVM | |
| | DHILLGVFMLVCIIGTLAVFAGRLIELNQQG | |
| 164 | MEPWPLLLLFSLCSAGLVLGSEHETRLVAKLFKDYSSVVRPVEDHR | CHRNA1 protein |
| | QVVEVTVGLQLIQLINVDEVNQIVTTNVRLKQQWVDYNLKWNPDD | (ENSP00000386611) encoded |
| | YGGVKKIHIPSEKIWRPDLVLYNNADGDFAIVKFTKVLLQYTGHITW | by Transcript ID |
| | TPPAIFKSYCEIIVTHFPFDEQNCSMKLGTWTYDGSVVAINPESDQPD | ENST00000409219 from Gene |
| | LSNFMESGEWVIKESRGWKHSVTYSCCPDTPYLDITYHFVMQRLPL | ID ENSG00000138435; Homo |
| | YFIVNVIIPCLLFSFLTGLVFYLPTDSGEKMTLSISVLLSLTVFLLVIVE | sapiens |
| | LIPSTSSAVPLIGKYMLFTMVFVIASIIITVIVINTHHRSPSTHVMPNW | |
| | VRKAAAEWKYVAMVMDHILLGVFMLVCIIGTLAVFAGRLIELNQQ | |
| | G | |
| 165 | MEPWPLLLLFSLCSAGLVLGSEHETRLVAKLFKDYSSVVRPVEDHR | CHRNA1 protein |
| | QVVEVTVGLQLIQLINVDEVNQIVTTNVRLKQQWVDYNLKWNPDD | (ENSP00000386684) encoded |
| | YGGVKKIHIPSEKIWRPDLVLYNNADGDFAIVKFTKVLLQYTGHITW | by Transcript ID |
| | TPPAIFKSYCEIIVTHFPFDEQNCSMKLGTWTYDGSVVAINPESDQPD | ENST00000409323 from Gene |
| | LSNFMESGEWVIKESRGWKHSVTYSCCPDTPYLDITYHFVMQRLPL | ID ENSG00000138435; Homo |
| | YFIVNVIIPCLLFSFLTGLVFYLPTDSGGCGCHDCCC | sapiens |
| 166 | MEPWPLLLLFSLCSAGLVLGSEHETRLVAKLFKDYSSVVRPVEDHR | CHRNA1 protein |
| | QVVEVTVGLQLIQLINVDEVNQIVTTNVRLKQNCSMKLGTWTYDGS | (ENSP00000387026) encoded |
| | VVAINPESDQPDLSNFMESGEWVIKESRGWKHSVTYSCCPDTPYLDI | by Transcript ID |
| | TYHFVMQRLPLYFIVNVIIPCLLFSFLTGLVFYLPTDSGEKMTLSISVL | ENST00000409542 from Gene |
| | LSLTVFLLVIVELIPSTSSAVPLIGKYMLFTMVFVIASIIITVIVINTHHR | ID ENSG00000138435; Homo |
| | SPSTHVMPNWVRKVFIDTIPNIMFFSTMKRPSREKQDKKIFTEDIDIS | sapiens |
| | DISGKPGPPPMGFHSPLIKHPEVKSAIEGIKYIAETMKSDQESNNAAA | |
| | EWKYVAMVMDHILLGVFMLVCIIGTLAVFAGRLIELNQQG | |
| 167 | MEMPLLLLFSLCSAGLVLGSEHETRLVAKLFKDYSSVVRPVEDHR | CHRNA1 protein |
| | QVVEVTVGLQLIQLINVDEVNQIVTTNVRLKQCRW | (ENSP00000395805) encoded |
| | | by Transcript ID |
| | | ENST00000435083 from Gene |
| | | ID ENSG00000138435; Homo |
| | | sapiens |
| 168 | MWTKVKGRNSLTGLGLDLSKTVSPHWAAGLVLGSEHETRLVAKLF | CHRNA1 protein |
| | KDYSSVVRPVEDHRQVVEVTVGLQLIQLINVDEVNQIVTTNVRLKQ | (ENSP00000490338) encoded |
| | QWVDYNLKWNPDDYGGVKKIHIPSEKIWRFDLVLYNNADGDFAIV | by Transcript ID |
| | KFTKVLLQYTGHITWTPPAIFKSYCEIIVTHFPFDEQNCSMKLGTWT | ENST00000636168 from Gene |
| | YDGSVVAINPESDQPDLSNFMESGEWVIKESRGWKHSVTYSCCPDT | ID ENSG00000138435; Homo |
| | PYLDITYHFVMQRLPLYFIVNVIIPCLLFSFLTGLVFYLPTDSGEKMTL | sapiens |
| | SISVLLSLTVFLLVIVELIPSTSSAVPLIGKYMLFTMVFVIASIIITVIVIN | |
| | THHRSPSTHVMPNWVRKVFIDTIPNIMFFSTMKRPSREKQDKKIFTE | |
| | DIDISDISGKPGPPPMGFHSPLIKHPEVKSAIEGIKYIAETMKSDQESN | |
| | NAAAEWKYVAMVMDHILLGVFMLVCIIGTLAVFAGRLIELNQQG | |
| 169 | MTPGALLMLLGALGAPLAPGVRGSEAEGRLREKLFSGYDSSVRPAR | CHRNB1 protein |
| | EVGDRVRVSVGLILAQLISLNEKDEEMSTKVYLDLEWTDYRLSWDP | (ENSP00000304290) encoded |
| | AEHDGIDSLRITAESVWLPDVVLLNNNDGNFDVALDISVVVSSDGS | by Transcript ID |
| | VRWQPPGIYRSSCSIQVTYFPFDWQNCTMVFSSYSYDSSEVSLQTGL | ENST00000306071 from Gene |
| | GPDGQGHQEIIIIIIEGTFIENGQWEIIHKPSRLIQPPGDPRGGREGQRQ | ID ENSG00000170175; Homo |
| | EVIFYLIIRRKPLFYLVNVIAPCILITLLAIFVFYLPPDAGEKMGLSIFA | sapiens |
| | LLTLTVFLLLLADKVPETSLSVPIIIKYLMFTMVLVTFSVILSVVVLNL | CHRNB1 protein |
| | HHRSPHTHQMPLWVRQIFIHKLPLYLRLKRPKPERDLMPEPPHCSSP | (ENSP00000492221) encoded |
| | GSGWGRGTDEYFIRKPPSDFLFPKPNRFQPELSAPDLRRFIDGPNRAV | by Transcript ID |
| | ALLPELREVVSSISYIARQLQEQEDHDALKEDWQFVAMVVDRLFLW | ENST00000639692 from Gene |
| | TFIIFTSVGTLVIFLDATYHLPPPDPFP | ID ENSG00000283946; Homo |
| | | sapiens |
| 170 | MSTKVYLDLEWTDYRLSWDPAEHDGIDSLRITAESVWLPDVVLLN | CHRNB1 protein |
| | NNDGNFDVALDISVVVSSDGSVRWQPPGIYRSSCSIQVTYFPFDWQN | (ENSP00000439209) encoded |
| | CTMVFSSYSYDSSEVSLQTGLGPDGQGHQEIHIHEGTFIENGQWEIIH | by Transcript ID |
| | KPSRLIQPPGDPRGGREGQRQEVIFYLIIRRKPLFYLVNVIAPCILITLL | ENST00000536404 from Gene |
| | AIFVFYLPPDAGEKMGLSIFALLTLTVFLLLLADKVPETSLSVPIIIKY | ID ENSG00000170175; Homo |
| | LMFTMVLVTFSVILSVVVLNLHHRSPHTHQMPLWVRQIFIHKLPLYL | sapiens |
| | RLKRPKPERDLMPEPPHCSSPGSGWGRGTDEYFIRKPPSDFLFPKPNR | CHRNB1 protein |
| | FQPELSAPDLRRFIDGPNRAVALLPELREVVSSISYIARQLQEQEDHD | (ENSP00000491113) encoded |
| | ALKEDWQFVAMVVDRLFLWTFIIFTSVGTLVIFLDATYHLPPPDPFP | by Transcript ID |
| | | ENST00000639993 from Gene |
| | | ID ENSG00000283946; Homo |
| | | sapiens |
| 171 | MSTKVYLDLEWTDYRLSWDPAEHDGIDSLRITAESVWLPDVVLLN | CHRNB1 protein |
| | NNDGNFDVALDISVVVSSDGSVRWQPPGIYRSSCSIQVTYFPFDWQN | (ENSP00000459092) encoded |
| | CTMVFSSYSYDSSEVSLQTGLGPDGQGHQEIHIHEGTFIENGQWEIIH | by Transcript ID |
| | KPSRLIQPPGDPRGGREGQRQEVIFYLIIRRKPLFYLVNVIAPCILITLL | ENST00000576360 from Gene |
| | AIFVFYLPPDAVILSVVVLNLHHRSPHTHQMPLWVRQIFIHKLPLYLR | ID ENSG00000170175; Homo |
| | LKRPKPERDLMPEPPHCSSPGSGWGRGTDEYFIRKPPSDFLFPKPNRF | sapiens |
| | QPELSAPDLRRFIDGPNRAVALLPELREVVSSISYIARQLQEQEDHDA | CHRNB1 protein |
| | LKEDWQFVAMVVDRLFLWTFIIFTSVGTLVIFLDATYHLPPPDPFP | (ENSP00000491764) encoded |
| | | by Transcript ID |
| | | ENST00000639509 from Gene |
| | | ID ENS000000283946; Homo |
| | | sapiens |
| 172 | XPAREVGDRVRVSVGLILAQLISLNEKDEEMSTKVYLDLEWTDYRL | CHRNB1 protein |
| | SWDPAEHDGIDSLRITAESVWLPDVVLLNNNDGNFDVALDISVVVS | (ENSP00000460648) encoded |
| | SDGSVRWQPPGIYRSSCSIQVTYFPFDWQNCTMVFSSYSYDSSEVSL | by Transcript ID |
| | QTGLGPDGQGHQEIHIHEGTFIGEKMGLSIFALLTLTVFLLLLADKVP | ENST00000570557 from Gene |
| | ETSLSVPIIIKYLMFTMVLVTFSVILSVVVLNLHHRSPHTHQMPLWV | ID ENSG00000170175; Homo |
| | RQIFIHKLPLYLRLKRPKPERDLMPEPPHCSSPGSGWGRGTDEYFIRK | sapiens |
| | PPSDFLFPKPNRFQPELSAPDLRRFIDGPNRAVA | CHRNB1 protein |
| | | (ENSP00000491163) encoded |
| | | by Transcript ID |
| | | ENST00000638943 from Gene |
| | | ID ENSG00000283946; Homo |
| | | sapiens |
| 173 | MTPGALLMLLGALGAPLAPGVRGSEAEGRLREKLFSGYDSSVRPAR | CHRNB1 protein |
| | EVGDRVRVSVGLILAQLISLNEKDEEMSTKVYLDLAVLECGGSSHPP | (ENSP00000461402) encoded |
| | ASGGTSTSAEITAEWTDYRLSWDPAEHDGIDSLRITAESVWLPDVVL | by Transcript ID |
| | LNNNDGNFDVALDISVVV | ENST00000572857 from Gene |
| | | ID ENSG00000170175; Homo |
| | | sapiens |
| | | CHRNB1 protein |
| | | (ENSP00000491344) encoded |
| | | by Transcript ID |
| | | ENST00000638556 from Gene |
| | | ID ENSG00000283946; Homo |
| | | sapiens |
| 174 | MVVDRLFLWITIIFTSVGTLVIFLDATYHLPPPDPFP | CHRNB1 protein |
| | | (ENSP00000461751) encoded |
| | | by Transcript ID |
| | | ENST00000575379 from Gene |
| | | ID ENSG00000170175; Homo |
| | | sapiens |
| | | CHRNB1 protein |
| | | (ENSP00000492078) encoded |
| | | by Transcript ID |
| | | ENST00000638662 from Gene |
| | | ID ENSG00000283946; Homo |
| | | sapiens |
| 175 | MARAPLGVLLLLGLLGRGVGKNEELRLYHHLFNNYDPGSRPVREPE | CHRNE protein |
| | DTVTISLKVTLTNLISLNEKEETLTTSVWIGIDWQDYRLNYSKDDFG | (ENSP00000293780) encoded |
| | GIETLRVPSELVWLPEIVLENNIDGQFGVAYDANVLVYEGGSVTWL | by Transcript ID |
| | PPAIYRSVCAVEVTYFPFDWQNCSLIFRSQTYNAEEVEFTFAVDNDG | ENST00000293780 from Gene |
| | KTINKIDIDTEAYTENGEWAIDFCPGVIRRHHGGATDGPGETDVIYSL | ID ENSG00000108556; Homo |
| | IIRRKPLFYVINIIVPCVLISGLVLLAYFLPAQAGGQKCTVSINVLLAQ | sapiens |
| | TVFLFLIAQKIPETSLSVPLLGRFLIFVMVVATLIVMNCVIVLNVSQRT | |
| | PTTHAMSPRLRHVLLELLPRLLGSPPPPEAPRAASPPRRASSVGLLLR | |
| | AEELILKKPRSELVFEGQRHRQGTWTAAFCQSLGAAAPEVRCCVDA | |
| | VNFVASTRDQEATGEEVSDWVRMGNALDNICFWAALVLFSVGSS | |
| | LIFLGAYFNRVPDLPYAPCIQP | |
| 176 | MVVATLIVMNCVIVLNVSQRTPTTHAMSPRLRHVLLELLPRLLGSPP | CHRNE protein |
| | PPEAPRAASPPRRASSVGLLLRAEELILKKPRSELVFEGQRHRQGTW | (ENSP00000496907) encoded |
| | TAAFCQSLGAAAPEVRCCVDAVNFVAESTRDQEATGEVGQEPEAG | by Transcript ID |
| | GARQRPTTVSHAAGSCSCLFTDCADPSPGSVRLGAHGECP | ENST00000649830 from Gene |
| | | ID ENSG00000108556; Homo |
| | | sapiens |
| 177 | MWWRLWWLLLLLLLLWPMVWADYKDHDGDYKDHDIDYKDDDD | Fusion protein produced from |
| | KGGGSgnstmGSGGGGGSGGGGSLELNLTDSENATCLYAKWQMNFT | Vector 91 |
| | VRYETTNKTYKTVTISDHGTVTYNGSICGDDQNGPKIAVQFGPGFS | Artificial Sequence |
| | WIANFTKAASTYSIDSVSFSYNTGDNTTFPDAEDKGILTVDELLAIRI | |
| | PLNDLFRCNSLSTLEKNDVVQHYWDVLVQAFVQNGTVSTNEFLCD | |
| | KDKTSTVAPTIHTTVPSPTTTPTPKEKPEAGTYSVNNGNDTCLLATM | |
| | GLQLNITQDKVASVININPNTTHSTGSCRSHTALLRLNSSTIKYLDFV | |
| | FAVKNENRFYLKEVNISMYLVNGSVFSIANNNLSYWDAPLGSSYMC | |
| | NKEQTVSVSGAFQINTFDLRVQPFNVTQGKYSTAQECSLDDDTILIPI | |
| | IVGAGLSGLIIVIVIAYVIGRRKSYAGYQTL | |
| 178 | MWWRLWWLLLLLLLLWPMVWATHRPPMWSPVWPGGGSDYKDH | Fusion protein produced from |
| | DGDYKDHDIDYKDDDDKgnstmGSGGGGGSGGGGSARVNKHKPWL | Vector 112 |
| | EPTYHGIVTENDNTVLLDPPLIALDKDAPLRFAESFEVTVTKEGEICG | Artificial Sequence |
| | FKIHGQNVPFDAVVVDKSTGEGVIRSKEKLDCELQKDYSFTIQAYDC | |
| | GKGPDGTNVKKSHKATVHIQVNDVNEYAPVFKEKSYKATVIEGKQ | |
| | YDSILRVEAVDADCSPQFSQICSYEIITPDVPFTVDKDGYIKNTEKLN | |
| | YGKEHQYKLTVTAYDCGKKRATEDVLVKISIKPTCTPGWQGWNNR | |
| | IEYEPGTGALAVFPNIHLETCDEPVASVQATVELETSHIGKGCDRDT | |
| | YSEKSLHRLCGAAAGTAELLPSPSGSLNWTMGLPTDNGHDSDQVFE | |
| | FNGTQAVRIPDGVVSVSPKEPFTISVWMRHGPFGRKKETILCSSDKT | |
| | DMNRHHYSLYVHGCRLIFLFRQDPSEEKKYRPAEFHWKLNQVCDE | |
| | EWHHYVLNVEFPSVTLYVDGTSHEPFSVTEDYPLHPSKIETQLVVGA | |
| | CWQEFSGVENDNETEPVTVASAGGDLHMTQFFRGNLAGLTLRSGK | |
| | LADKKVIDCLYTCKEGLDLQVLEDSGRGVQIQAHPSQLVLTLEGED | |
| | LGELDKAMQHISYLNSRQFPTPGIRRLKITSTIKCFNEATCISVPPVDG | |
| | YVMVLQPEEPKISLSGVHHFARAASEFESSEGVFLFPELRIISTITREV | |
| | EPEGDGAEDPTVQESLVSEEIVHDLDTCEVTVEGEELNHEQESLEVD | |
| | MARLQQKGIEVSSSELGMTFTGVDTMASYEEVLHLLRYRNWHARS | |
| | LLDRKFKLICSELNGRYISNEFKVEVNVIHTANPMEHAAAAAAQPQF | |
| | VHPEHRSFVDLSGHNLANPHPFAVVPSTATVVIVVCVSFLVFMIILG | |
| | VFRIRAAHRRTMRDQDTGKENEMDWDDSALTITVNPMETYEDQHS | |
| | SEEEEEEEEEEESEDGEEEDDITSAESESSEEEEGEQGDPQNATRQQQ | |
| | LEWDDSTLSY | |
| 179 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 135 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIASSHWCCKKEVQETRRERRRLM | |
| | SMEMD | |
| 180 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 140 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIAKCGFFKRARTRALYEAKRQKA | |
| | EMKSQPSETERUTDDY | |
| 181 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 141 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIAVMQRLFPRIPHMKDPIGDSFQN | |
| | DKLVVWEAGKAGLEECLVTEVQVVQKT | |
| 182 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 142 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIARLSRKGHMYPVRNYSPTEMVCI | |
| | SSLLPDGGEGPSATANGGLSKAKSPGLTPEPREDREGDDLTLHSFLP | |
| 183 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 143 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIALLMIIHDRREFAKFEKEKMNAK | |
| | WDTGENPIYKSAVTTVVNPKYEGK | |
| 184 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 144 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIARIRAAHRRTMRDQDTGKENEM | |
| | DWDDSALTITVNPMETYEDQHSSEEEEEEEEEEESEDGEEEDDITSA | |
| | ESESSEEEEGEQGDPQNATRQQQLEWDDSTLSY | |
| 185 | MWWRLWWLLLLLLLLWPMVWADYKDHDGDYKDHDIDYKDDDD | Fusion protein produced from |
| | KTHVSPNQGGLPSGGGSgnstmGSGGGGGSGGGGSLELNLTDSENAT | Vector 145 |
| | CLYAKWQMNFTVRYETTNKTYKTVTISDHGTVTYNGSICGDDQNG | Artificial Sequence |
| | PKIAVQFGPGFSWIANFTKAASTYSIDSVSFSYNTGDNTTFPDAEDK | |
| | GILTVDELLAIRIPLNDLFRCNSLSTLEKNDVVQHYWDVLVQAFVQN | |
| | GTVSTNEFLCDKDKTSTVAPTIHTTVPSPTTTPTPKEKPEAGTYSVNN | |
| | GNDTCLLATMGLQLNITQDKVASVININPNTTHSTGSCRSHTALLRL | |
| | NSSTIKYLDFVFAVKNENRFYLKEVNISMYLVNGSVFSIANNNLSYW | |
| | DAPLGSSYMCNKEQTVSVSGAFQINTFDLRVQPFNVTQGKYSTAQE | |
| | CSLDDDTILIPIIVGAGLSGLIIVIVIAKKPR | |
| 186 | MWWRLWWLLLLLLLLWPMVWAGSEVQLVESGGGVVQPGGSLRL | Fusion protein produced from |
| | SCAASGFTFSRYAMSWVRQTPGKGLEWVSYISSNSLAIYYKDSMKG | Vector 177 |
| | RFTISRDNSKNTVYLQLSSLRGEDTALYFCARLTGYKLSYFDLWGQ | Artificial Sequence |
| | GTLVTVSSSSGGSSRSSSSGGGGSGGGGELVMTQSPGTLSLSPGERA | |
| | TLSCRASQSVSSTYLAWYQQKAGQAPRLLIHGASTRATGIPDRFSGS | |
| | GSGTDFTLTISRLEPEDFAVYFCQQYGSLSYIFGQGTRLEIKGGGSDY | |
| | KDHDGDYKDHDIDYKDDDDKgnstmGSGGGGGSGGGGSREVLVPEG | |
| | PLYRVAGTAVSISCNVTGYEGPAQQNFEWFLYRFEAPDTALGIVSTK | |
| | DTQFSYAVFKSRVVAGEVQVQRLQGDAVVLKIARLQAQDAGIYEC | |
| | HTPSTDIRYLGSYSGKVELRVLPDVLQVSAAPPGPRGRQAPTSPPRM | |
| | TVHEGQELALGCLARTSTQKHTHLAVSFGRSVPEAPVGRSTLQEVV | |
| | GIRSDLAVEAGAPYAERLAAGELRLGKEGTDRYRMVVGGAQAGDA | |
| | GTYHCTAAEWIQDPDGSWAQIAEKRAVLAHVDVQTLSSQLAVTVG | |
| | PGERRIGPGEPLELLCNVSGALPPAGRHAAYSVGWEMAPAGAPGPG | |
| | RLVAQLDTEGVGSLGPGYEGRHIAMEKVASRTYRLRLEAARPGDA | |
| | GTYRCLAKAYVRGSGTRLREAASARSRPLPVHVREEGVVLEAVAW | |
| | LAGGTVYRGETASLLCNISVRGGPPGLRLAASWWVERPEDGELSSV | |
| | PAQLVGGVGQDGVAELGVRPGGGPVSVELVGPRSHRLRLHSLGPED | |
| | EGVYHCAPSAWVQHADYSWYQAGSARSGPVTVYPYMHALDTLFV | |
| | PLLVGTGVALVTGATVLGTITCCFMKRLRKR | |
| 187 | MWWRLWWLLLLLLLLWPMVWAGSEVQLVESGGGVVQPGSEVQL | Fusion protein produced from |
| | VESGGGVVQPGGSLRLSCAASGFSFDRYAMSWVRQTPGRGLEWVA | Vector 178 |
| | YISSNSLAIYYKDSVKGRFTISRDNSKKTVYLHVSSLRDEDTALYFC | Artificial Sequence |
| | ARLTGYKLSYFDLWGQGTLVTVSSSSGGSSRSSSSGGGGSGGGGEL | |
| | VMTQSPGTLSLSPGERATLSCRASQTVDSTYLAWYQQKPGQAPRLL | |
| | IHTASSRAIGIPDRFSGSGSGTDFTLTIDTLEPEDFAVYYCQQYGSASY | |
| | IFGQGTRLEIKGGGSDYKDHDGDYKDHDIDYKDDDDKgnstmGSGG | |
| | GGGSGGGGSREVLVPEGPLYRVAGTAVSISCNVTGYEGPAQQNFEW | |
| | FLYRPEAPDTALGIVSTKDTQFSYAVFKSRVVAGEVQVQRLQGDAV | |
| | VLKIARLQAQDAGIYECHTPSTDTRYLGSYSGKVELRVLPDVLQVS | |
| | AAPPGPRGRQAPTSPPRMTVHEGQELALGCLARTSTQKHTHLAVSF | |
| | GRSVPEAPVGRSTLQEVVGIRSDLAVEAGAPYAERLAAGELRLGKE | |
| | GTDRYRMVVGGAQAGDAGTYHCTAAEWIQDPDGSWAQIAEKRAV | |
| | LAHVDVQTLSSQLAVTVGPGERRIGPGEPLELLCNVSGALPPAGRHA | |
| | AYSVGWEMAPAGAPGPGRLVAQLDTEGVGSLGPGYEGRHIAMEKV | |
| | ASRTYRLRLEAARPGDAGTYRCLAKAYVRGSGTRLREAASARSRPL | |
| | PVHVREEGVVLEAVAWLAGGTVYRGETASLLCNISVRGGPPGLRLA | |
| | ASWWVERPEDGELSSVPAQLVGGVGQDGVAELGVRPGGGPVSVEL | |
| | VGPRSHRLRLHSLGPEDEGVYHCAPSAWVQHADYSWYQAGSARSG | |
| | PVTVYPYMHALDTLFVPLLVGTGVALVTGATVLGTITCCFMKRLRK | |
| | R | |
| 188 | MWWRLWWLLLLLLLLWPMVWAHGPGAATGVGGGLVEPGGSLRL | Fusion protein produced from |
| | SCAASGFKSTDYYMAWVRQAPGRGLEWVSFISGRVFTNYTASVRG | Vector 179 |
| | RFTVFREDDNTSVYLQMSRLRVEDTAVYYCARLRGIFRGPLKPLEY | Artificial Sequence |
| | YFDLWGRGTLVTVSSESGGGGSGGGGSGGGGSELTQPHSVSESPCK | |
| | TVTISCTRSSGSIASNYVQWYQQRPGSSPTTVIYEDNQRPSGVPDRFS | |
| | GSIDSSSNSASLTISGLKTEDEADYYCQSYDSNSGGRVFGGGTKLTV | |
| | LGAAAGGGSDYKDHDGDYKDHDIDYKDDDDKgnstmGSGGGGGSG | |
| | GGGSREVLVPEGPLYRVAGTAVSISCNVTGYEGPAQQNFEWFLYRP | |
| | EAPDTALGIVSTKDTQFSYAVFKSRVVAGEVQVQRLQGDAVVLKIA | |
| | RLQAQDAGIYECHTPSTDTRYLGSYSGKVELRVLPDVLQVSAAPPG | |
| | PRGRQAPTSPPRMTVHEGQELALGCLARTSTQKHTHLAVSFGRSVP | |
| | EAPVGRSTLQEVVGIRSDLAVEAGAPYAERLAAGELRLGKEGTDRY | |
| | RMVVGGAQAGDAGTYHCTAAEWIQDPDGSWAQIAEKRAVLAHVD | |
| | VQTLSSQLAVTVGPGERRIGPGEPLELLCNVSGALPPAGRHAAYSVG | |
| | WEMAPAGAPGPGRLVAQLDTEGVGSLGPGYEGRHIAMEKVASRTY | |
| | RLRLEAARPGDAGTYRCLAKAYVRGSGTRLREAASARSRPLPVHVR | |
| | EEGVVLEAVAWLAGGTVYRGETASLLCNISVRGGPPGLRLAASWW | |
| | VERPEDGELSSVPAQLVGGVGQDGVAELGVRPGGGPVSVELVGPRS | |
| | HRLRLHSLGPEDEGVYHCAPSAWVQHADYSWYQAGSARSGPVTV | |
| | YPYMHALDTLFVPLLVGTGVALVTGATVLGTITCCFMKRLRKR | |
| 189 | MWWRLWWLLLLLLLLWPMVWAHGPGAATGVGGGLVEPGGSLRL | Fusion protein produced from |
| | SCAASGFKSTDYYMAWVRQAPGRGLEWVSFISGRVFTNYTASVRG | Vector 180 |
| | RFTVFREDDNTSVYLQMSRLRVEDTAVYYCARLRGIFRGPLKPLEY | Artificial Sequence |
| | YFDLWGRGTLVTVSSESGGGGSGGGGSGGGGSGGGGSELTQPHSVS | |
| | ESPGKTVTISCTRSSGSIASNYVQWYQQRPGSSPTTVIYEDNQRPSGV | |
| | PDRFSGSIDSSSNSASLTISGLKTEDEADYYCQSYDSNSGGRVFGGGT | |
| | KLTVLGAAAGGGSDYKDHDGDYKDHDIDYKDDDDKgnstmGSGGG | |
| | GGSGGGGSREVLVPEGPLYRVAGTAVSISCNVTGYEGPAQQNFEWF | |
| | LYRPEAPDTALGIVSTKDTQFSYAVFKSRVVAGEVQVQRLQGDAVV | |
| | LKIARLQAQDAGIYECHTPSTDTRYLGSYSGKVELRVLPDVLQVSA | |
| | APPGPRGRQAPTSPPRMTVHEGQELALGCLARTSTQKHTHLAVSFG | |
| | RSVPEAPVGRSTLQEVVGIRSDLAVEAGAPYAERLAAGELRLGKEG | |
| | TDRYRMVVGGAQAGDAGTYHCTAAEWIQDPDGSWAQIAEKRAVL | |
| | AHVDVQTLSSQLAVTVGPGERRIGPGEPLELLCNVSGALPPAGRHA | |
| | AYSVGWEMAPAGAPGPGRLVAQLDTEGVGSLGPGYEGRHIAMEKV | |
| | ASRTYRLRLEAARPGDAGTYRCLAKAYVRGSGTRLREAASARSRPL | |
| | PVHVREEGVVLEAVAWLAGGTVYRGETASLLCNISVRGGPPGLRLA | |
| | ASWWVERPEDGELSSVPAQLVGGVGQDGVAELGVRPGGGPVSVEL | |
| | VGPRSHRLRLHSLGPEDEGVYHCAPSAWVQHADYSWYQAGSARSG | |
| | PVTVYPYMHALDTLFVPLLVGTGVALVTGATVLGTITCCFMKRLRK | |
| | R | |
| 190 | MWWRLWWLLLLLLLLWPMVWAHGPGAATGVGGGLVEPGGSLRL | Fusion protein produced from |
| | SCAASGFKSTDYYMAWVRQAPGRGLEWVSFISGRVFTNYTASVRG | Vector 181 |
| | RFTVFREDDNTSVYLQMSRLRVEDTAVYYCARLRGIFRGPLKPLEY | Artificial Sequence |
| | YFDLWGRGTLVTVSSESGGGGSGGGGSGGGGSGGGGSELTQPHSVS | |
| | ESPGKTVTISCTRSSGSIASNYVQWYQQRPGSSPTTVIYEDNQRPSGV | |
| | PDRFSGSIDSSSNSASLTISGLKTEDEADYYCQSYDSNSGGRVFGGGT | |
| | KLTVLGAAAGGGSDYKDHDGDYKDHDIDYKDDDDKgnstmGSGGG | |
| | GGSGGGGSLELNLTDSENATCLYAKWQMNFTVRYETTNKTYKTVT | |
| | ISDHGTVTYNGSICGDDQNGPKIAVQFGPGFSWIANFTKAASTYSIDS | |
| | VSFSYNTGDNTTFPDAEDKGILTVDELLAIRIPLNDLFRCNSLSTLEK | |
| | NDVVQHYWDVLVQAFVQNGTVSTNEFLCDKDKTSTVAPTIHTTVP | |
| | SPTTTPTPKEKPEAGTYSVNNGNDTCLLATMGLQLNITQDKVASVIN | |
| | INPNTTHSTGSCRSHTALLRLNSSTIKYLDFVFAVKNENRFYLKEVNI | |
| | SMYLVNGSVFSIANNNLSYWDAPLGSSYMCNKEQTVSVSGAFQINT | |
| | FDLRVQPFNVTQGKYSTAQECSLDDDTILIPIIVGAGLSGLIIVIVIASS | |
| | HWCCKKEVQETRRERRRLMSMEMD | |
| 191 | MDPKGSLSWRILLFLSLAFELSYG | signal sequence |
| | | Artificial Sequence |
| 192 | MASASGAMAKHEQILVLDPPTDLKFKGPFTDVVTTNLKLRNPSDRK | VAPA protein |
| | VCFKVKTTAPRRYCVRPNSGIIDPGSTVTVSVMLQPFDYDPNEKSKH | Artificial Sequence |
| | KFMV | |
| | QTIFAPPNTSDMEAVWKEAKPDELMDSKLRCVFEMPNENDKLGITP | |
| | PGNAPTVTSMSSINNTVATPASYHTKDDPRGLSVLKQEKQKNDMEP | |
| | SKAVP | |
| | LNASKQDGPMPKPHSVSLNDTETRKLMEECKRLQGEMMKLSEENR | |
| | HLRDEGLRLRKVAHSDKPGSTSTASFRDNVTSP | |
| 193 | GSGGSGGSGGSGGTGGSGGSGGSG | Linker |
| | | Artificial Sequence |
| 194 | MVSKGEEDNMASLPATHELHIFGSINGVDFDMVGQGTGNPNDGYE | Neon green fluorescent protein |
| | ELNLKSTKGDLQFSPWILVPHIGYGFHQYLPYPDGMSPFQAAMVDG | for visualization. |
| | SGYQVH | Artificial Sequence |
| | RTMQFEDGASLTVNYRYTYEGSHIKGEAQVKGTGFPADGPVMTNS | |
| | LTAADWCRSKKTYPNDKTIISTFKWSYTTGNGKRYRSTARTTYTFA | |
| | KPMAAN | |
| | YLKNQPMYVFRKTELKHSKTELNFKEWQKAFTDVMGMDELYK | |
| 195 | GGGSGGGS | Linker |
| | | Artificial Sequence |
| 196 | MWVTGVVTQGASRLASHEYLKAFKVAYSLNGHEFDFIHDVNKKHK | C1C2 domain |
| | EFVGNWNKNAVHVNLFETPVEAQYVRLYPTSCHTACTLRFELLGCE | Artificial Sequence |
| | LNGCAN | |
| | PLGLKNNSIPDKQITASSSYKTWGLHLFSWNPSYARLDKQGNFNAW | |
| | VAGSYGNDQWLQVDLGSSKEVTGIITQGARNFGSVQFVASYKVAY | |
| | SNDSAN | |
| | WTEYQDPRTGSSKIFPGNWDNHSHKKNLFETPILARYVRILPVAWH | |
| | NRIALRLELLGC | |
| 197 | MDPKGSLSWRILLFLSLAFELSYG | VAPA_NG_C1C2 construct |
| | MASASGAMAKHEQILVLDPPTDLKFKGPFTDVVYTTNLKLRNPSDRK | Artificial sequence |
| | VCFKVKTTAPRRYCVRPNSGIIDPGSTVTVSVMLQPFDYDPNEKSKH | |
| | KFMV | |
| | QTIFAPPNTSDMEAVWKEAKPDELMDSKLRCVFEMPNENDKLGITP | |
| | PGNAPTVTSMSSINNTVATPASYHTKDDPRGLSVLKQEKQKNDMEP | |
| | SKAVP | |
| | LNASKQDGPMPKPHSVSLNDTETRKLMEECKRLQGEMMKLSEENR | |
| | HLRDEGLRLRKVAHSDKPGSTSTASFRDNVTSP | |
| | GSGGSGGSGGSGGTGGSGGSGGSG | |
| | MVSKGEEDNMASLPATHELHIFGSINGVDFDMVGQGTGNPNDGYE | |
| | ELNLKSTKGDLQFSPWILVPHIGYGFHQYLPYPDGMSPFQAAMVDG | |
| | SGYQVH | |
| | RTMQFEDGASLTVNYRYTYEGSHIKGEAQVKGTGFPADGPVMTNS | |
| | LTAADWCRSKKTYPNDKTIISTFKWSYTTGNGKRYRSTARTTYTFA | |
| | KPMAAN | |
| | YLKNQPMYVFRKTELKHSKTELNFKEWQKAFTDVMGMDELYK | |
| | GGGSGGGS | |
| | MWVTGVVTQGASRLASHEYLKAFKVAYSLNGHEFDFIHDVNKKHK | |
| | EFVGNWNKNAVHVNLFETPVEAQYVRLYPTSCHTACTLRFELLGCE | |
| | LNGCAN | |
| | PLGLKNNSIPDKQITASSSYKTWGLHLFSWNPSYARLDKQGNFNAW | |
| | VAGSYGNDQWLQVDLGSSKEVTGIITQGARNFGSVQFVASYKVAY | |
| | SNDSAN | |
| | WTEYQDPRTGSSKIFPGNWDNHSHKKNLFETPILARYVRILPVAWH | |
| | NRIALRLELLGC | |
| 198 | LELNLTDSENATCLYAKWQMNFTVRYETTNKTYKTVTISDHGTVT | Surface domain of fusion |
| | YNGSICGDDQNGPKIAVQFGPGFSWIANFTKAASTYSIDSVSFSYNT | protein produced from Vectors |
| | GDNTTFPDAEDKGILTVDELLAIRIPLNDLFRCNSLSTLEKNDVVQH | 91, 135, 140, 141,142, 143, |
| | YWDVLVQAFVQNGTVSTNEFLCDKDKTSTVAPTIHTTVPSPTTTPTP | 144, and 145 |
| | KEKPEAGTYSVNNGNDTCLLATMGLQLNITQDKVASVININPNTTH | Artificial Sequence |
| | STGSCRSHTALLRLNSSTIKYLDFVFAVKNENRFYLKEVNISMYLVN | |
| | GSVFSIANNNLSYWDAPLGSSYMCNKEQTVSVSGAFQINTFDLRVQ | |
| | PFNVTQGKYSTAQECSLDDDT | |
| 199 | ILIPIIVGAGLSGLIIVIVIA | Transmembrane domain of |
| | | fusion protein produced from |
| | | Vectors 91, 135, 140, 141,142, |
| | | 143, 144, and 145 |
| | | Artificial Sequence |
| 200 | YVIGRRKSYAGYQTL | Cytosolic domain of fusion |
| | | protein produced from Vector |
| | | 91 |
| | | Artificial Sequence |
| 201 | ARVNKHKPWLEPTYHGIVTENDNTVLLDPPLIALDKDAPLRFAESFE | Surface domain of fusion |
| | VTVTKEGEICGFKINGQNVPFDAVVVDKSTGEGVIRSKEKLDCELQK | protein produced from Vector |
| | DYSFTIQAYDCGKGPDGTNVKKSHKATVHIQVNDVNEYAPVFKEKS | 112 |
| | YKATVIEGKQYDSILRVEAVDADCSPQFSQICSYEIITPDVPFTVDKD | Artificial Sequence |
| | GYIKNTEKLNYGKEHQYKLTVTAYDCGKKRATEDVLVKISIKPTCT | |
| | PGWQGWNNRIEYEPGTGALAVFPNIHLETCDEPVASVQATVELETS | |
| | HIGKGCDRDTYSEKSLHRLCGAAAGTAELLPSPSGSLNWTMGLPTD | |
| | NGHDSDQVFEFNGTQAVRIPDGVVSVSPKEPFTISVWMRHGPFGRK | |
| | KETILCSSDKTDMNRHHYSLYVHGCRLIFLFRQDPSEEKKYRPAEFH | |
| | WKLNQVCDEEWHHYVLNVEFPSVTLYVDGTSHEPFSVTEDYPLHPS | |
| | KIETQLVVGACWQEFSGVENDNETEPVTVASAGGDLHMTQFFRGN | |
| | LAGLTLRSGKLADKKVIDCLYTCKEGLDLQVLEDSGRGVQIQAHPS | |
| | QLVLTLEGEDLGELDKAMQHISYLNSRQFPTPGIRRLKITSTIKCFNE | |
| | ATCISVPPVDGYVMVLQPEEPKISLSGVHHFARAASEFESSEGVFLFP | |
| | ELRIISTITREVEPEGDGAEDPTVQESLVSEEIVHDLDTCEVTVEGEEL | |
| | NHEQESLEVDMARLQQKGIEVSSSELGMTFTGVDTMASYEEVLHLL | |
| | RYRNWHARSLLDRKFKLICSELNGRYISNEFKVEVNVIHTANPMEH | |
| | AAAAAAQPQFVHPEHRSFVDLSGHNLANPHPFAVVPST | |
| 202 | ATVVIVVCVSFLVFMIILGVF | Transmembrane domain of |
| | | fusion protein produced from |
| | | Vector 112 |
| | | Artificial Sequence |
| 203 | RIRAAHRRTMRDQDTGKENEMDWDDSALTITVNPMETYEDQHSSE | Cytosolic domain of fusion |
| | EEEEEEEEEESEDGEEEDDITSAESESSEEEEGEQGDPQNATRQQQLE | protein produced from Vector |
| | WDDSTLSY | 112 |
| | | Artificial Sequence |
| 204 | SSHWCCKKEVQETRRERRRLMSMEMD | Cytosolic domain of fusion |
| | | protein produced from Vector |
| | | 135 |
| | | Artificial Sequence |
| 205 | KCGFFKRARTRALYEAKRQKAEMKSQPSETERLTDDY | Cytosolic domain of fusion |
| | | protein produced from Vector |
| | | 140 |
| | | Artificial Sequence |
| 206 | VMQRLFPRIPHMKDPIGDSFQNDKLVVWEAGKAGLEECLVTEVQV | Cytosolic domain of fusion |
| | VQKT | protein produced from Vector |
| | | 141 |
| | | Artificial Sequence |
| 207 | RLSRKGHMYPVRNYSPTEMVCISSLLPDGGEGPSATANGGLSKAKS | Cytosolic domain of fusion |
| | PGLTPEPREDREGDDLTLHSFLP | protein produced from Vector |
| | | 142 |
| | | Artificial Sequence |
| 208 | LLMIIHDRREFAKFEKEKMNAKWDTGENPIYKSAVITVVNPKYEGK | Cytosolic domain of fusion |
| | | protein produced from Vector |
| | | 143 |
| | | Artificial Sequence |
| 209 | RIRAAHRRTMRDQDTGKENEMDWDDSALTITVNPMETYEDQHSSE | Cytosolic domain of fusion |
| | EEEEEEEEEESEDGEEEDDITSAESESSEEEEGEQGDPQNATRQQQLE | protein produced from Vector |
| | WDDSTLSY | 144 |
| | | Artificial Sequence |
| 210 | KKPR | Cytosolic domain of fusion |
| | | protein produced from Vector |
| | | 145 |
| | | Artificial Sequence |
| 211 | TIIRPPMWSPVWP | Affinity Peptide of Vector 112 |
| | | Artificial Sequence |
| 212 | THVSPNQGGLPS | Affinity Peptide of Vector 135, |
| | | 140-145 |
| | | Artificial Sequence |

¹ENSP00000###### for protein sequence, Transcript ID ENST00000###### and Gene ID ENSG00000###### refer to sequence annotation in Ensembl (EMBI-EBI; Cunningham, F. et al. (2019) Ensembl 2019 Nucleic Acids Res. 47 (D1): D745-D751; Zerbino, D.R. et al. (2018) Ensembl 2018 Nucleic Acids Res. 46 (D1): D754-D761; Aken, B.L. et al. (2017) Ensembl 2017. Nucleic Acids Res. 45 (D1): D635-D642; Aken, B.L. etal. (2016) The Ensembl gene annotation system. Database 2016: baw093) based on assembled sequence in Genome Reference Consortium Human Build 38 patch release 12 (GRCh38.p12; GenBank assembly accession GCA_000001405.27 and Ref-Seq assembly accession GCF_000001405.38).

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: Preparation of Vesicles with Markers

A cassette is made for cloning nucleic acids encoding one or more targeting moieties of interest. The cassette includes a polynucleotide encoding the 3′-C1C2 localization domain for display on exosomes (XStamp, System Biosciences), a polynucleotide encoding a 5′-signal sequence to target the polypeptide marker to the secretion machinery in a cell, and optionally, a polynucleotide encoding a linker peptide between the marker and the C1C2 localization domain. A polynucleotide encoding a skeletal muscle targeting moiety of interest is cloned into the cassette such that it is operably linked to the signal sequence, linker, and C1C2 localization domain.

A cell line such as HEK293, PER.C6, CHO-K1 or Hs 235.Sk (ATCC® CRL-7201™ is transfected with vectors including the cassette with a desired marker. Positive transfectants are obtained by flow cytometry or other cell sorting methods. In other cases, positive transfectants are enriched through antibiotic selection. Transfected cells are grown in exosome depleted or chemically defined media, suitable for exosome isolation. Following a period of culture, EVs are isolated from the conditioned media.

Any cells in the conditioned media are cleared by centrifugation and filtration, and the EVs in the clarified media are concentrated using ultrafiltration. After concentration the exosomes are isolated using liquid chromatography using an appropriate column (e.g., Sephacryl S-300, Capto-Core 700, etc.)

### Example 2: Protocol for Labeling EVs Chemically with Fluorescent Dye

Bodipy Labeling of EVs:
1. Bodipy-TR Ceramide preparation. Resuspend lyophilized BODIPY-TR Ceramide (250 ug, 705.7085 Daltons) in 354.2539 uL DMSO to a final stock concentration of 1 mM.
2. Bodipy labeling:
   a. Add PBS to EV isolated to bring final volume of each sample up to 1 mL.
   b. Add 20 μL of the stock Bodipy solution (1 mM) to 1 mL EV sample and mix. The final dye concentration in the EV sample is 20 μM.
   c. Incubate at 37° C. for 1 hr (protected from light).
3. Free Bodipy clean up from the EV sample:
   a. At the end of incubation, filter the BodipyEV through a 0.22 um 33 mm PES PBS pre-cleaned Milex filter.
   b. Pipet BODIPY-EV solution into pre-cleaned (1× sterile PBS wash) Amicon 4 100 kDa.
   c. Perform 3× buffer exchange (3000 g for 7 minutes). Caution: Do not overspin because this will dry the membrane and products, causing additional loss.
   d. Add sterile PBS to the collection chamber to bring up volume to 0.5 mL.
   e. Use P200 to gently wash product off membrane pipet out the solution.
   f. Filter the BodiyEV sample through a 0.22 um 33 mm PES Millex filter.
4. BodipyEV characterization:
   a. Analyze the efficiency of EV labeling via absorption spectra measurement
   b. Analyze the particle concentration via NTA measurement
   c. Store the EVs in the dark at 4° C.

As an alternative to the labeling of EVs by chemical dyes, EVs may be labeled through the use of a fluorescent protein fusion, such as green fluorescent protein (GFP) and its variants, or protein reporters. This alternative method often involves creation of fusion proteins to generate a vesicle targeting moiety-protein reporter gene constructs and cellular expression of these fusion proteins to obtain exosomes.

### Example 3: Protocol for Fluorescent-Based Analysis of EV Uptake by Skeletal Muscle Cell

Cell-based in vitro uptake assay: A skeletal muscle cell line labeled with a fluorescent dye and containing a skeletal muscle target protein and a negative cell line not containing the skeletal muscle cell target are co-cultured. Cell viability is confirmed to be >95% after 24 hours, and confluency between 40-90%, to confirm that both cell types in co-culture are representative of their functional capabilities in standalone monoculture. The co-culture is then “dosed” with EVs for an indicated period. The EVs have been engineered with a targeting motif that targets the nicotinic acetylcholine receptor found in skeletal muscle; this receptor is only present on the skeletal muscle cell line, but not the negative cell line. Cell uptake is assessed by labeling the EVs before dosing with a fluorescent dye, and then measuring fluorescence via flow cytometry, which also simultaneously permits distinguishing the labeled skeletal muscle cell from the negative cell line.

1. Preparation of Cell Coculture (24 Hrs Before the Assay):
a. CellTracker Violet BMOC preparation:
   i. Thaw CellTracker Violet BMQC dye at room temperature 10 mins before use.
   ii. Add 59 uL DMSO to achieve 5 mM stock concentration, vortex and spin.
   iii. Add 40 uL CellTracker Violet BMQC to 40 mL complete media (DMEM high glucose+10% FBS) to make 5 uM working concentration.
b. Cell labeling for coculture system (label only one cell line in a 2 cell line coculture system):
   i. Remove existing cell media for the Hs 235.Sk (ATCC® CRL-7201™) or HskMC (ATCC® PCS-950-010™) cells and add 20 mL CellTracker Violet BMQC containing media to each 1 T-175. Incubate at 37° C. for 45 mins.
c. Set up coculture system:
   i. Harvest both CellTracker Violet BMQC labeled cells (Hs 235.Sk (ATCC® CRL-7201™, or HskMC (ATCC® PCS-950-010™) and unlabeled cells (HEK293) with Trypsin-EDTA. Caution: Add excess coculture media (DMEM low glucose+10% FBS) and pipet aggressively to achieve single cell suspension.
   ii. Count and measure viability of cells using trypan blue stain under the cell counter.
   iii. Plate cells at the desired concentration (3.4E5 cells in monoculture, 1.7E5 cells of each cell line in coculture) in each well of a 6-well plate. Each sample will have 2 replicates' in coculture and 1 replicate for monoculture.
   iv. Allow the coculture to grow overnight before the experiment.

2. Incubation of Bodipy-Labeled EV with Cell Coculture:
a. Prepare 1 mL of BodipyEV formulations in appropriate media (DMEM low glucose+10% FBS) to have a working concentrations (1.02E9 particles/mL) per well.
b. Remove culture media and add 1 mL BodipyEV/media to each well of the 6-well plate.
c. Incubate for the desired amount of time (i.e., 1 hr and 2 hr in this experiment)
d. Harvest the cells with Trypsin EDTA.
e. Transfer the cells to microcentrifuge tubes. Spin cells at 3000 rpm for 7 mins to aspirate media.
f. Wash the cells with 1 mL ice cold PBS. Spin cells at 3000 rpm for 5 mins to remove PBS.
g. Re-suspend the cells in 200 uL PBS. The cells are now ready for flow cytometry analysis.

### Example 4: Vesicle Delivery In Vitro to Skeletal Muscle Cells

EVs are obtained from the conditioned media supernatant of cultured HEK293 cells. The EVs are isolated using ultracentrifugation (size selection to enrich for a general EV population). The EVs are loaded with a reporter (e.g., CPSD) or mRNA encoding a reporter (e.g., GFP).

Skeletal muscle cell line such as Hs 235.Sk (ATCC® CRL-7201™) is grown to confluence and then the EVs with reporter are added to the skeletal muscle cell line. After incubating the skeletal muscle Hs235.Sk cells with the EVs, the excess EVs are washed away. The cells are then subjected to fluorescence microscopy to identify those cells that have obtained a reporter from the EVs. EV delivery to the cells is identified by reporter activity in cells.

### Example 5: Vesicle Delivery In Vivo to Skeletal Muscle Cells

EVs are obtained from the media of Hs 235.Sk (ATCC® CRL-7201™). The EVs are isolated using ultracentrifugation (size selection to enrich for a general EV population). The EVs are loaded with a reporter (e.g., CPSD) or mRNA encoding a reporter (e.g., GFP). The animal model B6.129X1-Nfe212^{tm1Ywk} mice are used for this study.

B6.129X1-Nfe212^{tm1Ywk} mice are injected with EVs containing the reporter CPSD. After 24 hours, the mice are sacrificed and the animal's skeletal myocytes are examined with fluorescence microscopy. EV delivery to skeletal muscle tissue is identified by reporter activity in the skeletal muscle cells.

### Example 6: Functional Assay Determining Expression of VAPA on Engineered Exosomal Surface

Exosomes presenting targeting moieties of interest are engineered as described in herein. The isolated exosomes are validated for presentation of VAPA or a marker of interest using nanoparticle tracking analysis.

NTA measurements are obtained with a NanoSight NS300 instrument equipped with the NTA 3.3 analytical software. Samples are diluted to achieve a particle count in the linear range of the instrument: between 20 and 150 particles on the screen at one time. Samples are loaded using the NanoSight Sample Assistant to automate the measurement of up to 96 samples in one run. Multiple 30 second videos of each sample flowing at a slow constant flow are obtained. These measurements are then analyzed using the batch process function.

### Example 7: Introducing Payloads into Engineered Exosomes Carrying Markers of Interest

An exosome is engineered to express a binding partner of a skeletal muscle marker as described herein [See Table 1], such as a subunit or multiple subunits of the nicotinic acetylcholine receptor found in skeletal muscle. Alternatively, an exosome is engineered to express any one of the following markers ENO2, JSRP1, VAPA, TMOD1 or peptides described herein [See Table 1]. The engineered or isolated exosome is loaded with fenretinide. The loaded exosome is then used to reduce obesity in a subject.

Exosome protein input of −300 μg (from about 1×10\hat{ }7 exosomes) is suspended in 50 μμl of sterile PBS. A reaction mixture consisting of exosomes, 10 μl of Exo-Fect Reagent and nucleic acid of interest (20 pmol si/miRNA, 1 ug mRNA or 5 ug plasmid DNA) is put together and mixed by inversion. The transfection solution is incubated in a shaker for 10 minutes at 37C and then placed on ice. To stop the reaction, 30 μl of ExoQuick-TC reagent provided in the kit is added to the exosome sample suspension and mixed by inverting. The transfected exosome sample is placed on ice for 30 minutes. The sample is centrifuged at 13000-14000 rpm to pellet the exosomes. The transfected exosomes are then resuspended in PBS and can be added to target cells or used in vivo for further applications.

### Example 8: Construction of Chimeric Vesicle Targeting Moiety

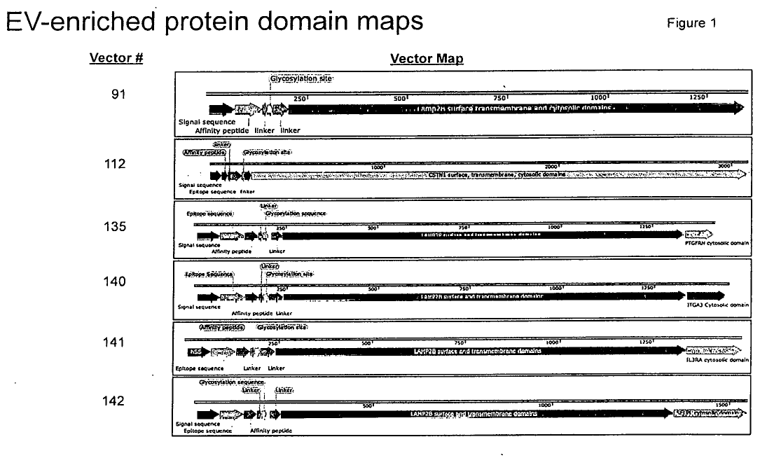

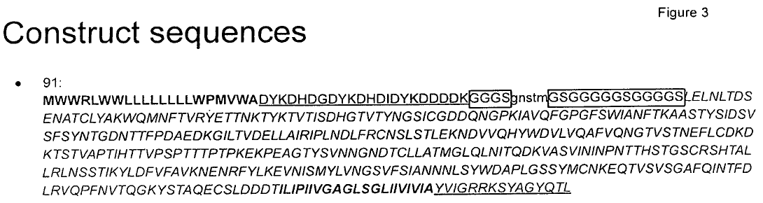

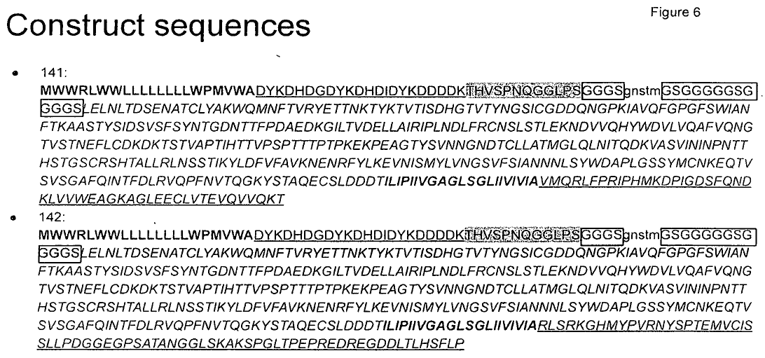

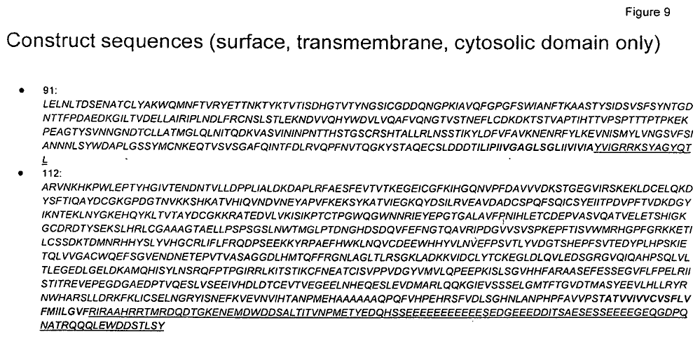

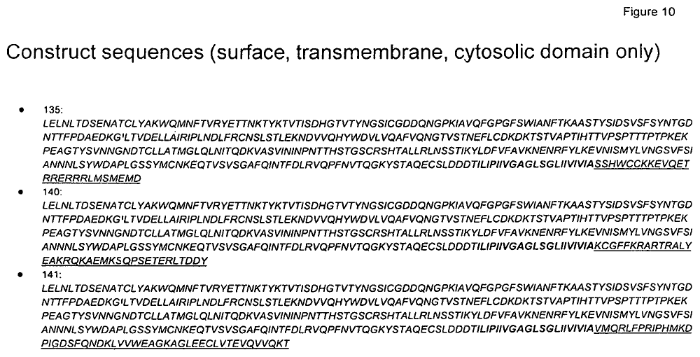

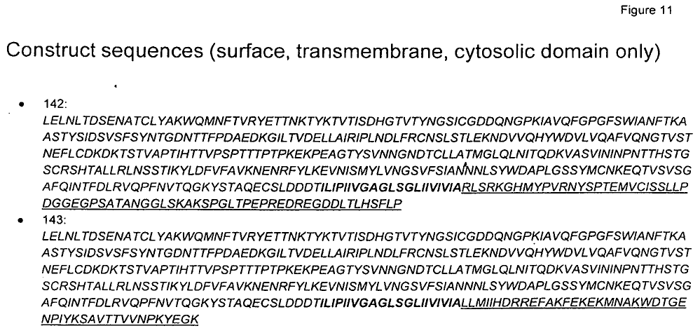

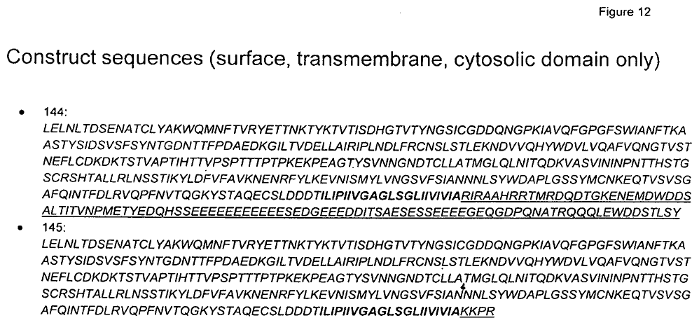

To improve performance of vesicle targeting moiety with respect to extracellular vesicle localization, chimeric vesicle targeting moieties were constructed as schematically presented in Figures (slide 1 and 2). The targeting moiety binds to a subunit or multiple subunits of the nicotinic acetylcholine receptor, which is found in skeletal muscle. Vector #91 construct when introduced into HEK293F cells produces a fusion protein comprising from amino-to-carboxyl terminus in the order: a signal sequence (for improved expression and endoplasmic reticulum association)-glycosylation site (for stabilization of fusion protein)-full length LAMP2B (Lysosome-associated membrane protein 2) protein with surface, transmembrane and cytosolic domains (for localization to exosomes). The full length LAMP2B protein used lacks its natural signal sequence—the first 28 amino acids found at N-terminal of the LAMP2B protein. However, the fusion protein additional comprises peptide linkers. Such peptide linkers rich in glycine and serine amino acids may be found between the signal sequence, glycosylation site and LAMP2B protein. In addition, epitope sequence (such as that corresponding to 3×FLAG epitope tag) and affinity peptide sequence may be found in between the signal sequence and the glycosylation site. Examples of suitable affinity peptides include, but are not limited to, THRPPMWSPVWP (SEQ ID NO.: 211), and THVSPNQGGLPS (SEQ ID NO.: 212). This full length LAMP2B fusion protein serves as one parental vesicle targeting moiety (see FIG. 1, vector #91 for a schematic; see FIG. 3, vector #91 for the sequence of the parental LAMP2B fusion protein produced and FIG. 9, vector #91 for the sequence of the full length LAMP2B with the surface, transmembrane and cytosolic domain which lacks the first 28 amino acids corresponding to its signal sequence). In FIGS. 9-12, amino acid sequences are extracted from the sequences provided in FIGS. 3-8 so that only the vesicle targeting moiety amino acid sequences are shown. The surface domain (italic text) precedes the transmembrane domain (italic and bold) which is found between the surface and cytosolic domains (italic and underline).

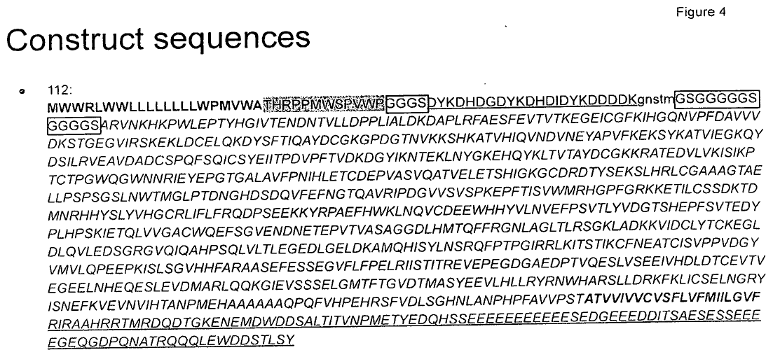

A second parental vesicle targeting moiety was constructed with full length CSTN1 coding sequence lacking the first 28 codons encoding its natural signal sequence, as schematically shown in FIG. 1 by the vector #112 construct. The full length CSTN1 protein (minus its normal signal sequence) has the surface, transmembrane and cytosolic domains. Like the full length LAMP2B protein produced from the expression of vector #91 in mammalian cells (HEK293F), the full length CSTN1 fusion protein has a similar arrangement of the same signal sequence at the amino terminus of the fusion protein along with epitope sequence and glycosylation site. Linkers are similarly present and in addition an affinity peptide is present in the CSTN1 fusion protein (see FIG. 1, vector #112 for vector map over the coding sequences; FIG. 4 for the sequence of the parental CSTN1 fusion protein produced from vector #112 and FIG. 9 for the sequence of the full length CSTN1 protein without the first 28 amino acid corresponding to its signal sequence).

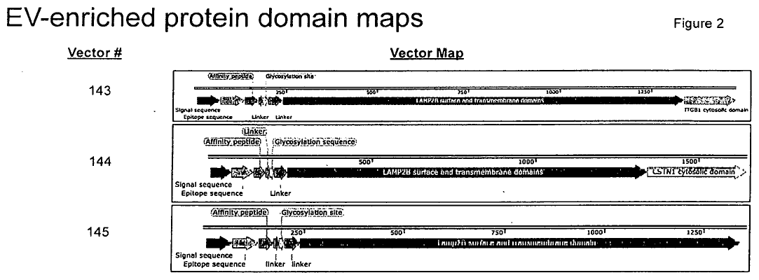

Chimeric vesicle targeting moiety were prepared primarily based on the surface domain and transmembrane domain of LAMP2B (surface-and-transmembrane domain of LAMP2B) and cytosolic domain from other transmembrane proteins. In particular, the cytosolic domain of LAMP2B is replaced with the cytosolic domain of PTGFRN (vector #135), ITGA3 (vector #140), IL3RA (vector #141), SELPL (vector #142), ITGB1 (vector #143) and CSTN1 (vector #144), as schematically represented in FIGS. 1 and 2. Amino acid sequence of the fusion proteins produced comprising the chimeric vesicle targeting moieties are shown in FIG. 3-8. The sequences are shown in capital letter and bold for signal sequence, capital letter and underline for epitope sequence, shaded capital letter for affinity peptide, open boxed capital letter for peptide linker sequence, small letter for glycosylation site, capital letter and italic for surface domain, capital letter and bold italic for transmembrane domain and capital letter and underlined italic for cytosolic domain in FIGS. 3-8.

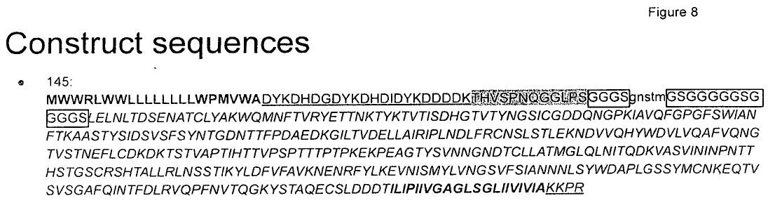

To assess the effect of LAMP2B cytosolic domain on the ability of LAMP2B protein to localize at extracellular vesicles, sequences encoding the cytosolic domain of LAMP2B was excluded in the construction of an expression vector to produce a fusion protein comprising a truncated LAMP2B protein, where the fusion protein contains the LAMP2B surface and transmembrane domains but lacks its natural signal sequence (first 28 amino acids at the amino terminus of LAMP2B protein) and its C-terminal cytosolic domain (see FIG. 2, vector #145 for a map showing schematics of the coding region for the fusion protein and FIG. 8, #145 for the amino acid sequence of the fusion protein of the truncated LAMP2B lacking a cytosolic domain).

### Example 9: Extracellular Vesicle (EV) Production and Isolation

HEK293F cells were maintained in serum-free media suspension cultures in shake flasks. Upon reaching a culture density of 2×10⁶ cells/mL, each shake flask culture was transfected with individual plasmids corresponding to vector constructs provided in FIGS. 1-2 to produce the fusion proteins with the amino acid sequence provided in FIGS. 3-8, using PEI (MW: 25,000-1 mg/mL). 24 hours after transfection, the transfection media was exchanged for fresh media, and the cells were grown for an additional 96 hours. 96 hours following media exchange, the cultures were transferred into 50 mL conical tubes and centrifuged at 3,220×g for 30 min. The supernatant from these cultures were transferred to Amicon Centrifugal Filter Units (100 Kda cutoff), concentrated and buffer exchanged into PBS. EVs are then filtered using CaptoCore700 Size Exclusion Chromatography (SEC) resin to remove non-EV-associated protein. Finally, the EVs are sterile filtered, using a 0.22 μM centrifugal filter column unit.

### Example 10: Recombinant Protein Detection on the EV Surface

To ensure that EVs are displaying the construct encoded by the transfected plasmid, and that it is oriented in the correct transmembrane topology, isolated EVs are stained with Flag antibody and a membrane stain. The stained vesicles are evaluated using vesicle flow cytometry (Cytoflex—Beckman Coulter). EVs are identified as membrane stain-positive particles. The amount of recombinant protein on each EV is detected using an fluorophore-conjugated antibody that binds specifically to the epitope sequence included in the primary sequence of the protein, and would only be available on the EV surface if the protein were oriented in the intended topology (C-terminal domain in the lumen; N-terminal domain on the EV surface). The amount of recombinant protein on each evaluated EV is determined by the antibody signal/membrane stained particle.

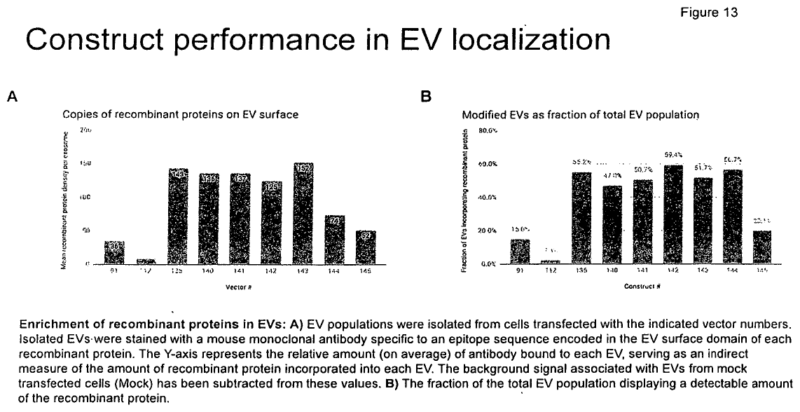

In FIG. 13A, EV populations were isolated from cells transfected with the indicated vector numbers. Isolated EVs were stained with a mouse monoclonal antibody specific to an epitope sequence encoded in the EV surface domain of each recombinant protein. The Y-axis represents the relative amount (on average) of antibody bound to each EV, serving as an indirect measure of the amount of recombinant protein incorporated into each EV. The background signal associated with EVs from mock transfected cells (Mock) has been subtracted from these values. The fraction of the total EV population displaying a detectable amount of the recombinant protein is shown in FIG. 13B.

### Example 11: Chimeric Vesicle Targeting Moiety with a Surface-and-Transmembrane Domain of First Vesicle Targeting Moiety and a Cytosolic Domain can Increase EV Localization

Transient transfection of the different expression vector constructs for the production of the fusion proteins shown in FIGS. 1 and 2 and having the sequence provided in FIG. 3-8 and analysis of the fusion protein localization at EVs isolated from culture media showed surprising alteration in the efficiency of accumulation of the fusion protein at an extracellular vesicle. Producing a fusion protein comprising a chimeric vesicle targeting moiety having a LAMP2B surface-and-transmembrane domain and a non-native cytosolic domain from a number of different vesicle targeting moieties PTGFRN (vector #135), ITGA3 (vector #140), IL3RA (vector #141), SELPL (vector #142), ITGB1 (vector #143) and CSTN1 (vector #144) showed dramatic improvement in the ability of the fusion protein to localize at an extracellular vesicle, increasing both the density or concentration of the fusion protein at an EV (FIG. 13A) as well as the fraction or percent of EVs positive for the fusion protein (FIG. 13B). In every case, replacement of the cytoplasmic domain of one vesicle targeting moiety, LAMP2B protein, with that of a second vesicle targeting moiety, PTGFRN (vector #135), ITGA3 (vector #140), IL3RA (vector #141), SELPL (vector #142), ITGB1 (vector #143) and CSTN1 (vector #144), resulted in both an increase in the density of fusion protein present on EV surface (FIG. 13A) and an increase in the fraction or percent of total EV population positive for the fusion protein (FIG. 13B).

FIGS. 13A and B show that not only do chimeric vesicle targeting moieties localize to EVs but localization of the fusion proteins is improved when a chimeric vesicle targeting moiety is used in place of its non-chimeric counterpart (compare #135, 140, 141, 142, 143 and 144 with #91 or 112). Furthermore, while deleting the cytosolic domain of LAMP2B modestly improves localization of LAMP2B surface-and-transmembrane domain to EV (compare #145 with #91), the improvement in EV localization by transplanted cytosolic domains from a variety of vesicle targeting moiety is much more significant—indicating that while the cytosolic domain may not be required for EV localization of a surface-transmembrane domain of a vesicle targeting moiety (such as LAMP2B), the cytosolic domain can modulate EV localization, affecting the efficiency of EV localization.

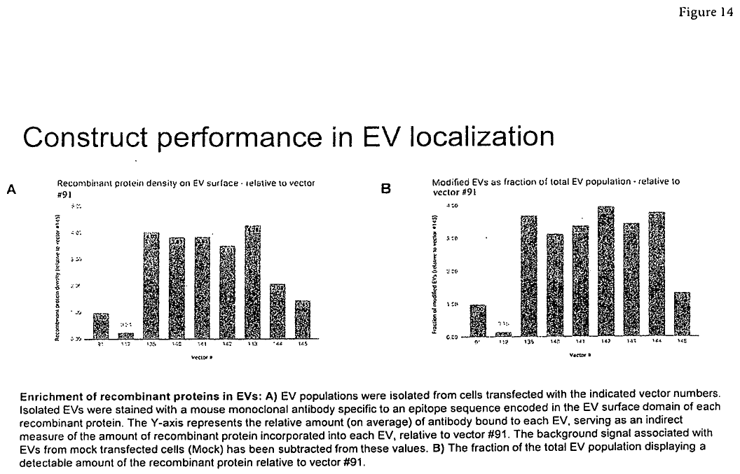

Normalization of the data in FIG. 13A to illustrate fold increase in fusion protein density or concentration on EV surface relative to the fusion protein comprising a mature full length LAMP2B (processed full length LAMP2B lacking its native signal sequence, the first 28 amino acids at the amino terminus of the nascent protein; vector #91 construct) is shown in FIG. 14A. Similarly, normalization of the data in FIG. 13B to illustrate fold increase in percent or fraction of EVs positive for the fusion protein relative to the fusion protein comprising a mature LAMP2B protein produced by vector #145 construct is shown in FIG. 14B. Replacing the cytosolic domain of the mature LAMP2B protein with the cytosolic domain of a variety of other vesicle targeting moiety results in about a 4-fold increase in fusion protein density at an EV for a number of cytosolic domain examined obtained from PTGFRN (vector #135), ITGA3 (vector #140), IL3RA (vector #141), SELPL (vector #142), and ITGB1 (vector #143), as seen in FIG. 14A. Similar to this increase in the concentration of the fusion protein at an EV, fraction of total EVs positive for the various fusion proteins with a chimeric vesicle targeting moiety (vector #135, 140, 141, 142, 143, and 144) increases 3-4 fold over the fusion protein comprising a non-chimeric vesicle targeting moiety, namely the parental LAMP2B vesicle targeting moiety (vector #91) which provided its LAMP2B surface-and-transmembrane domain to the various chimeric vesicle targeting moieties (vector #135, 140, 141, 142, 143, and 144).

### Example 12: Chimeric Vesicle Targeting Moiety can Dramatically Improve EV Localization Over Parental Vesicle Targeting Moieties

FIG. 14A shows fold increase in fusion protein density (or concentration) on EV surface relative to fusion protein produced by vector #91 construct (fusion protein with a mature LAMP2B protein having a contiguous surface-transmembrane-and-cytosolic domain but no LAMP2B signal sequence). Compared to the fusion protein produced by vector #91, the fusion protein produced by vector #112 (fusion protein with a mature CSTN1 protein having its surface-transmembrane-and-cytosolic domain but no CSTN1 signal sequence) concentrates at a much lower level, about 25% the density of the mature LAMP2B-containing fusion protein (compare value of #91 and #112 in FIG. 14A). Surprisingly, when the cytosolic domain of the mature LAMP2B is replaced with the cytosolic domain of the mature CSTN1, the new chimeric vesicle targeting moiety increases by about 2-fold the density of the fusion protein over its parental LAMP2B (compare value of #91 and #144) or over 8-fold the density of the fusion protein over its parental CSTN1 (compare value of #112 and #144), indicative of synergistic interaction between the surface-and-transmembrane domain of LAMP2B and the cytosolic domain of CSTN1.

Synergistic interaction leading to the increased concentration of the fusion protein at an EV is also observed when analyzing fraction of total EV population positive for a fusion protein (FIG. 14B). In particular, fusion protein comprising the parental LAMP2B vesicle targeting moiety is better at associating with total EV population having a normalized value of 1.00 (#91) than the fusion protein comprising the parental CSTN1 vesicle targeting moiety with a normalized value 0.15 (#112). In contrast, a fusion protein comprising a chimeric vesicle domain produced from the two parental vesicle targeting moieties (#144) has a normalized value of 3.79, reflecting over 3.5-fold increase over the parental LAMP2B vesicle targeting moiety and over 25-fold over the parental CSTN1 vesicle targeting moiety. Such a dramatic increase in association with total EV population which reaches about 55% (see FIG. 14, #144) by a fusion protein comprising a chimeric vesicle targeting moiety is unexpected. The observed increase in EV localization is not unique to the use of CSTN1 cytosolic domain to replace the LAMP2B cytosolic domain. A number of other cytosolic domains also increase EV localization beyond that of the parental LAMP2B vesicle targeting moiety, indicating that the cytosolic domain of PTGFRN, ITGA3, IL3RA, SELPL, and ITGB1 may function in a similar manner as the cytosolic domain of CSTN1 to synergistically increase EV localization, both concentrating at a single EV as well as associating with the total EV population.

Thus, analyses of fusion protein density (or concentration) on an EV surface and fraction (or percent) of total EV population positive for fusion protein showed that in a chimeric vesicle targeting moiety comprising a surface-and-transmembrane domain of a first vesicle targeting moiety and a cytosolic domain of a second vesicle targeting moiety can interact synergistically to increase accumulation at an extracellular vesicle. Such a finding provides an approach not only to improve EV localization but potentially to change the composition of EVs as the chimeric vesicle targeting moiety may interact with a different set of proteins or has altered affinity to the set of protein recruited to an extracellular vesicle by the two native vesicle targeting moieties.

### Example 13: Fusion Proteins Comprising a Skeletal Muscle Cell Targeting Moiety (ScFv) and a Vesicle Targeting Moiety for Production of Extracellular Vesicles

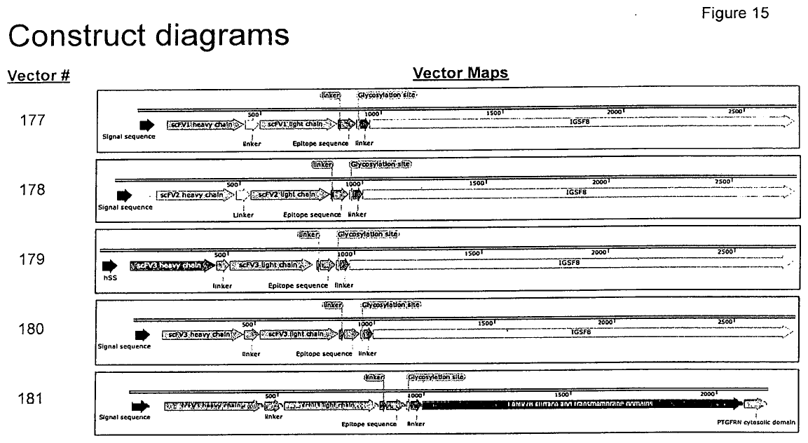

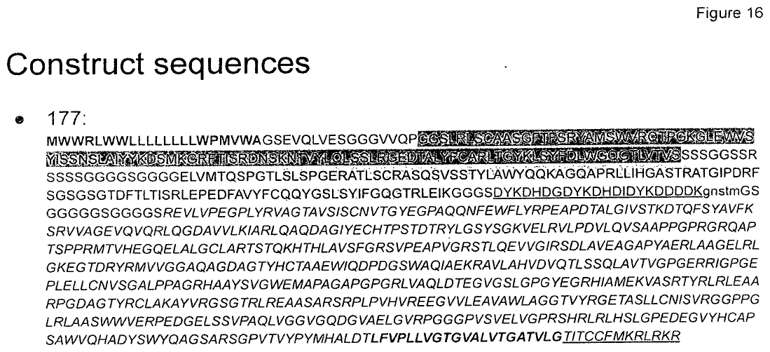

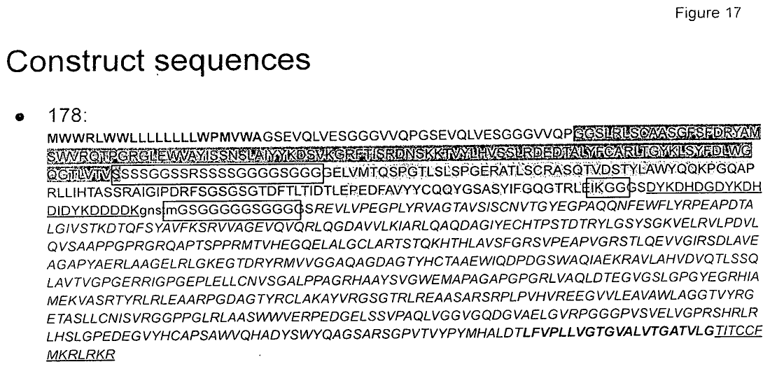

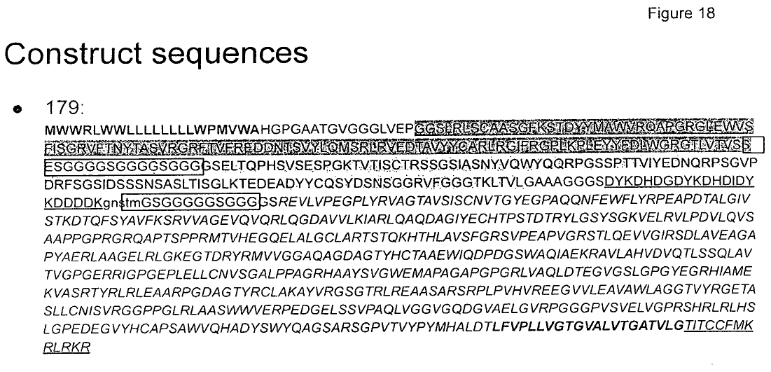

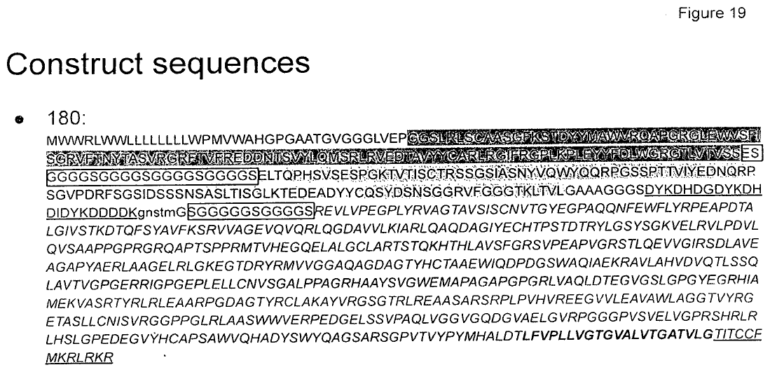

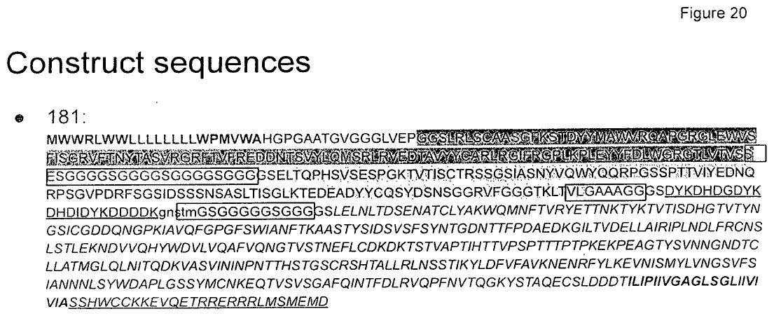

FIG. 15 provides maps of fusion proteins produced by vector #177-181 comprising in the order from a signal sequence, a skeletal muscle cell targeting moiety (scFv), and either a vesicle targeting moiety (IGSF8, Immunoglobulin Superfamily Member 8) or a chimeric vesicle targeting moiety (a truncated LAMP2B with contiguous surface-and-transmembrane domain coupled at its C-terminus to a PTGFRN cytosolic domain). In addition, a glycosylation site is present between the scFv skeletal muscle cell targeting moiety that binds to a subunit, multiple subunits, or the entire nicotinic acetylcholine receptor found in skeletal muscle and the vesicle targeting moiety. Expression of the fusion constructs in HEK293F cells results in the localization of the fusion proteins targeting skeletal muscle cells into EVs. Amino acid sequence corresponding to the fusion protein used to produce exosomes are provided in FIGS. 16-20.

### Example 14: Sandwich ELISA of VAPA-Tagged EVs Formed from a Fusion Protein Comprising VAPA, Neon GFP and a Vesicle Targeting Moiety (C1C2 Domain of Lactadherin)

An expression vector for the production of a fusion protein having the sequence as provided in SEQ ID NO: 197 and comprising a signal sequence, VAPA (VAMP-A Associated Protein A) without its transmembrane domain, neon green fluorescent protein (mNeonGreen) and C1C2 domain of lactadherin was expressed in a HEK293 (FreeStyle™ 293-F) cells. Conditioned media collected from these transfected cells (Harvest) were analyzed as well as EVs isolated from the conditioned media (Isolate), flow throughs after EV isolation (Flow-through) and extract prepared from cell pellet lysed by two freeze-thaw cycles (Cells) for the presence of the fusion protein by a custom developed sandwich ELISA with colorimetric readout using anti-VAPA antibodies. Matched pair of anti-VAPA antibodies were used to capture the purified protein standard or intact vesicles. Capture antibody immunogen is a synthetic peptide corresponding to region between amino acids 125 to 175 of human VAPA. Primary antibody immunogen is a recombinant protein fragment produced in E. coli corresponding to amino acids 2 to 227 of human VAPA. As a positive control, purified recombinant protein with N-terminal His-tag and corresponding to the amino acids 1-227 of human VAPA were similarly analyzed. As a negative control, conditioned media was prepared from HEK293 cells transfected with an expression vector for the production of a fusion protein comprising neon GFP and C1C2 domain of lactadherin but no VAPA as well as conditioned media produced by untransfected HEK293 cells.

### Results of ELISA Plate

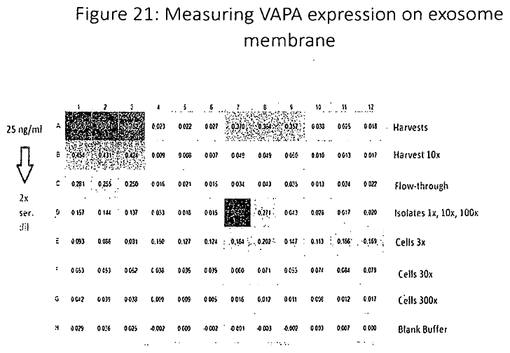

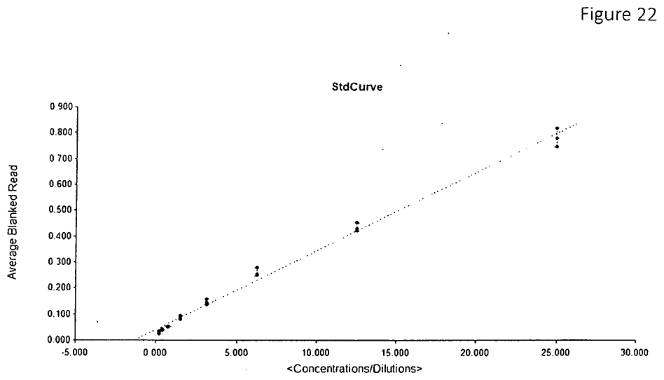

FIG. 21 shows results of the ELISA plate with the reading of the plate columns as follow: Columns 1-3: standard (purified recombinant protein in blocking buffer); Columns 4-6: NG_C1C2—neon green protein with C1C2 domain transfected into HEK293 control; columns 7-9: VAPA_NG_C1C2: VAPA construct without its transmembrane domain, neon green protein with C1C2 domain transfected into HEK293 (engineered EV protein). Columns 10-12: Standard HEK293 cells and isolates with no transfection. 0.271 OD=7.552 ng/mL based off of standard curve (1.088 out of range); and the standard curve for the ELISA is shown in FIG. 22 and is based on columns 1-3 of FIG. 21.

Thus, from the antibody capture sandwich ELISA analysis transfection of expression vector construct1 (VAPA_NG_C1C2: a VAPA construct (without the transmembrane domain) fused with neon green fluorescent protein and C1C2 domain of lacdtadherin) results in a significant increase in the amount of EVs displaying VAPA. Furthermore, the EV isolation procedure is effective at removing VAPA displaying EVs from conditioned media as reflected by a 10-fold drop in the ELISA signal in the flow through compared to the condition media and as also reflected by the strong signals of the undiluted and 10× diluted EVs isolated fraction.

All publications, gene transcript identifiers, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A vesicle that selectively targets a skeletal muscle cell or tissue comprising one or more skeletal muscle targeting moieties comprising:
(a) a binding partner of a skeletal muscle marker, wherein the skeletal muscle marker is selected from the group consisting of: ART1, CACNA2D1, CHRNA1, CHRNB1, CHRND, CHRNE, CHRNG, and FGF6 or a homologue and fragment thereof; and
(b) a chimeric vesicle targeting moiety comprising a surface-and-transmembrane domain of a first vesicle targeting moiety and a cytosolic domain of a second vesicle targeting moiety.

2-3. (canceled)

4. The vesicle of claim 1, wherein the first and second vesicle targeting moieties are distinct proteins and not isoforms.

5. The vesicle of claim 1, wherein the first vesicle targeting moiety is selected from a group consisting of SELPL, IL3RA, LIRB4, FPRP, IGSF8, ITA3, CSTN1, ITA5, ITGB1, ADAM10, CD81, CD9, CD63, FASN, sodium/potassium-transporting ATPase subunit alpha-1, sodium/potassium-transporting ATPase subunit beta-3, and FN1.

6. The vesicle of claim 1, wherein the second vesicle targeting moiety is selected from a group consisting of SELPL, IL3RA, LIRB4, FPRP, IGSF8, ITA3, CSTN1, ITA5, ITGB1, ADAM10, CD81, CD9, CD63, FASN, sodium/potassium-transporting ATPase subunit alpha-1, sodium/potassium-transporting ATPase subunit beta-3, and FN1.

7. The vesicle of claim 1, wherein the binding partner is attached to said chimeric vesicle-targeting moiety.

8. The vesicle of claim 7, wherein the binding partner is attached to said chimeric vesicle-targeting moiety through an hydrophobic group.

9. The vesicle of claim 8, wherein the attachment of a hydrophobic group is myristoylation for attachment of myristate, palmitoylation for attachment of palmitate, prenylation for attachment of a prenyl group, farnesylation for attachment of a farnesyl group, geranylgeranylation for attachment of a geranylgeranyl group or glycosylphosphatidylinositol (GPI) anchor formation for attachment of a glycosylphosphatidylinositol comprising a phosphoethanolamine linker, glycan core and phospholipid tail.

10. The vesicle of claim 8, wherein the attachment of a hydrophobic group is performed by chemical synthesis in vitro or is performed enzymatically in a post-translational modification reaction.

11.-15. (canceled)

16. The vesicle of claim 1, wherein the surface-and-transmembrane domain of the first vesicle targeting moiety is a surface-and-transmembrane domain of LAMP2B.

17. The vesicle of claim 1, wherein the cytosolic domain of the second vesicle targeting moiety is the cytosolic domain selected from the group consisting of PTGFRN, ITGA3, IL3RA, SELPL, ITGB1, CSTN1, IGSF8 and PDGFR or a homologue thereof.

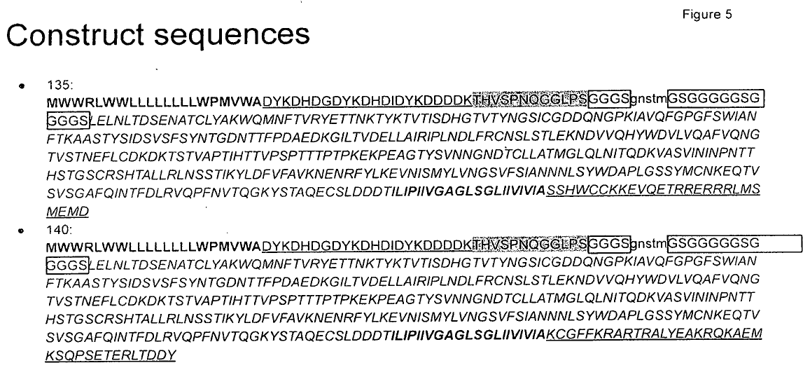

18. The vesicle of claim 17, wherein the cytosolic domain of PTGFRN has an amino acid sequence as provided in FIG. 5, 10 or 20 or a homologue or portion thereof, wherein the homologue or portion retains at least 80% of cytosolic domain activity of FIG. 5, 10 or 20 in accumulating at an extracellular vesicle, wherein accumulating at an extracellular vesicle is assessed on the basis of the percent of extracellular vesicle positive for the chimeric vesicle targeting moiety, total number of extracellular vesicle positive for the targeting moiety, and/or the concentration of targeting moiety in an extracellular vesicle.

19.-23. (canceled)

24. The vesicle of claim 3, wherein the chimeric vesicle targeting moiety is incorporated into an extracellular vesicle.

25. The vesicle of claim 1, wherein the extracellular vesicle is an exosome.

26. The vesicle of claim 25, wherein the exosome has an average diameter in the range of about 50 nm-200 nm.

27. The vesicle of claim 24, wherein greater than 40% of the extracellular vesicles are positive for the chimeric protein.

28. The vesicle of claim 24, wherein greater than 50% of the extracellular vesicles are positive for the chimeric protein.

29. The vesicle of claim 24, wherein greater than 60% of the extracellular vesicles are positive for the chimeric protein.

30.-63. (canceled)

64. The vesicle of claim 1, wherein the one or more skeletal muscle targeting moieties is an antibody or a fragment or variant thereof, a peptide, an aptamer, a ligand, or protein or protein fragment.

65.-94. (canceled)

95. A fusion protein comprising: a vesicle targeting moiety coupled to one or more skeletal muscle targeting moieties comprising:
A. a binding partner of skeletal muscle marker which is selected from the group consisting of: ART1, CACNA1C, CACNA1D, CACNA1F, CACNA1S, CACNA2D1, CHRNA1, CHRNB1, CHRND, CHRNE, CHRNG, and FGF6 or a homologue or fragment thereof; and
B. a chimeric vesicle targeting moiety comprising a surface-and-transmembrane domain of a first vesicle targeting moiety and a cytosolic domain of a second vesicle targeting moiety.

96.-133. (canceled)

134. A kit comprising a vesicle that selectively targets a skeletal muscle cell or tissue and instructions for use and/or storage, the vesicle comprising one or more skeletal muscle targeting moieties comprising: a binding partner of a skeletal muscle marker, wherein the skeletal muscle marker is selected from the group consisting of: ART1, CACNA2D1, CHRNA1, CHRNB1, CHRND, CHRNE, CHRNG, and FGF6 or a homologue and fragment thereof; and a chimeric vesicle targeting moiety comprising a surface-and-transmembrane domain of a first vesicle targeting moiety and a cytosolic domain of a second vesicle targeting moiety.

135.-138. (canceled)
